(19) United States
(12) Patent Application Publication
(10) Pub. No.: US 2023/0348854 A1
(43) Pub. Date: Nov. 2, 2023

(54) CHIMERIC ANTIGEN RECEPTORS (CARS) TARGETING NATURAL KILLER CELLS

(71) Applicant: Seattle Children's Hospital d/b/a Seattle Children's Research Institute, Seattle, WA (US)

(72) Inventors: Thor Wagner, Seattle, WA (US); Floriane Ngako Kameni, Seattle, WA (US)

(73) Assignee: Seattle Children's Hospital d/b/a Seattle Children's Research Institute, Seattle, WA (US)

(21) Appl. No.: 17/998,206

(22) PCT Filed: May 7, 2021
(86) PCT No.: PCT/US2021/031421

§ 371 Date: Nov. 8, 2022

Related U.S. Application Data
(60) Provisional application No. 63/022,149, filed on May 8, 2020.

(51) Int. Cl. C12N 5/0783; C07K 14/725; A61P 35/00; C12N 15/86; C07K 16/28

(52) U.S. Cl. CPC C12N 5/0636; C07K 14/7051; A61P 35/00; C12N 15/86; C07K 16/2896; C07K 2317/565; C07K 2319/03; C07K 2317/622; C07K 2317/53

(57) ABSTRACT

The current disclosure provides chimeric antigen receptors (CARs) that bind a natural killer (NK) cell surface marker, resulting in destruction of the bound NK cell. The NK cell surface markers include an activating NK cell receptor, and an inhibitory NK cell receptor. Cells that are genetically modified to express these CARs and uses of the CAR modified cells are also described.

### CROSS-REFERENCE TO RELATED APPLICATION

This application is a U.S. National Phase Application based on International Patent Application No. PCT/US2021/031421, filed on May 7, 2021, which claims priority to U.S. Provisional Patent Application No. 63/022,149, filed on May 8, 2020, each of which is incorporated herein by reference in its entirety as if fully set forth herein.

### STATEMENT REGARDING SEQUENCE LISTING

The Sequence Listing associated with this application is provided in text format in lieu of a paper copy and is hereby incorporated by reference into the specification. The name of the text file containing the Sequence Listing is 2SF5626_ST25.txt. The text file is 273 KB, was created on Oct. 28, 2022, and is being submitted electronically via Patent Center.

### FIELD OF THE DISCLOSURE

The current disclosure provides chimeric antigen receptors (CARs) that bind natural killer (NK) cell surface markers resulting in destruction of the bound NK cell. The NK cell surface markers include an activating NK cell receptor, and an inhibitory NK cell receptor. Cells that are genetically modified to express these CARs and uses of the CAR modified cells are also described.

### BACKGROUND OF THE DISCLOSURE

Natural killer (NK) cells are a type of white blood cell that play a crucial role in immune system responses to tumor cells and virally infected cells. NK cells are activated in response to inflammatory mediators such as interferons or cytokines. NK cells also express a variety of activating and inhibitory receptors, as well as co-stimulatory receptors, to recognize and respond to inflamed or infected tissues. These receptors bind cellular stress ligands, which can lead to NK cell responses. Importantly, some of these receptors prevent activation of NK cells, so that NK cells do not target healthy tissue.

Major histocompatibility complex class I (MHC I) and related molecules are part of the biology that allows NK cells to distinguish healthy cells from stressed, infected, or neoplastic cells. MHC refers to a complex encoded by multiple genes and multiple variants of these genes that allow an organism to immunologically recognize ‘healthy self cells’ (e.g., non-virally infected cells and non-cancerous cells of the organism) from ‘unhealthy or non-self cells’ (e.g., virally infected cells and cancerous cells of the organism or exogenous cells from a pathogen). The molecules encoded by the MHC also determine an organism's overall immune response to parts of proteins, toxins, and/or foreign substances presented to immune cells by the MHC molecules.

NK cells carry out their cytotoxic functions on target cells with the release of molecules from the NK cell interior, such as perforin and granzymes. When an NK cell contacts a cell targeted for destruction, perforin released from the NK cell forms pores in the cell membrane of the target cell through which the granzymes and associated molecules can enter, inducing cell death of the target cell.

While normally beneficial, NK cells can be associated with NK cell-based cancers. NK cell neoplasms, including NK/T cell lymphoma and aggressive NK cell leukemia, are rare malignancies. The incidence is 0.8 per 1,000,000 in the U.S. and this incidence is 3 to 10 times higher in South America and Asia. High mortality is observed, with a 17- to 20-month overall median survival, but the outcomes become much worse for relapsed/refractory NK/T lymphomas and aggressive NK cell leukemias. These malignancies are usually associated with Epstein-Barr Virus (EBV) infection. The malignancies can further be divided into nasal, non-nasal, and aggressive lymphoma/leukemia subtypes. Most nasal NK cell lymphomas present with Stage I/II disease. Stage I/II disease can be treated with radiotherapy; however, concomitant chemotherapy may be necessary if radiotherapy fails or if the malignancy has advanced beyond Stage I/II. High-dose chemotherapy combined with hematopoietic stem cell transplantation may be beneficial for some patients.

NK cells can also have an inhibitory phenotype, which may limit the immune response to some malignancies and infections in some situations. An NK cell inhibitory phenotype can include: more expression of inhibitory receptors on an NK cell; inhibition of NK cell activity; a decrease in the ability of an NK cell to lyse a target cell; and/or a decrease in NK cell immune response against inflamed, infected, tumor, and/or other cells that need to be destroyed, as compared to a normal functioning NK cell or an NK cell without an inhibitory phenotype. Tumor cells can take advantage of this inhibitory phenotype, for example, by overexpressing a ligand (human leukocyte antigen E, HLA-E) that binds an inhibitory receptor, NGK2A, on NK cells and thus block tumor cell killing by the NK cells. HLA-E expression on tumor cells is associated with poor prognosis.

Some non-NK cell malignancies aberrantly express NK cell receptors. For example, patients who have Sezary syndrome, an aggressive form of cutaneous T-cell lymphoma, express the NKp46 NK receptor on the cell surface of their peripheral blood malignant CD4+ T lymphocytes (Bensussan et al. (2011) J Invest Dermatol. 131: 969-976). As another example, it has been observed that 20% of mature T cell neoplasms, including T cell large granular lymphocytic leukemia, mycosis fungoides, and ALK+ anaplastic large cell lymphoma, aberrantly express NKp46 receptor (Freud et al. (2013) Am J Clin Pathol. 140:853-866).

Changes in circulating or tissue NK cells can also be associated with NK mediated auto- and alloimmunity, where healthy self or non-self tissues are attacked by a person's immune system. Autoimmune disorders linked to NK cell function include: Sjogren's disease; antiphospholipid syndrome; Pemphigus vulgaris; spondylarthropathies; skin diseases including psoriasis; multiple sclerosis; systemic sclerosis; Type I diabetes; juvenile idiopathic arthritis; rheumatoid arthritis; inflammatory bowel disease; autoimmune liver diseases; and systemic lupus erythematosus (SLE). Alloimmune disorders linked to NK cell function include: fetal/neonatal alloimmune thrombocytopenia (FNAIT) where maternal immune responses to fetal platelet antigens can lead to complications such as miscarriage and intrauterine growth restriction; hematopoietic stem cell rejection; solid tissue transplant rejection; and chronic allograft injury after organ transplantation.

The main treatments for auto- and alloimmunity include modifying a subject's immune system to ameliorate inflammation and control the overactive immune response. Examples include immune-suppressing drugs, steroids, nonsteroidal anti-inflammatory drugs, plasmapheresis, immunoadsorption, B cell reduction, and splenectomy. Medications that treat symptoms such as pain, swelling, fatigue, and skin rashes are also often used.

Given the role of NK cells in a variety of pathological conditions, there is a need to develop therapies where elimination or reduction of NK cells would be of interest, such as in NK cell malignancies, non-NK malignancies that express NK cell receptors, conditions in which NK cell inhibition prevents an effective immune response and where there could be benefit to increasing NK cell function, and in NK cell-mediated autoimmune or alloimmune disorders.

### SUMMARY OF THE DISCLOSURE

The current disclosure provides chimeric antigen receptors (CARs) including antigen binding domains that bind natural killer (NK) cell surface markers. The NK cell surface markers include an activating NK cell receptor, an inhibitory NK cell receptor, or both. In particular embodiments, an activating NK cell receptor includes NKp30, Nkp44, and NKp46. In particular embodiments, an activating NK cell receptor includes NKp46. In particular embodiments, an inhibitory NK cell receptor includes NKG2A and killer immunoglobulin receptor (KIR). In particular embodiments, an inhibitory NK cell receptor includes NKG2A. In particular embodiments, an NKp46 binding domain includes a single chain variable fragment (scFv) derived from an NKp46 antibody. In particular embodiments, an NKG2A binding domain includes an artificial HLA-E mimetic. Immune cells can be genetically modified to express CARs to target NK cells. In particular embodiments, the genetically modified immune cells are T cells, NK cells, macrophages, hematopoietic stem cells (HSC), or a hematopoietic progenitor cells (HPC).

Immune cells modified to express anti-NK CARs can be used to deplete NK cells expressing an activating NK receptor and/or an inhibitory NK receptor in vivo. In particular embodiments, an activating NK cell receptor includes NKp30, Nkp44, and NKp46. In particular embodiments, an activating NK cell receptor includes NKp46. In particular embodiments, an inhibitory NK cell receptor includes NKG2A and KIR. In particular embodiments, an inhibitory NK cell receptor includes NKG2A. In particular embodiments, the anti-NK CAR modified cells can be used to treat NK cell malignancies. In particular embodiments, the anti-NK CAR modified cells can be used to treat NK cell malignancies in combination with anti-viral treatments. Particular embodiments also provide for treating conditions in which NK cell inhibition prevents an effective immune response. In particular embodiments, a non-NK cell-based cancer (e.g., breast cancer) is treated by depleting NK cells expressing an inhibitory NK receptor. In particular embodiments, the inhibitory NK cell receptor includes NKG2A.

Particular embodiments also provide for treating NK cell mediated autoimmune or alloimmune disorders with anti-NK CAR modified cells. In particular embodiments, an NK cell mediated autoimmune or alloimmune disorder is treated by depleting NK cells expressing an activating NK receptor. In particular embodiments, an activating NK cell receptor includes NKp46.

In particular embodiments, anti-NK CAR modified cells co-express a CAR including a binding domain that binds an activating receptor on NK cells and a CAR including a binding domain that binds an inhibitory receptor on NK cells to prevent or ameliorate killing of the anti-NK CAR modified cells by target NK cells. In particular embodiments, anti-NK CAR modified cells express a CAR including an NKp46 binding domain and a CAR including an NKG2A binding domain. In particular embodiments, an NKp46 binding domain includes a single chain variable fragment (scFv) derived from an NKp46 antibody. In particular embodiments, an NKG2A binding domain includes an artificial HLA-E mimetic. In particular embodiments, an NKG2A binding domain includes an scFv derived from an anti-NGK2A antibody.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. Natural killer (NK) cell neoplasms classifications. Adapted from Kwong, Y. L. (2005) Nature Leukemia, 19(12) 2186-2194.

FIGS. 2A-2C. Anti-natural killer (NK) cell chimeric antigen receptor (CAR) constructs and CAR expression. (FIG. 2A) Cartoon representation of CAR constructs at the cell surface. NKp46 is also referred to as CD335. Amino acid sequences for these CAR constructs include SEQ ID NOs: 249 (amino acid sequence of an anti-NKp46 CAR), 251 (amino acid sequence of an anti-NKG2A (HLA-E) CAR), 253 (amino acid sequence of an anti-NKG2A (HLA-E) CAR), 255 (amino acid sequence of an anti-NKG2A (HLA-E) CAR), and 257 (amino acid sequence of an anti-NKG2A (HLA-E) CAR). Nucleotide sequences encoding these CAR constructs include SEQ ID NOs: 250, 252, 254, 256, and 258. (FIG. 2B) Schematic diagram of CAR constructs in the pRRL lentiviral (LV) backbone containing the γ-retrovirus-derived promoter-enhanced MND. (FIG. 2C) Expression of the anti-NKp46 (anti-CD335) CAR construct demonstrated by flow cytometry assessing blue fluorescent protein (BFP) expression.

FIG. 3A, 3B. Summary of anti-NKp46 CAR T cell activation in the presence of media, non-specific cells (K562 cells), and target cells engineered to express either NKp46 or CD19. Anti-NKp46 CAR T cells are activated (increased CD137 expression) in the presence of cells that express NKp46 but not CD19. Whereas anti-CD19 CAR T cells are activated (increased CD137 expression) in the presence of cells that express CD19 but not NKp46. The magnitude of the CAR T cell activation was similar for the anti-NKp46 CAR and the anti-CD19 CAR. The results with donor A (FIG. 3A) and donor B (FIG. 3B) are similar.

FIGS. 4A, 4B. Summary of anti-NKp46 CAR T cell function in the presence of media, non-specific cells (K562 cells), and target cells engineered to express either NKp46 or CD19. Anti-NKp46 CAR T cells express appropriate intracellular cytokines (increased IL-2, TNF-α, and IFG-γ) in the presence of cells that express NKp46 but not CD19. Whereas anti-CD19 CAR T cells express appropriate cytokines in the presence of cells that express CD19 but not NKp46. The magnitude of intracellular cytokine expression was similar for the anti-NKp46 CAR and the anti-CD19 CAR. The results with donor A (FIG. 4A) and donor B (FIG. 4B) are similar.

FIG. 5. Killing of NKp46+ cells by anti-NKp46 CAR T cells. Flow cytometry plot of NKp46-expressing K562 cells alone (left). NKp46-expressing K562 cells mixed with anti-CD19 CAR T cells at a 2:1 ratio×24 hours before analysis by flow cytometry (center). NKp46-expressing K562 cells mixed with anti-NKp46 CAR T cells at a 2:1 ratio×24 hours before analysis by flow cytometry (right). The target cells are to the right side of the plots)(CD3^{lo}). The T cells are further to the right, the upper T cells express the CAR as assessed by BFP expression. The percentage of live target cells in the presence of CAR T cells decreases from 16.2% in the presence of the anti-CD19 CAR T cells to 4.85% in the presence of the anti-NKp46 CAR T cells. This represents a 70% reduction in NKp46+ target cells in the presence of the anti-NKp46 CAR. Target cells circled in black. T cells expressing BFP are presumptive CAR T cells, circled in black and denoted with an asterisk.

FIG. 6. Anti-NKp46 CAR T cells are activated when mixed with autologous NK cells. FIG. 6 shows upregulation of CD137 expression on anti-NKp46 CAR T cells when anti-NKp46 CAR T cells are mixed with autologous NK cells as evaluated by flow cytometry. CAR T cells are mixed with target cells at a 2:1 ratio and evaluated by flow cytometry after 6 hours. CD137 expression is measured in CAR cells by gating on single live CD3+ lymphocytes that express BFP. Target cells were autologous cells enriched for NK cells (40-50% NK cells).

FIG. 7. Anti-NKp46 CAR T cells kill autologous NK cells. Autologous NK cells were enriched and stained with cell tracker and then mixed with anti-NKp46 CAR T cells, control CAR T cells (anti-CD19 CAR T cells), or mock T cells at a 2 CAR to 1 target cell ratio for 24 hours and then evaluated by flow cytometry. The NK cells were quantified by gating on CD3 negative cell tracker orange cells. In the presence of anti-NKp46 CAR T cells the percentage of NK cells was reduced by 72% in the presence of anti-NKp46 CAR T cells versus only 9% in the presence of control CAR T cells (anti-CD19 CAR). The percentage of cells shown in indicated plots: lymphocytes=70.4 (y-axis is SSC-A); singlets (single cells)=92.2 (y-axis is FSC-H); live cells=89.4 (y-axis is SSC-A); target population=100.0 (y-axis is Comp-PE-A); and NK cells (CD3 negative)=45 (y-axis is Comp-PE-A).

FIG. 8. NK cells are activated by contact with anti-NKp46 CAR T cells. Autologous target cells enriched for NK cells were mixed with anti-NKp46 CAR T cells at a 2 CAR cell:1 target cell ratio for 24 hours and assessed for intracellular cytokine expression by flow cytometry. Gating on NK cells demonstrates that cytokine expression increases when the NK cells are mixed with anti-NKp46 CAR T cells but does not increase when NK cells are mixed with mock T cells or anti-CD19 CAR T cells. The percentage of NK cells expressing IFN-γ or TNF-α is increased 2-3 fold, whereas IL-2 doesn't seem to increase. IFN-γ or TNF-α are cytokines typically produced by activated NK cells.

FIG. 9. Autologous NK cells kill anti-NKp46 CAR T cells. Anti-NKp46 or anti-CD19 CAR T cells were mixed with autologous NK cells and assessed by flow cytometry at 6 hours. The autologous NK cells were loaded with cell tracker to help differentiate the target cells. The CAR T cells were evaluated by gating on cell tracker negative cells (non-target), CD3+ (non-NK cells), and BFP (CAR+ cells). Plots show side scatter (SSC) versus BFP. The percentage of BFP+ anti-NKp46 cells decreased 35% (10.2% to 6.6%) in the presence of autologous NK cells (upper panels), whereas the percentage of BFP+ anti-CD19 CAR T cells didn't decrease in the presence of autologous NK cells (lower panels). The decrease in anti-NKp46 CAR T cells seems to be most pronounced in cells that express the most BFP.

FIGS. 10A-10C. Anti-NK CAR design and construction for CARs described in Example 2. (FIG. 10A) Diagrams of the extracellular and transmembrane domains of the anti-NKp46 and αNKG2A CARs. In the depicted embodiments, while the αNKp46 CAR is anchored in the membrane by the CD8 alpha chain hinge and transmembrane domain, the αNKG2A CAR uses the HLA-E transmembrane and intracellular domain. TM, transmembrane; scFv, single-chain variable region specific to the NKp46 receptor protein; b2M, beta-2-microglobulin. (FIG. 10B) Schematic representation of the complete αNKp46 and αNKG2A CAR constructs cloned into the pRRL backbone under the MND enhancer-promoter derived from the gamma-retrovirus. CD8a hinge & TM, CD8a hinge and transmembrane domain; 4-1BB, intracellular co-stimulatory domain; CD3z, stimulatory domain; 2A, self-cleaving peptide; BFP, blue-fluorescent protein. (FIG. 10C) Representative flow plots depicting BFP reporter expression in Mock, αNKp46 CAR, αNKG2A CAR, and αCD19 control CAR.

FIGS. 11A-11F. αNKp46 CAR T cell phenotype and cytotoxicity against NKp46⁺ K562 cells. (FIG. 11A) Histograms showing NKp46 and CD19 expression in the myelogenous cell line K562 separately transduced to express either receptor. FMO=Fluorescence Minus One control. (FIG. 11B) Summary of αNKp46 CAR T cell surface CD137 and (FIG. 11C) intracellular cytokine upregulation against NKp46⁺ K562 cells. BFP-expressing αNKp46 CAR T cells were cultured with NKp46⁺ K562 cells at a 2:1 E:T ratio for 24 hours and stained for CD137 or cultured for 6 hours and stained for TNFα, IFNγ and IL-2 expression. Data represents mean+−S.D. of three (CD137) or five (cytokines) separate experiments across three donors. Significance was calculated with a two-sided Student's t test. (FIG. 11D) Pie charts representing the cytokine expression profiles of αCD19 or αNKp46 CAR T cells cultured with NKp46+ K562 cells. Each shade represents a particular combination of cytokines expressed. Each area of the pie chart represents the proportion of CAR T cells with the corresponding cytokine expression profile. Arc length represents the frequency of expression of each measured cytokine. (FIG. 11E) Left, dose-response curves showing percentage of lysed NKp46⁺ K562 cells after 24-hour co-cultures with αNKp46 and αCD19 CAR T cells. Right, ratio of NKp46⁺ to CD19⁺ K562 cells remaining after 24-hour co-cultures with either αNKp46 or αCD19 CAR T cells at increasing effector doses. In these assays, NKp46⁺ and CD19⁺ K562 cells were plated together at equal numbers with αNKp46 or αCD19 CAR T cells at 2:1 E:T ratios. (FIG. 11F) Representative flow plots of CAR T cell specific killing of target cells. NKp46⁺ and CD19⁺ K562 cells were cultured alone, with mock, αNKp46 or αCD19 CAR T cells at 2:1 E:T ratios for 24 hours.

FIGS. 12A-12H. αNKp46, αNKG2A CAR T cell phenotype and cytotoxicity against NK cells. (FIG. 12A) Top: schematic of autologous NK cell isolation and expansion occurring concurrently with CAR T cell production. Bottom: Dot plots from one representative experiment showing NK cell purity through NK expansion protocol with NK MACS medium. (FIG. 12B) Representative histograms depicting NKp46+ of PBMCs and NKG2A expression on NK cells selected from PBMC by 14 days of growth in NK MACS media. (FIG. 12C) Representative histograms depicting NKp46+ and NKG2A expression on NK92 cells. (FIG. 12D) Percent CAR T cells expressing surface CD137 or (FIG. 12E) intracellular cytokines when cultured with an autologous primary NK/T target mix or NK92 cells after 24 hours (CD137) or 6 hours (cytokines). (FIG. 12F) Pie charts representing the cytokine expression profiles of CAR T cells cultured with autologous NK/T cell mixture or NK92 cells. Each shade represents a particular combination of cytokines expressed. Each area of the pie chart represents the proportion of CAR T cells with the corresponding cytokine expression profile. Arc length represents the frequency of expression of each measured cytokine. (FIG. 12G) Dose-response curves showing the percentage of lysed autologous NK cells after co-culture with CAR T cells for 6 hours. (FIG. 12H) Representative dot plots depicting clearance of CellTracker-stained autologous NK cells when cultured with CAR T cells.

FIG. 13A-13E. NK cell cytotoxicity and αNKp46 CAR protection. (FIG. 13A) Schematic representation of NK cell cytotoxicity against αNKp46 CAR T cells and mitigation with αNKG2A CAR co-expression. (FIG. 13B) Top, fraction of CAR T cells (% BFP T cells) remaining after 4-hour co-cultures with autologous primary NK cells. Values are normalized such that the percent of BFP+ cells in the CAR T cells only control equates to 1. Bottom, relative BFP mean fluorescence intensity (MFI) of CAR T cells after 4-hour co-cultures with autologous primary NK cells. Values are normalized such that the relative BFP MFI in the CAR T cell alone condition is 1. (FIG. 13C) Top, fraction of αNKp46 CAR T cells (% BFP T cells) remaining after 4-hour co-cultures with NKp46⁺ K562 cells. Values are normalized such that the percent of BFP+ cells in the αNKp46 CAR T cells only control equates to 1. Bottom, relative BFP MFI of αNKp46 CAR T cells after 4-hour co-cultures with NKp46⁺ K562 cells. Values are normalized such that the relative BFP MFI in the αNKp46 CAR T cell alone condition is 1. (FIG. 13D) Representative density plots showing BFP expression of CAR T cells cultured alone (top) or with autologous primary NK cells at a 2:1 E:T ratio (bottom) after 4 hours. The plots show a decrease in both % BFP expression and BFP MFI after adding NK cells with αNKp46 CAR T cells only. (FIG. 13E) Percent of autologous primary NK cells expressing intracellular cytokines against CAR T cells when cultured at 2:1 T:NK cell ratios.

FIG. 14. Exemplary sequences of the disclosure including:

| SEQ ID | |
|---|---|
| NO: | Description |
| 1 | Amino acid sequence of Human NKp46 (UniProt ID O76036) |
| 2 | Coding sequence for human NKp46 (NCBI Ref: NM_004829) |
| 120 | Coding sequence for scFv of antibody NKp46-1 |
| 121 | Coding sequence for scFv of antibody NKp46-2 |
| 122 | Coding sequence for scFv of antibody NKp46-3 |
| 123 | Coding sequence for scFv of antibody NKp46-4 |
| 124 | Coding sequence for scFv of antibody NKp46-6 |
| 125 | Coding sequence for scFv of antibody NKp46-9 |
| 126 | Coding sequence for scFv of antibody BAB281 |
| 127 | Amino acid sequence for a signal peptide of HLA-G (‘G peptide’) |
| 128 | Coding sequence of G peptide including most likely codons |
| 129 | Coding sequence of G peptide including consensus codons |
| 130 | Amino acid sequence for human beta-2-microglobulin (B2M) signal peptide |
| | (residues 1-20 of UniProt ID P61769) |
| 131 | Coding sequence of human B2M signal peptide including most likely codons |
| | based upon SEQ ID NO: 130 |
| 132 | Coding sequence of human B2M signal peptide including consensus codons |
| | based upon SEQ ID NO: 130 |
| 133 | Amino acid sequence for B2M without signal peptide (residues 21-119 of UniProt |
| | ID P61769) |
| 134 | Coding sequence of human B2M without signal sequence including most likely |
| | codons based upon SEQ ID NO: 133 |
| 135 | Coding sequence of human B2M without signal sequence including consensus |
| | codons based upon SEQ ID NO: 133 |
| 136 | Amino acid sequence of human HLA-E 01:03 heavy chain without signal peptide |
| | (residues 22-358 of UniProt ID P13747) |
| 137 | Coding sequence of human HLA-E 01:03 heavy chain without signal peptide |
| | (nucleotides 87-1097 of NCBI Ref: NM_005516.6) |
| 138 | Amino acid sequence of human CD8 alpha chain transmembrane domain |
| | (residues 183-203 of UniProt ID P01732-1) |
| 139 | Coding sequence of human CD8 alpha chain transmembrane domain (nucleotides |
| | 1578 to 1640 of NCBI Ref: NM_001145873.1) |
| 140 | Amino acid sequence of human CD8 alpha chain hinge (residues 135-182 of |
| | UniProt ID P01732-1) |
| 141 | Coding sequence of human CD8 alpha chain hinge (nucleotides 1434 to 1577 of |
| | NCBI Ref: NM_001145873.1) |
| 142-145 | Exemplary glycine-serine linker sequences |
| 146 | Amino acid sequence of cytoplasmic domain of human CD3 zeta chain (residues |
| | 52-164 of UniProt ID P20963-1) |
| 147 | Coding sequence of cytoplasmic domain of human CD3 zeta chain (nucleotides |
| | 299 to 637 of NCBI Ref: NM_198053.2) |
| 148 | Amino acid sequence of cytoplasmic domain of human 4-1BB (residues 214-255 |
| | of UniProt ID Q07011) |
| 149 | Coding sequence of cytoplasmic domain of human 4-1BB (nucleotides 901 to |
| | 1026 of NCBI Ref: NM_001561.5) |
| 150 | IgG4 linker sequence |
| 151 | CD28 linker sequence |
| 152 | His tag sequence |
| 153-155 | Flag tag sequences |
| 156 | Xpress tag sequence |
| 157 | Avi tag sequence |
| 158 | Calmodulin binding peptide (CBP) tag sequence |
| 159 | Polyglutamate tag sequence |
| 160-162 | HA tag sequences |
| 163 | Myc tag sequence |
| 164, 165 | Strep tag sequence |
| 166, 167 | Softag sequences |
| 168 | V5 tag sequence |
| 169-174 | BFP sequences |
| 175 | Nucleotide sequence of myeloproliferative sarcoma virus enhancer, negative |
| | control region deleted, dl587rev primer-binding site substituted (MND) promoter |
| 176-185 | 2A peptide sequences |
| 186 | Amino acid sequence of human NKG2A (UniProt ID P26715) |
| 187 | Coding sequence of human NKG2A (nucleotides 215-916 of NCBI Ref: |
| | NM_002259.4) |
| 188 | Amino acid sequence of cytoplasmic domain of human CD28 (residues 180-220 of |
| | UniProt ID P10747) |
| 189 | Coding sequence of cytoplasmic domain of human CD28 (nucleotides 223 to 882 |
| | of NCBI Ref: NM_006139.3) |
| 207 | Coding sequence of G peptide in anti-NKG2A (HLA-E) CAR |
| 208 | Coding sequence of human B2M signal peptide in anti-NKG2A (HLA-E) CAR |
| 209 | Coding sequence of mature human B2M without signal sequence in anti-NKG2A |
| | (HLA-E) CAR |
| 210 | Coding sequence of human HLA-E 01:03 heavy chain without signal peptide in |
| | anti-NKG2A (HLA-E) CAR |
| 211 | Coding sequence of (G₄S)₃ linker |
| 212 | Coding sequence of (G₄S)₄ linker |
| 230 | Amino acid sequence of 4-1BB/CD3z intracellular signaling domain in CAR |
| | constructs |
| 231 | Coding sequence of 4-1BB/CD3z intracellular signaling domain in CAR constructs |
| 232 | Coding sequence of CD8 hinge in CAR constructs |
| 233 | Coding sequence of CD8 transmembrane domain in CAR constructs |
| 234 | Amino acid sequence of CD8 hinge and CD8 transmembrane domain in CAR |
| | constructs |
| 235 | Coding sequence of CD8 hinge and CD8 transmembrane domain in CAR |
| | constructs of the disclosure |
| 236 | Amino acid sequence of mTagBFP (blue fluorescent protein) in CAR constructs |
| 237 | Coding sequence of mTagBFP in CAR constructs |
| 238 | Amino acid sequence of CD8 signal peptide in CAR constructs |
| 239 | Coding sequence of CD8 signal peptide in CAR constructs |
| 240 | 2A peptide sequence in CAR constructs |
| 241 | Coding sequence of exemplary 2A peptide sequence in CAR constructs |
| 242 | Amino acid sequence of an HLA-E mimetic |
| 243 | Coding sequence of HLA-E mimetic of SEQ ID NO: 242 |
| 244 | Amino acid sequence of an HLA-E mimetic |
| 245 | Coding sequence of HLA-E mimetic of SEQ ID NO: 244 |
| 246 | Amino acid sequence of an HLA-E mimetic |
| 247 | Coding sequence of HLA-E mimetic of SEQ ID NO: 246 |
| 248 | Coding sequence of anti-NKp46 scFv of SEQ ID NO: 206 |
| 249 | Amino acid sequence of an anti-NKp46 CAR |
| 250 | Coding sequence of anti-NKp46 CAR of SEQ ID NO: 249 |
| 251 | Amino acid sequence of an anti-NKG2A (HLA-E) CAR |
| 252 | Coding sequence of anti-NKG2A (HLA-E) CAR of SEQ ID NO: 251 |
| 253 | Amino acid sequence of an anti-NKG2A (HLA-E) CAR |
| 254 | Coding sequence of anti-NKG2A (HLA-E) CAR of SEQ ID NO: 253 |
| 255 | Amino acid sequence of an anti-NKG2A (HLA-E) CAR |
| 256 | Coding sequence of anti-NKG2A (HLA-E) CAR of SEQ ID NO: 255 |
| 257 | Amino acid sequence of an anti-NKG2A (HLA-E) CAR |
| 258 | Coding sequence of anti-NKG2A (HLA-E) CAR of SEQ ID NO: 257 |
| 266 | Amino acid sequence of VH domain of an anti-NKp46 antibody (from |
| | U.S. Pat. No. 10,716,864) |
| 267 | Amino acid sequence of VL domain of an anti-NKp46 antibody (from |
| | U.S. Pat. No. 10,716,864) |
| 268 | Amino acid sequence of human NKp30 (UniProt ID O14931) |
| 269 | Amino acid sequence of human NKp44 (UniProt ID O95944) |
| 270-275 | Amino acid sequences of human KIR |
| 288 | Amino acid sequence of a heavy chain of an anti-KIR antibody |
| | (from U.S. Pat. No. 9,879,082) |
| 289 | Amino acid sequence of a light chain variable domain of anti-KIR antibody |
| | (from U.S. Pat. No. 9,879,082) |

### DETAILED DESCRIPTION

Natural killer (NK) cells are a type of white blood cell that play a crucial role in immune system responses to tumor cells and virally infected cells. NK cells are activated in response to inflammatory mediators such as interferons or cytokines. NK cells also express a variety of activating and inhibitory receptors, as well as co-stimulatory receptors, to recognize and respond to inflamed or infected tissues. These receptors bind cellular stress ligands, which can lead to NK cell responses.

NK cells carry out their cytotoxic functions on target cells with release of special molecules from the NK cell interior, such as perforin and granzymes. When an NK cell contacts a cell targeted for destruction, perforin released from the NK cell forms pores in the cell membrane of the target cell through which the granzymes and associated molecules can enter, inducing cell death of the target cell.

NK cells do not indiscriminately kill normal cells because NK cells express several receptors that recognize Major Histocompatibility Complex (MHC) class I molecules expressed on normal cells. Importantly, some of these receptors can prevent activation of NK cells, so that NK cells do not target healthy tissue. Major histocompatibility complex class I (MHC I) and related molecules are part of the biology that allows NK cells to distinguish healthy self cells from stressed, infected, exogenous, or neoplastic cells. The lack of expression of one or more MHC class I alleles on a target cell leads to NK-mediated target cell lysis. For example, mismatch of MHC I molecules between a donor and recipient in cell, tissue, or organ transplantation, can lead to antibodies recognizing the mismatch and destruction of donor cell, tissue, or organ by NK cells through antibody dependent cellular cytotoxicity.

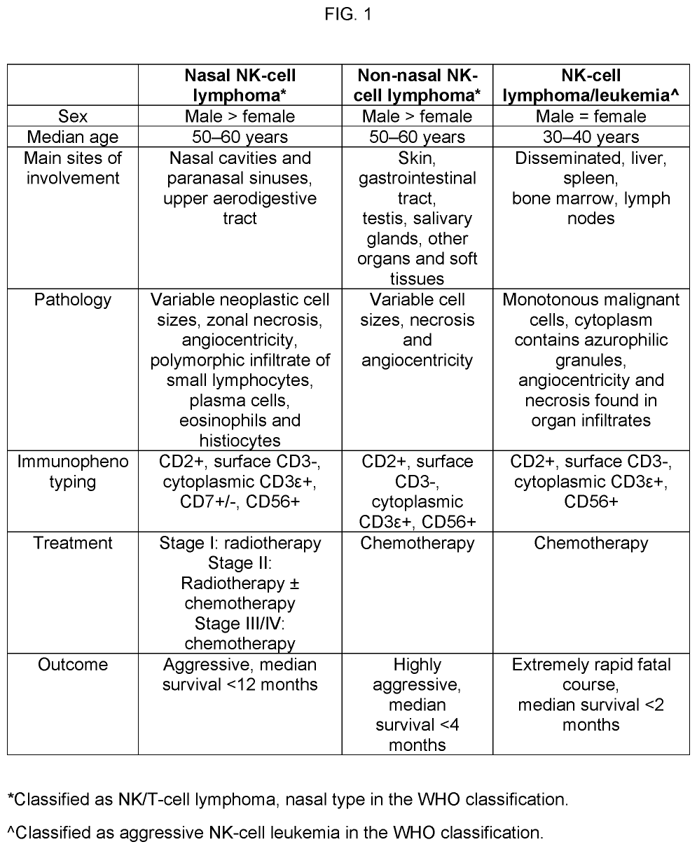

NK cells are normally beneficial; however, NK cell malignancies also occur. Natural Killer (NK) cell neoplasms have limited effective treatment options and high mortality (Campo et al. Blood 117, 5019-5032 (2011); Matutes, E. Int. J. Lab. Hematol. 40, 97-103 (2018)). The World Health Organization (WHO) classifies two main types of NK cell malignancies: extranodal NK/T cell lymphoma, nasal type (ENK/TL) and aggressive NK cell leukemia (ANKL), both strongly associated with the Epstein-Barr virus (EBV) (Campo et al. Blood 117, 5019-5032 (2011); Matutes, E. Int. J. Lab. Hematol. 40, 97-103 (2018); Saleem & Natkunam. Int. J. Mol. Sci. 21, 1501 (2020)). NK cell neoplasms classifications are shown in FIG. 1. These tumors can be characterized by angioinvasion and/or angiodestruction; cytoplasmic azurophilic granules (by Romanowsky staining); and/or CD2+/CD3−/cCD3ε+/CD56+ phenotype. These account for ≥2% of all non-Hodgkin lymphomas globally, but are 3-10× more prevalent in regions of Asia and South America (Perry et al. Haematologica 101, 1244-1250 (2016)). In nonendemic, low resource countries, they are associated with a disproportionate burden of disease (Sanchez-Romero et al. Head Neck Pathol. 13, 624-634 (2019)). Although prognosis with low risk stage I/II ENKTL has become more favorable (90% durable remission), treatment of higher risk and later stage ENKTL remains challenging (Tse & Kwong. Best Pract. Res. Clin. Haematol. 32, 253-261 (2019)), with relapsed/refractory ENKTL retaining a median survival of 6 months (Lim et al. (2017) Annals of Oncology, 28(9), 2199-2205). The prognosis with ANKL is dismal, with median survival reported at 2 months (Kwong, Y.-L. Leukemia 19, 2186-2194 (2005); Suzuki et al. (2004). Leukemia, 18(4), 763-770) in the absence of stem cell transplantation (Hamadani et al. 2017) Biology of Blood and Marrow Transplantation, 23(5), 853-856). To this end, there is a significant need for innovative and more effective treatments for NK cell malignancies.

Changes in circulating or tissue NK cells have also been implicated in autoimmune and alloimmune diseases. Autoimmunity is an immune response to the self's own antigens. Autoimmune disorders linked to NK cell function include: Sjogren's disease; antiphospholipid syndrome; Pemphigus vulgaris; spondylarthropathies; skin diseases including psoriasis; multiple sclerosis; systemic sclerosis; Type I diabetes (Gur et al. Nat. Immunol. 11, 121-128 (2010)); juvenile idiopathic arthritis; rheumatoid arthritis; inflammatory bowel disease; autoimmune liver diseases; and systemic lupus erythematosus (SLE).

Alloimmunity (sometimes called isoimmunity) is an immune response to non-self antigens from members of the same species. The non-self antigens are known as alloantigens or isoantigens. Two major types of alloantigens are blood group antigens and histocompatibility antigens. In alloimmunity, the body creates antibodies against the alloantigens, attacking transfused blood, allotransplanted tissue, and fetus. An alloimmune response can result in graft rejection, manifested as deterioration or complete loss of graft function. NK cells are associated with fetal/neonatal alloimmune thrombocytopenia (FNAIT), where maternal immune responses to fetal platelet antigens can lead to complications such as miscarriage and intrauterine growth restriction (Yougbare et al. (2017) Nat Commun. 8(1): 224). It was observed that uterine natural killer (uNK) cell recruitment and survival beyond mid-gestation lead to elevated NKp46 and CD107 expression, perforin release and trophoblast apoptosis. NK cells are also associated with hematopoietic stem cell rejection, solid tissue transplant rejection, and chronic allograft injury after kidney transplantation.

Although NKp46 expression is normally restricted to NK cells, aberrant NKp46 expression has been reported on non-NK cell malignancies. For example, patients who have Sezary syndrome, an aggressive form of cutaneous T-cell lymphoma, express the NKp46 NK receptor on the cell surface of their peripheral blood malignant CD4+ T lymphocytes (Bensussan et al. (2011) J Invest Dermatol. 131: 969-976). As another example, it has been observed that 20% of mature T cell neoplasms, including T cell large granular lymphocytic leukemia, mycosis fungoides, and ALK+ anaplastic large cell lymphoma, aberrantly express NKp46 receptor (Freud et al. (2013) Am J Clin Pathol. 140:853-866).

Given the role of NK cells in a variety of pathological conditions, there is a need to develop therapies where elimination or reduction of NK cells would be of interest, such as in NK cell malignancies, non-NK malignancies that express NK cell receptors, NK cell-mediated autoimmune or alloimmune disorders, and in conditions where inhibitor NK cell phenotype dominates and there could be benefit to increasing NK cell function (such as malignancies, especially tumor microenvironments, and in some chronic infections).

The present disclosure provides chimeric antigen receptors (CARs) and cells genetically modified to express the CARs as therapies for NK cell associated diseases. A CAR refers to a fusion polypeptide or a set of polypeptides, which when expressed in an immune cell, provides the cell with specificity for a target cell (e.g., NK cell), and with intracellular signal generation. In particular embodiments, engagement of a binding domain of the CAR with its target antigen on the surface of a target cell results in clustering of the CAR and delivering of an activation stimulus to the CAR-expressing cell. The main characteristic of CARs is their ability to redirect immune cell specificity, thereby triggering proliferation, cytokine production, phagocytosis, or production of molecules that can mediate cell death of the target antigen expressing cell in an MHC independent manner, exploiting the cell specific targeting abilities of monoclonal antibodies, soluble ligands, or cell specific co-receptors.

In particular embodiments, a CAR includes: an extracellular component linked to an intracellular component through a transmembrane domain. The extracellular component includes a binding domain that specifically binds an antigen expressed by a targeted cell, here an NK cell. NK cells do not express a uniform cell surface marker (Hudspeth et al. (2013) Frontiers in immunology 4: 69), so two approaches were developed. One is a CAR with a binding domain that specifically binds an activating NK receptor. In particular embodiments, an activating NK receptor includes NKp46 (also known as NCR1, or CD335) and the second is a CAR with a binding domain that specifically binds an inhibitory NK receptor. In particular embodiments, an inhibitory NK receptor includes CD94/NKG2A.

In particular embodiments, the binding domain includes a single chain variable fragment (scFv). For example, a binding domain can include an scFv derived from an anti-NKp46 antibody (Gauthier et al. (2019) Cell 177(7): 1701-1713; WO 2016/207278). A binding domain that binds CD94/NKG2A can include an artificially-designed HLA-E mimetic as described in Gornalusse et al. (2017) Nature biotechnology 35(8): 765.

The extracellular component of a CAR often includes additional components such as a spacer region to enhance conformational flexibility and/or a tag sequence to control the CAR during cell manufacturing and/or after delivery to a subject.

In particular embodiments, the intracellular component includes a cytoplasmic signaling domain (also referred to herein as “an intracellular signaling domain”) derived from one or more of a stimulatory molecule and/or a costimulatory molecule as defined herein. In particular embodiments, the stimulatory molecule is the CD3 zeta (CD3ζ) chain associated with the T cell receptor complex. In particular embodiments, the cytoplasmic signaling domain further includes one or more functional signaling domains derived from at least one costimulatory molecule as defined herein. In particular embodiments, the costimulatory molecule includes 4-1BB (i.e. CD137) or CD28.

In particular embodiments, a CAR that binds an inhibitory NK receptor (e.g., an anti-NKG2A CAR) of the disclosure includes an intracellular component that lacks an intracellular signaling domain. A CAR that binds an inhibitory NK receptor (e.g., an anti-NKG2A CAR) that includes an HLA-E or an HLA-E mimetic lacking an intracellular signaling domain may confer benefits in the development of off-the-shelf CAR T cell therapies. Allogeneic CAR T cell products lacking expression of HLA-A, HLA-B, and/or HLA-C, but overexpressing HLA-E or an HLA-E mimetic can avoid both host T and NK cell-mediated rejection (Gornalusse et al. Nat. Biotechnol. 35, 765-772 (2017); Torikai et al. Blood 122, 1341-1349 (2013)). In particular embodiments, a cell expressing a CAR that binds an inhibitory NK receptor is inherently protected from NK-mediated cell lysis.

In particular embodiments, a cell can co-express a CAR that binds an inhibitory NK receptor and a CAR that binds an activating NK receptor to mitigate cell lysis of the CAR modified cell due to target NK cells activated by the CAR that binds an activating NK receptor. In particular embodiments, an anti-NK CAR modified cell expressing a CAR that binds an activating NK receptor can avoid cell lysis by target NK cells in the presence of a cell genetically modified to express a CAR that binds an inhibitory NK receptor. In particular embodiments, the CAR that binds an activating NK receptor includes an anti-NKp46 CAR. In particular embodiments, the CAR that binds an inhibitory NK receptor includes an anti-NKG2A CAR. In particular embodiments, an anti-NKG2A CAR of the disclosure includes an extracellular component including an HLA-E or HLA-E mimetic that binds an NKG2A receptor and has an intracellular signaling domain. In particular embodiments, an anti-NKG2A CAR of the disclosure includes an extracellular component including an HLA-E or HLA-E mimetic that binds an NKG2A receptor and lacks an intracellular signaling domain. In particular embodiments, an anti-NKG2A CAR of the disclosure includes an extracellular component including an anti-NKG2A scFv that binds an NKG2A receptor and has an intracellular signaling domain. In particular embodiments, an anti-NKG2A CAR of the disclosure includes an extracellular component including an anti-NGK2A scFv that binds an NKG2A receptor and lacks an intracellular signaling domain.

In particular embodiments, the transmembrane domain includes a CD8 transmembrane domain. In particular embodiments, the transmembrane domain is linked to the intracellular component by a linker.

In particular embodiments, a CAR includes an optional leader (i.e. signal) sequence at the amino-terminus (N-terminus) of the CAR protein. In particular embodiments, a CAR includes a leader sequence at the N-terminus of the extracellular binding domain, wherein the leader sequence is optionally cleaved from the binding domain during cellular processing and localization of the CAR to the cellular membrane. In particular embodiments, signal sequences of CARs of the disclosure include: a CD8 signal peptide sequence set forth in SEQ ID NO: 238; a CD8 signal peptide sequenced encoded by SEQ ID NOs: 239; a beta-2-microglobulin (B2M) signal peptide sequence set forth in SEQ ID NO: 130; and a B2M signal peptide sequence encoded by SEQ ID NOs: 131, 132, and 208. In particular embodiments, molecules can be co-expressed with the CAR, including, for example, transduction markers; gene products that act as safety switches; homing receptors; chemokine receptors; and/or cytokine receptors.

Targeted NK cells can be identified by certain characteristics and biological properties, including by: large granular lymphocyte (LGL) morphology; the expression of specific surface antigens including CD16, CD56, CD57, NKR-P1, Ly49D receptor, and/or gp42; the absence of the α/β or γ/δ TCR complex on the cell surface; the absence of myeloperoxidase (MPO); the ability to bind to and kill cells that fail to express self MHC/HLA antigens by the activation of specific cytolytic enzymes; the ability to kill tumor cells or other diseased cells that express a ligand for NK activating receptors; and/or the ability to release protein molecules called cytokines that stimulate or inhibit the immune response. Any of these characteristics and activities can be used to identify NK cells, using methods well known in the art.

In particular embodiments, the present disclosure describes CAR T cells that target NK malignancies. NKp46 and/or NKG2A expression is varied across different subsets of NK cells. In NK cell malignancies, the majority appear to express NKp46 (Freud et al. (2013) American journal of clinical pathology 140(6): 853-866; Uemura et al. (2018) Cancer Science 109(4): 1254-1262). NKG2A is expressed on many NK malignancies, but the percentage of NK malignancies that express NKG2A is not as well-defined (Haedicke et al. (2000) Blood 95(11): 3628-3630; Dukers et al. J. Clin. Path 54(3): 224-228)). In addition, NK cells play a role in some autoimmune and alloimmune inflammation and disease (Poggi & Zocchi (2014) Frontiers in Immunology 5: 27), which makes them amenable to treatment with anti-NK CAR T-cells. Autoimmunity or alloimmunity due to NK cells can be selectively dampened by depleting NK cells that express activating receptors, such as NKp46. In contrast, when NK and T cell immunity is desired, NK and T cell immunity can also be boosted by depleting the NK and T cells which express high levels of inhibitory receptors, somewhat analogous to anti-NKG2A monoclonal antibodies, such as monalizumab, which are being tested as checkpoint inhibitors to treat cancer (Creelan & Antonia (2019) Nat. Rev. Clin. Onc. 16(5): 277-278; André et al. (2018) Cell 175(7): 1731-1743). Monalizumab is a humanized IgG4 antibody targeting NKG2A inhibitory receptors expressed on tumor infiltrating cytotoxic NK and CD8 T lymphocytes (Innate Pharma SA, Marseille, France).

The compositions and methods described herein could completely or partially deplete NK cells, potentially for a prolonged period of time. However, individuals can tolerate periods of NK deficiency, e.g. during stem cell transplant and other procedures. There are also individuals known to have NK cell deficiencies (reviewed in Orange J Allergy Clin Immunol (2013) 132(3): 515-525). However, these individuals eventually develop viral infections (especially with human herpes viruses), and viral associated malignancies. Accordingly, once a diseased population of NK cells is eradicated, re-introducing a healthy NK cell population can be beneficial. In particular embodiments, therapy with anti-NK CAR modified cells can also be performed in conjunction with anti-viral treatment or prophylaxis. In particular embodiments, anti-NK CAR modified cells are regulated (i.e., turned on or off) to manage toxicity, for example utilizing a molecular safety switch.

The following aspects and options related to the current disclosure are now described in additional detail as follows: (i) CAR Binding Domains and Targeted NK Cellular Markers; (ii) CAR Intracellular Components; (iii) CAR Transmembrane Domains; (iv) CAR Linkers; (v) CAR Detection and Control Elements; (vi) Cells Genetically Modified to Express a CAR; (vii) Methods to Collect and Modify Cells Ex Vivo and In Vivo; (viii) Production of CARs; (ix) Assays to Characterize CAR Expressing Cells; (x) Assays to Characterize Target NK Cells; (xi) Compositions and Formulations; (xii) Methods of Use; (xiii) Kits; (xiv) Variants; (xv) Exemplary Embodiments; (xvi) Experimental Examples; and (xvii) Closing Paragraphs. These headings are provided for organizational purposes only and do not limit the scope or interpretation of the disclosure.

(i) CAR Binding Domains and Targeted NK Cellular Markers. In particular embodiments, a CAR can include an extracellular antigen binding domain (i.e. binding domain) that binds a cellular marker expressed by an NK cell. In particular embodiments, the cellular marker is an activating NK receptor. An activating NK receptor includes any molecule on the surface of NK cells that, when stimulated, causes a measurable increase in any property or activity known in the art as associated with NK activity, such as cytokine production (e.g., interferon gamma (IFNγ) and tumor necrosis factor alpha (TNFα)), increases in intracellular free calcium levels, and/or the ability to target cells in a redirected killing assay. In particular embodiments, an NK activating receptor can signal through cytosolic Tyrosine-based motifs. In particular embodiments, NK activating receptors include: CD2; CD44; fractalkine receptor; CD27; CD160; CD137; natural killer group 2D (NKG2D); DNAX accessory molecule-1 (DNAM1); activating killer cell immunoglobulin-like receptors (KAR); C-type lectin-like activating receptor NKp80; signaling lymphocytic activation molecule (SLAM) family of receptors including SLAMF3, SLAMF4, SLAMF5, SLAMF6, and SLAMF7; and natural cytotoxicity receptors (NCRs) including NKp30, NKp44, and NKp46. NCRs are selectively expressed on NK cells and are involved in recognition and activation of NK cells against pathogens, tumor cells, virally infected cells, and self-cells in autoimmune conditions.

In particular embodiments, a CAR can include an extracellular binding domain that binds the NKp46 activating receptor (also known as Natural Cytotoxicity Triggering Receptor 1 (NCR1), or Cluster of Differentiation (CD)335) on an NK cell. NKp46 is a 46 kDa type I transmembrane glycoprotein with extracellular immunoglobulin (Ig) domains, a transmembrane domain containing a positively charged amino acid residue, and a short cytoplasmic tail. An exemplary NKp46 amino acid sequence includes SEQ ID NO: 1 (UniProt ID 076036). An exemplary NKp46 nucleotide sequence that encodes NKp46 includes SEQ ID NO: 2 (NCBI Ref: NM_004829). The cross-linking of NKp46, by specific monoclonal antibodies, leads to a strong NK cell activation resulting in increased intracellular Ca⁺⁺ levels, the triggering of cytotoxicity, and lymphokine release (Sivori et al. (1997) J. Exp. Med. 186: 1129-1136). WO 2005/105848 describes a monoclonal anti-NKp46 antibody (BAB281) that is capable of activating NK cells and that is also specifically bound by human Fc receptors, e.g., Fc gamma (Fcγ) receptors, present on the surface of target cells. In particular embodiments, a monoclonal anti-NKp46 antibody includes an IgG1 antibody raised by immunizing mice with an NK cell clone S192 described by Sivori et al. (1997) J. Exp. Med. 186: 1129-1136. In particular embodiments, a binding domain of a CAR that targets NKp46 includes an scFv derived from an anti-NKp46 antibody (Gauthier et al. (2019) Cell, 177(7), 1701-1713; WO 2016/207278; WO 2017/016805). In particular embodiments, a binding domain of a CAR that targets NKp46 includes complementarity determining regions (CDRs), heavy chain variable (VH) domains, and light chain variable (VL) domains of anti-NKp46 antibodies in Tables 1 and 2.

TABLE 1
Exemplary sequences of CDRs of anti-NKp46 antibodies that target NKp46

| | | CDRH1 | | CDRH2 | | CDRH3 | |
|---|---|---|---|---|---|---|---|
| | | SEQ | | SEQ | | SEQ | |
| Anti- | CDR | ID | | ID | | ID | |
| NKp46ab | definition | NO: | Sequence | NO: | Sequence | NO: | Sequence |
| 8E5B | Kabat | 190 | DTYFH | 193 | RIDPANGNT | 196 | NRYGY |
| | | | | | KYDPKFHD | | |
| | Chothia | 191 | GFNIKDT | 194 | PANG | | RYG |
| | IMGT | 192 | GFNIKDTY | 195 | IDPANGNT | 197 | AANRYGY |
| NKp46- | Kabat | 3 | DYVIN | 6 | EIYPGSGTN | 9 | RGRYGLYA |
| 1 | | | | | YYNEKFKA | | MDY |
| | Chothia | 4 | GYTFTDY | 7 | PGSG | 10 | GRYGLYAM |
| | | | | | | | D |
| | IMGT | 5 | GYTFTDYV | 8 | IYPGSGTN | 11 | ARRGRYGL |
| | | | | | | | YAMDY |
| NKp46- | Kabat | 19 | SDYAWN | 22 | YITYSGSTS | 24 | GGYYGSSW |
| 2 | | | | | YNPSLES | | GVFAY |
| | Chothia | 20 | GYSITSDY | | YSG | 25 | GYYGSSWG |
| | | | | | | | VFA |
| | IMGT | 21 | GYSITSDYA | 23 | ITYSGST | 26 | ARGGYYGS |
| | | | | | | | SWGVFAY |
| NKp46- | Kabat | 33 | EYTMH | 36 | GISPNIGGT | 39 | RGGSFDY |
| 3 | | | | | SYNQKFKG | | |
| | Chothia | 34 | GYTFTEY | 37 | PNIG | 40 | GGSFD |
| | IMGT | 35 | GYTFTEYT | 38 | ISPNIGGT | 41 | ARRGGSFD |
| | | | | | | | Y |
| NKp46- | Kabat | 48 | SFTMH | 51 | YINPSSGYT | 54 | GSSRGFDY |
| 4 | | | | | EYNQKFKD | | |
| | Chothia | 49 | GYTFTSF | 52 | PSSG | 55 | SSRGFD |
| | IMGT | 50 | GYTFTSFT | 53 | INPSSGYT | 56 | VRGSSRGF |
| | | | | | | | DY |
| NKp46- | Kabat | 63 | SSWMH | 66 | HIHPNSGIS | 69 | GGRFDD |
| 6 | | | | | NYNEKFKG | | |
| | Chothia | 64 | GYTFTSS | 67 | PNSG | 70 | GRFD |
| | IMGT | 65 | GYTFTSSW | 68 | IHPNSGIS | 71 | ARGGRFDD |
| NKp46- | Kabat | 74 | SDYAWN | 77 | YITYSGSTN | 78 | CWDYALYA |
| 9 | | | | | YNPSLKS | | MDC |
| | Chothia | 75 | GYSITSDY | | YSG | 79 | WDYALYAM |
| | | | | | | | D |
| | IMGT | 76 | GYSITSDYA | 23 | ITYSGST | 80 | ARCWDYAL |
| | | | | | | | YAMDC |
| BAB281 | Kabat | 84 | NYGMN | 87 | WINTNTGEP | 90 | DYLYYFDY |
| | | | | | TYAEEFKG | | |
| | Chothia | 85 | GYTFTNY | 88 | TNTG | 91 | YLYYFD |
| | IMGT | 86 | GYTFTNYG | 89 | INTNTGEP | 92 | ARDYLYYFD |
| | | | | | | | Y |
| 8E5B | Kabat | 198 | RSSKSLLYIN | 201 | RMSNLAS | 202 | MQHLEYPFT |
| | | | GNTHLF | | | | |
| | Chothia | 199 | SKSLLYING | | RMS | 203 | HLEYPF |
| | | | NTH | | | | |
| | IMGT | 200 | KSLLYINGN | | RMS | 202 | MQHLEYPFT |
| | | | TH | | | | |
| NKp46- | Kabat | 12 | RASQDISNY | 15 | YTSRLHS | 16 | QQGNTRPW |
| 1 | | | LN | | | | T |
| | Chothia | 13 | SQDISNY | | YTS | 17 | YTSGNTRP |
| | | | | | | | W |
| | IMGT | 14 | QDISNY | | YTS | 18 | YTSQQGNT |
| | | | | | | | RPWT |
| NKp46- | Kabat | 27 | RVSENIYSY | 30 | NAKTLAE | 31 | QHHYGTPW |
| 2 | | | LA | | | | T |
| | Chothia | 28 | SENIYSY | | NAK | 32 | HYGTPW |
| | IMGT | 29 | ENIYSY | | NAK | 31 | QHHYGTPW |
| | | | | | | | T |
| NKp46- | Kabat | 42 | RASQSISDY | 45 | YASQSIS | 46 | QNGHSFPLT |
| 3 | | | LH | | | | |
| | Chothia | 43 | SQSISDY | | YAS | 47 | GHSFPL |
| | IMGT | 44 | QSISDY | | YAS | 46 | QNGHSFPLT |
| NKp46- | Kabat | 57 | RASENIYSN | 60 | AATNLAD | 61 | QHFWGTPR |
| 4 | | | LA | | | | T |
| | Chothia | 58 | SENIYSN | | AAT | 62 | FWGTPR |
| | IMGT | 59 | ENIYSN | | AAT | 61 | QHFWGTPR |
| | | | | | | | T |
| NKp46- | Kabat | 42 | RASQSISDY | 45 | YASQSIS | 72 | QNGHSFLM |
| 6 | | | LH | | | | YT |
| | Chothia | 43 | SQSISDY | | YAS | 73 | GHSFLMY |
| | IMGT | 44 | QSISDY | | YAS | 72 | QNGHSFLM |
| | | | | | | | YT |
| NKp46- | Kabat | 81 | RTSENIYSY | 30 | NAKTLAE | 82 | QHHYDTPLT |
| 9 | | | LA | | | | |
| | Chothia | 28 | SENIYSY | | NAK | 83 | HYDTPL |
| | IMGT | 29 | ENIYSY | | NAK | 82 | QHHYDTPLT |
| BAB281 | Kabat | 93 | KASENVVTY | 96 | GASNRYT | 97 | GQGYSYPY |
| | | | VS | | | | T |
| | Chothia | 94 | SENVVTY | | GAS | 98 | GYSYPY |
| | IMGT | 95 | ENVVTY | | GAS | 97 | GQGYSYPY |
| | | | | | | | T |

ab = antibody

TABLE 2
Exemplary amino acid sequences of heavy chain variable (VH) and light
chain variable (VL) domains of anti-NKp46 antibodies that target NKp46

| | SEQ | |
|---|---|---|
| Variable | ID | |
| domain | NO: | Amino acid sequence |
| 8E5B VH | 204 | EIQLQQSGAELVKPGASVKLSCTASGFNIKDTYFHWVKQRPEQGLE |
| | | WIGRIDPANGNTKYDPKFHDKATIIADISSNTAYLQFSSLTSEDTAVY |
| | | YCAANRYGYWGQGTTLTVSS |
| 8E5B VL | 205 | DIVMTQAAPSIPVTPGESVSISCRSSKSLLYINGNTHLFWFLQRPGQ |
| | | SPQLLIYRMSNLASGVPDRFSGSGSGTAFTLRISRVEAEDVGVYYC |
| | | MQHLEYPFTFGSGTKLEIK |
| NKp46-1 VH | 99 | QVQLQQSGPELVKPGASVKMSCKASGYTFTDYVINWGKQRSGQG |
| | | LEWIGEIYPGSGTNYYNEKFKAKATLTADKSSNIAYMQLSSLTSEDS |
| | | AVYFCARRGRYGLYAMDYWGQGTSVTVSS |
| NKp46-1 VL | 100 | DIQMTQTTSSLSASLGDRVTISCRASQDISNYLNWYQQKPDGTVKL |
| | | LIYYTSRLHSGVPSRFSGSGSGTDYSLTINNLEQEDIATYFCQQGNT |
| | | RPWTFGGGTKLEIK |
| NKp46-2 VH | 101 | EVQLQESGPGLVKPSQSLSLTCTVTGYSITSDYAWNWIRQFPGNKL |
| | | EWMGYITYSGSTSYNPSLESRISITRDTSTNQFFLQLNSVTTEDTAT |
| | | YYCARGGYYGSSWGVFAYWGQGTLVTVSA |
| NKp46-2 VL | 102 | DIQMTQSPASLSASVGETVTITCRVSENIYSYLAWYQQKQGKSPQL |
| | | LVYNAKTLAEGVPSRFSGSGSGTQFSLKINSLQPEDFGSYYCQHHY |
| | | GTPWTFGGGTKLEIK |
| NKp46-3 VH | 103 | EVQLQQSGPELVKPGASVKISCKTSGYTFTEYTMHWVKQSHGKSL |
| | | EWIGGISPNIGGTSYNQKFKGKATLTVDKSSSTAYMELRSLTSEDSA |
| | | VYYCARRGGSFDYWGQGTTLTVSS |
| NKp46-3 VL | 104 | DIVMTQSPATLSVTPGDRVSLSCRASQSISDYLHWYQQKSHESPRL |
| | | LIKYASQSISGIPSRFSGSGSGSDFTLSINSVEPEDVGVYYCQNGHS |
| | | FPLTFGAGTKLELK |
| NKp46-4 VH | 105 | QVQLQQSAVELARPGASVKMSCKASGYTFTSFTMHWVKQRPGQG |
| | | LEWIGYINPSSGYTEYNQKFKDKTTLTADKSSSTAYMQLDSLTSDD |
| | | SAVYYCVRGSSRGFDYWGQGTLVTVSA |
| NKp46-4 VL | 106 | DIQMIQSPASLSVSVGETVTITCRASENIYSNLAWFQQKQGKSPQLL |
| | | VYAATNLADGVPSRFSGSGSGTQYSLKINSLQSEDFGIYYCQHFW |
| | | GTPRTFGGGTKLEIK |
| NKp46-6 VH | 107 | LVRPGASVKLSCKASGYTFTSSWMHWAKQRPGQGLEWIGHIHPNS |
| | | GISNYNEKFKGKATLTVDTSSSTAYVDLSSLTSEDSAVYYCARGGR |
| | | FDDWGAGTTVTVSS |
| NKp46-6 VL | 108 | DIVMTQSPATLSVTPGDRVSLSCRASQSISDYLHWYQQKSHESPRL |
| | | LIKYASQSISGIPSRFSGSGSGSDFTLSINSVEPEDVGVYYCQNGHS |
| | | FLMYTFGGGTKLEIK |
| NKp46-9 VH | 109 | DVQLQESGPGLVKPSQSLSLTCTVTGYSITSDYAWNWIRQFPGNKL |
| | | EWMGYITYSGSTNYNPSLKSRISITRDTSKNQFFLQLNSVTTEDTAT |
| | | YYCARCWDYALYAMDCWGQGTSVTVSS |
| NKp46-9 VL | 110 | DIQMTQSPASLSASVGETVTITCRTSENIYSYLAWCQQKQGKSPQL |
| | | LVYNAKTLAEGVPSRFSGSGSGTHFSLKINSLQPEDFGIYYCQHHY |
| | | DTPLTFGAGTKLELK |
| BAB281 VH | 111 | QIQLVQSGPELQKPGETVKISCKASGYTFTNYGMNWKQAPGKGL |
| | | KWMGWINTNTGEPTYAEEFKGRFAFSLETSASTAYLQINNLKNEDT |
| | | ATYFCARDYLYYFDYWGQGTTLTVSS |
| BAB281 VL | 112 | NIVMTQSPKSMSMSVGERVTLTCKASENVVTYVSWYQQKPEQSPK |
| | | LLIYGASNRYTGVPDRFTGSGSATDFTLTISSVQAEDLADYHCGQG |
| | | YSYPYTFGGGTKLEIK |

In particular embodiments, a binding domain of a CAR that targets NKp46 includes single chain variable fragments (scFvs) of anti-NKp46 antibodies in Table 3. In particular embodiments, an scFv derived from an anti-NKp46 antibody that targets NKp46 is encoded by a nucleotide sequence including SEQ ID NOs: 120-126, and 248.

TABLE 3
Exemplary amino acid sequences of scFvs derived from anti-NKp46
antibodies that target NKp46

| | SEQ | |
|---|---|---|
| scFv | ID | |
| Anti-NKp46 | NO: | Amino acid sequence |
| 8E5B | 206 | DIVMTQAAPSIPVTPGESVSISCRSSKSLLYINGNTHLFWFLQRPGQ |
| | | SPQLLIYRMSNLASGVPDRFSGSGSGTAFTLRISRVEAEDVGVYYC |
| | | MQHLEYPFTFGSGTKLEIKGGGGSGGGGSGGGGSEIQLQQSGAEL |
| | | VKPGASVKLSCTASGFNIKDTYFHWVKQRPEQGLEWIGRIDPANGN |
| | | TKYDPKFHDKATIIADISSNTAYLQFSSLTSEDTAVYYCAANRYGYW |
| | | GQGTTLTVSS |
| NKp46-1 | 113 | STGSQVQLQQSGPELVKPGASVKMSCKASGYTFTDYVINWGKQR |
| | | SGQGLEWIGEIYPGSGTNYYNEKFKAKATLTADKSSNIAYMQLSSLT |
| | | SEDSAVYFCARRGRYGLYAMDYWGQGTSVTVSSVEGGSGGSGG |
| | | SGGSGGVDDIQMTQTTSSLSASLGDRVTISCRASQDISNYLNWYQ |
| | | QKPDGTVKLLIYYTSRLHSGVPSRFSGSGSGTDYSLTINNLEQEDIA |
| | | TYFCQQGNTRPWTFGGGTKLEIK |
| NKp46-2 | 114 | STGSEVQLQESGPGLVKPSQSLSLTCTVTGYSITSDYAWNWIRQFP |
| | | GNKLEWMGYITYSGSTSYNPSLESRISITRDTSTNQFFLQLNSVTTE |
| | | DTATYYCARGGYYGSSWGVFAYWGQGTLVTVSAVEGGSGGSGG |
| | | SGGSGGVDDIQMTQSPASLSASVGETVTITCRVSENIYSYLAWYQQ |
| | | KQGKSPQLLVYNAKTLAEGVPSRFSGSGSGTQFSLKINSLQPEDFG |
| | | SYYCQHHYGTPWTFGGGTKLEIK |
| NKp46-3 | 115 | STGSEVQLQQSGPELVKPGASVKISCKTSGYTFTEYTMHWKQSH |
| | | GKSLEWIGGISPNIGGTSYNQKFKGKATLTVDKSSSTAYMELRSLTS |
| | | EDSAVYYCARRGGSFDYWGQGTTLTVSSVEGGSGGSGGSGGSG |
| | | GVDDIVMTQSPATLSVTPGDRVSLSCRASQSISDYLHWYQQKSHE |
| | | SPRLLIKYASQSISGIPSRFSGSGSGSDFTLSINSVEPEDVGVYYCQ |
| | | NGHSFPLTFGAGTKLELK |
| NKp46-4 | 116 | STGSQVQLQQSAVELARPGASVKMSCKASGYTFTSFTMHWKQR |
| | | PGQGLEWIGYINPSSGYTEYNQKFKDKTTLTADKSSSTAYMQLDSL |
| | | TSDDSAVYYCVRGSSRGFDYWGQGTLVTVSAVEGGSGGSGGSG |
| | | GSGGVDDIQMIQSPASLSVSVGETVTITCRASENIYSNLAWFQQKQ |
| | | GKSPQLLVYAATNLADGVPSRFSGSGSGTQYSLKINSLQSEDFGIY |
| | | YCQHFWGTPRTFGGGTKLEIK |
| NKp46-6 | 117 | STGSQVQLQQPGSVLVRPGASVKLSCKASGYTFTSSWMHWAKQR |
| | | PGQGLEWIGHIHPNSGISNYNEKFKGKATLTVDTSSSTAYVDLSSLT |
| | | SEDSAVYYCARGGRFDDWGAGTTVTVSSVEGGSGGSGGSGGSG |
| | | GVDDIVMTQSPATLSVTPGDRVSLSCRASQSISDYLHWYQQKSHE |
| | | SPRLLIKYASQSISGIPSRFSGSGSGSDFTLSINSVEPEDVGVYYCQ |
| | | NGHSFLMYTFGGGTKLEIK |
| NKp46-9 | 118 | STGSDVQLQESGPGLVKPSQSLSLTCTVTGYSITSDYAWNWIRQFP |
| | | GNKLEWMGYITYSGSTNYNPSLKSRISITRDTSKNQFFLQLNSVTTE |
| | | DTATYYCARCWDYALYAMDCWGQGTSVTVSSVEGGSGGSGGSG |
| | | GSGGVDDIQMTQSPASLSASVGETVTITCRTSENIYSYLAWCQQKQ |
| | | GKSPQLLVYNAKTLAEGVPSRFSGSGSGTHFSLKINSLQPEDFGIYY |
| | | CQHHYDTPLTFGAGTKLELK |
| BAB281 | 119 | STGSQIQLVQSGPELQKPGETVKISCKASGYTFTNYGMNWVKQAP |
| | | GKGLKWMGWINTNTGEPTYAEEFKGRFAFSLETSASTAYLQINNLK |
| | | NEDTATYFCARDYLYYFDYWGQGTTLTVSSVEGGSGGSGGSGGS |
| | | GGVDNIVMTQSPKSMSMSVGERVTLTCKASENVVTYVSWYQQKP |
| | | EQSPKLLIYGASNRYTGVPDRFTGSGSATDFTLTISSVQAEDLADYH |
| | | CGQGYSYPYTFGGGTKLEIK |

In particular embodiments, a binding domain of a CAR that targets NKp46 includes a VH domain including a CDRH1 having the sequence of TYGIGVG (SEQ ID NO: 260), a CDRH2 having the sequence of HIWWNDNEYYNIDLKS (SEQ ID NO: 261), and a CDRH3 having the sequence of GNYRYARGYVMDY (SEQ ID NO: 262); and a VL domain including: a CDRL1 having the sequence of RASESVEYYGTSLMQ (SEQ ID NO: 263), a CDRL2 having the sequence of AASNVES (SEQ ID NO: 264), and a CDRL3 having the sequence of QQNRKVPVPWT (SEQ ID NO: 265). In particular embodiments, a binding domain of a CAR that targets NKp46 includes an antigen binding fragment of a heavy chain as set forth in SEQ ID NO: 266 and an antigen binding fragment of a light chain as set forth in SEQ ID NO: 267. In particular embodiments, a binding domain of a CAR that targets NKp46 includes an antigen binding fragment of a commercially available antibody including: mouse monoclonal IgG2b anti-human NKp46 clone #195314 (R&D Systems, cat #MAB1850, Minneapolis, MN); and mouse monoclonal IgG1 anti-human NKp46 clone 9E2 (Biolegend, cat #331902, San Diego, CA).

In particular embodiments, a CAR can include an extracellular binding domain that binds the NKp30 activating receptor. In particular embodiments, NKp30 includes an amino acid sequence of SEQ ID NO: 268 (NCR3, UniProt ID 014931). In particular embodiments, a binding domain of a CAR that targets NKp30 includes an antigen binding fragment of antibody AZ20, antibody A76, and antibody Z25 described in JP2008502322. In particular embodiments, a binding domain of a CAR that targets NKp30 includes an antigen binding fragment of a hamster anti-NKp30 antibody including 15E1, 9G1, 15H6, 9D9, 3A12, and 12D10 described in WO2020172605. In particular embodiments, a binding domain of a CAR that targets NKp30 includes an antigen binding fragment of a commercially available antibody including: mouse monoclonal IgG2a anti-human NKp30 clone #210845 (R&D Systems, cat #MAB1849, Minneapolis, MN); mouse monoclonal IgG1 anti-human NKp30 clone p30-15 (BD Biosciences, cat #563384, Franklin Lakes, NJ); and mouse monoclonal IgG1 anti-NKp30 clone AF29-4D12 (ThermoFisher Scientific, cat #501122309, Waltham, MA).

In particular embodiments, a CAR can include an extracellular binding domain that binds the NKp44 activating receptor. In particular embodiments, NKp44 includes an amino acid sequence of SEQ ID NO: 269 (NCR2/CD336, UniProt ID 095944). In particular embodiments, a binding domain of a CAR that targets NKp44 includes an antigen binding fragment of mouse monoclonal antibody Z231 described in JP2008502322. In particular embodiments, a binding domain of a CAR that targets NKp44 includes an antigen binding fragment of a commercially available antibody including: mouse monoclonal IgG2a anti-human NKp44 Clone #253415 (R&D Systems, cat #MAB22491, Minneapolis, MN); mouse monoclonal IgG2b anti-human NKp44 clone 44.189 (ThermoFisher Scientific, cat #17-3369-42, Waltham, MA); mouse monoclonal IgG2a anti-human NKp44 clone 1G6 (Novus Biologicals, cat #NBP242683, Littleton, CO); and mouse monoclonal IgG1 anti-human NKp44 clone P44-8 (BioLegend, San Diego, CA).

In particular embodiments, a CAR can include an extracellular binding domain that binds an inhibitory NK receptor. In particular embodiments, the extracellular binding domain that binds an inhibitory NK receptor includes an scFv derived from an antibody that binds an inhibitory NK receptor. In humans, two major classes of inhibitory receptors are expressed by NK cells, killer immunoglobulin-like receptor (KIR) and CD94-NKG2A heterodimers. KIR is a type I transmembrane receptor belonging to the immunoglobulin (Ig) superfamily and is characterized by two or three extracellular Ig-like domains. Different KIR specific for groups of HLA-C, HLA-B, or HLA-A alleles have been identified. In particular embodiments, KIR inhibitory receptors include amino acid sequences of SEQ ID NO: 270 (UniProt ID Q8N743; KIR3DL3), SEQ ID NO: 271 (UniProt ID Q99706; KIR2DL4), SEQ ID NO: 272 (UniProt ID P43628; KIR2DL3), SEQ ID NO: 273 (UniProt ID P43630; KIR3DL2), SEQ ID NO: 274 (UniProt ID P43626; KIR2DL1), and SEQ ID NO: 275 (UniProt ID P43629; KIR3DL1). A second type of HLA-specific receptor is formed by the association of lectin-like CD94 molecule with NKG2A. The CD94-NKG2A complex recognizes the nonclassical MHC molecule, HLA-E. HLA-E is considered nonclassical in part because it presents only peptides derived from classical MHC I molecules. In particular embodiments, HLA-E forms a trimeric complex with beta-2-microglobulin (B2M, described below) and a nonamer self-peptide from the signal sequence of a classical MHC class I molecule. In particular embodiments, a classical MHC class I molecule includes HLA-A, HLA-B, HLA-C, and HLA-G. In particular embodiments, this feature allows NK cells to sense changes in expression of MHC I molecules in the environment of a putative target cell. NKG2A contains an immunoreceptor tyrosine-based inhibitory motif (ITIM) that allows the CD94-NGK2A complex to elicit an inhibitory signal upon binding to HLA-E. In particular embodiments, an HLA-E includes an HLA-E heavy (alpha) chain and binds an NKG2A receptor. In particular embodiments, an HLA-E includes an HLA-E heavy (alpha) chain and a beta-2 microglobulin (B2M) light chain and binds an NKG2A receptor. In particular embodiments, an HLA-E includes an HLA-E heavy (alpha) chain, a B2M light chain, and an HLA-E binding peptide from a signal sequence of a classical MHC class I molecule, and binds an NKG2A receptor. In particular embodiments, an HLA-E heavy (alpha) chain includes an HLA-E 01:03 heavy (alpha) chain.

In particular embodiments, a peptide from the signal sequence of a classical MHC class I molecule included in an HLA-E or HLA-E mimetic can include an amino acid consensus sequence of VMAPRTLLL (SEQ ID NO: 276) (Borst et al. Clin Cancer Res 2020; 26:5549-5556). In particular embodiments, an HLA-E binding peptide included in an HLA-E or HLA-E mimetic can include a signal peptide of a classical MHC class I molecule: a peptide of VMAPRTLVL (SEQ ID NO: 277) from HLA-A; a peptide of VMAPRTVLL (SEQ ID NO: 278) from HLA-B; a peptide of VMAPRTLIL (SEQ ID NO: 279) from HLA-C; a peptide of VMAPRTVFL (SEQ ID NO: 280) from HLA-G; a peptide of VMPPRTLLL (SEQ ID NO: 281) from HLA-A; a peptide of VTAPRTVLL (SEQ ID NO: 282) from HLA-B; a peptide of VTAPRTLLL (SEQ ID NO: 283) from HLA-B; a peptide of VMAPRALLL (SEQ ID NO: 284) from HLA-C; and a peptide of VMAPQALLL (SEQ ID NO: 285) from HLA-C (Borst et al. Clin Cancer Res 2020; 26:5549-5556). The signal peptide from a virus may have evolved to bind HLA-E. In particular embodiments, an HLA-E binding peptide included in an HLA-E or HLA-E mimetic can be from a virus including human immunodeficiency virus (HIV) (peptides described in Hannoun et al. Immunol Lett 2018; 202:65-72), cytomegalovirus (CMV) (peptide described in Ulbrecht et al. J Immunol 2000; 164(10):5019-5022), and Epstein-Barr virus (EBV) (peptide described in Jorgensen et al. PLoS one. 2012; 7(9):e46120). In particular embodiments, an HLA-E binding peptide included in an HLA-E or HLA-E mimetic can include: a peptide of multidrug resistance-associated protein 7 including an amino acid sequence of ALALVRMLI (SEQ ID NO: 286) (Wooden et al. J Immunol 2005; 175:1383-1387); and a leader peptide of HSP60 including an amino acid sequence of QMRPVSRVL (SEQ ID NO: 287) (Michaelsson et al. J Exp Med 2002; 196:1403-1414).

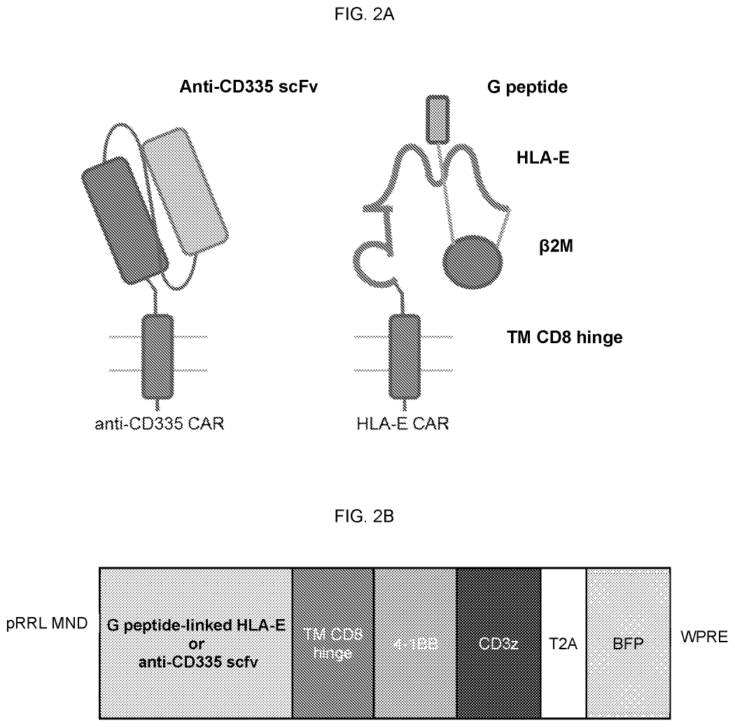

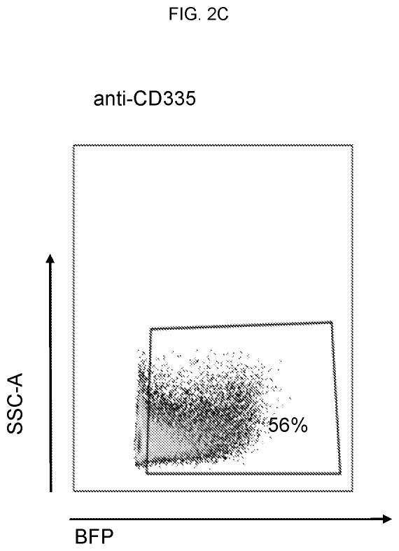

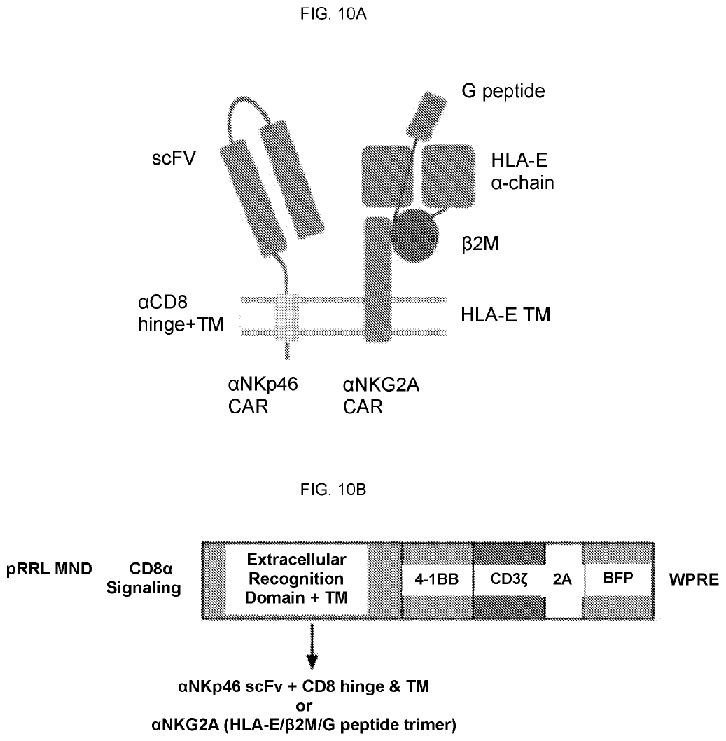

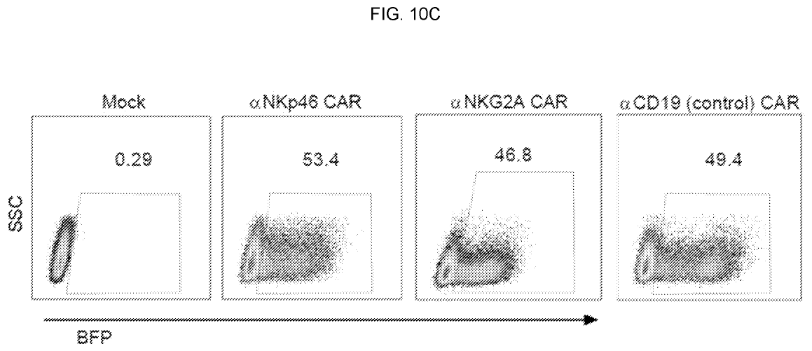

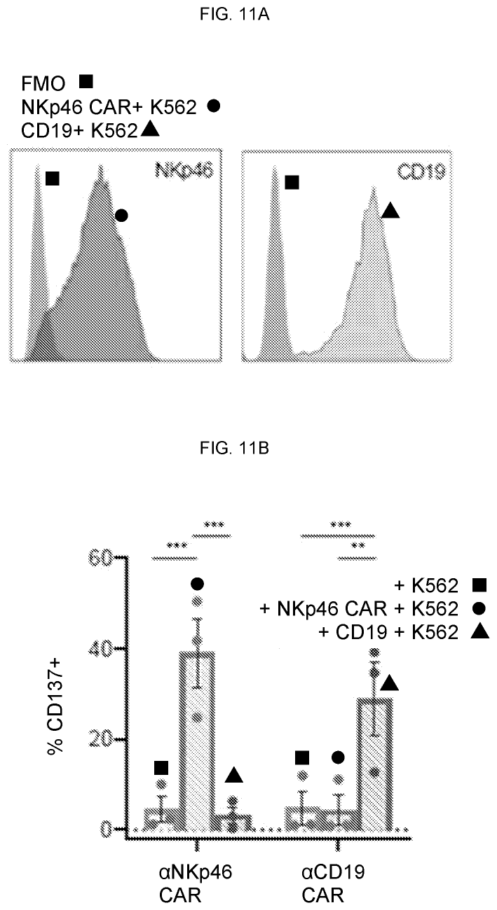

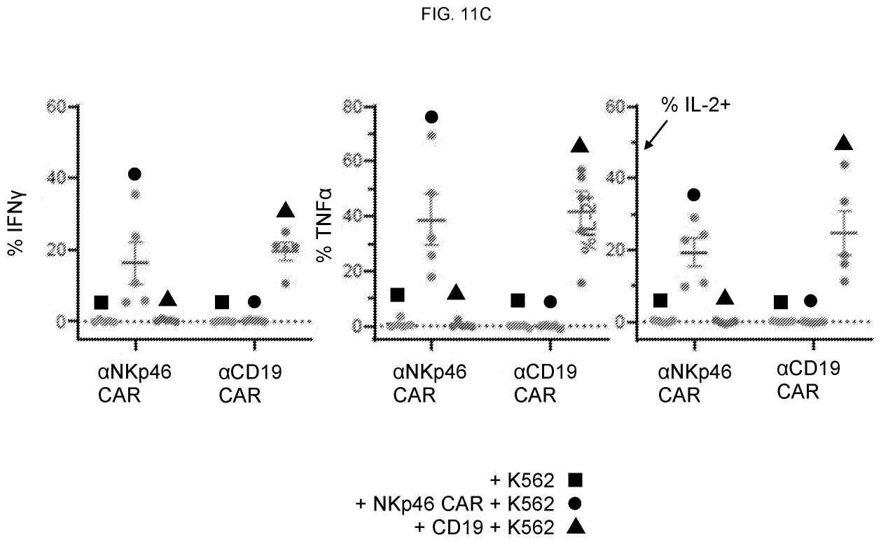

In particular embodiments, a CAR can include an extracellular binding domain that binds the NK cell surface inhibitory receptor CD94/NKG2A. In particular embodiments, a human NKG2A includes SEQ ID NO: 186. In particular embodiments, a human NKG2A is encoded by SEQ ID NO: 187. In particular embodiments, the binding domain that targets NKG2A includes an artificial HLA-E mimetic (Gornalusse et al. (2017) Nature biotechnology, 35(8), 765). In particular embodiments, the HLA-E mimetic includes a heterotrimer of: 1) a signal peptide of HLA-G (another HLA class I molecule); 2) mature beta-2 microglobulin (B2M); and 3) HLA-E 01:03 heavy chain (FIGS. 2A, 10A).

In particular embodiments, a signal peptide of HLA-G (i.e., ‘G peptide’ in FIGS. 2A, 10A) includes a non-polymorphic peptide normally presented by HLA-E that inhibits NK cell-dependent lysis through its binding to CD94/NKG2A. In particular embodiments, an artificial HLA-E mimetic includes a signal peptide of HLA-G including VMAPRTLFL (SEQ ID NO: 127). In particular embodiments, a signal peptide of HLA-G (i.e., ‘G peptide’ in FIGS. 2A, 10A) of an artificial HLA-E mimetic is encoded by SEQ ID NOs: 128, 129, and 207.

B2M is a non-polymorphic gene that encodes a common protein subunit required for surface expression of all polymorphic HLA class I heavy chains. Like other HLA class I molecules, HLA-E can form a heterodimer with a B2M subunit. In particular embodiments, an artificial HLA-E mimetic includes a B2M signal peptide as set forth in SEQ ID NO: 130. In particular embodiments, a B2M signal peptide of an artificial HLA-E mimetic is encoded by SEQ ID NOs: 131, 132, and 208. In particular embodiments, an artificial HLA-E mimetic includes a B2M polypeptide as set forth in SEQ ID NO: 133. In particular embodiments, the B2M polypeptide is a mature polypeptide that does not include the B2M signal peptide. In particular embodiments, a mature B2M polypeptide of an artificial HLA-E mimetic is encoded by SEQ ID NOs: 134, 135, and 209.

In particular embodiments, an artificial HLA-E mimetic includes an HLA-E heavy (alpha) chain. In particular embodiments, an artificial HLA-E mimetic includes an HLA-E 01:03 heavy (alpha) chain as set forth in SEQ ID NO: 136. In particular embodiments, an HLA-E 01:03 heavy (alpha) chain of an artificial HLA-E mimetic is encoded by SEQ ID NOs: 137 and 210.

In particular embodiments, an artificial HLA-E mimetic includes from amino-terminal to carboxy-terminal: 1) a B2M signal peptide set forth in SEQ ID NO: 130 and/or encoded by SEQ ID NOs: 131, 132, or 208; 2) an HLA-G signal peptide of VMAPRTLFL (SEQ ID NO: 127) and/or encoded by SEQ ID NOs: 128, 129, or 207; 3) a (GGGGS)₃ flexible linker set forth in SEQ ID NO: 142 and/or encoded by SEQ ID NO: 211; 4) a B2M mature protein set forth in SEQ ID NO: 133 and/or encoded by SEQ ID NOs: 134, 135, or 209; 5) a (GGGGS)₄ flexible linker set forth in SEQ ID NO: 143 and/or encoded by SEQ ID NO: 212; and 6) an HLA-E 01:03 heavy chain set forth in SEQ ID NO: 136 and/or encoded by SEQ ID NO: 137 or 210.

In particular embodiments, an artificial HLA-E mimetic includes from amino-terminal to carboxy-terminal: 1) a B2M signal peptide set forth in SEQ ID NO: 130 and/or encoded by SEQ ID NO: 208; 2) an HLA-G signal peptide of VMAPRTLFL (SEQ ID NO: 127) and/or encoded by SEQ ID NO: 207; 3) a (GGGGS)₃ flexible linker set forth in SEQ ID NO: 142 and/or encoded by SEQ ID NO: 211; 4) a B2M mature protein set forth in SEQ ID NO: 133 and/or encoded by SEQ ID NO: 209; 5) a (GGGGS)₄ flexible linker set forth in SEQ ID NO: 143 and/or encoded by SEQ ID NO: 212; and 6) an HLA-E 01:03 heavy chain set forth in SEQ ID NO: 136 and/or encoded by SEQ ID NO: 210. In particular embodiments, an artificial HLA-E mimetic includes an amino acid sequence set forth in SEQ ID NO: 242. In particular embodiments, an artificial HLA-E mimetic is encoded by a nucleotide sequence set forth in SEQ ID NO: 243.

In particular embodiments, an artificial HLA-E mimetic includes from amino-terminal to carboxy-terminal: 1) a CD8 signal peptide set forth in SEQ ID NO: 238 and/or encoded by SEQ ID NO: 239; 2) an HLA-G signal peptide of VMAPRTLFL (SEQ ID NO: 127) and/or encoded by SEQ ID NO: 207; 3) a (GGGGS)₃ flexible linker set forth in SEQ ID NO: 142 and/or encoded by SEQ ID NO: 211; 4) a B2M mature protein set forth in SEQ ID NO: 133 and/or encoded by SEQ ID NO: 209; 5) a (GGGGS)₄ flexible linker set forth in SEQ ID NO: 143 and/or encoded by SEQ ID NO: 212; and 6) an HLA-E 01:03 heavy chain set forth in SEQ ID NO: 136 and/or encoded by SEQ ID NO: 210. In particular embodiments, an artificial HLA-E mimetic includes an amino acid sequence set forth in SEQ ID NO: 244. In particular embodiments, an artificial HLA-E mimetic is encoded by a nucleotide sequence set forth in SEQ ID NO: 245.

In particular embodiments, an artificial HLA-E mimetic includes from amino-terminal to carboxy-terminal: 1) a CD8 signal peptide set forth in SEQ ID NO: 238 and/or encoded by SEQ ID NO: 239; 2) a B2M signal peptide set forth in SEQ ID NO: 130 and/or encoded by SEQ ID NO: 208; 3) an HLA-G signal peptide of VMAPRTLFL (SEQ ID NO: 127) and/or encoded by SEQ ID NO: 207; 4) a (GGGGS)₃ flexible linker set forth in SEQ ID NO: 142 and/or encoded by SEQ ID NO: 211; 5) a B2M mature protein set forth in SEQ ID NO: 133 and/or encoded by SEQ ID NO: 209; 6) a (GGGGS)₄ flexible linker set forth in SEQ ID NO: 143 and/or encoded by SEQ ID NO: 212; and 7) an HLA-E 01:03 heavy chain set forth in SEQ ID NO: 136 and/or encoded by SEQ ID NO: 210. In particular embodiments, an artificial HLA-E mimetic includes an amino acid sequence set forth in SEQ ID NO: 246. In particular embodiments, an artificial HLA-E mimetic is encoded by a nucleotide sequence set forth in SEQ ID NO: 247.

In particular embodiments, an artificial HLA-E mimetic can exclude one or more of the following: a CD8 signal peptide, a B2M signal peptide, an HLA-G signal peptide, a (GGGGS)₃ flexible linker, a (GGGGS)₄ flexible linker, a B2M mature protein, and/or the transmembrane domain of an HLA-E alpha chain. In particular embodiments, an artificial HLA-E mimetic can exclude an HLA-G signal peptide. In particular embodiments, an artificial HLA-E mimetic can exclude a B2M mature protein. In particular embodiments, an artificial HLA-E mimetic can exclude the transmembrane domain of an HLA-E alpha chain. In particular embodiments, the transmembrane domain of an HLA-E alpha chain includes the sequence VGIIAGLVLLGSVVSGAVVAAVIW (SEQ ID NO: 259) found in mature B2M set forth in SEQ ID NO: 136.

In particular embodiments, an unexpected benefit of using an artificial HLA-E mimetic to bind CD94-NKG2A inhibitory receptor on an NK cell includes allowing HLA disrupted allogeneic anti-NK CAR modified cells to be used without resulting in NK-mediated cell lysis of the anti-NK CAR modified cells. In particular embodiments, anti-NK CAR modified cells expressing CARs with an artificial HLA-E mimetic can avoid NK-mediated cell lysis because the NK cells are inhibited by the artificial HLA-E mimetic. In particular embodiments, the artificial HLA-E mimetic lacks an intracellular signaling domain. In particular embodiments, an anti-NK CAR modified cell expressing an anti-NKp46 CAR can avoid cell lysis by target NK cells by co-expressing a CAR with an artificial HLA-E mimetic. In particular embodiments, an anti-NK CAR modified cell expressing an anti-NKp46 CAR can avoid cell lysis by target NK cells in the presence of a cell genetically modified to express a CAR with an artificial HLA-E mimetic. Co-expression includes expressing a CAR in a cell where the CAR has both an activating NK receptor binding domain and an inhibitory NK receptor binding domain or expressing two separate CARs in a cell, one CAR including an activating NK receptor binding domain and the other CAR including an inhibitory NK receptor binding domain.

In particular embodiments, the binding domain that targets NKG2A includes an scFv derived from an antibody that binds NKG2A. In particular embodiments, the binding domain that targets NKG2A includes monalizumab or can be derived from monalizumab. Monalizumab is a humanized IgG4 monoclonal antibody targeting NKG2A receptors expressed on tumor infiltrating cytotoxic NK and CD8 T lymphocytes (WO 2016/041947; Innate Pharma, Marseilles, France).

In particular embodiments, the binding domain that targets NKG2A includes CDRs (according to Kabat numbering), VH domains, heavy chains, and a light chain of monalizumab disclosed in Tables 4-6.

TABLE 4
Exemplary sequences of CDRs of monalizumab

| CDRH1 | | CDRH2 | | CDRH3 | |
|---|---|---|---|---|---|
| SEQ | | SEQ | | SEQ | |
| ID | | ID | | ID | |
| NO: | Sequence | NO: | Sequence | NO: | Sequence |
| 213 | SYWMN | 214 | RIDPYDSETHY | 215 | GGYDFDVGTLYWFFDV |
| CDRL1 | | CDRL2 | | CDRL3 | |
| SEQ | | SEQ | | SEQ | |
| ID | | ID | | ID | |
| NO: | Sequence | NO: | Sequence | NO: | Sequence |
| 216 | RASENIYSYLA | 217 | NAKTLAE | 218 | QHHYGTPRT |

TABLE 5
Exemplary amino acid sequences of variable heavy domains (VH) of
monalizumab

| | SEQ | |
|---|---|---|
| Variable | ID | |
| domain | NO: | Amino acid sequence |
| VH6 | 219 | EVQLVQSGAEVKKPGESLKISCKGSGYSFTSYWMNWVRQMPGKG |
| | | LEWMGRIDPYDSETHYSPSFQGQVTISADKSISTAYLQWSSLKASD |
| | | TAMYYCARGGYDFDVGTLYWFFDVWGQGTTVTVS |
| VH1 | 220 | QVQLVQSGAEVKKPGASVKVSCKASGYTFTSYWMNWRQAPGQ |
| | | GLEWMGRIDPYDSETHYAQKLQGRVTMTTDTSTSTAYMELRSLRS |
| | | DDTAVYYCARGGYDFDVGTLYWFFDVWGQGTTVTVS |
| VH5 | 221 | EVQLVQSGAEVKKPGESLRISCKGSGYSFTSYWMNWVRQMPGKG |
| | | LEWMGRIDPYDSETHYSPSFQGHVTISADKSISTAYLQWSSLKASD |
| | | TAMYYCARGGYDFDVGTLYWFFDVWGQGTTVTVS |
| VH7 | 222 | EVQLVQSGAEVKKPGATVKISCKVSGYTFTSYWMNWQQAPGKG |
| | | LEWMGRIDPYDSETHYAEKFQGRVTITADTSTDTAYMELSSLRSED |
| | | TAVYYCATGGYDFDVGTLYWFFDVWGQGTTVTVS |
| VH8 | 223 | QVQLVQSGAEVKKPGASVKVSCKASGYTFTSYWMNWRQAPGQ |
| | | GLEWMGRIDPYDSETHYAQKFQGRVTMTRDTSTSTVYMELSSLRS |
| | | EDTAVYYCARGGYDFDVGTLYWFFDVWGQGTTVTVS |

TABLE 6
Exemplary amino acid sequences of heavy chains and a light chain of
monalizumab

| | SEQ | |
|---|---|---|
| | ID | |
| Chain | NO: | Amino acid sequence |
| VH6 heavy | 224 | EVQLVQSGAEVKKPGESLKISCKGSGYSFTSYWMNWVRQMPGKG |
| chain | | LEWMGRIDPYDSETHYSPSFQGQVTISADKSISTAYLQWSSLKASD |
| | | TAMYYCARGGYDFDVGTLYWFFDVWGQGTTVTVSSASTKGPSVF |
| | | PLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQD |
| | | WLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSF |
| | | FLYSRLTVDKSRWQEGNVFSCSVMHEALHNHYTQKSLSLSLGK |
| VH1 heavy | 225 | QVQLVQSGAEVKKPGASVKVSCKASGYTFTSYWMNWVRQAPGQ |
| chain | | GLEWMGRIDPYDSETHYAQKLQGRVTMTTDTSTSTAYMELRSLRS |
| | | DDTAVYYCARGGYDFDVGTLYWFFDVWGQGTTVTVSSASTKGPS |
| | | VFPLAPCSRSTSESTAALGCLVKDYFPEPVTVSWNSGALTSGVHTF |
| | | PAVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLG |
| | | K |
| VH5 heavy | 226 | EVQLVQSGAEVKKPGESLRISCKGSGYSFTSYWMNWVRQMPGKG |
| chain | | LEWMGRIDPYDSETHYSPSFQGHVTISADKSISTAYLQWSSLKASD |
| | | TAMYYCARGGYDFDVGTLYWFFDVWGQGTTVTVSSASTKGPSVF |
| | | PLAPCSRSTSESTAALGCLVKDYFPEPVTVSWNSGALTSGVHTFPA |
| | | VLQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQD |
| | | WLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSF |
| | | FLYSRLTVDKSRWQEGNVFSCSVMHEALHNHYTQKSLSLSLGK |
| VH7 heavy | 227 | EVQLVQSGAEVKKPGATVKISCKVSGYTFTSYWMNWQQAPGKG |
| chain | | LEWMGRIDPYDSETHYAEKFQGRVTITADTSTDTAYMELSSLRSED |
| | | TAVYYCATGGYDFDVGTLYWFFDVWGQGTTVTVSSASTKGPSVFP |
| | | LAPCSRSTSESTAALGCLVKDYFPEPVTVSWNSGALTSGVHTFPAV |
| | | LQSSGLYSLSSVVTVPSSSLGTKTYTCNVDHKPSNTKVDKRVESKY |
| | | GPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQD |
| | | WLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSF |
| | | FLYSRLTVDKSRWQEGNVFSCSVMHEALHNHYTQKSLSLSLGK |
| VH8 heavy | 228 | QVQLVQSGAEVKKPGASVKVSCKASGYTFTSYWMNWRQAPGQ |
| chain | | GLEWMGRIDPYDSETHYAQKFQGRVTMTRDTSTSTVYMELSSLRS |
| | | EDTAVYYCARGGYDFDVGTLYWFFDVWGQGTTVTVSSASTKGPS |
| | | VFPLAPCSRSTSESTAALGCLVKDYFPEPVTVSWNSGALTSGVHTF |
| | | PAVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLG |
| | | K |
| Light chain | 229 | DIQMTQSPSSLSASVGDRVTITCRASENIYSYLAWYQQKPGKAPKL |
| | | LIYNAKTLAEGVPSRFSGSGSGTDFTLTISSLQPEDFATYYCQHHYG |
| | | TPRTFGGGTKVEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |

In particular embodiments, the binding domain that targets NKG2A can be derived from: anti-NKG2A antibody Z270 (WO2006070286; WO2008/009545; U.S. Pat. No. 8,206,709); humanized anti-NKG2A antibody Z199 (WO2009/092805; Carretero et al. Eur J Immunol 1997; 27:563-567; commercially available via Beckman Coulter, Inc., Product No. IM2750, Brea, CA)); rat anti-murine NKG2-antibody 20D5 (Vance et al. (1999) J Exp Med 190: 1801-1812; commercially available via BD Biosciences Pharmingen, Catalog No. 550518, USA); and murine antibody 3S9 (US 2003/0095965). In particular embodiments, the binding domain that targets NKG2A includes a VH domain including a CDRH1 having the sequence of SYAMS (SEQ ID NO: 290), a CDRH2 having the sequence of EISSGGSYTYYADSVKG (SEQ ID NO: 291), and a CDRH3 having the sequence of HGDYPRFFDV (SEQ ID NO: 292); and a VL domain including: a CDRL1 having the sequence of SASSSVSSYIY (SEQ ID NO: 293), a CDRL2 having the sequence of LTSNLAS (SEQ ID NO: 294), and a CDRL3 having the sequence of QQWSGNPYT (SEQ ID NO: 295), as described in U.S. Pat. No. 9,422,368.

In particular embodiments, a CAR can include an extracellular binding domain that binds KIR. In particular embodiments, a binding domain of a CAR that targets KIR includes an antigen binding fragment of a heavy chain as set forth in SEQ ID NO: 288 and an antigen binding fragment of a light chain variable domain as set forth in SEQ ID NO: 289. In particular embodiments, a binding domain of a CAR that targets KIR includes an antigen binding fragment of antibody A210 or antibody A803g described in U.S. Pat. No. 8,119,775 and Shin et al. Hybridoma 1999; 18:521-527. In particular embodiments, a binding domain of a CAR that targets KIR includes an antigen binding fragment of a commercially available antibody including: mouse monoclonal IgG2b anti-human KIR clone #180704 (R&D Systems, cat #MAB1848, Minneapolis, MN); and mouse monoclonal IgG1 anti-human KIR clone NKVFS1 (Bio-Rad, cat #MCA2243, Hercules, CA; Spaggiari et al. Blood 2002; 99:1706-1714 and Blood 2002; 100:4098-4107).

Immune cells modified to express CARs targeting activating NK receptors, such as NKp46, can kill NK cells but can also induce activation of NK cells leading to NK-mediated killing of the CAR T cells. In particular embodiments, a binding domain that binds an inhibitory NK receptor can be expressed or over-expressed in anti-NKp46 CAR modified cells to help prevent lysis of the anti-NKp46 CAR modified cells by NK cells that are activated because of anti-NKp46 CAR modified cells binding to NKp46. In particular embodiments, the binding domain that binds an inhibitory NK receptor includes HLA-E or an HLA-E mimetic. This contrasts from the use of the HLA-E mimetic by Gornalusse et al. 2017 (Nature biotechnology, 35(8), 765), where the HLA-E mimetic was used to prevent lysis of cells that were engineered not to express HLA. In the present disclosure, by contrast, the HLA-E helps prevent lysis of CAR cells in which HLA and beta-2-microglobulin are still intact. In particular embodiments, the binding domain that binds an inhibitory NK receptor includes monalizumab. In particular embodiments, the binding domain that binds an inhibitory NK receptor includes a binding domain derived from an anti-NKG2A antibody described herein.

Expression or overexpression of a binding domain that binds an inhibitory NK receptor to confer resistance on CAR expressing cells (i.e., anti-NK activating receptor CAR expressing cells) to NK cell killing can occur in a number of ways. In particular embodiments, a cell can co-express: (a) a CAR wherein the extracellular component includes a binding domain that binds an activating NK receptor (e.g., NKp46); and (b) a CAR wherein the extracellular component includes an inhibitory NK receptor binding domain (e.g., HLA-E mimetic). In particular embodiments, a cell can co-express: (a) a CAR wherein the extracellular component includes a binding domain that binds an activating NK receptor (e.g., NKp46); and (b) a CAR wherein the extracellular component includes an inhibitory NK receptor binding domain (e.g., HLA-E mimetic) and the intracellular component lacks the one or more intracellular signaling domains. In particular embodiments, a cell can co-express: (a) a CAR wherein the extracellular component includes a binding domain that binds an activating NK receptor (e.g., NKp46); and (b) a molecule on the surface of the cell including an inhibitory NK receptor binding domain (e.g., HLA-E mimetic) that does not include intracellular signaling components. In particular embodiments, a cell can express a single CAR including an extracellular component including activating NK receptor (e.g., NKp46) and an inhibitory NK receptor binding domain (e.g., HLA-E mimetic). In particular embodiments, a first cell population can express a CAR wherein the extracellular component includes a binding domain that binds an activating NK receptor (e.g., NKp46), and a second cell population can express a CAR wherein the extracellular component includes an inhibitory NK receptor binding domain (e.g., HLA-E mimetic) (with or without one or more intracellular signaling domains). In particular embodiments, a first cell population can express a CAR wherein the extracellular component includes a binding domain that binds an activating NK receptor (e.g., NKp46), and a second cell population can express a molecule on the surface of each cell including an inhibitory NK receptor binding domain (e.g., HLA-E mimetic) that does not include intracellular signaling domains. Szivi|tviwwmsr [symbol] spigypi$i2k [symbol] sp}titxmhi [symbol] EV- [symbol] r [symbol] i [symbol] hiw [symbol] gvie [symbol] k$ xli [symbol] |tviwwmsr [symbol] i [symbol] spigypi [symbol] zi [symbol] viexi [symbol] er$xli [symbol] i [symbol] svq [symbol] vshygi [symbol] zivi|tviww [symbol] r$ s [symbol] spigypi$,i [symbol] spjtit [symbol] i [symbol] EV- [symbol] r [symbol] i [symbol] rgpyhiw [symbol] |tviww [symbol] j [symbol] spigypi [symbol] rgshjh [symbol] f} [symbol] iri [symbol] igxs [symbol] ex [symbol] xcshygih [symbol] xs

[symbol]

In particular embodiments, a binding domain can include an antibody or a binding fragment thereof. As would be understood by the skilled person and as described elsewhere herein, a complete antibody includes two heavy chains and two light chains. Each heavy chain includes a variable region and a first, second, and third constant region, while each light chain includes a variable region and a constant region. Mammalian heavy chains are classified as α, δ, ε, γ, and p, and mammalian light chains are classified as A or K. Immunoglobulins including the α, δ, ε, γ, and p heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains p and c have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity determining regions” or “CDRs”. The CDRs can be defined or identified by conventional methods, such as by sequence according to Kabat et al. (Wu and Kabat (1970) J Exp Med. 132(2):211-50; Borden and Kabat (1987) PNAS, 84: 2440-2443; Kabat et al., Sequences of Proteins of Immunological Interest, U.S. Department of Health and Human Services, 1991), or by structure according to Chothia et al (Chothia and Lesk (1987) J Mol. Biol., 196(4): 901-917; Chothia et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an antibody, Fv, scFv, dsFv, Fab, or other antibody fragment as disclosed herein. References to “V_{L}” or “VL” refer to the variable region of an immunoglobulin light chain, including that of an antibody, Fv, scFv, dsFv, Fab, or other antibody fragment as disclosed herein. In particular embodiments, a “VH domain” includes the variable region of an immunoglobulin heavy chain and includes the CDRs of the heavy chain. In particular embodiments, a “VL domain” includes the variable region of an immunoglobulin light chain and includes the CDRs of the light chain.

The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include Fab, Fab′, F(ab′)₂, Fv fragments, single chain variable (scFv) antibody fragments, disulfide-linked Fvs (sdFv), a Fd fragment including VH and constant CH1 domains, linear antibodies, single domain antibodies such as sdAb (either VL or VH), camelid variable heavy only (VHH) domains, multi-specific antibodies formed from antibody fragments such as a bivalent fragment including two Fab fragments linked by a disulfide bridge at the hinge region, and an isolated CDR or other epitope binding fragments of an antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson (2005) Nature Biotechnology 23:1126-1136). Antigen binding fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein including at least one antibody fragment including a variable region of a light chain and at least one antibody fragment including a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. In particular embodiments, a linker connecting the variable regions can include glycine-serine linkers, including SEQ ID NOs: 142-145. In particular embodiments, an scFv may have the VL and VH variable regions in either order, e.g., with respect to the N-terminal and C-terminal ends of the polypeptide, the scFv may include VL-linker-VH or may include VH-linker-VL.

A recombinant antibody includes an antibody which is generated using recombinant DNA technology, such as, for example, an antibody expressed by a bacteriophage or yeast expression system. A recombinant antibody also includes an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In particular embodiments, a binding domain can include humanized forms of non-human (e.g., murine) antibodies or antigen binding fragments thereof. A humanized antibody includes an antibody in which the constant and variable framework region of one or more human immunoglobulins is fused with the binding region, e.g., the CDR, of an animal (non-human) immunoglobulin. Such humanized antibodies are designed to maintain the binding specificity of the non-human antibody from which the binding regions are derived but avoid an immune reaction against the non-human antibody. In particular embodiments, a binding domain can include a fully human antibody or antibody fragment thereof, where the whole molecule is of human origin or includes an amino acid sequence identical to a human form of the antibody or immunoglobulin.

In particular embodiments, a binding domain can include a chimeric antibody or antigen binding fragment thereof. A chimeric antibody can include an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

(ii) CAR Intracellular Components. An intracellular component of a CAR includes one or more intracellular signaling domains. In particular embodiments, the intracellular signaling domain generates a signal that promotes an immune effector function of the CAR modified cell, e.g., an anti-NK CAR modified immune cell. Examples of immune effector function, e.g., in an anti-NK CAR modified immune cell, include cytolytic activity and helper activity, including the secretion of cytokines.

A signaling domain refers to the functional portion of a protein which acts by transmitting information within the cell to regulate cellular activity via defined signaling pathways by generating second messengers or functioning as effectors by responding to such messengers. Stimulation refers to a primary response induced by binding of a stimulatory molecule (e.g., a CAR) with its cognate ligand (e.g., an NK cell surface marker), thereby mediating a signal transduction event, such as signal transduction via appropriate signaling domains of the CAR. Stimulation can mediate altered expression of certain molecules. A stimulatory molecule refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In particular embodiments, the signal is a primary signal that is initiated by, for instance, binding of a CAR to an NK cell surface marker, which leads to mediation of an immune cell response, including proliferation, activation, differentiation, and the like.

An intracellular signaling domain can include the entire intracellular portion, or the entire native intracellular signaling domain, of the molecule from which it is derived, or a functional fragment or derivative thereof. In particular embodiments, an intracellular signaling domain can include a primary intracellular signaling domain. In particular embodiments, primary intracellular signaling domains include those derived from the molecules responsible for primary stimulation, or antigen dependent stimulation. In particular embodiments, the intracellular signaling domain can include a costimulatory intracellular domain.

A primary intracellular signaling domain can include a signaling motif which is known as an immunoreceptor tyrosine-based activation motif or ITAM. Examples of ITAM containing primary cytoplasmic signaling sequences include those derived from CD3ζ, common FcR γ (FCER1G), Fc γ RIIa, FcR β (Fc ε R1b), CD3 γ, CD3 δ, CD3 ε, CD79a, CD79b, DAP10, and DAP12, or a combination thereof.

In particular embodiments, a CD3ζ (CD247) stimulatory domain can include amino acid residues from the cytoplasmic domain of the T cell receptor zeta chain, or functional derivatives thereof, that are sufficient to functionally transmit an initial signal necessary for cell activation. In particular embodiments, a CD3ζ stimulatory domain can include human CD3ζ stimulatory domain or functional orthologs thereof. In particular embodiments, a human CD3ζ stimulatory domain includes SEQ ID NO: 146. In particular embodiments, a human CD3ζ stimulatory domain is encoded by SEQ ID NO: 147. In particular embodiments, in the case of an intracellular signaling domain that is derived from a CD3ζ molecule, the intracellular signaling domain retains sufficient CD3ζ structure such that it can generate a signal under appropriate conditions.

In particular embodiments, the intracellular signaling domain can include a costimulatory intracellular domain. In particular embodiments, costimulatory intracellular signaling domains include those derived from molecules responsible for costimulatory signals, or antigen independent stimulation. In particular embodiments, a costimulatory intracellular signaling domain can be the intracellular portion of a costimulatory molecule. A costimulatory molecule refers to a cognate binding partner on an immune cell that specifically binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1BB (CD137), OX40, GITR, CD30, CD40, ICOS (CD278), BAFFR, HVEM (LIGHTR), ICAM-1, lymphocyte function-associated antigen-1 (LFA-1; CD11a/CD18), CD2, CDS, CD7, CD287, LIGHT, NKG2C, NKG2D, SLAMF7, NKp80 (KLRF1), NKp30, NKp44, NKp46, CD160 (BY55), B7-H3 (CD276), CD19, CD4, CD8α, CD8β, IL2Rβ, IL2Rγ, IL7Rα, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, and the like, or a combination thereof.

In particular embodiments, a costimulatory intracellular signaling domain includes 4-1BB (CD137, TNFRSF9). 4-1BB refers to a member of the tumor necrosis factor receptor (TNFR) superfamily. In particular embodiments, a 4-1BB costimulatory domain includes a human 4-1BB costimulatory domain or a functional ortholog thereof. In particular embodiments, a human 4-1BB costimulatory domain includes SEQ ID NO: 148. In particular embodiments, a human 4-1BB costimulatory domain is encoded by SEQ ID NO: 149.

In particular embodiments, a costimulatory intracellular signaling domain includes CD28. CD28 is a T cell-specific glycoprotein involved in T cell activation, the induction of cell proliferation and cytokine production, and promotion of T cell survival. In particular embodiments, a CD28 costimulatory domain includes a human CD28 costimulatory domain or a functional ortholog thereof. In particular embodiments, a human CD28 costimulatory domain includes SEQ ID NO: 188. In particular embodiments, a human CD28 costimulatory domain is encoded by SEQ ID NO: 189.

In particular embodiments, an intracellular signaling domain includes a combination of one or more stimulatory domains and one or more costimulatory domains described herein. In particular embodiments, an intracellular signaling domain includes a 4-1BB costimulatory domain and a CD3ζ stimulatory domain. In particular embodiments, an intracellular signaling domain including a 4-1BB costimulatory domain and a CD3ζ stimulatory domain is set forth in SEQ ID NO: 230. In particular embodiments, an intracellular signaling domain including a 4-1BB costimulatory domain and a CD3ζ stimulatory domain is encoded by a sequence set forth in SEQ ID NO: 231.

(iii) CAR Transmembrane Domains. A CAR can be designed to include a transmembrane domain that links the extracellular component to the intracellular component of the CAR. A transmembrane domain can anchor a CAR molecule to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In particular embodiments, the transmembrane domain may be from the same protein that the signaling domain, costimulatory domain, or the hinge domain is derived from. In particular embodiments, the transmembrane domain is not derived from the same protein that any other domain of the CAR is derived from. In particular embodiments, the transmembrane domain can be selected or modified by amino acid substitution to avoid binding of or to minimize interactions with other domains in the CAR.

In particular embodiments, a transmembrane domain has a three-dimensional structure that is thermodynamically stable in a cell membrane, and generally ranges in length from 15 to 30 amino acids. The structure of a transmembrane domain can include an alpha helix, a beta barrel, a beta sheet, a beta helix, or any combination thereof.

The transmembrane domain may be derived either from a natural or from a recombinant source. Where the source is natural, the domain may be derived from any membrane-bound or transmembrane protein. In particular embodiments, the transmembrane domain is capable of signaling to the intracellular domain(s) whenever the CAR has bound to a target. In particular embodiments, a transmembrane domain may include at least the transmembrane region(s) of: the α, β, or ζ chain of the T-cell receptor; CD28; CD27; CD3ε; CD45; CD4; CD5; CD8; CD9; CD16; CD22; CD33; CD37; CD64; CD80; CD86; CD134; CD137; and/or CD154. In particular embodiments, a transmembrane domain may include at least the transmembrane region(s) of: KIRDS2; OX40; CD2; LFA-1; ICOS; 4-1BB; GITR; CD40; BAFFR; HVEM; SLAMF7; NKp80; NKp44; NKp30; NKp46; CD160; CD19; IL2Rβ; IL2Rγ; IL7Rα; ITGA1; VLA1; CD49a; ITGA4; IA4; CD49D; ITGA6; VLA-6; CD49f; ITGAD; CDI Id; ITGAE; CD103; ITGAL; CDI Ia; ITGAM; CDI Ib; ITGAX; CDI Ic; ITGB1; CD29; ITGB2; CD18; ITGB7; TNFR2; DNAM1; SLAMF4; CD84; CD96; CEACAM1; CRT AM; Ly9; CD160; PSGL1; CD100; SLAMF6 (NTB-A, LyI08); SLAM; BLAME; SELPLG; LTBR; PAG/Cbp; NKG2D; NKG2C; or a combination thereof. In particular embodiments, a transmembrane domain may include a transmembrane domain from CD8a chain. In particular embodiments, a CD8 transmembrane domain includes SEQ ID NO: 138. In particular embodiments, a CD8 transmembrane domain is encoded by SEQ ID NO: 139 or 233. In particular embodiments, a transmembrane domain may include a transmembrane domain from an HLA-E alpha chain. In particular embodiments, the HLA-E alpha chain includes the sequence VGIIAGLVLLGSVVSGAVVAAVIW (SEQ ID NO: 259) found in mature B2M set forth in SEQ ID NO: 136.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain. In particular embodiments, a CD8 hinge is juxtaposed on the extracellular side of the transmembrane domain. In particular embodiments, a CAR disclosed herein includes an amino acid sequence of a CD8 hinge and a CD8 transmembrane domain set forth in SEQ ID NO: 234 and/or encoded by a sequence set forth in SEQ ID NO: 235.

(iv) CAR Linkers. As used herein, a linker can be any portion of a CAR molecule that serves to connect two subcomponents or domains of the molecule. In particular embodiments, linkers can provide flexibility for a CAR or portion of a CAR. Linkers in the context of linking VH and VL of antibody derived binding domains of scFv are described above. Linkers can include spacer regions and junction amino acids. In particular embodiments, a linker includes a glycine-serine linker of amino acid sequence set forth in SEQ ID NO: 142 and/or encoded by a sequence set forth in SEQ ID NO: 211. In particular embodiments, a linker includes a glycine-serine linker of amino acid sequence set forth in SEQ ID NO: 143 and/or encoded by a sequence set forth in SEQ ID NO: 212.

Spacer regions are a type of linker region that are used to create appropriate distances and/or flexibility from other linked components. In particular embodiments, the length of a spacer region can be customized for individual cellular markers on NK cells to optimize NK cell recognition and destruction. The spacer can be of a length that provides for increased responsiveness of the CAR expressing cell following antigen binding, as compared to in the absence of the spacer. In particular embodiments, a spacer region length can be selected based upon the location of a cellular marker epitope, affinity of a binding domain for the epitope, and/or the ability of the CAR modified cells to destroy target NK cells ex vivo and/or in vivo in response to NK cellular marker recognition. Spacer regions can also allow for high expression levels in CAR modified cells. In particular embodiments, an extracellular spacer region of a CAR is located between a transmembrane domain and the extracellular binding domain.

Exemplary spacers include those having 10 to 250 amino acids, 10 to 200 amino acids, 10 to 150 amino acids, 10 to 100 amino acids, 10 to 50 amino acids, or 10 to 25 amino acids. In particular embodiments, a spacer region is 12 amino acids, 20 amino acids, 21 amino acids, 26 amino acids, 27 amino acids, 45 amino acids, or 50 amino acids. In particular embodiments, a longer spacer is greater than 119 amino acids, an intermediate spacer is 13-119 amino acids, and a short spacer is 10-12 amino acids.

In particular embodiments, a spacer region includes an immunoglobulin hinge region. An immunoglobulin hinge region may be a wild-type immunoglobulin hinge region or an altered wild-type immunoglobulin hinge region. In particular embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. In particular embodiments, the spacer region can include all or a portion of a hinge region sequence from IgG1, IgG2, IgG3, IgG4 or IgD alone or in combination with all or a portion of a CH2 region; all or a portion of a CH3 region; or all or a portion of a CH2 region and all or a portion of a CH3 region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

Exemplary spacers include IgG4 hinge alone, IgG4 hinge linked to CH2 and CH3 domains, or IgG4 hinge linked to the CH3 domain. In particular embodiments, the spacer includes an IgG4 linker of the amino acid sequence: ESKYGPPCPPC (SEQ ID NO: 150). Hinge regions can be modified to avoid undesirable structural interactions such as dimerization with unintended partners. Other examples of hinge regions that can be used in CARs described herein include the hinge region present in extracellular regions of type 1 membrane proteins, such as CD8α, CD4, CD28, and CD7, which may be wild-type or variants thereof. In particular embodiments, a hinge includes a CD8α hinge set forth in SEQ ID NO: 140, and/or encoded by a sequence set forth in SEQ ID NO: 141 or 232. In particular embodiments, the spacer region can be a CD28 linker of the amino acid sequence PSPLFPGPSKP (SEQ ID NO: 151).

In particular embodiments, a spacer region includes a hinge region of a type II C-lectin interdomain (stalk) region or a cluster of differentiation (CD) molecule stalk region. A “stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids between the stalk region and the transmembrane region or the CTLD. In another example, the 233 amino acid human NKG2A protein (UniProt ID P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

Junction amino acids can be a linker which can be used to connect the sequences of CAR domains when the distance provided by a spacer is not needed and/or wanted. In particular embodiments, junction amino acids are short amino acid sequences that can be used to connect intracellular signaling domains. In particular embodiments, junction amino acids are 9 amino acids or less.

Junction amino acids can be a short oligo- or protein linker, preferably between 2 and 9 amino acids (e.g., 2, 3, 4, 5, 6, 7, 8, or 9 amino acids) in length to form the linker. In particular embodiments, a glycine-serine doublet can be used as a suitable junction amino acid linker. In particular embodiments, a single amino acid, e.g., an alanine, a glycine, can be used as a suitable junction amino acid.

In particular embodiments, a short oligo- or polypeptide linker, between 2 and 9 amino acids in length may link the transmembrane domain and the intracellular component of the CAR. A glycine-serine doublet provides a particularly suitable linker. In particular embodiments, a linker can include SEQ ID NOs: 142-145.

(v) CAR Detection and Control Elements. In particular embodiments, a CAR can include one or more tags to activate, promote proliferation of, detect, enrich for, isolate, track, deplete and/or eliminate genetically modified cells in vitro, in vivo and/or ex vivo. A tag refers to a unique synthetic peptide sequence fused to a CAR, or that is part of a CAR, to which a cognate binding molecule (e.g., ligand, antibody, or other binding partner) is capable of specifically binding, where the binding property can be used to activate, promote proliferation of, detect, enrich for, isolate, track, deplete and/or eliminate cells expressing the tagged CAR.

Tags that can be included in a CAR include, for example, His tag (SEQ ID NO: 152), Flag tag (SEQ ID NOs: 153-155), Xpress tag (SEQ ID NO: 156), Avi tag (SEQ ID NO: 157), Calmodulin binding peptide (CBP) tag (SEQ ID NO: 158), Polyglutamate tag (SEQ ID NO: 159), HA tag (SEQ ID NOs: 160-162), Myc tag (SEQ ID NO: 163), Strep tag (which refers to the original STREP® tag (SEQ ID NO: 164), STREP® tag II (SEQ ID NO: 165) (IBA Institut fur Bioanalytik, Germany); see, e.g., U.S. Pat. No. 7,981,632), Softag 1 (SEQ ID NO: 166), Softag 3 (SEQ ID NO: 167), and V5 tag (SEQ ID NO: 168).

Conjugate binding molecules that specifically bind tag sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies, and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal, and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, one or more transduction markers can be co-expressed with the CAR using a skipping element that allows expression of the transduction marker and the CAR as distinct molecules to track cells that have integrated a vector carrying a CAR and transduction marker. In particular embodiments, a transduction marker can be used to activate, promote proliferation of, detect, enrich for, isolate, track, deplete and/or eliminate genetically modified cells in vitro, in vivo and/or ex vivo. Transduction markers can include any suitable fluorescent protein including: blue fluorescent proteins (e.g., BFP, eBFP, eBFP2, Azurite, mKalamal, GFPuv, Sapphire, T-sapphire); cyan fluorescent proteins (e.g., eCFP, Cerulean, CyPet, AmCyanl, Midoriishi-Cyan); green fluorescent proteins (e.g., GFP-2, tagGFP, turboGFP, eGFP, Emerald, Azami Green, Monomeric Azami Green, CopGFP, AceGFP, ZsGreenl); orange fluorescent proteins (e.g., mOrange, mKO, Kusabira-Orange, Monomeric Kusabira-Orange, mTangerine, tdTomato); red fluorescent proteins (e.g., mKate, mKate2, mPlum, DsRed monomer, mCherry, mRFP1, DsRed-Express, DsRed2, DsRed-Monomer, HcRed-Tandem, HcRedI, AsRed2, eqFP611, mRaspberry, mStrawberry, Jred); yellow fluorescent proteins (e.g., YFP, eYFP, Citrine, Venus, YPet, PhiYFP, ZsYellowl); and any other suitable fluorescent proteins, including, for example, firefly luciferase. In particular embodiments, the transduction marker can include any cell surface displayed marker that can be detected with an antibody that binds to that marker and allows sorting of cells that have the marker. In particular embodiments, the transduction marker can include the magnetic sortable marker streptavidin binding peptide (SBP) displayed at the cell surface by a truncated Low Affinity Nerve Growth Receptor (LNGFRF) and one-step selection with streptavidin-conjugated magnetic beads (Matheson et al. (2014) PloS one 9(10): e111437) or a truncated human epidermal growth factor receptor (EGFR) (tEGFR; see Wang et al., Blood 118: 1255, 2011).

In particular embodiments, the transduction marker includes a BFP including SEQ ID NOs: 169-174 (Heim and Tsien (1996) Current Biology, 6(2): 178-182; Yang et al. (1998) Journal of Biological Chemistry, 273(14): 8212-8216; Ai et al. (2007) Biochemistry, 46(20): 5904-5910; and Constantini et al. (2015) Nature Communications, 6(1): 7670). In particular embodiments, a BFP in a CAR of the disclosure includes an amino acid sequence set forth in SEQ ID NO: 236 and/or is encoded by a sequence set forth in SEQ ID NO: 237.

In particular embodiments, anti-NK CAR modified cells may be detected or tracked in vivo by using cognate binding molecules (e.g., antibodies) that bind with specificity to a tag or transduction marker and that are conjugated to a fluorescent dye, radio-tracer, iron-oxide nanoparticle or other imaging agent known in the art for detection by X-ray, CT-scan, MRI-scan, PET-scan, ultrasound, flow cytometry, near infrared imaging systems, or other imaging modalities (see, e.g., Yu et al. (2012) Theranostics 2:3).

In particular embodiments, an anti-NK CAR modified cell or CAR molecule can include elements to regulate the activity of the CAR to manage toxicity or tune the magnitude of CAR activity. For example, a bispecific (or tandem) CAR may be utilized, which incorporates two distinct antigen-recognition domains within one receptor (e.g., an anti-NKp46 binding domain and an anti-NKG2A binding domain). In particular embodiments, cells expressing these tandem CARs recognize target cells expressing either one of the antigens. In particular embodiments, molecular safety switches in the form of suicide genes, such as inducible-caspase-9 (iCASP9), herpes simplex virus thymidine kinase (HSV-TK), or truncated surface receptors (e.g., tEGFR) can be used. A suicide gene encodes a molecule which allows selective destruction of cells expressing this molecule upon administration of a nontoxic prodrug or antibody (Jones et al. (2014) Front. Pharmacol., 5: 254; Sun et al. (2018) J. Immunol. Res.). For example, the iCASP9 system is based on the fusion of caspase 9 and a drug-sensitive FK-modified binding protein. Upon being exposed to the synthetic molecule AP1903, the fusion protein dimerizes and leads to the rapid apoptosis of cells expressing the fusion protein. In particular embodiments, the use of an inhibitory CAR (iCAR) can selectively limit cytokine secretion, cytotoxicity, and/or proliferation induced through an activating CAR and can be utilized to protect healthy tissue from anti-NK CAR-mediated destruction (Fedorov et al. (2013) Sci. Transl. Med., 5: 215ra172).

In particular embodiments, CAR expressing cells administered to a subject can be controlled by depleting the CAR expressing cells at a desired time after administration. A CAR including at least one tag and/or transduction marker can be depleted using a respective cognate binding molecule that binds the tag or transduction marker. In particular embodiments, the present disclosure provides a method for depleting an anti-NK CAR modified cell by using a cognate binding molecule specific for a tag or transduction marker (e.g., an antibody) or by using a second modified cell expressing a CAR that has specificity for the tag or transduction marker. In particular embodiments, a cognate binding molecule includes a depletion agent specific for a tag or transduction marker. For example, if tEFGR is used as the transduction marker, then an anti-tEFGR binding domain (e.g., antibody, scFv) fused to or conjugated to a cell-toxic reagent (such as a toxin or radiometal) may be used, or an anti-tEFGR/anti-CD3 bispecific scFv, or an anti-tEFGR CAR T cell may be used.

Particular embodiments provide for elements that can be used to turn on, induce, or increase the activity of a CAR. Systems have been developed that allow pharmacologic induction of CAR expression. In particular embodiments, a system can include a bipartite receptor system containing separate antigen-targeting and signal transduction polypeptides, each containing an extracellular dimerization domain. T cell activation is antigen dependent but can only be achieved in the presence of a dimerizing drug, rapamycin (Leung et al. (2019) JCI Insight 4(11): e124430).

Regulation of CAR T cell activity is reviewed in Brandt et al. (2020) Frontiers in Immunology, 11: 326.

(vi) Cells Genetically Modified to Express a CAR. The present disclosure includes cells genetically modified to express a CAR. As used herein, the term “genetically modified” or “genetically engineered” refers to the addition of extra genetic material in the form of DNA or RNA into the total genetic material in a cell. The terms “genetically modified cells” and “modified cells” are used interchangeably. In particular embodiments, a cell genetically modified to express a CAR includes an immune effector cell. An “immune effector cell” includes any cell of the immune system that has one or more effector functions (e.g., cytotoxic cell killing activity, secretion of cytokines, induction of antibody-dependent cell cytotoxicity (ADCC) and/or complement-dependent cytotoxicity (CDC). Immune effector cells are a subtype of immune cells.

Immune cells of the disclosure can be autologous/autogeneic (“self”) or non-autologous (“non-self,” e.g., allogeneic, syngeneic or xenogeneic). “Autologous” refers to cells from the same subject. “Allogeneic” refers to cells of the same species that differ genetically to a cell in comparison. “Syngeneic” refers to cells of a different subject that are genetically identical to the cell in comparison. “Xenogeneic” refers to cells of a different species to the cell in comparison. In particular embodiments, modified cells of the disclosure are autologous or allogeneic.

In particular embodiments, genetically modified cells include lymphocytes. In particular embodiments, genetically modified cells include T cells, B cells, natural killer (NK) cells, monocytes/macrophages, and HSPC.

Most T cells have a T-cell receptor (TCR) composed of two separate peptide chains (the α- and β-TCR chains). γδ T cells represent a small subset of T cells that possess a distinct T cell receptor (TCR) made up of one γ-chain and one δ-chain.

CD3 is expressed on all mature T cells. T cells can further be classified into cytotoxic T cells (CD8+ T cells, also referred to as CTLs) and helper T cells (CD4+ T cells).

Cytotoxic T cells destroy virally infected cells and tumor cells and are also implicated in transplant rejection. These cells recognize their targets by binding to antigen associated with MHC class I, which is present on the surface of nearly every cell of the body.

Central memory T cells (TCM) refer to antigen experienced CTL that express CD62L or CCR7 and CD45RO and does not express or has decreased expression of CD45RA as compared to naive cells.

Effector memory T cells (TEM) refer to an antigen experienced T-cell that does not express or has decreased expression of CD62L as compared to central memory cells and does not express or has decreased expression of CD45RA as compared to a naive cell. In particular embodiments, effector memory T cells are negative for expression of CD62L and CCR7, compared to naive cells or central memory cells, and have variable expression of CD28 and CD45RA. Effector T cells are positive for granzyme B and perforin as compared to memory or naive T cells.

Helper T cells assist other immune cells such as activating of cytotoxic T cells and macrophages and facilitating the maturation of B cells, among other functions. Helper T cells become activated when they are presented with peptide antigens by MHC class II molecules that are expressed on the surface of antigen presenting cells (APCs). Once activated, they divide rapidly and secrete cytokines that regulate or assist in the active immune response.

Natural killer T (NKT) cells are a subset of T cells that co-express an αβ T-cell receptor, but also express a variety of molecular markers that are typically associated with natural killer cells, such as NK1.1 (CD161), CD16, and/or CD56.

Natural killer cells (also known as K cells and killer cells) express CD8, CD16 and CD56 but do not express CD3. NK cells also express activating receptors such as NKp46 and inhibitory receptors such as NKG2A that regulate NK cell cytotoxic function against tumor and virally infected cells.

Macrophages (and their precursors, monocytes) reside in every tissue of the body where they engulf apoptotic cells, pathogens and other non-self-components. Monocytes/macrophages express CD11b, F4/80, CD68, CD11c, IL-4Rα, and/or CD163.

Immature dendritic cells (i.e., pre-activation) engulf antigens and other non-self-components in the periphery and subsequently, in activated form, migrate to T cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic stem cells (HSC) refer to undifferentiated hematopoietic cells that are capable of self-renewal and differentiation into all other hematopoietic cell types. HSC are CD34+.

Hematopoietic progenitor cells (HPC) are derived from HSC and are capable of further differentiation into mature cell types. HPC can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T cells, B cells, and NK cells. HPC are CD24^{lo} Lin⁻ CD117⁺.

HSPC refer to a cell population having HSC and HPC. HSPC cell populations can be positive for CD34, CD43, CD45RO, CD45RA, CD59, CD90, CD109, CD117, CD133, CD166, HLA DR, or a combination thereof.

Particular embodiments of the disclosure provide for simultaneous targeting of NKp46 and NKG2A on a target NK cell by a CAR modified cell. The simultaneous targeting may minimize tumor escape due to downregulation of either one of the target antigens on NK cells. In particular embodiments, a cell can be genetically modified to co-express an anti-NKp46 CAR and an anti-NKG2A CAR. In particular embodiments, a first cell population can be genetically modified to express an anti-NKp46 CAR and a second cell population can be genetically modified to express an anti-NKG2A CAR.

In particular embodiments, the simultaneous targeting of an activating NK receptor and an inhibitory NK receptor on a target NK cell by a cell genetically modified to express both an anti-activating NK receptor CAR and an anti-inhibitory NK receptor CAR may mitigate killing of the CAR modified cell by activated target NK cells. In particular embodiments, a cell can be genetically modified to co-express an anti-activating NK receptor CAR and an anti-inhibitory NK receptor CAR. In particular embodiments, a cell can be genetically modified to co-express an anti-activating NK receptor CAR and an inhibitory NK receptor binding domain. In particular embodiments, a first cell population can be genetically modified to express an anti-activating NK receptor CAR and a second cell population can be genetically modified to express an anti-inhibitory NK receptor CAR. In particular embodiments, a first cell population can be genetically modified to express an anti-activating NK receptor CAR and a second cell population can be genetically modified to express an inhibitory NK receptor binding domain. In particular embodiments, non-autologous CAR T cells engineered to lack expression of HLA class I molecules (e.g., HLA-A, HLA-B, HLA-C) can also be engineered to co-express: i) an anti-activating NK receptor CAR and an anti-inhibitory NK receptor CAR, or ii) an anti-activating NK receptor CAR and an inhibitory NK receptor binding domain, to reduce NK-mediated killing of the non-autologous CAR T cells, allowing the use of off-the-shelf CAR T cells. In particular embodiments, non-autologous CAR T cells engineered to lack expression of HLA class I molecules have a disruption in both copies of the B2M gene (Gornalusse et al. (2017) Nature biotechnology 35(8): 765). In particular embodiments, the anti-activating NK receptor CAR is an anti-NKp46 CAR. In particular embodiments, the anti-inhibitory NK receptor CAR is an anti-NKG2A CAR. In particular embodiments, the inhibitory NK receptor binding domain is an NKG2A binding domain. In particular embodiments, the NKG2A binding domain is an HLA-E. In particular embodiments, the NKG2A binding domain is an artificial HLA-E mimetic.

Off-the-shelf CAR T cells would be useful to reduce rejection of CAR T cells in a recipient. Immune rejection occurs when a transplanted cell, tissue, or organ is not accepted by the body of a recipient (host). Immune rejection is mediated by T cells and B cells of the adaptive immune system, along with NK cells of the innate immune system. For example, parts of the CAR can be recognized as foreign by T cells and NK cells of the host and targeted for destruction. Immune rejection of transplants can include hyperacute rejection, acute rejection, and chronic rejection. In particular embodiments, hyperacute rejection occurs shortly after transplantation. In particular embodiments, hyperacute rejection includes pre-existing antibodies reactive to donor tissue. In particular embodiments, hyperacute rejection includes severe systemic inflammatory responses following by blood clotting. In particular embodiments, acute rejection occurs within one week after transplantation due to HLA antigen mismatch. In particular embodiments, chronic rejection includes mismatched minor histocompatibility complex, resulting in long-term rejection of the transplant. In particular embodiments, treatment for acute rejection includes re-transplantation or administration of chemotherapeutic immune suppressants (e.g., corticosteroids and calcineurin inhibitors). However, immune suppressants can lead to immunocompromise complications. In particular embodiments, rejection of CAR modified cells by a recipient includes no therapeutic response to the CAR therapy. In particular embodiments, rejection of CAR modified cells by a recipient includes destruction of the CAR modified cells by cytotoxic T cells and/or NK cells of the recipient. In particular embodiments, administration of anti-inhibitory NK receptor CAR (e.g., an anti-NKG2A CAR) expressing cells can reduce rejection of CAR modified cells by a recipient as compared to rejection of CAR modified cells by a recipient prior to administration of the anti-inhibitory NK receptor CAR expressing cells.

In particular embodiments, an anti-NKp46 CAR includes: an NKp46 scFv including CDRs of SEQ ID NOs: 3-98, 260-265; an NKp46 scFv including VH and VL of SEQ ID NOs: 99-112; an NKp46 scFv including SEQ ID NOs: 113-119; an antigen binding fragment of a heavy chain including SEQ ID NO: 266; and/or an antigen binding fragment of a light chain including SEQ ID NO: 267. In particular embodiments, an anti-NKp46 CAR includes: an NKp46 scFv including CDRs of SEQ ID NOs: 190-203; an NKp46 scFv including VH and VL of SEQ ID NOs: 204 and 205; and/or an NKp46 scFv including SEQ ID NO: 206. In particular embodiments, the binding domain that binds NKG2A includes an artificial HLA-E mimetic including SEQ ID NOs: 127, 130, 133, 136, 142, and 143. In particular embodiments, the binding domain that binds NKG2A includes an artificial HLA-E mimetic including SEQ ID NOs: 242, 244, and 246. In particular embodiments, the binding domain that binds NKG2A includes: CDRs of SEQ ID NOs: 213, 214, 215, 216, 217, 218, 290, 291, 292, 293, 294, and 295; VH of SEQ ID NOs: 219, 220, 221, 222, and 223; an antigen binding fragment of a heavy chain of SEQ ID NOs: 224, 225, 226, 227, and 228; and/or an antigen binding fragment of a light chain of SEQ ID NO: 229.

(vii) Methods to Collect and Modify Cells Ex Vivo and In Vivo. The present disclosure provides methods for collecting, enriching for, culturing, and modifying cells to express a CAR.

In particular embodiments, T cells are isolated from a sample such as blood or a blood-derived sample, an apheresis or a leukapheresis product. Exemplary samples include whole blood, peripheral blood mononuclear cells (PBMCs), leukocytes, bone marrow, thymus, cancer tissue, lymphoid tissue, spleen, or other appropriate sources.

Sources of HSPC include umbilical cord blood, placental blood, and peripheral blood (see U.S. Pat. Nos. 5,004,681; 7,399,633; and U.S. Pat. No. 7,147,626; Craddock, et al., 1997, Blood 90(12):4779-4788; Jin, et al., 2008, Journal of Translational Medicine 6:39; Pelus, 2008, Curr. Opin. Hematol. 15(4):285-292; Papayannopoulou, et al., 1998, Blood 91(7):2231-2239; Tricot, et al., 2008, Haematologica 93(11):1739-1742; and Weaver et al., 2001, Bone Marrow Transplantation 27(2):523-529).

Methods regarding collection, anti-coagulation and processing, etc. of blood samples can be found in, for example, Alsever, et al., 1941, N.Y. St. J. Med. 41:126; De Gowin, et al., 1940, J. Am. Med. Ass. 114:850; Smith, et al., 1959, J. Thorac. Cardiovasc. Surg. 38:573; Rous and Turner, 1916, J. Exp. Med. 23:219; and Hum, 1968, Storage of Blood, Academic Press, New York, pp. 26-160.

In particular embodiments, collected cells are washed, centrifuged, and/or incubated in the presence of one or more reagents, for example, to remove unwanted components, enrich for desired components, lyse or remove cells sensitive to particular reagents. The isolation can include one or more of various cell preparation and separation steps, including separation based on one or more properties, such as size, density, sensitivity or resistance to particular reagents, and/or affinity, e.g., immunoaffinity, to antibodies or other binding partners.

In particular embodiments, one or more of the cell populations enriched, isolated and/or selected from a sample by the provided methods are cells that are positive for (marker+) or express high levels (marker^{hi}) of one or more particular markers, such as surface markers, or that are negative for (marker-) or express relatively low levels (marker^{lo}) of one or more markers. In particular embodiments, the cell populations (such as T cells) are enriched for cells that are positive or expressing high surface levels of cell markers described elsewhere herein.

In particular embodiments, T cells can be isolated from peripheral blood mononuclear cells (PBMCs) by lysing the red blood cells and depleting the monocytes, for example, by centrifugation through a PERCOLL™ gradient. In particular embodiments, a specific subpopulation of T cells, expressing CD3, CD28, CD4, CD8, CD45RA, and CD45RO is further isolated by positive or negative selection techniques. For example, enrichment of a T cell population by negative selection can be accomplished with a combination of antibodies directed to surface markers unique to the negatively selected cells. In particular embodiments, cell sorting and/or selection occurs via negative magnetic immunoadherence or flow cytometry using a cocktail of monoclonal antibodies directed to cell surface markers present on the cells negatively selected. For example, to enrich for CD4⁺ cells by negative selection, a monoclonal antibody cocktail that typically includes antibodies to CD14, CD20, CD11b, CD16, HLA-DR, and CD8 can be used. Flow cytometry and cell sorting may also be used to isolate cell populations of interest for use in the present disclosure.

Following isolation and/or enrichment, cells can be expanded to increase the number of cells. In particular embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 2006/0121005.

Generally, the T cells are expanded by contact with a surface having attached thereto an agent that stimulates a CD3 TCR complex associated signal and a ligand that stimulates a co-stimulatory molecule on the surface of the T cells. In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines (see Berg et al., Transplant Proc. 30(8):3975-3977, 1998; Haanen et al., J. Exp. Med. 190(9): 13191328, 1999; Garland et al., J. Immunol Meth. 227(1-2):53-63, 1999). In particular embodiments, anti-CD3 and anti-CD28 antibodies attached to the same bead serve as a “surrogate” antigen presenting cell (APC). In particular embodiments, the T cells may be activated and stimulated to proliferate with feeder cells and appropriate antibodies and cytokines using methods such as those described in U.S. Pat. Nos. 6,040,177; 5,827,642; and WO 2012/129514.

In particular embodiments, artificial APC (aAPC) can be made by engineering K562, U937, 721.221, T2, and C1R cells to direct the stable expression and secretion of a variety of co-stimulatory molecules and cytokines. In particular embodiments, K32 or U32 aAPCs are used to direct the display of one or more antibody-based stimulatory molecules on the aAPC cell surface. Expression of various combinations of genes on the aAPC enables the precise determination of human T cell activation requirements, such that aAPCs can be tailored for the optimal propagation of T cell subsets with specific growth requirements and distinct functions. The aAPCs support ex vivo growth and long-term expansion of functional human T cells without requiring the addition of exogenous cytokines, in contrast to the use of natural APCs. Populations of T cells can be expanded by aAPCs expressing a variety of costimulatory molecules including CD137L (4-1BBL), CD134L (OX40L), and/or CD80 or CD86. Finally, the aAPCs provide an efficient platform to expand genetically modified T cells and to maintain CD28 expression on T cells. aAPCs are described in WO 03/057171 and US 2003/0147869.

In particular embodiments, HSPCs can be isolated and/or expanded following methods described in, for example, U.S. Pat. Nos. 7,399,633; 5,004,681; US 2010/0183564; WO2006/047569; WO2007/095594; WO 2011/127470; and WO 2011/127472; Vamum-Finney, et al., 1993, Blood 101:1784-1789; Delaney, et al., 2005, Blood 106:2693-2699; Ohishi, et al., 2002, J. Clin. Invest. 110:1165-1174; Delaney, et al., 2010, Nature Med. 16(2): 232-236; and Chapter 2 of Regenerative Medicine, Department of Health and Human Services, August 2006, and the references cited therein. The collection and processing of other cell types described herein are known by one of ordinary skill in the art.

In particular embodiments, the isolating, incubating, expansion, and/or engineering steps are carried out in a sterile or contained environment and/or in an automated fashion, such as controlled by a computer attached to a device in which the steps are performed.

(viii) Production of CARs. A CAR according to the present disclosure can be produced by any means known in the art. In particular embodiments, a CAR is produced using recombinant DNA techniques. A nucleic acid sequence encoding the several regions of the CAR can be prepared and assembled into a complete coding sequence by standard techniques of molecular cloning (genomic library screening, polymerase chain reaction (PCR), primer-assisted ligation, scFv libraries from yeast and bacteria, site-directed mutagenesis, etc.). The resulting coding region can be inserted into an expression vector and used to transform a suitable expression cell line.

The term “gene” refers to a nucleic acid sequence (used interchangeably with polynucleotide or nucleotide sequence) that encodes an anti-NK CAR, components of an anti-NK CAR, or a molecule co-expressed with an anti-NK CAR as described herein. This definition includes various sequence polymorphisms, mutations, and/or sequence variants wherein such alterations do not substantially affect the function of the encoded protein. The term “gene” may include not only coding sequences but also regulatory regions such as promoters, enhancers, and termination regions. The term further can include all introns and other DNA sequences spliced from an mRNA transcript, along with variants resulting from alternative splice sites. Gene sequences encoding a molecule can be DNA or RNA that directs the expression of the molecule. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

The sequence of the open reading frame encoding, for example, a portion of a CAR, can be obtained from a genomic DNA source, a cDNA source, or can be synthesized (e.g., via PCR), or combinations thereof. Depending upon the size of the genomic DNA and the number of introns, it may be desirable to use cDNA, or a combination thereof, as introns can stabilize the mRNA or provide cell-specific expression. Also, it may be further advantageous to use endogenous or exogenous non-coding regions to stabilize the mRNA.

Polynucleotide gene sequences encoding more than one portion of an expressed CAR can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

In an exemplary nucleic acid construct (polynucleotide) employed in the present disclosure, the promoter is operably linked to the nucleic acid sequence encoding a CAR, i.e., they are positioned so as to promote transcription of mRNA from the DNA encoding the CAR. The promoter can be of genomic origin or synthetically generated. The promoters may or may not be associated with enhancers, wherein the enhancers may be naturally associated with the particular promoter or associated with a different promoter. A variety of promoters for use in cells are well-known in the art (e.g., a CD4 promoter). The promoter can be constitutive or inducible, where induction is associated with a specific cell type or a specific stage of development, for example. Alternatively, a number of well-known viral promoters are also suitable. Promoters of interest include: a viral simian virus 40 (SV40) (e.g., early or late) promoter; a Moloney murine leukemia virus (MoMLV) long terminal repeat (LTR) promoter; a Rous sarcoma virus (RSV) LTR promoter; a herpes simplex virus (HSV) (thymidine kinase) promoter; a glyceraldehyde 3-phosphate dehydrogenase (GAPDH) promoter; heat shock protein 70 kDa (HSP70) promoter; a Ubiquitin C (UBC) promoter; or a phosphoglycerate kinase-1 (PGK) promoter. Particular embodiments may utilize a myeloproliferative sarcoma virus enhancer, negative control region deleted, d1587rev primer-binding site substituted (MND) promoter (Challita et al. (1995) J. Virol. 69:748-755). In particular embodiments, a MND promoter is a synthetic promoter that contains the U3 region of a modified Moloney Murine Leukemia Virus (MoMuLV) long terminal repeat (LTR) with a myeloproliferative sarcoma virus enhancer. In particular embodiments, a MND promoter includes SEQ ID NO: 175.

For expression of a CAR, an exogenous transcriptional initiation region can be used that allows for constitutive or inducible expression, wherein expression can be controlled depending upon the target host, the level of expression desired, the nature of the target host, and the like.

Likewise, a signal sequence directing the CAR to the surface membrane can be an endogenous signal sequence of the N-terminal component of the CAR. Optionally, in some instances, it may be desirable to exchange this sequence for a different signal sequence. However, the signal sequence selected should be compatible with the secretory pathway of the CAR expressing cells so that the CAR is presented on the surface of the CAR expressing cell.

Similarly, a termination region may be provided by the naturally occurring or endogenous transcriptional termination region of the nucleic acid sequence encoding the C-terminal component of the CAR. Alternatively, the termination region may be derived from a different source. For the most part, the source of the termination region is generally not considered to be critical to the expression of a recombinant protein and a wide variety of termination regions can be employed without adversely affecting expression.

As will be appreciated by one of skill in the art, in some instances, a few amino acids at the ends of the binding domain in the CAR can be deleted, usually not more than 10, more usually not more than 5 residues, for example. Also, it may be desirable to introduce a small number of amino acids at the borders, usually not more than 10, more usually not more than 5 residues. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons.

In any of the embodiments described herein, a polynucleotide can include a polynucleotide that encodes a self-cleaving polypeptide, wherein the polynucleotide encoding the self-cleaving polypeptide is located between the polynucleotide encoding the CAR and a polynucleotide encoding a transduction marker (e.g., BFP). Exemplary self-cleaving polypeptides include 2A peptides from porcine teschovirus-1 (P2A), Thosea asigna virus (T2A), equine rhinitis A virus (E2A), foot-and-mouth disease virus (F2A), potyvirus 2A, cardiovirus 2A, or variant thereof. Further exemplary nucleic acid and amino acid sequences of 2A peptides are set forth in, for example, Kim et al. (PLOS One 6:e18556 (2011)) and Donnelly et al. (J. Gen. Virol. 82:1027-1041 (2001)). In particular embodiments, exemplary 2A peptide sequences include SEQ ID NOs: 176-185, and 240. In particular embodiments, an exemplary 2A peptide sequence is encoded by SEQ ID NO: 241.

Desired genes encoding CAR can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, mammalian artificial chromosomes (Vos, 1998, Curr. Op. Genet. Dev. 8:351-359), liposomes (Tarahovsky and Ivanitsky, 1998, Biochemistry (Mosc) 63:607-618), ribozymes (Branch and Klotman, 1998, Exp. Nephrol. 6:78-83), triplex DNA (Chan and Glazer, 1997, J. Mol. Med. 75:267-282), etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressed by the cell and, in certain instances, preferably heritable and expressed in its cell progeny.

In particular embodiments, a gene encoding a CAR can be introduced into cells in a vector. A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

Vectors derived from viruses can be used for gene delivery. Viruses that can be used include adenoviruses, adeno-associated viruses (AAV), and alphaviruses. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686.

In particular embodiments, vectors that can be used include retroviral vectors (see Miller, et al., 1993, Meth. Enzymol. 217:581-599). The term “retroviral vector” refers to a viral vector or plasmid containing structural and functional genetic elements, or portions thereof, that are primarily derived from a retrovirus. In particular embodiments, the transfer results in integration of the nucleic acid into the genome of the cell.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Where reference is made herein to elements such as cloning sites, promoters, regulatory elements, heterologous nucleic acids, etc., it is to be understood that the sequences of these elements are present in RNA form in the retroviral particles and are present in DNA form in the DNA plasmids.

In particular embodiments, a retroviral vector can include a lentiviral vector. The term “lentiviral vector” refers to a viral vector or plasmid containing structural and functional genetic elements, or portions thereof, that are primarily derived from a lentivirus. “Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV). A variety of lentiviral vectors are known in the art (Naldini et al. (1996) Science 272(5259): 263-267; Naldini et al. (1996) Proceedings of the National Academy of Sciences 93(21): 11382-11388; Zufferey et al. (1997) Nature biotechnology 15(9): 871-875; Dull et al. (1998) Journal of virology 72(11): 8463-8471; U.S. Pat. Nos. 6,013,516; and 5,994,136), many of which may be adapted to produce a viral vector or transfer plasmid.

In particular embodiments, a viral vector includes an MND promoter operably linked to a gene encoding a CAR including a binding domain that binds NKp46 or NKG2A, a CD8α hinge, a CD8α transmembrane domain, an intracellular CD3ζ signaling domain, and an intracellular 4-1BB co-stimulatory signaling domain. In particular embodiments, the viral vector includes a gene encoding a BFP transduction marker linked to the gene encoding the CAR by a 2A cleavable peptide. In particular embodiments, the binding domain that binds NKp46 includes: an NKp46 scFv including CDRs of SEQ ID NOs: 3-98, 260-265; an NKp46 scFv including VH and VL of SEQ ID NOs: 99-112; an NKp46 scFv including SEQ ID NOs: 113-119; an antigen binding fragment of a heavy chain including SEQ ID NO: 266; and/or an antigen binding fragment of a light chain including SEQ ID NO: 267. In particular embodiments, the binding domain that binds NKp46 includes: an NKp46 scFv including CDRs of SEQ ID NOs: 190, 193, 196, 198, 201, and 202; an NKp46 scFv including VH and VL of SEQ ID NOs: 204 and 205; and/or an NKp46 scFv including SEQ ID NO: 206. In particular embodiments, the binding domain that binds NKp46 includes an NKp46 scFv including CDRs of SEQ ID NOs: 190, 191, 192, 193, 194, 195, 196, 197, 198, 199, 200, 201, 202, and 203. In particular embodiments, the binding domain that binds NKG2A includes an artificial HLA-E mimetic including SEQ ID NOs: 127, 130, 133, 136, 142, and 143. In particular embodiments, the binding domain that binds NKG2A includes an artificial HLA-E mimetic including SEQ ID NOs: 242, 244, and 246. In particular embodiments, the binding domain that binds NKG2A includes: CDRs of SEQ ID NOs: 213, 214, 215, 216, 217, 218, 290, 291, 292, 293, 294, and 295; VH of SEQ ID NOs: 219, 220, 221, 222, and 223; an antigen binding fragment of a heavy chain of SEQ ID NOs: 224, 225, 226, 227, and 228; and/or an antigen binding fragment of a light chain of SEQ ID NO: 229.

In particular embodiments, a CAR of the disclosure includes from amino-terminal to carboxy-terminal: 1) a CD8 signal peptide set forth in SEQ ID NO: 238 and/or encoded by a nucleotide sequence set forth in SEQ ID NO: 239; 2) an anti-NKp46 scFv set forth in SEQ ID NO: 206 and/or encoded by a nucleotide sequence set forth in SEQ ID NO: 248; 3) a CD8 hinge and CD8 transmembrane domain set forth in SEQ ID NO: 234 and/or encoded by a nucleotide sequence set forth in SEQ ID NO: 235; 4) an intracellular signaling domain including a 4-1BB costimulatory domain and a CD3ζ stimulatory domain set forth in SEQ ID NO: 230 and/or encoded by a nucleotide sequence set forth in SEQ ID NO: 231; 5) a 2A peptide set forth in SEQ ID NO: 240 and/or encoded by a nucleotide sequence set forth in SEQ ID NO: 241; and 6) a BFP set forth in SEQ ID NO: 236 and/or encoded by a nucleotide sequence set forth in SEQ ID NO: 237. In particular embodiments, a CAR of the disclosure includes an amino acid sequence of an anti-NKp46 CAR set forth in SEQ ID NO: 249. In particular embodiments, a CAR of the disclosure includes an anti-NKp46 CAR encoded by a nucleotide sequence set forth in SEQ ID NO: 250.

In particular embodiments, a CAR of the disclosure includes from amino-terminal to carboxy-terminal: 1) a B2M signal peptide set forth in SEQ ID NO: 130 and/or encoded by SEQ ID NO: 208; 2) an HLA-G signal peptide of VMAPRTLFL (SEQ ID NO: 127) and/or encoded by SEQ ID NO: 207; 3) a (GGGGS)₃ flexible linker set forth in SEQ ID NO: 142 and/or encoded by SEQ ID NO: 211; 4) a B2M mature protein set forth in SEQ ID NO: 133 and/or encoded by SEQ ID NO: 209; 5) a (GGGGS)₄ flexible linker set forth in SEQ ID NO: 143 and/or encoded by SEQ ID NO: 212; and 6) an HLA-E 01:03 heavy chain set forth in SEQ ID NO: 136 and/or encoded by SEQ ID NO: 210; 7) an intracellular signaling domain including a 4-1BB costimulatory domain and a CD3ζ stimulatory domain set forth in SEQ ID NO: 230 and/or encoded by a nucleotide sequence set forth in SEQ ID NO: 231; 8) a 2A peptide set forth in SEQ ID NO: 240 and/or encoded by a nucleotide sequence set forth in SEQ ID NO: 241; and 9) a BFP set forth in SEQ ID NO: 236 and/or encoded by a nucleotide sequence set forth in SEQ ID NO: 237. In particular embodiments, a CAR of the disclosure includes an amino acid sequence of an anti-NKG2A (HLA-E) CAR set forth in SEQ ID NO: 251. In particular embodiments, a CAR of the disclosure includes an anti-NKG2A (HLA-E) CAR encoded by a nucleotide sequence set forth in SEQ ID NO: 252.

In particular embodiments, a CAR of the disclosure includes from amino-terminal to carboxy-terminal: 1) a B2M signal peptide set forth in SEQ ID NO: 130 and/or encoded by SEQ ID NO: 208; 2) an HLA-G signal peptide of VMAPRTLFL (SEQ ID NO: 127) and/or encoded by SEQ ID NO: 207; 3) a (GGGGS)₃ flexible linker set forth in SEQ ID NO: 142 and/or encoded by SEQ ID NO: 211; 4) a B2M mature protein set forth in SEQ ID NO: 133 and/or encoded by SEQ ID NO: 209; 5) a (GGGGS)₄ flexible linker set forth in SEQ ID NO: 143 and/or encoded by SEQ ID NO: 212; and 6) an HLA-E 01:03 heavy chain set forth in SEQ ID NO: 136 and/or encoded by SEQ ID NO: 210; 7) a CD8 hinge and CD8 transmembrane domain set forth in SEQ ID NO: 234 and/or encoded by a nucleotide sequence set forth in SEQ ID NO: 235; 8) an intracellular signaling domain including a 4-1BB costimulatory domain and a CD3ζ stimulatory domain set forth in SEQ ID NO: 230 and/or encoded by a nucleotide sequence set forth in SEQ ID NO: 231; 9) a 2A peptide set forth in SEQ ID NO: 240 and/or encoded by a nucleotide sequence set forth in SEQ ID NO: 241; and 10) a BFP set forth in SEQ ID NO: 236 and/or encoded by a nucleotide sequence set forth in SEQ ID NO: 237. In particular embodiments, a CAR of the disclosure includes an amino acid sequence of an anti-NKG2A (HLA-E) CAR set forth in SEQ ID NO: 253. In particular embodiments, a CAR of the disclosure includes an anti-NKG2A (HLA-E) CAR encoded by a nucleotide sequence set forth in SEQ ID NO: 254.

In particular embodiments, a CAR of the disclosure includes from amino-terminal to carboxy-terminal: 1) a CD8 signal peptide set forth in SEQ ID NO: 238 and/or encoded by SEQ ID NO: 239; 2) an HLA-G signal peptide of VMAPRTLFL (SEQ ID NO: 127) and/or encoded by SEQ ID NO: 207; 3) a (GGGGS)₃ flexible linker set forth in SEQ ID NO: 142 and/or encoded by SEQ ID NO: 211; 4) a B2M mature protein set forth in SEQ ID NO: 133 and/or encoded by SEQ ID NO: 209; 5) a (GGGGS)₄ flexible linker set forth in SEQ ID NO: 143 and/or encoded by SEQ ID NO: 212; and 6) an HLA-E 01:03 heavy chain set forth in SEQ ID NO: 136 and/or encoded by SEQ ID NO: 210; 7) an intracellular signaling domain including a 4-1BB costimulatory domain and a CD3ζ stimulatory domain set forth in SEQ ID NO: 230 and/or encoded by a nucleotide sequence set forth in SEQ ID NO: 231; 8) a 2A peptide set forth in SEQ ID NO: 240 and/or encoded by a nucleotide sequence set forth in SEQ ID NO: 241; and 9) a BFP set forth in SEQ ID NO: 236 and/or encoded by a nucleotide sequence set forth in SEQ ID NO: 237. In particular embodiments, a CAR of the disclosure includes an amino acid sequence of an anti-NKG2A (HLA-E) CAR set forth in SEQ ID NO: 255. In particular embodiments, a CAR of the disclosure includes an anti-NKG2A (HLA-E) CAR encoded by a nucleotide sequence set forth in SEQ ID NO: 256.

In particular embodiments, a CAR of the disclosure includes from amino-terminal to carboxy-terminal: 1) a CD8 signal peptide set forth in SEQ ID NO: 238 and/or encoded by SEQ ID NO: 239; 2) a B2M signal peptide set forth in SEQ ID NO: 130 and/or encoded by SEQ ID NO: 208; 3) an HLA-G signal peptide of VMAPRTLFL (SEQ ID NO: 127) and/or encoded by SEQ ID NO: 207; 4) a (GGGGS)₃ flexible linker set forth in SEQ ID NO: 142 and/or encoded by SEQ ID NO: 211; 5) a B2M mature protein set forth in SEQ ID NO: 133 and/or encoded by SEQ ID NO: 209; 6) a (GGGGS)₄ flexible linker set forth in SEQ ID NO: 143 and/or encoded by SEQ ID NO: 212; and 7) an HLA-E 01:03 heavy chain set forth in SEQ ID NO: 136 and/or encoded by SEQ ID NO: 210; 8) a CD8 hinge and CD8 transmembrane domain set forth in SEQ ID NO: 234 and/or encoded by a nucleotide sequence set forth in SEQ ID NO: 235; 9) an intracellular signaling domain including a 4-1BB costimulatory domain and a CD3ζ stimulatory domain set forth in SEQ ID NO: 230 and/or encoded by a nucleotide sequence set forth in SEQ ID NO: 231; 10) a 2A peptide set forth in SEQ ID NO: 240 and/or encoded by a nucleotide sequence set forth in SEQ ID NO: 241; and 11) a BFP set forth in SEQ ID NO: 236 and/or encoded by a nucleotide sequence set forth in SEQ ID NO: 237. In particular embodiments, a CAR of the disclosure includes an amino acid sequence of an anti-NKG2A (HLA-E) CAR set forth in SEQ ID NO: 257. In particular embodiments, a CAR of the disclosure includes an anti-NKG2A (HLA-E) CAR encoded by a nucleotide sequence set forth in SEQ ID NO: 258.

In particular embodiments, transduction of cells to express a CAR includes transfection of a viral vector encoding a CAR into packaging cells to obtain producer cells that make viral particles that are then used to transduce cells destined to express the CAR. A packaging cell line does not contain a packaging signal but does stably or transiently express viral structural proteins and replication enzymes (e.g., gag, pol and env) which, with elements on the viral vector encoding a CAR, are necessary for the correct packaging of viral particles. Any suitable cell line can be employed to prepare packaging cells. Generally, the cells are mammalian cells. The production of infectious viral particles and viral stock solutions may be carried out using conventional techniques (e.g., Soneoka et al. (1995) Nucl. Acids Res. 23:628-633, and Landau et al. (1992) J. Virol. 66:5110-5113). Infectious virus particles may be collected from the packaging cells using conventional techniques.

Targeted genetic engineering approaches may also be utilized to introduce a nuclei acid encoding a CAR into cells. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biot, 2013, 14(I):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Cells that have been successfully genetically modified to express a CAR can be sorted based on, for example, expression of a transduction marker, and further processed.

(ix) Assays to Characterize CAR Expressing Cells. Any relevant assay or test well known to those in the art may be used to determine whether an anti-NK CAR modified cell can bind and/or kill a target cell expressing an NK surface marker (e.g., NKp46 and/or NKG2A). In particular embodiments, an anti-NK CAR modified cell can bind and/or kill a target cell when the anti-NK CAR modified cell is activated. Assessment of anti-NK CAR modified cell activation includes: (i) induction of CD137 (4-1BB) expression on anti-NK CAR modified cells upon binding NK cell surface markers on target cells; (ii) secretion of cytokines including IL-2, IFNγ, tumor necrosis factors (e.g., TNFα), interleukin (IL)-5, and IL-13 (Xhangolli et al. (2019) Genomics, proteomics & bioinformatics, 17(2): 129-139), and/or (iii) cytotoxicity towards target cells expressing NK cell surface markers (e.g., NKp46 and/or NKG2A). For example, assessing anti-NK CAR modified cell function can include the following. Target cells genetically modified to express an NK cell surface marker such as NKp46 can be contacted with anti-NK CAR modified cells (effector cells) having an anti-NKp46 scFv binding domain in a ratio of effector cell:target cell of 2:1. In particular embodiments, a ratio of effector cell:target cell can include 1:1, 2:1, 3:1, 4:1, 5:1, 6:1, 7:1, 8:1, 9:1, and 10:1. In particular embodiments, target cells include K562 cells, which are derived from a human immortalized myelogenous leukemia cell line. K562 cells lack the MHC complex, lack any trace of EBV, and spontaneously develop characteristics similar to early-stage erythrocytes, granulocytes, and monocytes. In particular embodiments, target cells include an NK cell lymphoma cell line such as NK92. In particular embodiments, target cells include autologous primary NK cells obtained from PBMCs from a donor. In particular embodiments, the anti-NK CAR modified cells are anti-NK CAR modified T cells and can be selectively observed in a flow cytometer by, e.g., gating for CD3+ (T cell marker) and BFP+ (fluorescence emission wavelength indicative of a transduction marker co-expressed with the CAR) cells.

In particular embodiments, induction of CD137 expression on anti-NK CAR modified cells upon binding of target cells can be measured by flow cytometry using antibodies that recognize CD137. The % of CD137+ anti-NK CAR modified T cells can be measured by flow cytometry after the anti-NK CAR modified T cells have been contacted with a labeled antibody that binds CD137. Antibodies that bind CD137 are commercially available, including a rabbit monoclonal [BLR051F] anti-human CD137 antibody (Abcam, Cambridge, UK), a mouse monoclonal 4B4-1 anti-human CD137 antibody (Thermo Fisher, Waltham, MA), and mouse monoclonal BBK-2 anti-human CD137 antibody (Santa Cruz Biotechnology, Dallas, TX).

In particular embodiments, secretion of cytokines from anti-NK CAR modified cells upon binding of target cells can be measured by assays known in the art. For example, secretion of cytokines can be measured by ELISA, Western blot, flow cytometry, single cell multiplex cytokine profiling, and high-throughput single cell 3′ mRNA transcriptome sequencing. In particular embodiments, the assays use antibodies that bind specifically to particular cytokines. In particular embodiments, the assays use flow cytometry to measure intracellular staining of cytokines. In particular embodiments, the assays use cellular barcoding of 3′ mRNA and high-throughput sequencing. In particular embodiments, the cytokines measured include IL-2, IFNγ, and tumor necrosis factors (e.g., TNFα). Cytokine assays are further described in Wilkie et al. (2008) J Immunol. 180:4901-4909, Jena et al. (2014) Curr Hematol Malig Rep. 9:50-56, Kaiser et al. (2015) Cancer Gene Ther. 22:72-78, Xue et al. (2017) J Immunother Cancer 5:85, and Xhangolli et al. (2019) Genomics Proteomics Bioinformatics. 17(2): 129-139.

In particular embodiments, the cytotoxic activity of anti-NK CAR modified cells upon binding of target cells can be measured by flow cytometry. In particular embodiments, a labeled antibody against an NK cell surface marker expressed by a target cell can be used to quantify the amount of target cells in the presence or absence of anti-NK CAR modified cells by flow cytometry. In particular embodiments, cytotoxicity assays include chromium-51 release assay or similar assay using a non-radioactive reporter such as enhanced green fluorescent protein (GFP)-firefly luciferase fusion (Xiong et al. (2018) J Immunol 200(1): Supplement 1, 179.2), lactate dehydrogenase (LDH) release assay, real time cell analyzing systems that use gold microelectrode biosensors to quantify cell viability by electrical impedance (e.g., Sener et al. (2017) Exp Ther Med. 14(3): 1866-1870), and real time systems based on probes such as propidium iodide and SYTOX Green based on compromised cellular membrane integrity. In a release assay, target cell death can be quantified by the amount of chromium 51 (pre-loaded into target cells before contact with a CAR T cell) or LDH released from lysed cells. Target cell cytotoxicity can be calculated using the following formula: % of cytotoxicity=100×[(CAR-T:target cells−CAR-T cells alone−target cells alone)/(maximum target cell lysis−target cells alone without lysis buffer)]. In particular embodiments, the cytotoxic activity of anti-NK CAR modified cells upon binding of target cells can be assessed by measuring the decrease in the percentage of target cells pre-stained with a label or dye such as CellTracker Orange or Green (Thermo Fisher Scientific, Waltham, MA).

In particular embodiments, an activated anti-NK CAR modified cell can induce a statistically significant (e.g., p<0.05) increase in activation of the anti-NK CAR modified cell after contact of the anti-NK CAR modified cell with a target cell. In particular embodiments, activation of an anti-NK CAR modified cell after contact of the anti-NK CAR modified cell with a target cell can be assessed by the amount or fold increase of anti-NK CAR modified cells that are CD137+ as described herein. In particular embodiments, activation of an anti-NK CAR modified cell after contact of the anti-NK CAR modified cell with a target cell can be assessed by the amount or fold increase of anti-NK CAR modified cells producing cytokines or by the amount or fold increase of cytokines (e.g., IFNγ, TNFα, IL-2) produced as described herein. In particular embodiments, an activated anti-NK CAR modified cell has an increase in activation of 5%, 15%, 25%, 35%, 45%, 55%, 65%, 75%, 85%, 95%, 100%, or more, as compared to a control condition. In particular embodiments, an activated anti-NK CAR modified cell has an increase in activation of 10% to 50% as compared to a control condition. In particular embodiments, the increase in anti-NK CAR modified cell activation is 2-fold, 4-fold, 6-fold, 8-fold, 10-fold, 12-fold, 14-fold, or greater as compared to a control condition. In particular embodiments, the increase in anti-NK CAR modified cell activation is 1.5-fold to 6-fold as compared to a control condition. In particular embodiments, activated anti-NK CAR modified cells can induce a statistically significant (e.g., p<0.05) decrease in the amount of viable target cells after contact of the anti-NK CAR modified cells with target cells. In particular embodiments, a decrease in the amount of viable target cells measures depletion of target cells upon exposure to a CAR of the present disclosure. In particular embodiments, a decrease in the amount of viable target cells can be assessed by measuring a decrease in target cells marked with a label or dye as described herein. In particular embodiments, the amount of viable target cells decreases 5%, 15%, 25%, 35%, 45%, 55%, 65%, 75%, 85%, 95%, 100%, 125%, 150%, 175%, 200%, or more as compared to a control condition. In particular embodiments, the amount of viable target cells decreases 10% to 100% as compared to a control condition. In particular embodiments, the amount of viable target cells decreases 2-fold, 4-fold, 6-fold, 8-fold, 10-fold, or greater as compared to a control condition. In particular embodiments, the amount of viable target cells decreases 2-fold to 5-fold as compared to a control condition. In particular embodiments, a control condition can include: an assay performed with anti-NK CAR modified cells alone (no target cells present); an assay performed with target cells alone (no anti-NK CAR modified cells present); an assay performed with anti-NK CAR modified cells and target cells expressing an antigen not recognized by the binding domain of the CAR expressed by the anti-NK CAR modified cells (e.g., anti-NKp46 modified CAR T cells and K562 cells expressing CD19).

(x) Assays to Characterize Target NK Cells. Any number of assays known in the art can be used to characterize properties and/or behavior of NK cells targeted by anti-NK CAR expressing cells of the disclosure. NK cells can be characterized, for example, after contacting target NK cells with anti-NK CAR expressing cells. In particular embodiments, a ratio of NK target cell:anti-NK CAR expressing cell can include 1:1, 1:2, 1:3, 1:4, 1:5, 1:6, 1:7, 1:8, 1:9, and 1:10. Any parameter associated with NK cell activity can be measured or detected, such as markers of cytotoxicity (CD107, CD69), cytokine production (e.g., IFN-γ or TNF-α), increases in intracellular free calcium levels, and/or the ability to lyse cells. In particular embodiments, intracellular cytokine expression by NK cells can be assessed by flow cytometry. In particular embodiments, NK cells can be labeled with a cell tracker dye to differentiate the NK cells from the anti-NK CAR expressing cells. In particular embodiments, anti-NK CAR expressing cells can be identified by being cell tracker dye negative, CD3+, and/or selectable marker positive by flow cytometry. NK cell activity can be assessed by gene expression-based activities, cytotoxicity-based assays, and proliferation assays. Useful protocols for assays involving NK cells can be found in Natural Killer Cells Protocols (edited by Campbell K S and Colonna M. Humana Press, pp. 219-238 (2000)).

In particular embodiments, an NK cell can show a statistically significant (e.g., p<0.05) increase in activation after contact with an anti-NK CAR expressing cell. In particular embodiments, NK cell activation is reflected as an increase in cytokine production of 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, or more, as compared to a control condition. In particular embodiments, an NK cell can show a statistically significant (e.g., p<0.05) increase in inhibition after contact with an anti-NK CAR expressing cell. In particular embodiments, NK cell inhibition is reflected as a decrease in cytokine production of 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, or more, as compared to a control condition. In particular embodiments, an NK cell does not show any statistically significant (e.g., p>0.05) increase in activation or increase in inhibition after contact with an anti-NK CAR expressing cell. In particular embodiments, NK cells can induce a statistically significant (e.g., p<0.05) decrease in the amount of anti-NK CAR expressing cells after contact of the NK cells with anti-NK CAR expressing cells (e.g., as assessed by cell tracker dye negative, CD3+, and/or selectable marker positive for CAR expressing cells). In particular embodiments, the amount of anti-NK CAR expressing cells decreases 1%, 2%, 3%, 4%, 5%, 6%, 7%, 8%, 9%, 10%, 11%, 12%, 13%, 14% 15%, 20%, 25%, 30%, 35%, 40%, 45%, 50%, 55%, 60%, 65%, 70%, 75%, 80%, 85%, 90%, 95%, or more as compared to a control condition. In particular embodiments, NK cells do not induce a statistically significant (e.g., p<0.05) decrease in the amount of anti-NK CAR expressing cells after contact of the NK cells with anti-NK CAR expressing cells. In particular embodiments, a control condition can include: an assay performed with NK cells and cells not modified to express an anti-NK CAR (e.g., mock T cells); an assay performed with NK cells and cells modified to express an anti-activating NK receptor CAR (e.g., anti-NKp46 CAR cells); an assay performed with NK cells and cells modified to express an anti-inhibitory NK receptor CAR (e.g., anti-NKG2A CAR cells); an assay performed with NK cells and cells modified to express a CAR including a binding domain that does not recognize an antigen on an NK cell surface (e.g., cells expressing an anti-CD19 CAR).

(xi) Compositions and Formulations. Cells genetically modified ex vivo to express a CAR and/or nanoparticles that result in in vivo genetic modification of cells to express a CAR can be formulated for administration to subjects.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration (US FDA) as being acceptable for use in humans or domestic animals. Exemplary pharmaceutically acceptable carriers and formulations are disclosed in Remington's Pharmaceutical Sciences, 18th Ed. Mack Printing Company, 1990. Moreover, compositions and formulations can be prepared to meet sterility, pyrogenicity, general safety, and purity standards as required by the US FDA Office of Biological Standards and/or other relevant foreign regulatory agencies.

Exemplary carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof. In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols, amino acids, organic sugars or sugar alcohols, PEG, sulfur-containing reducing agents, bovine serum albumin, gelatin or immunoglobulins, polyvinylpyrrolidone, and saccharides.

Where necessary or beneficial, compositions or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, catechol, resorcinol, cyclohexanol, and 3-pentanol.

Therapeutically effective amounts of cells within compositions or formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions and formulations disclosed herein, cells are generally in a volume of a liter or less, 500 ml or less, 250 ml or less, or 100 ml or less. Hence the density of administered cells is typically greater than 10⁴ cells/ml, 10⁷ cells/ml, or 10⁸ cells/ml.

Therapeutically effective amounts of nanoparticles can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

In particular embodiments, compositions and formulations can include one or more genetically modified cell type (e.g., modified T cells, NK cells, or stem cells) or genetically modified cells with one or more CAR types (e.g., modified T cells including an anti-NKp46 CAR and modified T cells including an anti-NKG2A CAR). The different populations of genetically modified cells can be provided in different ratios.

The cell-based compositions and formulations can be prepared for administration by, e.g., injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

In some instances, it can be useful to cryopreserve cells or cell formulations of the disclosure. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to ameliorate or prevent cell damage due to freezing at low temperatures or warming to room temperature. Cryoprotective agents and optimal cooling rates can protect against cell injury. Cryoprotective agents which can be used include dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). In particular embodiments, the cooling rate is 1° to 3° C./minute. After at least two hours, the cells reach a temperature of −80° C. and can be placed directly into liquid nitrogen (−196° C.) for permanent storage such as in a long-term cryogenic storage vessel.

(xii) Methods of Use. The genetically modified cells described herein provide methods for treatment of an NK cell associated disease as well as treatment of other diseases that are associated with cells that express NK receptors. In particular embodiments, an NK cell associated disease includes any disease where NK cells play a role, including being the cause or progression of the disease (e.g., in NK cell malignancy, autoimmune disease, alloimmune disease, or infection) or functioning to ameliorate or prevent the disease (e.g., in cancer). In particular embodiments, an NK cell associated disease includes an NK cell malignancy, a cancer, an autoimmune disease, or an alloimmune disease. In particular embodiments, an NK cell associated disease includes malignancies or infections in which there would be therapeutic benefit to improving or boosting NK cell function by depleting NK cells expressing more inhibitory receptors or having an inhibitory phenotype. In particular embodiments, an NK cell inhibitory phenotype includes: more expression of inhibitory receptors on an NK cell; inhibition of NK cell activity; a decrease in the ability of an NK cell to lyse a target cell; and/or a decrease in NK cell immune response against inflamed, infected, tumor, and/or other cells that need to be destroyed, as compared to a normal functioning NK cell or an NK cell without an inhibitory phenotype. In particular embodiments, an NK cell inhibitory phenotype can be measured by assays as described herein.

In particular embodiments, diseases that are associated with cells that express NK cell receptors include malignancies with aberrant NK receptor expression. In particular embodiments, NK receptor expression on cells in a population of cells is aberrant when 5%, 10%, 15%, 20%, 25%, 30%, 35%, 40%, 45%, 50%, 55%, 60%, 65%, 70%, 75%, 80%, 85%, 90%, 95%, or 100% of cells in the population express a given NK receptor. The percentage of cells in a population of cells that express an NK receptor can be compared to a control condition or reference value. In particular embodiments, the control condition can include cells in a population of cells from a healthy subject or a pool of healthy subjects, or cells in a population of cells from a subject or a pool of subjects not afflicted with a malignancy with aberrant NK receptor expression. In particular embodiments, a reference value can be derived from cells in a population of cells from a healthy subject or a pool of healthy subjects, or from cells in a population of cells from a subject or a pool of subjects not afflicted with a malignancy with aberrant NK receptor expression. Malignancies with aberrant NK receptor expression include Sezary syndrome, mature T cell neoplasms, T cell large granular lymphocytic leukemia, mycosis fungoides, and ALK+ anaplastic large cell lymphoma. In particular embodiments, diseases that are associated with cells that express NK cell receptors include autoimmune or alloimmune conditions in which NK receptors are expressed on T cells or other cells.

In particular embodiments, anti-NK modified cells expressing a CAR that binds NKp46 and/or NKG2A allows for depletion of NK cells to treat NK cell malignancies. In particular embodiments, anti-NK modified cells expressing a CAR that binds an NKG2A inhibitory receptor allows for depletion of inhibitory NK cells to boost NK responses to cancer. In particular embodiments, cancer includes non-NK cell malignancies. In particular embodiments, anti-NK modified cells expressing a CAR that binds an NKG2A inhibitory receptor allows for depletion of inhibitory NK cells to boost NK responses to infection. In particular embodiments, anti-NK modified cells expressing a CAR that binds an NKp46 activating receptor allows for depletion of activating NK cells to reduce an immune response in autoimmune or alloimmune diseases.

In particular embodiments, the anti-NK CAR modified cells are administered to a subject in need thereof. The administered cells can target and destroy NK cells in the subject. In particular embodiments, the anti-NK CAR modified cells are anti-NK CAR modified T cells that are able to replicate in vivo, resulting in long-term persistence that can lead to sustained therapy. In particular embodiments, the anti-NK CAR modified T cells can undergo robust in vivo T cell expansion and can persist for an extended amount of time. In particular embodiments, the anti-NK CAR modified T cells evolve into specific memory T cells that can be reactivated to target and destroy NK cells. In particular embodiments, the anti-NK CAR modified cells are anti-NK CAR modified HSPC that differentiate into mature immune effector cells in vivo following administration to a subject.

In particular embodiments, a subject in need thereof is administered an effective amount of a composition to increase a cellular immune response to an NK cell malignancy, a cancer, an infection, an autoimmune disease, or an alloimmune disease in the subject. The immune response may include cellular immune responses mediated by regulatory T cells, helper T cell responses, and/or cytotoxic T cells capable of killing NK cells. Humoral immune responses, mediated primarily by helper T cells capable of activating B cells, thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In the case of T cell-mediated killing, CAR-antigen binding initiates CAR signaling in the T cell, resulting in activation of a variety of T cell signaling pathways that induce the T cell to produce or release proteins capable of inducing target cell apoptosis by various mechanisms. These T cell-mediated mechanisms include the transfer of intracellular cytotoxic granules from the T cell into the target cell, T cell secretion of pro-inflammatory cytokines that can induce target cell killing directly (or indirectly via recruitment of other killer effector cells), and upregulation of death receptor ligands (e.g. FasL) on the T cell surface that induce target cell apoptosis following binding to their cognate death receptor (e.g. Fas) on the target cell.

Methods include treating subjects (humans, veterinary animals (dogs, cats, reptiles, birds, etc.) livestock (horses, cattle, goats, pigs, chickens, etc.) and research animals (monkeys, rats, mice, fish, etc.) with compositions and formulations disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an anti-cancer, anti-infection, anti-autoimmune, or anti-alloimmune effect. Effective amounts are often administered for research purposes. Effective amounts can cause a statistically significant effect in an animal model or in vitro assay relevant to the assessment of development or progression of an NK cell malignancy, cancer, autoimmune disease, or alloimmune disease. An immunogenic composition can be provided in an effective amount, wherein the effective amount stimulates an immune response or dampens an immune response.

A “prophylactic treatment” includes a treatment administered to a subject who does not display signs or symptoms of an NK cell malignancy, a cancer, an infection, an autoimmune disease, or an alloimmune disease, or displays only early signs or symptoms of an NK cell malignancy, a cancer, an infection, an autoimmune disease, or an alloimmune disease, such that treatment is administered for the purpose of diminishing or decreasing the risk of developing the NK cell malignancy, the cancer, the infection, the autoimmune disease, or the alloimmune disease further. Thus, a prophylactic treatment functions as a preventative treatment against an NK cell malignancy, a cancer, an infection, an autoimmune disease, or an alloimmune disease. In particular embodiments, prophylactic treatments reduce, delay, or prevent further progression of an NK cell malignancy. In particular embodiments, prophylactic treatments reduce, delay, or prevent metastasis of a cancer. In particular embodiments, prophylactic treatments reduce, delay, or prevent an infection. In particular embodiments, prophylactic treatments reduce, delay, or prevent an overreactive immune response.

A “therapeutic treatment” includes a treatment administered to a subject who displays symptoms or signs of an NK cell malignancy, a cancer, an infection, an autoimmune disease, or an alloimmune disease and is administered to the subject for the purpose of diminishing or eliminating those signs or symptoms of the NK cell malignancy, the cancer, the infection, the autoimmune disease, or the alloimmune disease. The therapeutic treatment can reduce, control, or eliminate the presence or activity of the NK cell malignancy, the cancer, the infection, the autoimmune disease, or the alloimmune disease and/or reduce, control, or eliminate side effects of the NK cell malignancy, the cancer, the autoimmune disease, or the alloimmune disease.

NK cell malignancies include neoplasms arising from immature NK cells and mature NK cells. In particular embodiments, immature (or precursor) NK cell neoplasms include agranular CD4+/CD56+ hematodermic neoplasms, CD94 1A+/TCR− lymphoblastic lymphoma/leukemia (LBL), and myeloid/NK cell acute leukemia. In particular embodiments, mature NK cell neoplasms include: extranodal NK cell lymphoma, nasal type; aggressive NK cell leukemia; and chronic NK cell lymphocytosis. Mature NK cell neoplasms are generally CD2+/CD3−/cCD3ε+/CD56+/MPO− and cytotoxic molecule-positive. Mature NK cell neoplasms, unlike immature NK cell neoplasms, also have a strong association with EBV.

In particular embodiments, therapeutic treatments reduce, delay, or prevent nasal discharge, nasal obstruction, purulent rhinorrhea, epistaxis, and local swelling in a subject having nasal NK cell lymphoma. In particular embodiments, therapeutic treatments reduce, delay, or prevent ulceration of mucosal sites, angiocentric and angiodestructive growth of blood vessels, and skin lesions in a subject having extranasal NK cell lymphoma. In particular embodiments, therapeutic treatments reduce, delay, or prevent fever, systemic symptoms, liver dysfunction, hepatosplenomegaly, severe anemia, and thrombocytopenia in a subject having aggressive NK cell leukemia. In particular embodiments, therapeutic treatments reduce, delay, or prevent severe neutropenia, pure red cell aplasia, vasculitic syndromes, fever, and increase of peripheral blood NK cells in a subject having chronic NK cell lymphocytosis.

Cancers that can be treated by compositions and formulations include: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B cell lymphoma, T cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary cancers that can be treated according to the disclosure include hematopoietic tumors of lymphoid lineage, for example T cell and B cell tumors, including: T cell disorders such as T-prolymphocytic leukemia (T-PLL), including of the small cell and cerebriform cell type; large granular lymphocyte leukemia (LGL) of the T cell type; Sezary syndrome (SS); adult T cell leukemia lymphoma (ATLL); hepatosplenic T cell lymphoma; peripheral/post-thymic T cell lymphoma (pleomorphic and immunoblastic subtypes); angioimmunoblastic T cell lymphoma; angiocentric (nasal) T cell lymphoma; anaplastic (Ki 1+) large cell lymphoma; intestinal T cell lymphoma; and T-lymphoblastic lymphoma/leukemia (T-Lbly/T-ALL).

In particular embodiments, therapeutically effective amounts provide anti-cancer effects. Anti-cancer effects include a decrease in the number of malignant NK cells, decrease in the number of metastases, a decrease in tumor volume, an increase in life expectancy, induced chemo- or radio-sensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

Infections that can be treated by disclosed compositions and formulations include bacterial, viral, fungal, parasitic, and arthropod infections. In particular embodiments, the infections are chronic. In particular embodiments, bacterial infections can include infections caused by Staphylococcus spp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, and Treponema pallidum. In particular embodiments, viral infections can include infections caused by rhinovirus, influenza virus, respiratory syncytial virus (RSV), coronavirus, herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), Merkel cell polyomavirus (MCV), and HHV8 (Kaposi's sarcoma). In particular embodiments, fungal infections can include infections caused by Trychophyton spp. and Candida spp. In particular embodiments, parasitic infections can include infections caused by Giardia, toxoplasmosis, E. vermicularis, Trypanosoma cruzi, Echinococcosis, Cysticercosis, Toxocariasis, Trichomoniasis, and Amebiasis. In particular embodiments, arthropod infections can include infections spread by arthropods infected with viruses or bacteria, including California encephalitis, Chikungunya, dengue, Eastern equine encephalitis, Powassan, St. Louis encephalitis, West Nile, Yellow Fever, Zika, Lyme disease, and babesiosis.

In particular embodiments, therapeutically effective amounts provide anti-infection effects. Anti-infection effects include a decrease in: the amount or level of infective pathogen, fatigue, loss of appetite, weight loss, fevers, night sweats, chills, aches and pains, diarrhea, bloating, abdominal pain, skin rashes, coughing, and/or a runny nose.

Autoimmune diseases that can be treated by disclosed compositions and formulations include: Sjogren's disease; antiphospholipid syndrome; Pemphigus vulgaris; spondylarthropathies; skin diseases including psoriasis; multiple sclerosis; systemic sclerosis; Type I diabetes; rheumatoid arthritis; juvenile idiopathic arthritis; inflammatory bowel disease; autoimmune liver diseases; and systemic lupus erythematosus (SLE).

Alloimmune diseases that can be treated by disclosed compositions and formulations include: FNAIT; hematopoietic stem cell rejection, solid tissue transplant rejection; and chronic allograft injury after organ transplantation.

In particular embodiments, therapeutically effective amounts provide anti-autoimmune or anti-alloimmune effects. Anti-autoimmune or anti-alloimmune effects include a decrease in an immune response, a decrease in inflammation, a decrease in tissue injury, and/or an increase in NK cell tolerance of self or non-self tissue.

In particular embodiments, therapeutic treatments reduce, delay, or prevent: attack of cells, tissues, and joints by the immune system; swelling, stiffness, and pain in joints in rheumatoid arthritis, psoriatic arthritis, or juvenile idiopathic arthritis; numbness, weakness, and balance issues in multiple sclerosis; inflammation of the gastrointestinal tract in irritable bowel disease; dry eyes and dry mouth in Sjogren's disease; frequent clotting in arteries and veins and/or miscarriages in antiphospholipid syndrome; blisters on skin and mucous membranes in Pemphigus vulgaris; inflammatory spinal pain, sacroiliitis, chest wall pain, peripheral arthritis, peripheral enthesitis, dactylitis, lesions of the lung apices, conjunctivitis, uveitis, and aortic incompetence together with conduction disturbances in spondylarthropathies; high blood sugar level in Type I diabetes; fibrosis and vascular abnormalities in the skin, joints, and internal organs in systemic sclerosis; cirrhosis and liver failure in autoimmune liver diseases; fatigue, joint pain, rash, and fever in SLE; miscarriage and intrauterine growth restriction in FNAIT; hematopoietic stem cell transplant rejection; solid tissue transplant rejection; attack of allogeneic cells, tissues, and/or organs by the immune system; and development of de novo donor-specific antibodies, damage to the endothelium of vascular beds, and cellular hypertrophy in chronic allograft injury after kidney transplantation.

Prophylactic treatments and therapeutic treatments need not be mutually exclusive, and in particular embodiments, administered dosages may accomplish more than one treatment type.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of disease, stage of disease, previous or concurrent therapeutic interventions, idiopathy of the subject and route of administration.

Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Useful doses can range from 10⁶-10¹² cells/kg. Useful doses can include 10⁶ cells/kg, 10⁷ cells/kg, 10⁸ cells/kg, 10⁹ cells/kg, 10¹⁰ cells/kg, 10¹¹ cells/kg, 10¹² cells/kg, or more. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNFα, IL-18, and TNFβ, GM-CSF, IL-4, IL-13, Flt3-L, RANTES, MIP1α, etc.) to enhance induction of the immune response.

Useful doses to administer can range from, for example, 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, weekly, every 2 weeks, monthly, every 2 months, every 4 months, every 6 months, yearly, etc.).

As indicated, the compositions and formulations can be administered by injection, transfusion, implantation or transplantation. In particular embodiments, compositions and formulations are administered parenterally. The phrases “parenteral administration” and “administered parenterally” refer to modes of administration other than enteral and topical administration, usually by injection, and includes, intravascular, intravenous, intramuscular, intraarterial, intrathecal, intracapsular, intratumoral, intraperitoneal, and subcutaneous, injection and infusion. In particular embodiments, the compositions and formulations described herein are administered to a subject by direct injection into a tumor, lymph node, or site of disease.

In particular embodiments, compositions and formulations are administered to a patient in conjunction with (e.g., before, simultaneously or following) any number of relevant treatment modalities, such as chemotherapeutic agents, radiation, immunosuppressive or immunoablative agents, anti-inflammatory agents (e.g., steroids, glucocorticoids, nonsteroidal anti-inflammatory drugs (NSAIDS)), and/or cytokines.

In particular embodiments, compositions and formulations may be administered in combination with an anti-viral treatment or prophylaxis, for example, when treating NK cell malignancies or cancers. In particular embodiments, an anti-viral treatment or prophylaxis includes compounds used to treat EBV infections. In particular embodiments, an anti-viral treatment or prophylaxis includes nucleoside analogs including acyclovir, valacyclovir, famciclovir, ganciclovir, or valganciclovir.

(xiii) Kits. Any of the compositions described herein may be included in a kit. In particular embodiments, one or more of the following can be provided in a kit: cells to be genetically modified to express an anti-NK CAR, including immune cells; reagents suitable for expanding the cells to be modified, including media, aAPCs, growth factors, and antibodies; reagents suitable for introducing nucleic acids encoding an anti-NK CAR into cells, including reagents for transfection and/or transduction of cells; anti-NK CAR modified cells; reagents to cryopreserve cells; CAR expression constructs; reagents to generate CAR expression constructs including enzymes, polymerases, and primers; reagents suitable for characterizing the CAR modified cells including antibodies to sort or detect the CAR modified cells; and/or laboratory supplies useful for manipulation of nucleic acids and cells, including tissue culture plates, buffers, syringes, pipettes, etc.

In particular embodiments, cells for transfection of the CAR expression construct include 293 cells, 293T cells, or A549 cells. In particular embodiments, reagents for transduction of the CAR expression construct include fibronectin-coated plates, hexadimethrine bromide (Polybrene), and/or lipofectamine. In particular embodiments, the CAR expression construct includes a retroviral vector including a gene that encodes the CAR. In particular embodiments, the CAR expression construct includes an MND promoter operably linked to a CAR including a binding domain that binds an activating NK receptor and/or an inhibitory NK receptor. In particular embodiments, the CAR expression construct includes an MND promoter operably linked to a CAR including a binding domain that binds NKp46 and/or NKG2A. In particular embodiments, the CAR expression construct includes an MND promoter operably linked to a CAR including a binding domain that binds NKp46 or NKG2A, a CD8α hinge, a CD8α transmembrane domain, an intracellular CD3ζ signaling domain, and an intracellular 4-1BB co-stimulatory signaling domain. In particular embodiments, the CAR expression construct includes a gene encoding a BFP transduction marker linked to the gene encoding the CAR by a 2A cleavable peptide. In particular embodiments, the binding domain that binds NKp46 includes: an NKp46 scFv including CDRs of SEQ ID NOs: 3-98, 260-265; an NKp46 scFv including VH and VL of SEQ ID NOs: 99-112; an NKp46 scFv including SEQ ID NOs: 113-119; an antigen binding fragment of a heavy chain including SEQ ID NO: 266; and/or an antigen binding fragment of a light chain including SEQ ID NO: 267. In particular embodiments, the binding domain that binds NKp46 includes: an NKp46 scFv including CDRs of SEQ ID NOs: 190, 193, 196, 198, 201, and 202; an NKp46 scFv including VH and VL of SEQ ID NOs: 204 and 205; and/or an NKp46 scFv including SEQ ID NO: 206. In particular embodiments, the binding domain that binds NKp46 includes an NKp46 scFv including CDRs of SEQ ID NOs: 190, 191, 192, 193, 194, 195, 196, 197, 198, 199, 200, 201, 202, and 203. In particular embodiments, the binding domain that binds NKG2A includes an artificial HLA-E mimetic including SEQ ID NOs: 127, 130, 133, 136, 142, and 143. In particular embodiments, the binding domain that binds NKG2A includes an artificial HLA-E mimetic including SEQ ID NOs: 242, 244, and 246. In particular embodiments, the binding domain that binds NKG2A includes: CDRs of SEQ ID NOs: 213, 214, 215, 216, 217, 218, 290, 291, 292, 293, 294, and 295; VH of SEQ ID NOs: 219, 220, 221, 222, and 223; an antigen binding fragment of a heavy chain of SEQ ID NOs: 224, 225, 226, 227, and 228; and/or an antigen binding fragment of a light chain of SEQ ID NO: 229.

The kits may include one or more suitably aliquoted reagents to generate compositions of the disclosure. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also can generally contain a second, third, or other additional container into which the additional components may be separately placed. However, various combinations of components may be included in a container. The kits also will typically include a means for containing the CAR expression construct and any other reagent containers in close confinement for commercial sale. Such containers may include injection or blow molded plastic containers into which the desired vials are retained, for example.

(xiv) Variants. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants are conservative amino acid changes, i.e., substitutions of similarly charged or uncharged amino acids. A conservative amino acid change involves substitution of one of a family of amino acids which are related in their side chains.

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a binding domain VH region can be derived from or based on a VH of a known antibody and can optionally contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the known antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a VL region in a binding domain is derived from or based on a VL of a known antibody and optionally contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, SXSSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. SXSSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

### (xv) Exemplary Embodiments

1. A chimeric antigen receptor (CAR) that when expressed includes:
an extracellular component; and
an intracellular component,
wherein the extracellular component includes an activating NK receptor binding domain and/or an inhibitory NK receptor binding domain.
   2. The CAR of embodiment 1, further including a transmembrane domain linking the extracellular component to the intracellular component.
   3. The CAR of embodiment 1 or 2, wherein the activating NK receptor binding domain includes an NKp30 binding domain, an NKp44 binding domain, and/or an NKp46 binding domain.
   4. The CAR of embodiment 3, wherein the NKp30 binding domain includes an antigen binding fragment of antibody AZ20, antibody A76, antibody Z25, antibody 15E1, antibody 9G1, antibody 15H6, antibody 9D9, antibody 3A12, antibody 12D10, antibody clone #210845, antibody clone p30-15, or antibody clone AF29-4D12.
   5. The CAR of embodiment 3 or 4, wherein the NKp44 binding domain includes an antigen binding fragment of antibody Z231, antibody clone #253415, antibody clone 44.189, antibody clone 1G6, or antibody clone P44-8.
   6. The CAR of any one of embodiments 3-5, wherein the NKp46 binding domain includes:
(A) a heavy chain variable (VH) domain including: a CDRH1 having the sequence as set forth in SEQ ID NO: 190, a CDRH2 having the sequence as set forth in SEQ ID NO: 193, and a CDRH3 having the sequence as set forth in SEQ ID NO: 196; and a light chain variable (VL) domain including: a CDRL1 having the sequence as set forth in SEQ ID NO: 198, a CDRL2 having the as sequence set forth in SEQ ID NO: 201, and a CDRL3 having the sequence as set forth in SEQ ID NO: 202;
(B) a VH domain including: a CDRH1 having the sequence as set forth in SEQ ID NO: 3, a CDRH2 having the sequence as set forth in SEQ ID NO: 6, and a CDRH3 having the sequence as set forth in SEQ ID NO: 9; and a VL domain including: a CDRL1 having the sequence as set forth in SEQ ID NO: 12, a CDRL2 having the sequence as set forth in SEQ ID NO: 15, and a CDRL3 having the sequence as set forth in SEQ ID NO: 16;
(C) a VH domain including: a CDRH1 having the sequence as set forth in SEQ ID NO: 19, a CDRH2 having the sequence as set forth in SEQ ID NO: 22, and a CDRH3 having the sequence as set forth in SEQ ID NO: 24; and a VL domain including: a CDRL1 having the sequence as set forth in SEQ ID NO: 27, a CDRL2 having the sequence as set forth in SEQ ID NO: 30, and a CDRL3 having the sequence as set forth in SEQ ID NO: 31;
(D) a VH domain including: a CDRH1 having the sequence as set forth in SEQ ID NO: 33, a CDRH2 having the sequence as set forth in SEQ ID NO: 36, and a CDRH3 having the sequence as set forth in SEQ ID NO: 39; and a VL domain including: a CDRL1 having the sequence as set forth in SEQ ID NO: 42, a CDRL2 having the sequence as set forth in SEQ ID NO: 45, and a CDRL3 having the sequence as set forth in SEQ ID NO: 46;
(E) a VH domain including: a CDRH1 having the sequence as set forth in SEQ ID NO: 48, a CDRH2 having the sequence as set forth in SEQ ID NO: 51, and a CDRH3 having the sequence as set forth in SEQ ID NO: 54; and a VL domain including: a CDRL1 having the sequence as set forth in SEQ ID NO: 57, a CDRL2 having the sequence as set forth in SEQ ID NO: 60, and a CDRL3 having the sequence as set forth in SEQ ID NO: 61;
(F) a VH domain including: a CDRH1 having the sequence as set forth in SEQ ID NO: 63, a CDRH2 having the sequence as set forth in SEQ ID NO: 66, and a CDRH3 having the sequence as set forth in SEQ ID NO: 69; and a VL domain including: a CDRL1 having the sequence as set forth in SEQ ID NO: 42, a CDRL2 having the sequence as set forth in SEQ ID NO: 45, and a CDRL3 having the sequence as set forth in SEQ ID NO: 72;
(G) a VH domain including: a CDRH1 having the sequence as set forth in SEQ ID NO: 74, a CDRH2 having the sequence as set forth in SEQ ID NO: 77, and a CDRH3 having the sequence as set forth in SEQ ID NO: 78; and a VL domain including: a CDRL1 having the sequence as set forth in SEQ ID NO: 81, a CDRL2 having the sequence as set forth in SEQ ID NO: 30, and a CDRL3 having the sequence as set forth in SEQ ID NO: 82;
(H) a VH domain including: a CDRH1 having the sequence as set forth in SEQ ID NO: 84, a CDRH2 having the sequence as set forth in SEQ ID NO: 87, and a CDRH3 having the sequence as set forth in SEQ ID NO: 90; and a VL domain including: a CDRL1 having the sequence as set forth in SEQ ID NO: 93, a CDRL2 having the sequence as set forth in SEQ ID NO: 96, and a CDRL3 having the sequence as set forth in SEQ ID NO: 97;
(I) a VH domain including: a CDRH1 having the sequence as set forth in SEQ ID NO: 260, a CDRH2 having the sequence as set forth in SEQ ID NO: 261, and a CDRH3 having the sequence as set forth in SEQ ID NO: 262; and a VL domain comprising: a CDRL1 having the sequence as set forth in SEQ ID NO: 263, a CDRL2 having the sequence as set forth in SEQ ID NO: 264, and a CDRL3 having the sequence as set forth in SEQ ID NO: 265, each according to Kabat numbering;
(J) a VH domain including: a CDRH1 having the sequence as set forth in SEQ ID NO: 191, a CDRH2 having the sequence as set forth in SEQ ID NO: 194, and a CDRH3 having the sequence of RYG; and a VL domain including: a CDRL1 having the sequence as set forth in SEQ ID NO: 199, a CDRL2 having the sequence of RMS, and a CDRL3 having the sequence as set forth in SEQ ID NO: 203;
(K) a VH domain including: a CDRH1 having the sequence as set forth in SEQ ID NO: 4, a CDRH2 having the sequence as set forth in SEQ ID NO: 7, and a CDRH3 having the sequence as set forth in SEQ ID NO: 10; and a VL domain including: a CDRL1 having the sequence as set forth in SEQ ID NO: 13, a CDRL2 having the sequence of YTS, and a CDRL3 having the sequence as set forth in SEQ ID NO: 17;
(L) a VH domain including: a CDRH1 having the sequence as set forth in SEQ ID NO: 20, a CDRH2 having the sequence of YGS, and a CDRH3 having the sequence as set forth in SEQ ID NO: 25; and a VL domain including: a CDRL1 having the sequence as set forth in SEQ ID NO: 28, a CDRL2 having the sequence of NAK, and a CDRL3 having the sequence as set forth in SEQ ID NO: 32;
(M) a VH domain including: a CDRH1 having the sequence as set forth in SEQ ID NO: 34, a CDRH2 having the sequence as set forth in SEQ ID NO: 37, and a CDRH3 having the sequence as set forth in SEQ ID NO: 40; and a VL domain including: a CDRL1 having the sequence as set forth in SEQ ID NO: 43, a CDRL2 having the sequence of YAS, and a CDRL3 having the sequence as set forth in SEQ ID NO: 47;
(N) a VH domain including: a CDRH1 having the sequence as set forth in SEQ ID NO: 49, a CDRH2 having the sequence as set forth in SEQ ID NO: 52, and a CDRH3 having the sequence as set forth in SEQ ID NO: 55; and a VL domain including: a CDRL1 having the sequence as set forth in SEQ ID NO: 58, a CDRL2 having the sequence of AAT, and a CDRL3 having the sequence as set forth in SEQ ID NO: 62;
(O) a VH domain including: a CDRH1 having the sequence as set forth in SEQ ID NO: 64, a CDRH2 having the sequence as set forth in SEQ ID NO: 67, and a CDRH3 having the sequence as set forth in SEQ ID NO: 70; and a VL domain including: a CDRL1 having the sequence as set forth in SEQ ID NO: 43, a CDRL2 having the sequence of YAS, and a CDRL3 having the sequence as set forth in SEQ ID NO: 73;
(P) a VH domain including: a CDRH1 having the sequence as set forth in SEQ ID NO: 75, a CDRH2 having the sequence of YSG, and a CDRH3 having the sequence as set forth in SEQ ID NO: 79; and a VL domain including: a CDRL1 having the sequence as set forth in SEQ ID NO: 28, a CDRL2 having the sequence of NAK, and a CDRL3 having the sequence as set forth in SEQ ID NO: 83;
(Q) a VH domain including: a CDRH1 having the sequence as set forth in SEQ ID NO: 85, a CDRH2 having the sequence as set forth in SEQ ID NO: 88, and a CDRH3 having the sequence as set forth in SEQ ID NO: 91; and a VL domain including: a CDRL1 having the sequence as set forth in SEQ ID NO: 94, a CDRL2 having the sequence of GAS, and a CDRL3 having the sequence as set forth in SEQ ID NO: 98, each according to Chothia numbering;
(R) a VH domain including: a CDRH1 having the sequence as set forth in SEQ ID NO: 192, a CDRH2 having the sequence as set forth in SEQ ID NO: 195, and a CDRH3 having the sequence as set forth in SEQ ID NO: 197; and a VL domain including: a CDRL1 having the sequence as set forth in SEQ ID NO: 200, a CDRL2 having the sequence of RMS, and a CDRL3 having the sequence as set forth in SEQ ID NO: 202;
(S) a VH domain including: a CDRH1 having the sequence as set forth in SEQ ID NO: 5, a CDRH2 having the sequence as set forth in SEQ ID NO: 8, and a CDRH3 having the sequence as set forth in SEQ ID NO: 11; and a VL domain including: a CDRL1 having the sequence as set forth in SEQ ID NO: 14, a CDRL2 having the sequence of YTS, and a CDRL3 having the sequence as set forth in SEQ ID NO: 18;
(T) a VH domain including: a CDRH1 having the sequence as set forth in SEQ ID NO: 21, a CDRH2 having the sequence as set forth in SEQ ID NO: 23, and a CDRH3 having the sequence as set forth in SEQ ID NO: 26; and a VL domain including: a CDRL1 having the sequence as set forth in SEQ ID NO: 29, a CDRL2 having the sequence of NAK, and a CDRL3 having the sequence as set forth in SEQ ID NO: 31;
(U) a VH domain including: a CDRH1 having the sequence as set forth in SEQ ID NO: 35, a CDRH2 having the sequence as set forth in SEQ ID NO: 38, and a CDRH3 having the sequence as set forth in SEQ ID NO: 41; and a VL domain including: a CDRL1 having the sequence as set forth in SEQ ID NO: 44, a CDRL2 having the sequence of YAS, and a CDRL3 having the sequence as set forth in SEQ ID NO: 46;
(V) a VH domain including: a CDRH1 having the sequence as set forth in SEQ ID NO: 50, a CDRH2 having the sequence as set forth in SEQ ID NO: 53, and a CDRH3 having the sequence as set forth in SEQ ID NO: 56; and a VL domain including: a CDRL1 having the sequence as set forth in SEQ ID NO: 59, a CDRL2 having the sequence of AAT, and a CDRL3 having the sequence as set forth in SEQ ID NO: 61;
(W) a VH domain including: a CDRH1 having the sequence as set forth in SEQ ID NO: 65, a CDRH2 having the sequence as set forth in SEQ ID NO: 68, and a CDRH3 having the sequence as set forth in SEQ ID NO: 71; and a VL domain including: a CDRL1 having the sequence as set forth in SEQ ID NO: 44, a CDRL2 having the sequence of YAS, and a CDRL3 having the sequence as set forth in SEQ ID NO: 72;
(X) a VH domain including: a CDRH1 having the sequence as set forth in SEQ ID NO: 76, a CDRH2 having the sequence as set forth in SEQ ID NO: 23, and a CDRH3 having the sequence as set forth in SEQ ID NO: 80; and a VL domain including: a CDRL1 having the sequence as set forth in SEQ ID NO: 29, a CDRL2 having the sequence of NAK, and a CDRL3 having the sequence as set forth in SEQ ID NO: 82;
(Y) a VH domain including: a CDRH1 having the sequence as set forth in SEQ ID NO: 86, a CDRH2 having the sequence as set forth in SEQ ID NO: 89, and a CDRH3 having the sequence as set forth in SEQ ID NO: 92; and a VL domain including: a CDRL1 having the sequence as set forth in SEQ ID NO: 95, a CDRL2 having the sequence of GAS, and a CDRL3 having the sequence as set forth in SEQ ID NO: 97, each according to IMGT numbering.
   7. The CAR of any one of embodiments 3-6, wherein the NKp46 binding domain includes
(A) a VH domain having at least 98% sequence identity to the sequence as set forth in SEQ ID NO: 204 and a VL domain having at least 98% sequence identity to the sequence as set forth in SEQ ID NO: 205;
(B) a VH domain having at least 98% sequence identity to the sequence as set forth in SEQ ID NO: 99 and a VL domain having at least 98% sequence identity to the sequence as set forth in SEQ ID NO: 100;
(C) a VH domain having at least 98% sequence identity to the sequence as set forth in SEQ ID NO: 101 and a VL domain having at least 98% sequence identity to the sequence as set forth in SEQ ID NO: 102;
(D) a VH domain having at least 98% sequence identity to the sequence as set forth in SEQ ID NO: 103 and a VL domain having at least 98% sequence identity to the sequence as set forth in SEQ ID NO: 104;
(E) a VH domain having at least 98% sequence identity to the sequence as set forth in SEQ ID NO: 105 and a VL domain having at least 98% sequence identity to the sequence as set forth in SEQ ID NO: 106;
(F) a VH domain having at least 98% sequence identity to the sequence as set forth in SEQ ID NO: 107 and a VL domain having at least 98% sequence identity to the sequence as set forth in SEQ ID NO: 108;
(G) a VH domain having at least 98% sequence identity to the sequence as set forth in SEQ ID NO: 109 and a VL domain having at least 98% sequence identity to the sequence as set forth in SEQ ID NO: 110;
(H) a VH domain having at least 98% sequence identity to the sequence as set forth in SEQ ID NO: 111 and a VL domain having at least 98% sequence identity to the sequence as set forth in SEQ ID NO: 112; or
(I) an antigen binding fragment having at least 98% sequence identity to the sequence as set forth in SEQ ID NO: 266 and an antigen binding fragment having at least 98% sequence identity to the sequence as set forth in SEQ ID NO: 267.
   8. The CAR of any one of embodiments 3-7, wherein the NKp46 binding domain includes
(A) a VH domain having the sequence as set forth in SEQ ID NO: 204 and a VL domain having the sequence as set forth in SEQ ID NO: 205;
(B) a VH domain having the sequence as set forth in SEQ ID NO: 99 and a VL domain having the sequence as set forth in SEQ ID NO: 100;
(C) a VH domain having the sequence as set forth in SEQ ID NO: 101 and a VL domain having the sequence as set forth in SEQ ID NO: 102;
(D) a VH domain having the sequence as set forth in SEQ ID NO: 103 and a VL domain having the sequence as set forth in SEQ ID NO: 104;
(E) a VH domain having the sequence as set forth in SEQ ID NO: 105 and a VL domain having the sequence as set forth in SEQ ID NO: 106;
(F) a VH domain having the sequence as set forth in SEQ ID NO: 107 and a VL domain having the sequence as set forth in SEQ ID NO: 108;
(G) a VH domain having the sequence as set forth in SEQ ID NO: 109 and a VL domain having the sequence as set forth in SEQ ID NO: 110;
(H) a VH domain having the sequence as set forth in SEQ ID NO: 111 and a VL domain having the sequence as set forth in SEQ ID NO: 112; or
(I) an antigen binding fragment of a heavy chain having the sequence as set forth in SEQ ID NO: 266 and an antigen binding fragment of a light chain having the sequence as set forth in SEQ ID NO: 267.
   9. The CAR of any one of embodiments 3-8, wherein the NKp46 binding domain includes a single chain variable fragment (scFv) having at least 98% sequence identity to the sequence as set forth in SEQ ID NO: 206, SEQ ID NO: 113, SEQ ID NO: 114, SEQ ID NO: 115, SEQ ID NO: 116, SEQ ID NO: 117, SEQ ID NO: 118, or SEQ ID NO: 119.
   10. The CAR of any one of embodiments 3-9, wherein the NKp46 binding domain includes a single chain variable fragment (scFv) having the sequence as set forth in SEQ ID NO: 206, SEQ ID NO: 113, SEQ ID NO: 114, SEQ ID NO: 115, SEQ ID NO: 116, SEQ ID NO: 117, SEQ ID NO: 118, or SEQ ID NO: 119.
   11. The CAR of embodiment 1, wherein the inhibitory NK receptor binding domain includes a killer immunoglobulin receptor (KIR) binding domain and/or an NKG2A binding domain.
   12. The CAR of embodiment 11, wherein the KIR binding domain includes an antigen binding fragment of a heavy chain as set forth in SEQ ID NO: 288 and an antigen binding fragment of light chain as set forth in SEQ ID NO: 289.
   13. The CAR of embodiment 11 or 12, wherein the KIR binding domain includes an antigen binding fragment of antibody A210, antibody A803g, antibody clone #180704, and antibody clone NKVFS1.
   14. The CAR of any one of embodiments 11-13, wherein the NKG2A binding domain includes an artificial human leukocyte antigen (HLA)-E mimetic.
   15. The CAR of embodiment 14, wherein the HLA-E mimetic includes a heterotrimer of: i) an HLA-E binding signal peptide; ii) mature beta-2 microglobulin (B2M); and iii) an HLA-E heavy chain.
   16. The CAR of embodiment 15, wherein the HLA-E binding signal peptide includes: a signal peptide of HLA-A, HLA-B, HLA-C, or HLA-G; a peptide from a human immunodeficiency virus (HIV), a cytomegalovirus (CMV) or Epstein-Barr virus (EBV); a peptide of multidrug resistance-associated protein 7; or a leader peptide of HSP60.
   17. The CAR of embodiment 15 or 16, wherein the HLA-E binding signal peptide includes a sequence as set forth in SEQ ID NOs: 127, and 276-287.
   18. The CAR of any one of embodiments 15-17, wherein the HLA-E heavy chain includes an HLA-E 01:03 heavy chain.
   19. The CAR of any one of embodiments 14-18, wherein the HLA-E mimetic includes a sequence having at least 98% sequence identity to the sequence as set forth in SEQ ID NOs: 242, 244, or 246.
   20. The CAR of any one of embodiments 14-19, wherein the HLA-E mimetic includes the sequences as set forth in SEQ ID NOs: 242, 244, and 246.
   21. The CAR of any one of embodiments 11-20, wherein the NKG2A binding domain is an scFV derived from monalizumab, antibody Z270, antibody Z199, antibody 20D5, or antibody 3S9.
   22. The CAR of any one of embodiments 11-21, wherein the NKG2A binding domain includes:
a VH domain including: a CDRH1 having the sequence as set forth in SEQ ID NO: 213, a CDRH2 having the sequence as set forth in SEQ ID NO: 214, and a CDRH3 having the sequence as set forth in SEQ ID NO: 215; and a VL domain including: a CDRL1 having the sequence as set forth in SEQ ID NO: 216, a CDRL2 having the sequence as set forth in SEQ ID NO: 217, and a CDRL3 having the sequence as set forth in SEQ ID NO: 218, or
a VH domain comprising a CDRH1 having the sequence as set forth in SEQ ID NO: 290, a CDRH2 having the sequence as set forth in SEQ ID NO: 291, and a CDRH3 having the sequence as set forth in SEQ ID NO: 292; and a VL domain including: a CDRL1 having the sequence as set forth in SEQ ID NO: 293, a CDRL2 having the sequence as set forth in SEQ ID NO: 294, and a CDRL3 having the sequence as set forth in SEQ ID NO: 295,
according to Kabat numbering.
   23. The CAR of any one of embodiments 11-22, wherein the NKG2A binding domain includes a VH domain having at least 98% sequence identity to the sequence as set forth in SEQ ID NO: 219, 220, 221, 222, or 223.
   24. The CAR of any one of embodiments 11-23, wherein the NKG2A binding domain includes a VH domain having the sequence as set forth in SEQ ID NO: 219, 220, 221, 222, or 223.
   25. The CAR of any one of embodiments 11-24, wherein the NKG2A binding domain includes an antigen binding fragment of a heavy chain having at least 98% sequence identity to the sequence as set forth in SEQ ID NO: 224, 225, 226, 227, or 228, and/or an antigen binding fragment of a light chain having at least 98% sequence identity to the sequence as set forth in SEQ ID NO: 229.
   26. The CAR of any one of embodiments 11-25, wherein the NKG2A binding domain includes an antigen binding fragment of a heavy chain having the sequence as set forth in SEQ ID NO: 224, 225, 226, 227, or 228, and/or an antigen binding fragment of a light chain having the sequence as set forth in SEQ ID NO: 229.
   27. The CAR of any one of embodiments 1-26, wherein the extracellular component further includes a tag.
   28. The CAR of embodiment 27, wherein the tag includes His tag, Flag tag, Xpress tag, Avi tag, Calmodulin binding peptide (CBP) tag, Polyglutamate tag, HA tag, Myc tag, Strep tag, Softag 1, Softag 3, and/or V5 tag.
   29. The CAR of embodiment 27 or 28, wherein the tag has a sequence as set forth in SEQ ID NO: 152, 153, 154, 155, 156, 157, 158, 159, 160, 161, 162, 163, 164, 165, 166, 167, and/or 168.
   30. The CAR of any one of embodiments 1-29, wherein the extracellular component further includes a hinge.
   31. The CAR of embodiment 30, wherein the hinge includes a human Ig hinge, a KIR2DS2 hinge, or a CD8α hinge.
   32. The CAR of embodiment 30 or 31, wherein the hinge is a CD8α hinge.
   33. The CAR of any one of embodiments 30-32, wherein the hinge has the sequence as set forth in SEQ ID NO: 140.
   34. The CAR of any one of embodiments 1-33, wherein the extracellular component further includes a linker.
   35. The CAR of embodiment 34, wherein the linker is a glycine-serine linker, an IgG4 linker, or a CD28 linker.
   36. The CAR of embodiment 34 or 35, wherein the linker has the sequence as set forth in SEQ ID NO: 142, 143, 144, 145, 150, or 151.
   37. The CAR of any one of embodiments 2-36, wherein the transmembrane domain includes a transmembrane domain of: the α, β, or ζ chain of the T-cell receptor, CD28, CD27, CD3ε, CD45, CD4, CD5, CD8, CD9, CD16, CD22, CD33, CD37, CD64, CD80, CD86, CD134, CD137, CD154, KIRDS2, OX40, CD2, LFA-1, ICOS, 4-1BB, GITR, CD40, BAFFR, HVEM, SLAMF7, NKp80, NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1, SLAMF4, CD84, CD96, CEACAM1, CRT AM, Ly9, CD160, PSGL1, CD100, SLAMF6, SLAM, BLAME, SELPLG, LTBR, PAG/Cbp, NKG2D, NKG2C, or a combination thereof.
   38. The CAR of any one of embodiments 2-37, wherein the transmembrane domain includes a transmembrane domain of CD8α chain.
   39. The CAR of any one of embodiments 2-38, wherein the transmembrane domain has the sequence as set forth in SEQ ID NO: 138.
   40. The CAR of any one of embodiments 1-39, wherein the intracellular component includes an intracellular signaling domain.
   41. The CAR of embodiment 40, wherein the intracellular signaling domain includes CD3ζ, common FcR γ, Fc γ RIIa, FcR β, CD3 γ, CD3 δ, CD3 ε, CD79a, CD79b, DAP10, DAP12, or a combination thereof.
   42. The CAR of embodiment 40 or 41, wherein the intracellular signaling domain includes CD3ζ.
   43. The CAR of any one of embodiments 40-42, wherein the intracellular signaling domain has the sequence as set forth in SEQ ID NO: 146.
   44. The CAR of any one of embodiments 40-43, wherein the intracellular signaling domain includes a costimulatory signaling domain.
   45. The CAR of embodiment 44, wherein the costimulatory signaling domain includes an MHC class I molecule, B and T cell lymphocyte attenuator (BTLA), a Toll ligand receptor, OX40, CD27, CD28, CDS, ICAM-1, LFA-1, ICOS, 4-1BB, GITR, BAFFR, HVEM, SLAMF7, NKp80, NKp44, NKp30, NKp46, CD160, CD19, CD4, CD8α, CD8β, IL2Rβ, IL2Rγ, IL7Rα, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, NKG2D, NKG2C, TNFR2, TRANCE/RANKL, DNAM1, SLAMF4, CD84, CD96, CEACAM1, CRTAM, Ly9, CD160, PSGL1, CD100, CD69, SLAMF6, SLAM, BLAME, SELPLG, LTBR, LAT, GADS, SLP-76, PAG/Cbp, CD19a, or a combination thereof.
   46. The CAR of embodiment 44 or 45, wherein the costimulatory signaling domain includes 4-1BB.
   47. The CAR of any one of embodiments 44-46, wherein the costimulatory signaling domain has a sequence as set forth in SEQ ID NO: 148.
   48. The CAR of any one of embodiments 40-47, wherein the intracellular signaling domain includes a 4-1BB costimulatory domain and a CD3ζ stimulatory domain having an amino acid sequence as set forth in SEQ ID NO: 230.
   49. The CAR of any one of embodiments 1-48, wherein the CAR has an amino acid sequence having at least 98% sequence identity to the sequence as set forth in SEQ ID NO: 249, 251, 253, 255, or 257.
   50. The CAR of any one of embodiments 1-49, wherein the CAR has an amino acid sequence as set forth in SEQ ID NO: 249, 251, 253, 255, or 257.
   51. The CAR of any one of embodiments 1-50, wherein the CAR is encoded by a nucleotide sequence having at least 98% sequence identity to the sequence as set forth in SEQ ID NO: 250, 252, 254, 256, or 258.
   52. The CAR of any one of embodiments 1-51, wherein the CAR is encoded by a nucleotide sequence as set forth in SEQ ID NO: 250, 252, 254, 256, or 258.
   53. A vector including a nucleotide sequence encoding the CAR of any one of embodiments 1-52.
   54. The vector of embodiment 53, wherein the vector further includes a promoter operably linked to the nucleotide sequence encoding the CAR of any one of embodiments 1-52.
   55. The vector of embodiment 54, wherein the promoter is the MND promoter.
   56. The vector of embodiment 55, wherein the MND promoter has the sequence as set forth in SEQ ID NO: 175.
   57. The vector of any one of embodiments 53-56, wherein the vector further includes a nucleotide sequence encoding a transduction marker.
   58. The vector of embodiment 57, wherein the transduction marker is co-expressed with the CAR.
   59. The vector of embodiment 49 or 50, wherein the transduction marker is blue fluorescent protein (BFP).
   60. The vector of embodiment 59, wherein the BFP has the sequence as set forth in SEQ ID NO: 169, 170, 171, 172, 173, 174, or 236.
   61. The vector of any one of embodiments 57-60, wherein the nucleotide sequence encoding the transduction marker is linked to the nucleotide sequence encoding the CAR by a nucleotide sequence encoding a cleavable peptide.
   62. The vector of embodiment 61, wherein the cleavable peptide includes porcine teschovirus-1 (P2A), Thosea asigna virus (T2A), equine rhinitis A virus (E2A), foot-and-mouth disease virus (F2A), potyvirus 2A, or cardiovirus 2A.
   63. The vector of embodiment 61 or 62, wherein the cleavable peptide has the sequence as set forth in SEQ ID NO: 176, 177, 178, 179, 180, 181, 182, 183, 184, 185, or 240.
   64. The vector of any one of embodiments 53-63, wherein the vector further includes a molecular safety switch.
   65. The vector of embodiment 64, wherein the molecular safety switch includes a suicide gene.
   66. The vector of embodiment 65, wherein the suicide gene includes inducible-caspase-9 (iCASP9), herpes simplex virus thymidine kinase (HSV-TK), or truncated epidermal growth factor receptor (tEGFR).
   67. A cell genetically modified to express the CAR of any one of embodiments 1-52.
   68. A cell genetically modified to co-express: (a) the CAR of any one of embodiments 1-52, wherein the extracellular component includes an NKp46 binding domain; and (b) a CAR wherein the extracellular component includes an NKG2A binding domain.
   69. A cell genetically modified to co-express: (a) the CAR of any one of embodiments 1-52, wherein the extracellular component includes an NKp46 binding domain; and (b) a CAR wherein the extracellular component includes an NKG2A binding domain and the intracellular component lacks an intracellular signaling domain.
   70. The cell of embodiment 68 or 69, wherein the NKG2A binding domain includes human leukocyte antigen (HLA)-E or an artificial HLA-E mimetic.
   71. A cell including a vector of any one of embodiments 53-66.
   72. The cell of any one of embodiments 67-71, wherein the cell is a T cell, an NK cell, a macrophage, a hematopoietic stem cell (HSC), or a hematopoietic progenitor cell (HPC).
   73. A formulation including a cell of any one of embodiments 67-72 and a pharmaceutically acceptable carrier.
   74. A method of depleting natural killer (NK) cells expressing an activating NK receptor and/or an inhibitory NK receptor in a first population of NK cells, including:
exposing the first population of cells to a second population of cells genetically modified to express a CAR of any one of embodiments 1-52,
wherein the exposing results in depletion of NK cells in the first population.
   75. The method of embodiment 74, wherein the exposing includes administering the second population of CAR expressing cells to a subject having the first population of cells. 76. A method of depleting natural killer (NK) cells expressing activating receptor NKp46 in a first population of cells including NKp46 expressing NK cells, including:
exposing the first population of cells to a second population of cells genetically modified to co-express:
   (a) the CAR of any one of embodiments 1-52, wherein the extracellular component includes an NKp46 binding domain; and
   (b) a CAR wherein the extracellular component includes an NKG2A receptor binding domain, wherein the exposing results in depletion of NKp46 expressing NK cells in the first population.
      77. A method of depleting natural killer (NK) cells expressing activating receptor NKp46 in a first population of cells including NKp46 expressing NK cells, including:
exposing the first population of cells to a second population of cells genetically modified to co-express:
   (a) the CAR of any one of embodiments 1-52, wherein the extracellular component includes an NKp46 binding domain; and
   (b) a CAR wherein the extracellular component includes an NKG2A receptor binding domain and the intracellular component lacks an intracellular signaling domain.
      78. The method of embodiment 76 or 77, wherein the exposing includes administering the second population of CAR expressing cells to a subject having the first population of cells.
      79. The method of any one of embodiments 76-78, wherein the NKG2A binding domain includes human leukocyte antigen (HLA)-E or an artificial HLA-E mimetic.
      80. The method of embodiment 79, wherein the HLA-E mimetic includes a heterotrimer of: i) an HLA-E binding signal peptide; ii) mature beta-2 microglobulin (B2M); and iii) an HLA-E 01:03 heavy chain.
      81. The method of embodiment 80, wherein the HLA-E binding signal peptide includes: a signal peptide of HLA-A, HLA-B, HLA-C, or HLA-G; a peptide from a human immunodeficiency virus (HIV), a cytomegalovirus (CMV) or Epstein-Barr virus (EBV); a peptide of multidrug resistance-associated protein 7; or a leader peptide of HSP60.
      82. The method of embodiment 80 or 81, wherein the HLA-E binding signal peptide includes a sequence as set forth in SEQ ID NOs: 127, and 276-287.
      83. The method of any one of embodiments 79-82, wherein the HLA-E mimetic has at least 98% sequence identity to the sequence as set forth in SEQ ID NO: 242, 244, or 246.
      84. The method of any one of embodiments 79-83, wherein the HLA-E mimetic has the sequence as set forth in SEQ ID NO: 242, 244, or 246.
      85. The method of any one of embodiments 76-78, wherein the NKG2A binding domain is an scFV derived from monalizumab, antibody Z270, antibody Z199, antibody 20D5, or antibody 3S9.
      86. The method of any one of embodiments 76-78, and 85, wherein the NKG2A binding domain includes:
a heavy chain variable (VH) domain including: a CDRH1 having the sequence as set forth in SEQ ID NO: 213, a CDRH2 having the sequence as set forth in SEQ ID NO: 214, and a CDRH3 having the sequence as set forth in SEQ ID NO: 215; and a light chain variable (VL) domain including: a CDRL1 having the sequence as set forth in SEQ ID NO: 216, a CDRL2 having the sequence as set forth in SEQ ID NO: 217, and a CDRL3 having the sequence as set forth in SEQ ID NO: 218, or
a VH domain comprising a CDRH1 having the sequence as set forth in SEQ ID NO: 290, a CDRH2 having the sequence as set forth in SEQ ID NO: 291, and a CDRH3 having the sequence as set forth in SEQ ID NO: 292; and a VL domain including: a CDRL1 having the sequence as set forth in SEQ ID NO: 293, a CDRL2 having the sequence as set forth in SEQ ID NO: 294, and a CDRL3 having the sequence as set forth in SEQ ID NO: 295,
according to Kabat numbering.
   87. The method of any one of embodiments 76-78, 85, and 86, wherein the NKG2A binding domain includes a VH domain having at least 98% sequence identity to the sequence as set forth in SEQ ID NO: 219, 220, 221, 222, or 223.
   88. The method of any one of embodiments 76-78, and 85-87, wherein the NKG2A binding domain includes a VH domain having the sequence as set forth in SEQ ID NO: 219, 220, 221, 222, or 223.
   89. The method of any one of embodiments 76-78, and 85-88, wherein the NKG2A binding domain includes an antigen binding fragment of a heavy chain having at least 98% sequence identity to the sequence as set forth in SEQ ID NO: 224, 225, 226, 227, or 228, and/or an antigen binding fragment of a light chain having at least 98% sequence identity to the sequence as set forth in SEQ ID NO: 229.
   90. The method of any one of embodiments 76-78, and 85-89, wherein the NKG2A binding domain includes an antigen binding fragment of a heavy chain having a sequence as set forth in SEQ ID NO: 224, 225, 226, 227, or 228, and/or an antigen binding fragment of a light chain having a sequence set forth in SEQ ID NO: 229.
   91. The method of any one of embodiments 76-90, wherein expression of the CAR including the NKG2A binding domain in CAR expressing cells confer resistance on the CAR expressing cells to killing by the NKp46 expressing NK cells.
   92. A method of treating a natural killer (NK) cell associated disease or a disease associated with cells that express NK receptors in a subject in need thereof, including:
administering to the subject a therapeutically effective amount of a formulation including cells genetically modified to express the CAR of any one of embodiments 1-52,
thereby treating the NK cell associated disease or a disease associated with cells that express NK receptors in the subject.
   93. The method of embodiment 92, wherein the NK cell associated disease is an NK cell malignancy.
   94. The method of embodiment 93, wherein the NK cell malignancy includes immature NK cell neoplasms, agranular CD4+/CD56+ hematodermic neoplasms, CD94 1A+/TCR− lymphoblastic lymphoma/leukemia (LBL), myeloid/NK cell acute leukemia, mature NK cell neoplasms, extranodal NK cell lymphoma, nasal type, aggressive NK cell leukemia, and chronic NK cell lymphocytosis.
   95. The method of any one of embodiments 92-94, further including administering to the subject an anti-viral treatment or prophylaxis.
   96. The method of embodiment 95, wherein the anti-viral treatment treats Epstein-Barr Virus (EBV) infection.
   97. The method of embodiment 95 or 96, wherein the anti-viral treatment includes a nucleoside analog selected from acyclovir, valacyclovir, famciclovir, ganciclovir, and/or valganciclovir.
   98. The method of embodiment 92, wherein the disease associated with cells that express NK receptors include non-NK cell malignancies with aberrant NK receptor expression.
   99. The method of embodiment 98, wherein the disease includes Sezary syndrome, mature T cell neoplasms, T cell large granular lymphocytic leukemia, mycosis fungoides, and ALK+ anaplastic large cell lymphoma.
   100. The method of embodiment 92, wherein the disease associated with cells that express NK receptors include infections.
   101. method of embodiment 100, wherein the infections are chronic.
   102. The method of embodiment 100 or 101, wherein the infections are bacterial, viral, fungal, parasitic, and/or arthropod.
   103. The method of any one of embodiments 100-102, wherein the infections are caused by or include Staphylococcus spp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, Treponema pallidum, rhinovirus, influenza virus, respiratory syncytial virus (RSV), coronavirus, herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), Merkel cell polyomavirus (MCV), HHV8 (Kaposi's sarcoma), Trychophyton spp., Candida spp., Giardia, toxoplasmosis, E. vermicularis, Trypanosoma cruzi, Echinococcosis, Cysticercosis, Toxocariasis, Trichomoniasis, Amebiasis, California encephalitis, Chikungunya, dengue, Eastern equine encephalitis, Powassan, St. Louis encephalitis, West Nile, Yellow Fever, Zika, Lyme disease, and/or babesiosis.
   104. The method of embodiment 92, wherein the formulation includes cells expressing a CAR including an extracellular component including an NKG2A binding domain.
   105. The method of embodiment 104, wherein the NK cell associated disease is cancer.
   106. The method of embodiment 105, wherein the cancer includes: carcinoma of the bladder, head and neck, breast, colon, kidney, liver, lung, ovary, prostate, pancreas, stomach, cervix, thyroid or skin; squamous cell carcinoma; leukemia; acute lymphocytic leukemia; acute lymphoblastic leukemia; B cell lymphoma; T cell lymphoma; Hodgkin's lymphoma; non-Hodgkin's lymphoma; hairy cell lymphoma; Burkett's lymphoma; acute myelogenous leukemia; chronic myelogenous leukemia; promyelocytic leukemia; fibrosarcoma; rhabdomyosarcoma; osteosarcoma; neuroblastoma; glioma; astrocytoma; schwannomas; melanoma; xeroderma pigmentosum; keratoacanthoma; seminoma; thyroid follicular cancer; teratocarcinoma; T-prolymphocytic leukemia (T-PLL); large granular lymphocyte leukemia (LGL) of the T cell type; Sezary syndrome (SS); adult T cell leukemia lymphoma (ATLL); hepatosplenic T cell lymphoma; peripheral/post-thymic T cell lymphoma; angioimmunoblastic T cell lymphoma; angiocentric (nasal) T cell lymphoma; anaplastic (Ki 1+) large cell lymphoma; intestinal T cell lymphoma; and/or T-lymphoblastic lymphoma/leukemia (T-Lbly/T-ALL).
   107. The method of any one of embodiments 92-106, wherein the amount of NK cells expressing NKG2A inhibitory receptor in the subject is decreased, as compared to an amount of NK cells expressing NKG2A inhibitory receptor in the subject prior to the administering or in a reference population in need thereof not administered the formulation.
   108. A method of treating a natural killer (NK) cell associated disease or a disease associated with cells that express NK receptors in a subject in need thereof, including:
administering to the subject a therapeutically effective amount of a formulation,
wherein the formulation includes cells of
   (a) a first population genetically modified to express the CAR of any one of embodiments 1-52, wherein the extracellular component includes an NKp46 binding domain; and
   (b) a second population genetically modified to express a CAR wherein the extracellular component includes an NKG2A binding domain.
      109. A method of treating a natural killer (NK) cell associated disease or a disease associated with cells that express NK receptors in a subject in need thereof, including:
administering to the subject a therapeutically effective amount of a formulation,
wherein the formulation includes cells of
   (a) a first population genetically modified to express the CAR of any one of embodiments 1-52, wherein the extracellular component includes an NKp46 binding domain; and
   (b) a second population genetically modified to express a CAR wherein the extracellular component includes an NKG2A binding domain and the intracellular component lacks an intracellular signaling domain.
      110. A method of treating a natural killer (NK) cell associated disease ora disease associated with cells that express NK receptors in a subject in need thereof, including:
administering to the subject a therapeutically effective amount of a formulation,
wherein cells of the formulation co-express:
   (a) the CAR of any one of embodiments 1-52, wherein the extracellular component includes an NKp46 binding domain; and
   (b) a CAR wherein the extracellular component includes an NKG2A binding domain.
      111. A method of treating a natural killer (NK) cell associated disease or a disease associated with cells that express NK receptors in a subject in need thereof, including:
administering to the subject a therapeutically effective amount of a formulation,
wherein cells of the formulation co-express:
   (a) the CAR of any one of embodiments 1-52, wherein the extracellular component includes an NKp46 binding domain; and
   (b) a CAR wherein the extracellular component includes an NKG2A binding domain and the intracellular component lacks an intracellular signaling domain.
      112. The method of any one of embodiments 108-111, further including administering to the subject an anti-viral treatment or prophylaxis.
      113. The method of embodiment 112, wherein the anti-viral treatment treats Epstein-Barr Virus (EBV) infection.
      114. The method of embodiment 112 or 113, wherein the anti-viral treatment includes a nucleoside analog selected from acyclovir, valacyclovir, famciclovir, ganciclovir, and/or valganciclovir.
      115. The method of any one of embodiments 108-114, wherein the NKG2A binding domain includes human leukocyte antigen (HLA)-E or an artificial HLA-E mimetic. 116. The method of embodiment 115, wherein the HLA-E mimetic includes a heterotrimer of: i) an HLA-E binding signal peptide; ii) mature beta-2 microglobulin (B2M); and iii) the HLA-E 01:03 heavy chain.
      117. The method of embodiment 116, wherein the HLA-E binding signal peptide includes: a signal peptide of HLA-A, HLA-B, HLA-C, or HLA-G; a peptide from a human immunodeficiency virus (HIV), a cytomegalovirus (CMV) or Epstein-Barr virus (EBV); a peptide of multidrug resistance-associated protein 7; or a leader peptide of HSP60.
      118. The method of embodiment 116 or 117, wherein the HLA-E binding signal peptide includes a sequence as set forth in SEQ ID NOs: 127, and 276-287.
      119. The method of any one of embodiments 115-118, wherein the HLA-E mimetic has at least 98% sequence identity to the sequence as set forth in SEQ ID NO: 242, 244, or 246.
      120. The method of any one of embodiments 115-119, wherein the HLA-E mimetic has the sequence as set forth in SEQ ID NO: 242, 244, or 246.
      121. The method of any one of embodiments 108-114, wherein the NKG2A binding domain is an scFv derived from monalizumab, antibody Z270, antibody Z199, antibody 20D5, or antibody 3S9.
      122. The method of embodiment 121, wherein the NKG2A binding domain includes:
a heavy chain variable (VH) domain including: a CDRH1 having the sequence as set forth in SEQ ID NO: 213, a CDRH2 having the sequence as set forth in SEQ ID NO: 214, and a CDRH3 having the sequence as set forth in SEQ ID NO: 215; and a light chain variable (VL) domain including: a CDRL1 having the sequence as set forth in SEQ ID NO: 216, a CDRL2 having the sequence as set forth in SEQ ID NO: 217, and a CDRL3 having the sequence as set forth in SEQ ID NO: 218, or
a VH domain comprising a CDRH1 having the sequence as set forth in SEQ ID NO: 290, a CDRH2 having the sequence as set forth in SEQ ID NO: 291, and a CDRH3 having the sequence as set forth in SEQ ID NO: 292; and a VL domain including: a CDRL1 having the sequence as set forth in SEQ ID NO: 293, a CDRL2 having the sequence as set forth in SEQ ID NO: 294, and a CDRL3 having the sequence as set forth in SEQ ID NO: 295,
according to Kabat numbering.
   123. The method of embodiment 121 or 122, wherein the NKG2A binding domain includes a VH domain having at least 98% sequence identity to the sequence as set forth in SEQ ID NO: 219, 220, 221, 222, or 223.
   124. The method of any one of embodiments 121-123, wherein the NKG2A binding domain includes a VH domain having the sequence as set forth in SEQ ID NO: 219, 220, 221, 222, or 223.
   125. The method of any one of embodiments 121-124, wherein the NKG2A binding domain includes an antigen binding fragment of a heavy chain having at least 98% sequence identity to the sequence as set forth in SEQ ID NO: 224, 225, 226, 227, or 228, and/or an antigen binding fragment of a light chain having at least 98% sequence identity to the sequence as set forth in SEQ ID NO: 229.
   126. The method of any one of embodiments 121-125, wherein the NKG2A binding domain includes an antigen binding fragment of a heavy chain having a sequence as set forth in SEQ ID NO: 224, 225, 226, 227, or 228, and/or an antigen binding fragment of a light chain having a sequence as set forth in SEQ ID NO: 229.
   127. The method of any one of embodiments 108-126, wherein expression of the CAR including the NKG2A binding domain in CAR expressing cells confer resistance on the CAR expressing cells to killing by NK cells in the NK cell associated disease.
   128. The method of any one of embodiments 108-127, wherein expression of the CAR including the NKG2A binding domain in CAR expressing cells reduces rejection of the CAR expressing cells by the subject.
   129. The method of any one of embodiments 108-128, wherein the NK cell associated disease is an autoimmune disease or an alloimmune disease.
   130. The method of embodiment 129, wherein the autoimmune disease includes Sjogren's disease; antiphospholipid syndrome; Pemphigus vulgaris; spondylarthropathies; skin diseases including psoriasis; multiple sclerosis; systemic sclerosis; Type I diabetes; juvenile idiopathic arthritis; rheumatoid arthritis; inflammatory bowel disease; autoimmune liver diseases; and/or systemic lupus erythematosus (SLE).
   131. The method of embodiment 129, wherein the alloimmune disease includes fetal/neonatal alloimmune thrombocytopenia (FNAIT); hematopoietic stem cell rejection; solid tissue transplant rejection; and/or chronic allograft injury after kidney transplantation.
   132. The method of any one of embodiments 108-131, wherein the amount of NK cells expressing NKp46 activating receptor in the subject is decreased, as compared to an amount of NK cells expressing NKp46 activating receptor in the subject prior to the administering or in a reference population in need thereof not administered the formulation.
   133. A kit including: a nucleotide sequence encoding a CAR of any one of embodiments 1-52; a vector of any one of embodiments 53-66; and/or a cell of any one of embodiments 67-72.
   134. The kit of embodiment 133, further including cells to produce the vector.
   135. The kit of embodiment 133 or 134, wherein the vector is a viral vector.
   136. The kit of embodiment 134, wherein the cells to produce the vector include 293 cells, 293T cells, and/or A549 cells.
   137. The kit of any one of embodiments 133-136, further including media, fibronectin-coated plates, hexadimethrine bromide (Polybrene), lipofectamine, artificial presenting cells, growth factors, and/or antibodies.

(xvi) Experimental Examples. Example 1. This Example describes targeting of an activating NK receptor, NKp46, on natural killer (NK) cells with T cells genetically modified to express a chimeric antigen receptor (CAR) having an extracellular binding domain that binds to NKp46. Sequences of CARs described in this Example and in FIG. 2A include sequences set forth in SEQ ID NOs: 127, 130, 133, 136, 138, 140, 142, 143, 146, 148, 190, 193, 196, 198, 201, 202, 204, 205, 206, 207, 208, 209, 210, 211, 212, 230, 231, 232, 233, 234, 235, 236, 237, 238, 239, 240, 241, 242, 243, 244, 245, 246, 247, 248, 249, 250, 251, 252, 253, 254, 255, 256, 257, and 258.

NK cell neoplasms, including NK/T cell lymphoma and aggressive NK cell leukemia, are rare malignancies. The incidence is 0.8 per 1,000,000 in the U.S. and 3 to 10 times higher in South America and Asia. High mortality is observed, with a 17- to 20-month overall median survival, but much worse for relapsed/refractory NK/T lymphomas and aggressive NK cell leukemias. These malignancies are usually associated with Epstein-Barr Virus (EBV). To address this problem, CAR T cells were developed to target NK cell malignancies. However, NK cells do not express a uniformly expressed cell surface receptor that is unique to NK cells. NKp46 is an activating NK receptor that is expressed broadly by many NK cell subsets. Reports suggest NKp46 is expressed on 90% of NK cell malignancies. This study shows targeting of NKp46 with CAR T cells.

Methods: An anti-NKp46 antibody was synthesized as a scFv sequence and cloned into a lentiviral (LV) CAR backbone containing 4-1BB as the costimulatory domain and a downstream blue fluorescent protein (BFP) reporter (FIGS. 2A-2C). Primary peripheral blood mononuclear cells (PBMC) were stimulated with CD3/CD28, transduced with anti-NKp46 CAR LV, and expanded. CAR T cells were mixed with K-562 cells transduced with NKp46. Effector to target ratios of 2:1 were used, unless otherwise specified. CAR T cell function was evaluated by flow cytometry. CAR T cells were identified by BFP expression. CAR T cell activation was evaluated by expression of cell surface CD137 and intracellular cytokine expression and compared to CD19 CAR T cells. Killing of NKp46-expressing K562 cells and primary NK cells was also assessed by flow cytometry.

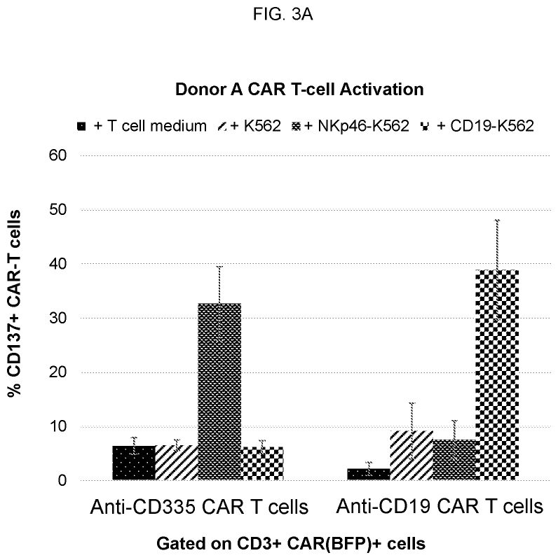

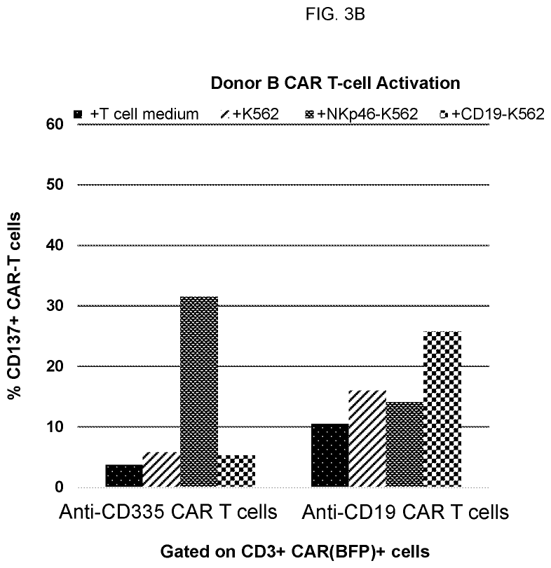

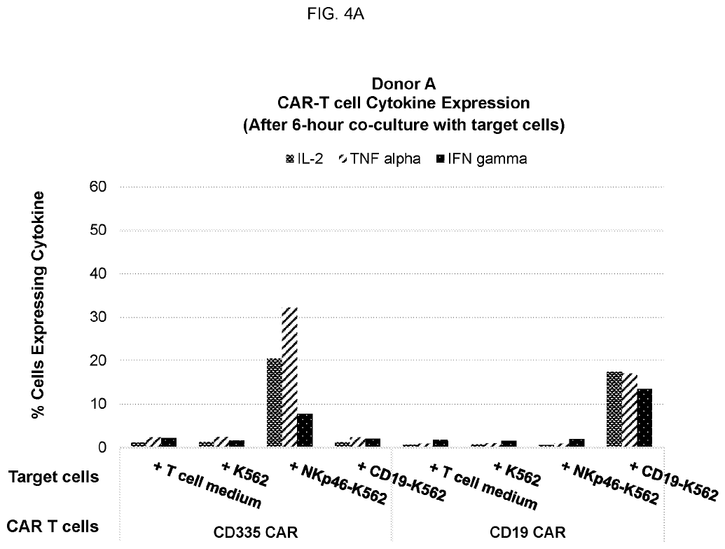

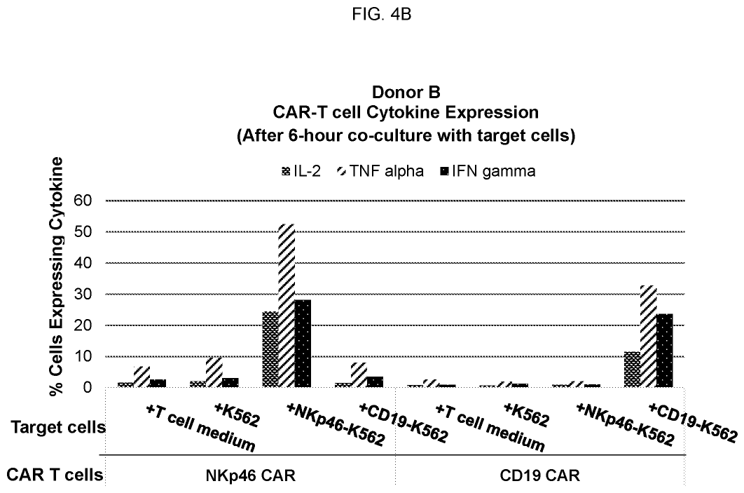

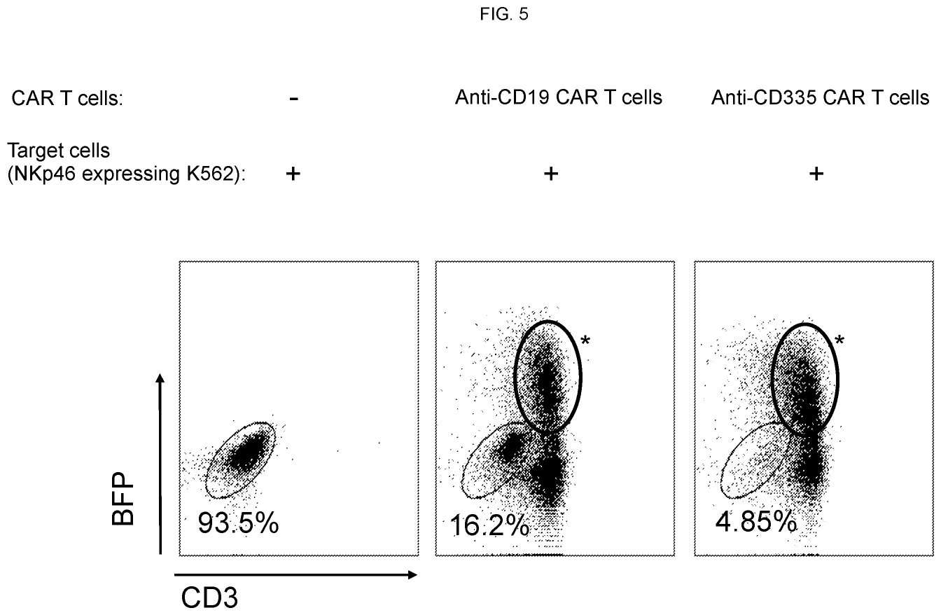

Results: Anti-NKp46 CAR T cells were made from three different donors. The percentage of BFP+ CAR T cells across three donors was a median of 56% (range 53-65%; FIG. 2C). When exposed to NKp46+ target cells, the percentage of CAR T (BFP+) cells expressing CD137 increased a median of 5-fold (range 4- to 6-fold) compared to CD19 CAR T cells mixed with same NKp46+ target cells (FIGS. 3A, 3B). Similarly, the percentage of anti-NKp46 CAR T cells with intracellular IL-2, TNF-α, and IFN-γ expression increased a median of 10.5-fold (range 10- to 11-fold), 11-fold (range 9- to 13-fold), and 9-fold (range 7- to 11-fold) relative to the CD19 CAR T cells mixed with NKp46+ target cells (FIGS. 4A, 4B). Activation in the anti-CD19 CAR T cells when mixed with CD19+ target cells showed similar CD137 activation and cytokine expression to the anti-NKp46 CAR T cells. When mixed for 24 hours, anti-NKp46 CAR T cells were able to reduce the percentage of NKp46+ K562 cells by 70% (FIG. 5). The anti-NKp46 CAR T cells also effectively depleted primary autologous NK cells. Anti-CD19 CAR T cells did not deplete NKp46⁺ cells.

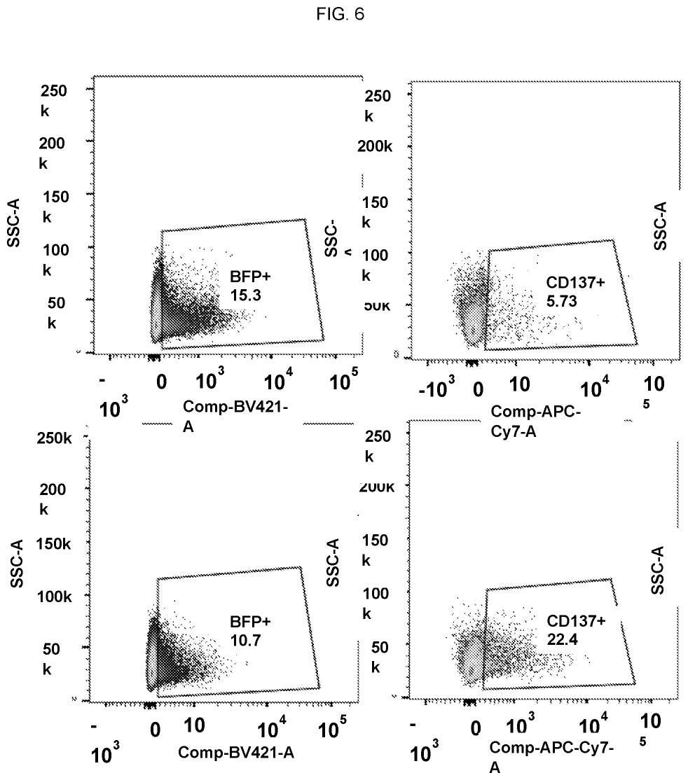

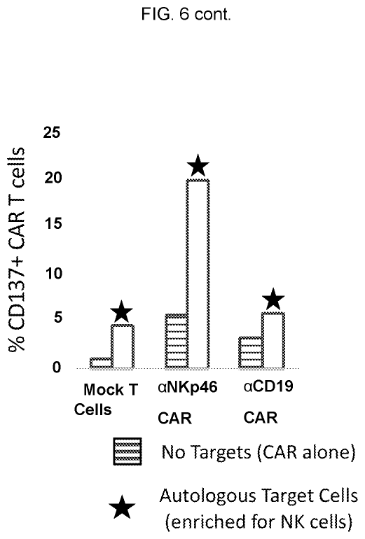

Anti-NKp46 CAR T cells were mixed with autologous target cells enriched for NK cells (40-50% NK cells) at a 2:1 ratio and evaluated by flow cytometry after 6 hours. CD137 expression was measured in anti-NKp46 CAR T cells by gating on single live CD3+ lymphocytes that express BFP. It was observed that anti-NKp46 CAR T cells were activated when mixed with autologous NK cells as measured by upregulation of CD137 expression on anti-NKp46 CAR T cells (FIG. 6).

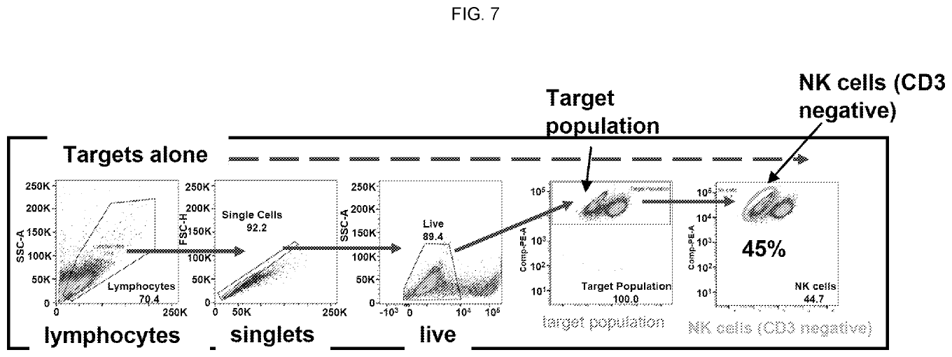

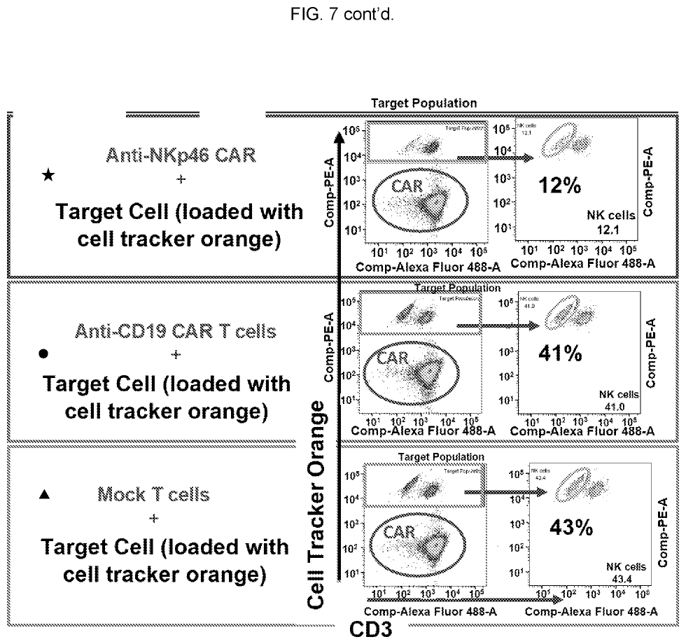

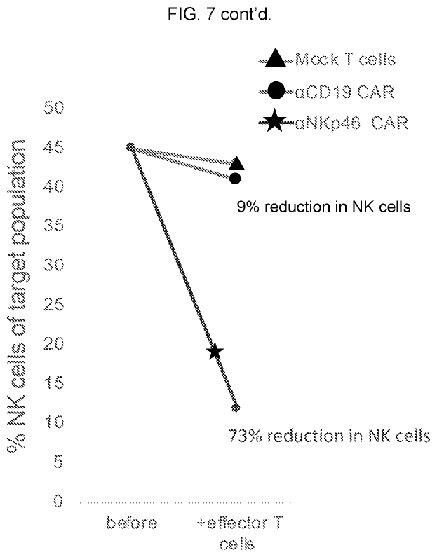

Autologous target NK cells were enriched and stained with cell tracker and then mixed with anti-NKp46 CAR T cells, control CAR T cells (anti-CD19 CAR T cells), or mock T cells at a 2 CAR to 1 target cell ratio for 24 hours and then evaluated by flow cytometry. The NK cells were quantified by gating on CD3 negative cell tracker orange cells. It was observed that anti-NKp46 CAR T cells killed autologous target NK cells (FIG. 7). In the presence of anti-NKp46 CAR T cells, the percentage of NK cells was reduced by 72% in the presence of anti-NKp46 CAR T cells versus only 9% in the presence of control CAR T cells (anti-CD19 CAR).

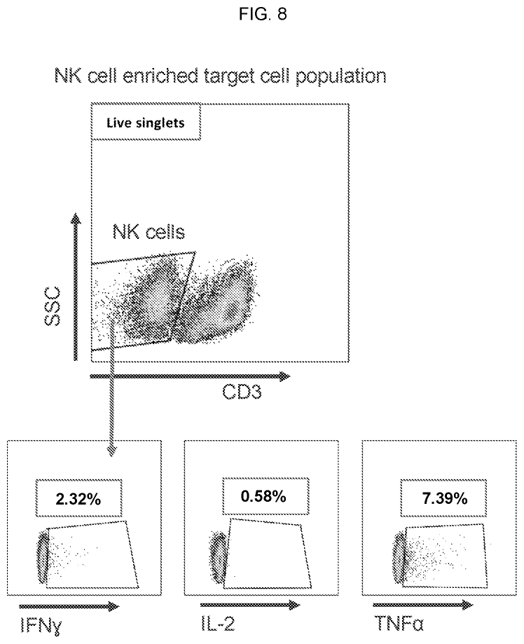

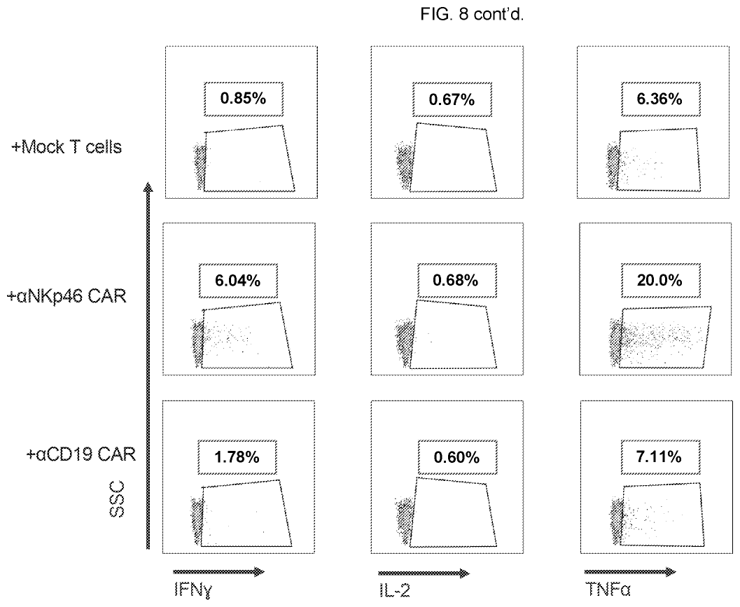

Autologous target cells enriched for NK cells were mixed with anti-NKp46 CAR T cells at a 2 CAR cell:1 target cell ratio for 24 hours and assessed for intracellular cytokine expression by flow cytometry. It was observed that autologous target NK cells were activated by contact with anti-NKp46 CAR T cells (FIG. 8). Gating on NK cells demonstrated that cytokine expression increased when the NK cells were mixed with anti-NKp46 CAR T cells but did not increase when NK cells were mixed with mock T cells or anti-CD19 CAR T cells. The percentage of NK cells expressing IFN-γ or TNF-α increased 2-3 fold, whereas IL-2 did not seem to increase. IFN-γ or TNF-α are cytokines typically produced by activated NK cells.

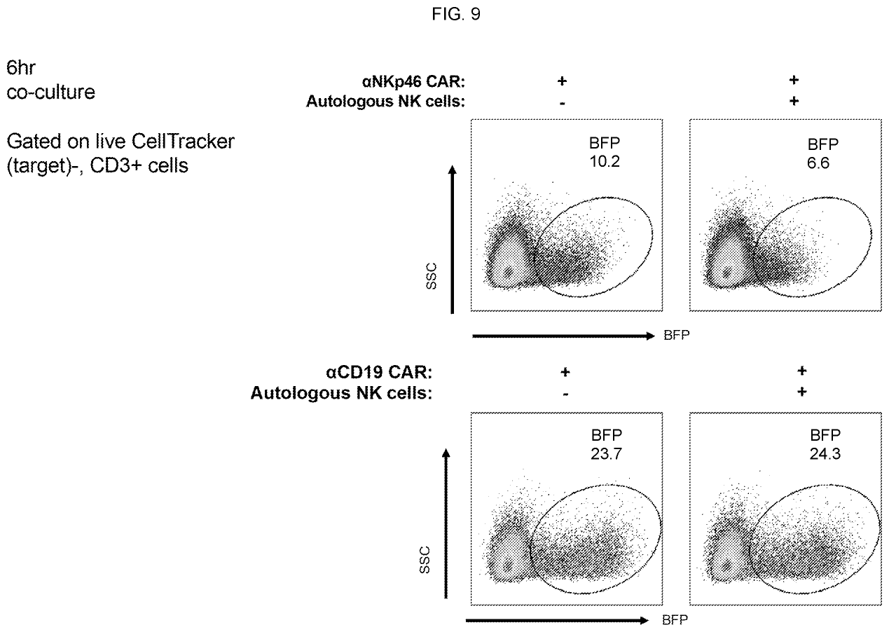

Anti-NKp46 or anti-CD19 CAR T cells were mixed with autologous NK cells and assessed by flow cytometry at 6 hours. The autologous NK cells were loaded with cell tracker to help differentiate the target cells. The CAR T cells were evaluated by gating on cell tracker negative cells (non-target), CD3+ (non-NK cells), and BFP (CAR+ cells). Plots show side scatter (SSC) versus BFP. It was observed that autologous target NK cells killed anti-NKp46 CAR T cells (FIG. 9). The percentage of BFP+ anti-NKp46 CAR T cells decreased 35% (10.2% to 6.6%) in the presence of autologous target NK cells (upper panels), whereas the percentage of BFP+ anti-CD19 CAR T cells didn't decrease in the presence of autologous NK cells (lower panels). The decrease in anti-NKp46 CAR T cells seems to be most pronounced in cells that express the most BFP.

A potent, scFv-based, second-generation anti-NKp46 CAR has been developed and expressed in cells. The anti-NKp46 CAR T-cells are functional, show robust and specific activation, inflammatory cytokine release, and cell killing in response to target cells modified to express the NKp46 receptor and to autologous primary NK cells. This approach can substantially deplete NK cells in vivo. The risk may be manageable short term with anti-viral prophylaxis, analogous to what is done during stem cell transplants. Technology to regulate the anti-NKp46 CAR T cells or consolidating anti-NKp46 CAR T-cell therapy with stem cell transplantation are potential avenues to address depletion of NK cells in vivo in the long term.

Example 2. This study demonstrates that T cells expressing two distinct anti-NK cell CARs targeting NKp46 (CD335) and NKG2A (CD159a) have specific and robust cytotoxic activity against cells engineered to express NKp46, against an NK cell lymphoma cell line, and against autologous primary NK cells. The study shows that CAR T cell therapy can be used to treat NK cell malignancies.

Chimeric antigen receptor (CAR) T cell therapy has paved a breakthrough in the treatment of certain refractory hematologic malignancies (Brentjens et al. Sci. Transl. Med. 5, 177ra38-177ra38 (2013); Maude et al. N. Engl. J. Med. 371, 1507-1517 (2014)). Antigen escape from the selective pressure of CAR T cells has been described and has motivated a variety of strategies targeting multiple epitopes (Shah et al. Front. Oncol. 9, (2019)). Lending additional support to a multi-epitope approach, a specific obstacle to targeting NK cells is the lack of an NK cell surface receptor that is both uniquely and uniformly expressed. NKp46 is an activating receptor that is expressed on a large majority (80%) of NK cell malignancies (Freud et al. Am. J. Clin. Pathol. 140, 853-866 (2013)). NKG2A is an inhibitory receptor expressed on a majority of CD3⁻CD56^{bright} NK cells, which comprise a major subset of NK cell tumors (Lima, M. Pathology (Phila.) 47, 503-514 (2015)). Therefore, two distinct anti-NK cell CARs targeting NKp46 (CD335) and NKG2A (CD159a) were developed, both derived from an identical 4-1 BB/CD3ζ second-generation CAR construct, and their specific activity tested in vitro.

Materials and Methods. CAR constructs and viral vectors. The control αCD19 CAR pRRL vector containing the gamma retrovirus-derived MND promoter and mTagBFP reporter has previously been described (Sather et al. Sci. Transl. Med. 7, 307ra156 (2015)). The CAR construct, situated between the MND promoter and mTagBFP reporter, includes the anti-CD19 scFv fused to the CD8α hinge and transmembrane domains, followed by the intracellular 4-1BB co-stimulatory and CD3ζ signaling domains. The CAR and mTagBFP reporter are separated by the T2A self-cleaving peptide. To create the αNKp46 CAR, a scFv was synthesized from an antibody targeting NKp46 developed by Innate Pharma S.A. (the αNKp46 scFv includes a sequence as set forth in SEQ ID NO: 206 and/or is encoded by a sequence as set forth in SEQ ID NO: 248, as disclosed herein). The scFv coding sequence was synthesized by IDT (Integrated DNA Technologies, Coralville, IA) and subsequently cloned to replace the anti-CD19 scFv from the control vector. The αNKp46 CAR is as set forth in SEQ ID NO: 249 and/or is encoded by a sequence as set forth in SEQ ID NO: 250, as disclosed herein. To create the αNKG2A CAR, the sequence encoding an HLA-E/B2M/G-peptide trimer previously described (Gornalusse et al. Nat. Biotechnol. 35, 765-772 (2017)) was likewise synthesized by IDT and cloned in place of the scFv, CD8 hinge and transmembrane domains of the anti-CD19 mTagBFP CAR construct. The HLA-E/B2M/G-peptide trimer includes SEQ ID NO: 242 and/or is encoded by a sequence as set forth in SEQ ID NO: 243. The αNKG2A CAR includes a sequence as set forth in SEQ ID NO: 251 and/or is encoded by a sequence as set forth in SEQ ID NO: 252.

The NKp46-expressing K562 cell line was generated by amplifying and cloning NKp46 recombinant DNA obtained from Sino Biologics (NCBI Ref Seq BC064806) into a pCVL backbone downstream of the sEF1a promoter and directly upstream of mCherry protein. K562 cells were transduced then enriched by magnetic bead isolation. Prior to lentiviral production, all new vectors were verified by both restriction digestion and sequencing. The GFP+ CD19-expressing K562 cells, used as controls, were transduced using a pRRL-MND-CD19-GFP LV vector.

Primary cell, cell line cultures. Primary T and NK cells were acquired from anonymous blood donors. PBMCs were either previously cryopreserved PBMCs isolated from donors at Seattle Children's Center for Global Infectious Disease (with approval from Seattle Children's Research Institute's Institutional Review Board), or purchased from Astarte Biologics (Bothell, WA). T cells were isolated via negative isolation using the human Pan T Cell Isolation Kit (Miltenyi Biotec, Bergisch Gladbach, Germany). T cells and PBMCs were cultured in T cell medium made of RPMI-1640 supplemented with 20% Fetal Bovine Serum (Gibco, Waltham, MA), 1×20 mM GlutaMAX Supplement (Gibco), 1×1M N-2-hydroxyethylpiperazine-N′-2-ethasulfonic acid (HEPES) (Gibco), 55 μM 2-mercaptoethanol (Gibco), and Penicillin/Streptomycin (Gibco). T cells specifically were expanded in T cell medium supplemented with 50 ng/mL of IL-2 (Peprotech, Rocky Hill, NJ), and 5 ng/mL of IL-7 (Peprotech) and IL-15. Cells were maintained at 1-1.5×10⁶ cells/mL and expanded every 1-2 days. PBMCs or T cells stored before use were cryopreserved in 10% dimethyl sulfoxide T cell medium.

Primary NK cells used in co-cultures were expanded directly from PBMCs in NK MACS medium (Miltenyi Biotec) supplemented with 5% AB serum, 500 IU/mL IL-2 and 140 IU/mL IL-15 at 4-5×10⁵ cells/mL and enriched via negative selection prior to use using the NK Cell Isolation Kit (Miltenyi Biotec). NK-92MI cells were purchased from ATCC and cultured as recommended. K562 cells from ATCC were maintained at 5-8×10⁵ in RPMI-1640 supplemented with 10% fetal bovine serum, 1×20 mM GlutaMAX and Penicillin/Streptomycin. All cell types were cultured at 37° C. 5% CO₂.

Lentiviral (LV) transduction of primary T cells with CAR vectors. T cells were stimulated with CD3/CD28 activator beads (Gibco Dynabeads) at a 1:1 ratio for 48 hours. Cells were then washed and rested for 24-48 hours in expansion T cell medium before being transduced with the CAR LVs at 2-4×10⁴ MOI. For each transduction, cells were seeded at a density of 2×10⁶ cells/mL in T cell medium supplemented with 4 μg of polybrene (Sigma-Aldrich). After 12 hours, medium was added to adjust the concentration to 1×10⁶ cells/mL. Cells were gradually expanded to larger culture volumes to maintain optimal density. Transduction efficiency was measured by flow cytometric assessment of BFP expression 5-7 days after transduction, and cells were used for co-culture experiments 7-9 days after transduction. The target K562-NKp46 cell line was generated using the same methods.

Surface CD137 and intracellular cytokine expression, and cytotoxicity assays. Three assays were performed to determine αNKp46 and αNKG2A CAR T cell activation and cytotoxic activity against NKp46-expressing K562s and primary autologous NK cells. CAR T cell activation was determined via CD137 surface expression and intracellular cytokine expression, while cytotoxicity was determined by measuring the decrease in the percentage of target cells pre-stained with CellTracker Orange or Green (Thermo Fisher Scientific, Waltham, MA). To determine CAR T cell CD137 expression in response to NKp46-expressing K562 cells, primary NK cells, and NK92 cells, αNKp46 CAR T cells, αNKG2A, or control αCD19 CAR T cells were incubated with targets at an effector-to-target (E:T) ratio of 2:1 for 24 h (K562 target) or 6 h (NK cell targets) at 37° C. Cells were then washed, stained for viability (Live/Dead), CD3 (HIT3A or OKT3), NKp46 (E3), and CD137 antibody (4B4-1), all from BioLegend (San Diego, CA), and analyzed by flow-cytometry. To determine intracellular cytokine expression against NKp46-expressing K562s and primary NK cells, αNKp46 CAR T cells or control αCD19 CAR T cells were incubated with target cells at a 2:1 E:T ratio for 6 h at 37° C. One hour into incubation, 1000× GolgiPlug Protein Transport Inhibitor (BD Biosciences, San Jose, CA) was added to the co-cultures to block cytokine transport. After incubation, cells were washed, stained for viability and CD3, fixed and permeabilized, stained for IL-2 (MQ1-17H12), TNFα (Mab11) and IFNγ (B27), and finally analyzed by flow cytometry.

In assays capturing αNKp46 and αNKG2A CAR cytotoxicity, NKp46-expressing K562s, primary NK cells were labeled with 2.5 μM CellTracker Orange or Green (Invitrogen, Carlsbad, CA) and added to effector cells at indicated E:T ratios. After 24 h of co-culture, cells were washed, stained for viability, CD3 and/or NKp46. Percent target lysis was calculated as [(% target cells with target cells alone−% target cells with effector)/% target cells with effectors×100].

For all assays involving primary NK cells, targets were added to effector cells (CAR T cells) from T cell-containing NK MACS cultures to a final effector-to-NK cell ratio of 2:1. In activation assays, 2×10⁵ CAR T cells were seeded per co-culture. In cytotoxicity assays, CAR T cells were added at increasing E:T ratios from 0.5:1 to 5:1 to 5×10⁴ target cells. All co-cultures were plated at a density of 1×10⁶ cells/mL at 37° C.

Analysis and statistics. All flow cytometry was performed on a BD LSR II. Cell sorting was performed on the BD FACSMelody. All data were analyzed in FlowJo. All statistical analyses were performed using the GraphPad software (8.4.2s).

Results. CAR design and CAR T cell production. A schematic representation of the anti-NKp46 (αNKp46) and anti-NKG2A (αNKG2A) CAR are shown in FIGS. 10A and 10B. All CAR constructs tested in this study contained a downstream T2A-cleaved BFP reporter. BFP expression was observed 5-9 days after transduction of αNKp46, αNKG2A, and control αCD19 CAR constructs (FIG. 10C). Mock T cells received no LV and expressed no BFP.

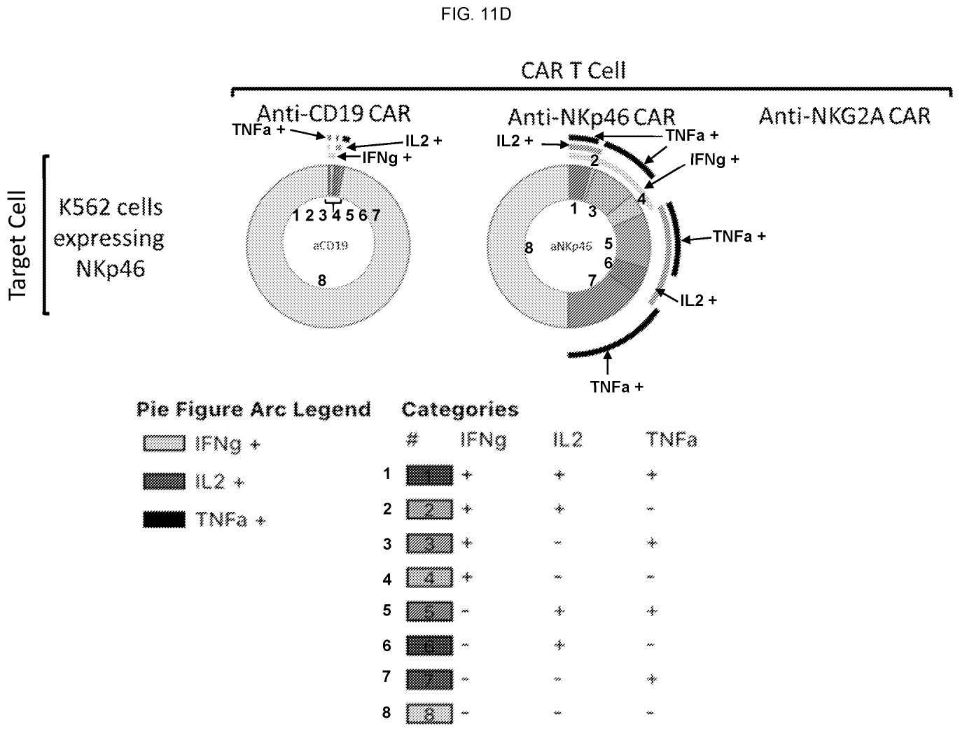

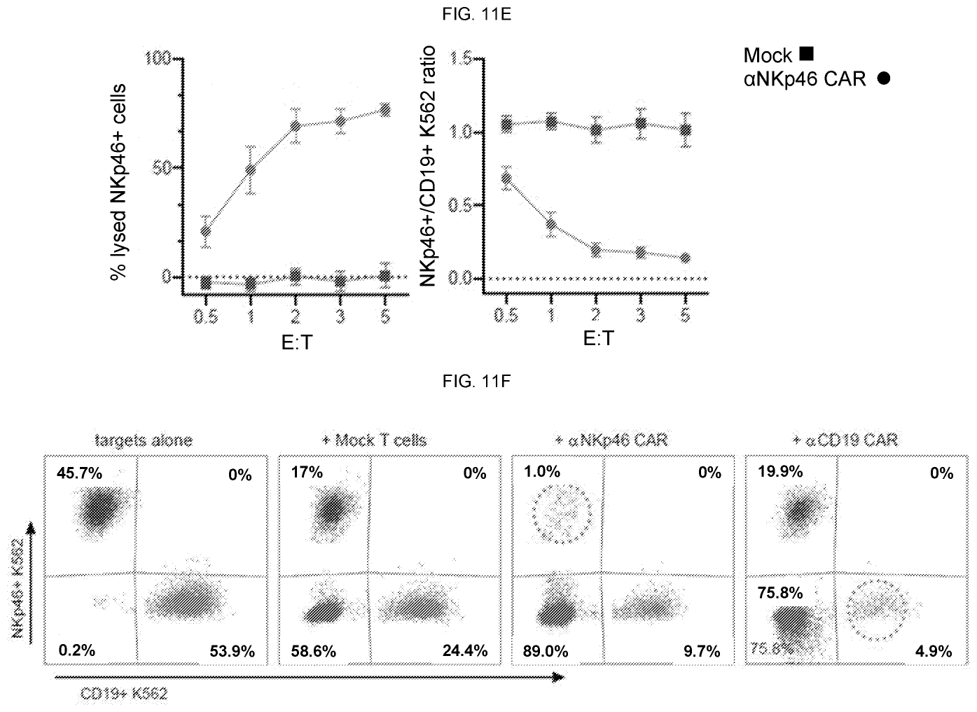

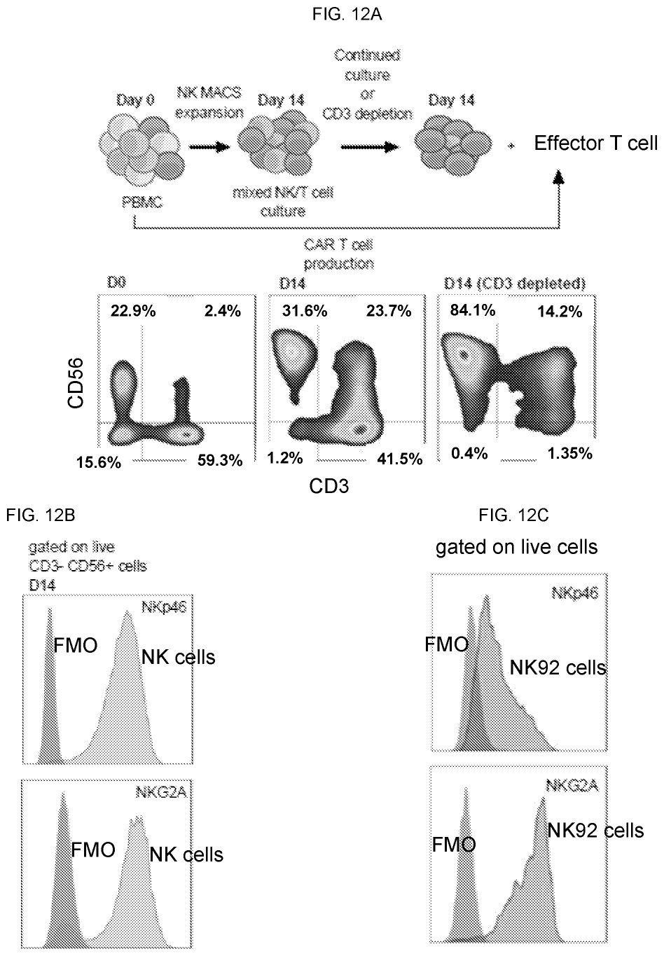

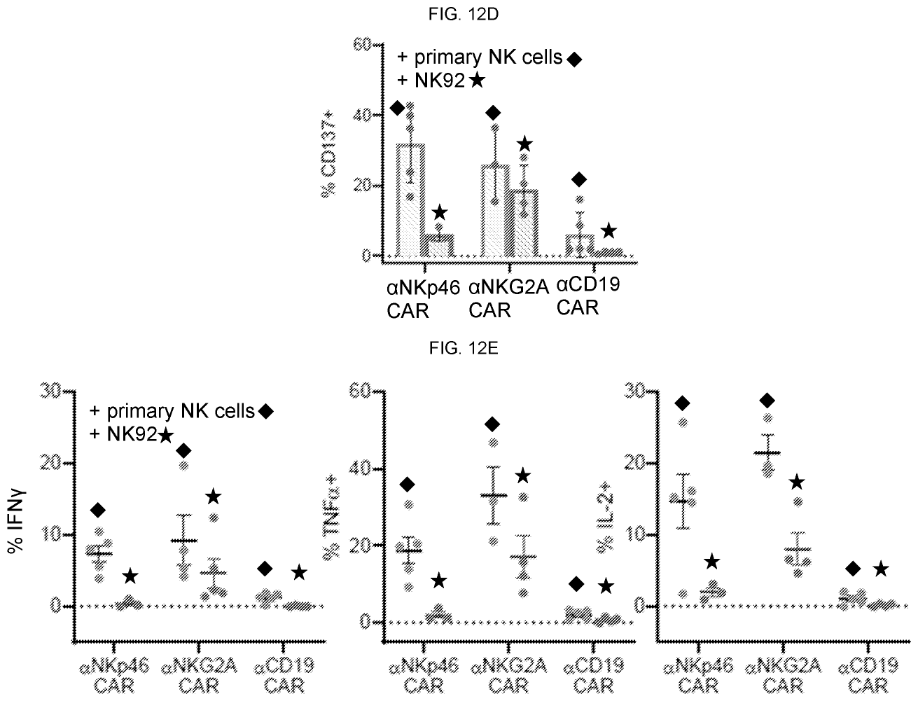

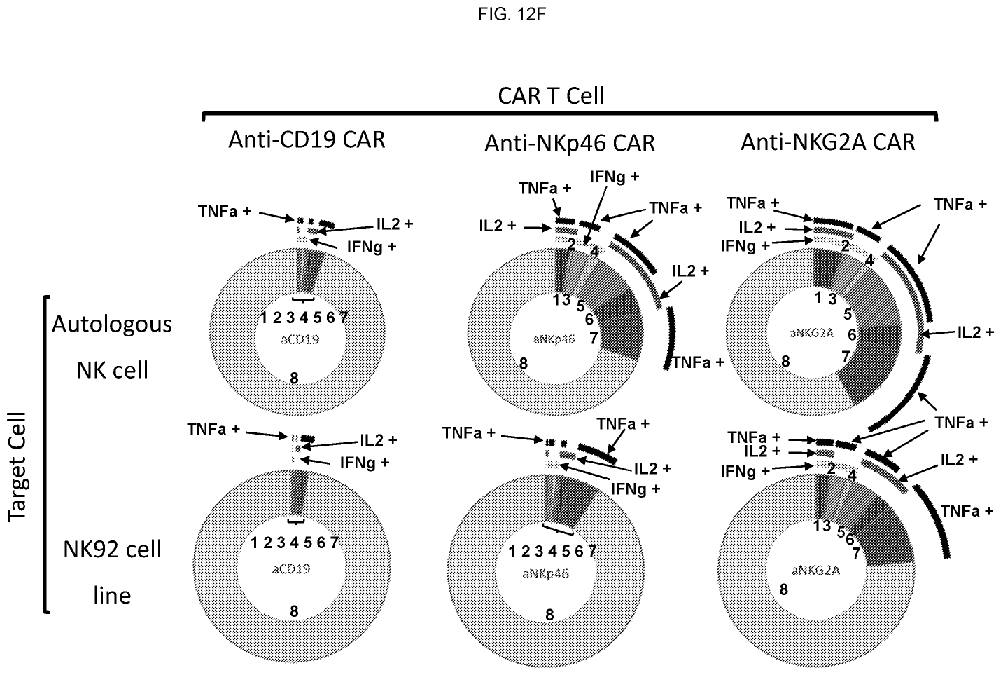

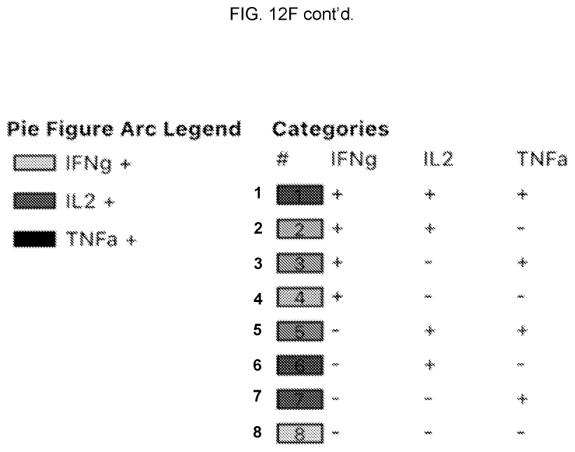

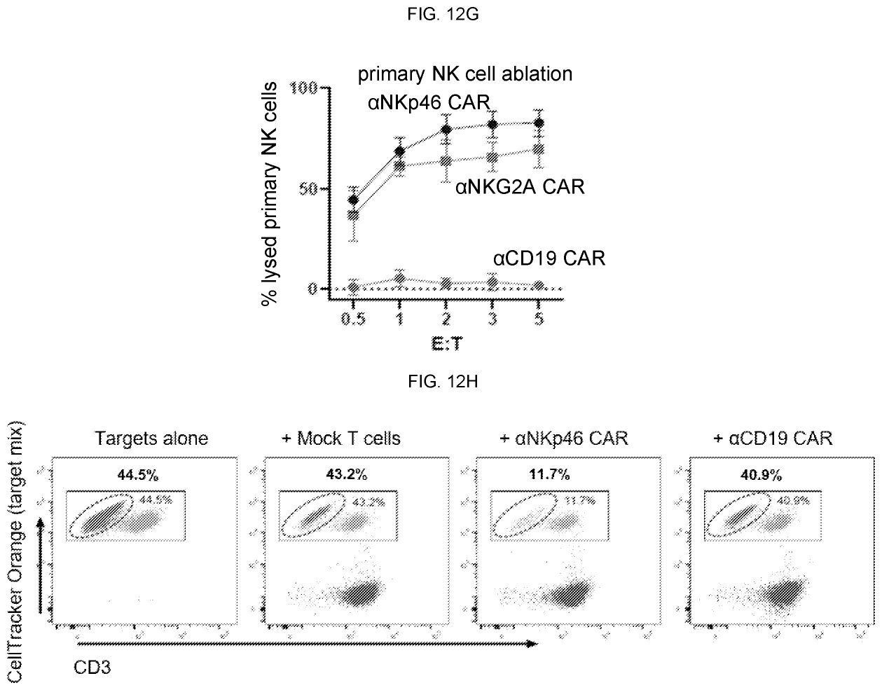

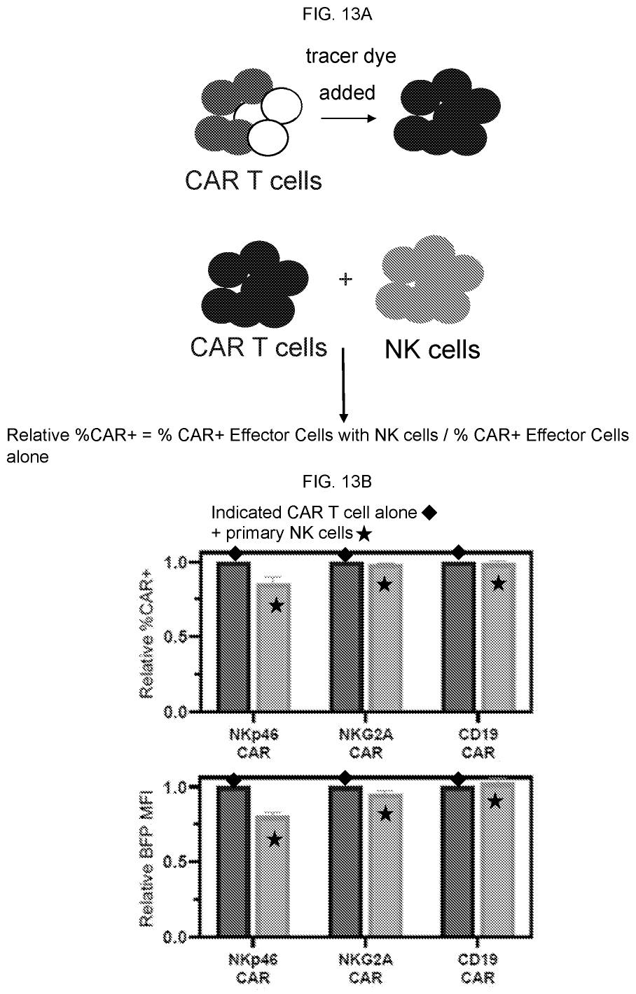

αNKp46 CAR T cells robustly activate against and kill NKp46-expressing K562 cells. The lone widely-characterized and accessible NK lymphoma cell line, NK92, expresses high levels of NKG2A⁺, but relatively low levels of NKp46 (FIG. 12C). Therefore, the αNKp46 CAR T cells were first tested against the myelogenous cell line K562 modified to express the NKp46 receptor. αCD19 CAR T cells and CD19⁺ K562 cells were used as negative control effectors and targets, respectively. Each target cell line was determined by flow cytometry to have near-homogeneous surface expression of its respective target antigen (FIG. 11A).

αNKp46 CAR T cell activation was evaluated as a measure of cell-surface CD137 and intracellular IL-2, TNFα, and IFNγ expression. αNKp46 CAR T cells were mixed with NKp46⁺, CD19⁺, or non-transduced (NT) K562 cells for 24 hours (CD137) or 6 hours (cytokines) at an effector-to-target ratio (E:T) of 2:1. Compared to control αCD19 CAR T cells, αNKp46 CAR T cells displayed specific CD137 upregulation (n=3) and increased IL-2, IFNγ, and TNFα expression (n=5) in response to NKp46⁺ K562 cells (FIGS. 11B, 11C). Nearly half of cytokine-expressing αNKp46 CAR T cells showed polyfunctionality (FIG. 11D). By both measures assayed, αNKp46 and αCD19 CAR T cells showed comparable levels of activation against their respective target.

To determine the killing efficiency of αNKp46 CAR T cells, either Mock or αNKp46 CAR T cells were cultured with an equal mixture of the NKp46+ and CD19+ K562 cells at increasing E:T ratios. Following 24-hour co-cultures, the specific lysis of NKp46⁺ K562 cells significantly increased with increasing doses of αNKp46 CAR T cells compared to mock T cells, nearly reaching 90% at a 5:1 E:T ratio (FIG. 11E). Nevertheless, specific lysis remained distinctly robust at a 2:1 E:T ratio (FIG. 11F).

αNKp46 and αNKG2A CAR T cell show activation in response to and lyse NK cells. The αNKp46 CAR T cells were next tested against NK92 cells despite the cell line's modest NKp46 expression. As expected, αNKp46 CAR T cell activation was minimal after 6 hours of co-culture, showing slight CD137 upregulation but no increase in cytokine production (n=3, FIGS. 12D, 12E). Notably, CD137 upregulation in αNKp46 CAR T cells (mean=6.13%) was still 6-fold greater than in αCD19 CAR T cells (mean=1.04%, n=4). Conversely, the αNKG2A CAR T cells showed elevated CD137 and cytokine expression against the lymphoma cell line (n=4, FIGS. 12D, 12E). CAR-favoring and thus suboptimal NK92 co-culture conditions may have unintentionally reduced αNKG2A CAR T cell activity by restraining NK92 survival. Nonetheless, nearly a third of cytokine-producing αNKG2A CAR T cells were polyfunctional (FIG. 12F).

Given their capacity to target cell lines in strong specific fashion, αNKp46 and αNKG2A CAR T cells were then tested against autologous primary NK cells. PBMCs from three donors were expanded in NK-selecting medium (NK MACS, Miltenyi Biotec, Bergisch Gladbach, Germany) for 14 days, generating mixed lymphocyte cultures of predominantly NK and T cells (FIG. 12A). At this juncture, NK cells almost completely expressed both NKG2A and NKp46 (FIG. 12B). It was observed that T-cell-depleted NK cell cultures activated αNKp46 and αNKG2A—though not αCD19—CAR T cells after 6 hours of co-culture at 2:1 E:T ratios. CD137 upregulation was highest in αNKp46 CAR T cells (n=5; FIG. 12D) while cytokine production was highest with the αNKG2A CAR (n=4; FIG. 12E). Compared to the previously tested cell lines, these autologous primary NK cells generated equal, if not greater, αNKp46 and αNKG2A CAR T cell polyfunctionality (FIG. 12F).

To further determine whether the CAR T cells could kill the NK cells, pre-labeled, mixed NK/T cell cultures were incubated with CAR T cells for 24 hours at a 2:1 E:T ratio. Unlike the αCD19 CAR, both anti-NK CAR constructs reduced the percentage of remaining NK cells in nearly identical dose-dependent trends (FIG. 12G). Robust killing was observed by 2:1 E:T ratios for both the αNKp46 (79% killed) and αNKG2A (64% killed) CARs, only reduced from 5:1 E:T ratios by 3% and 6%, respectively (FIG. 12E).

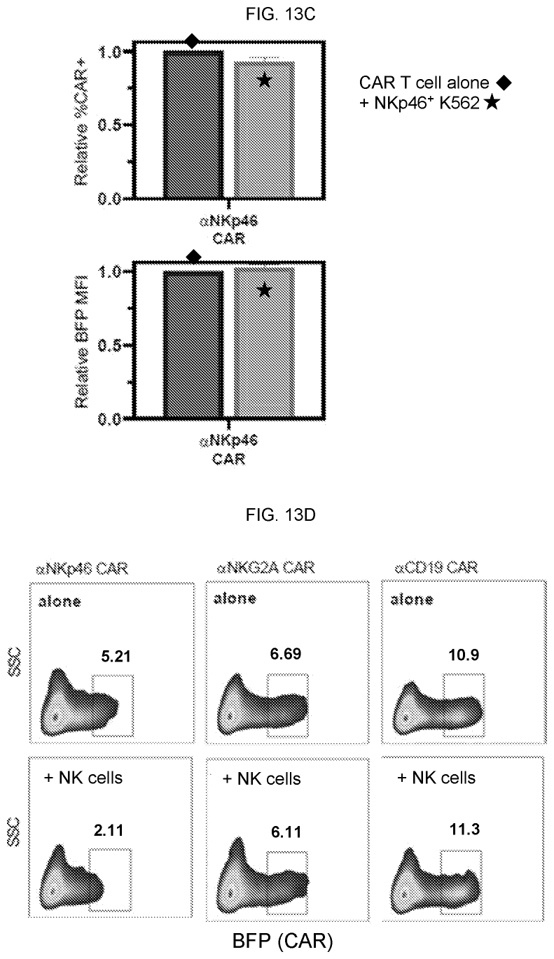

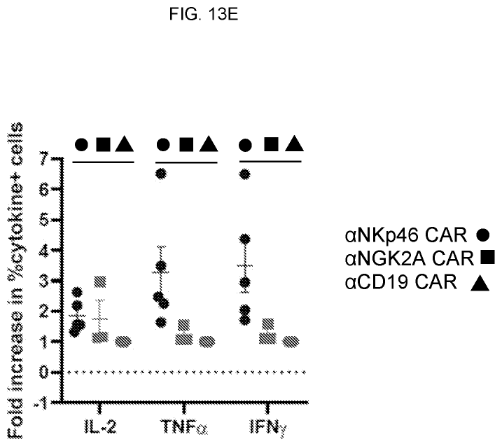

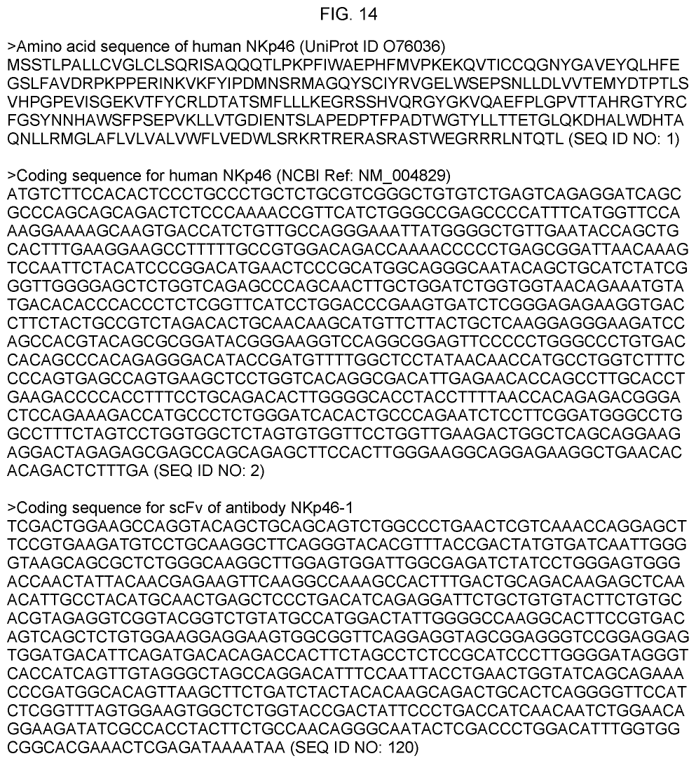

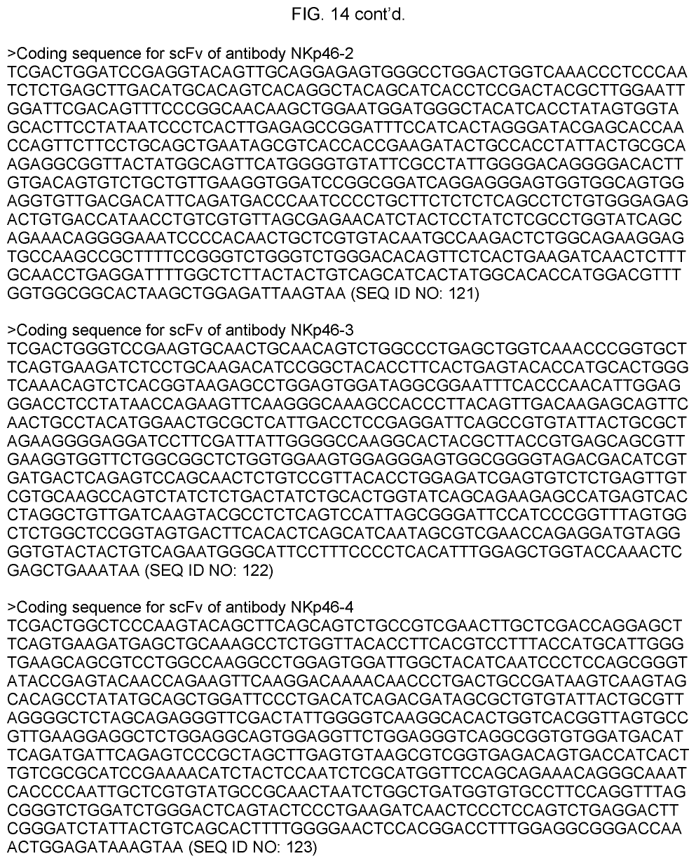

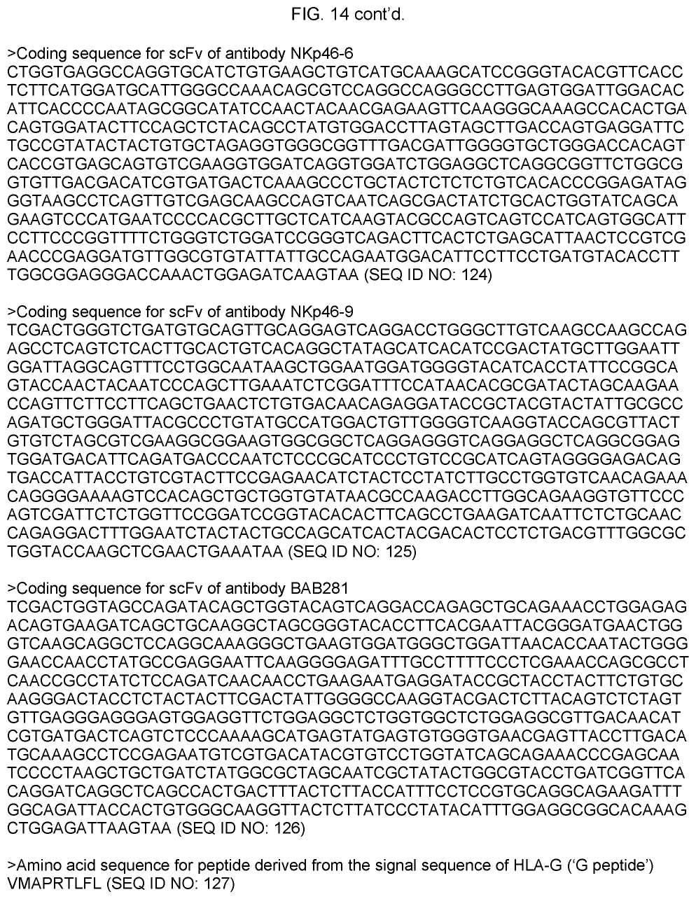

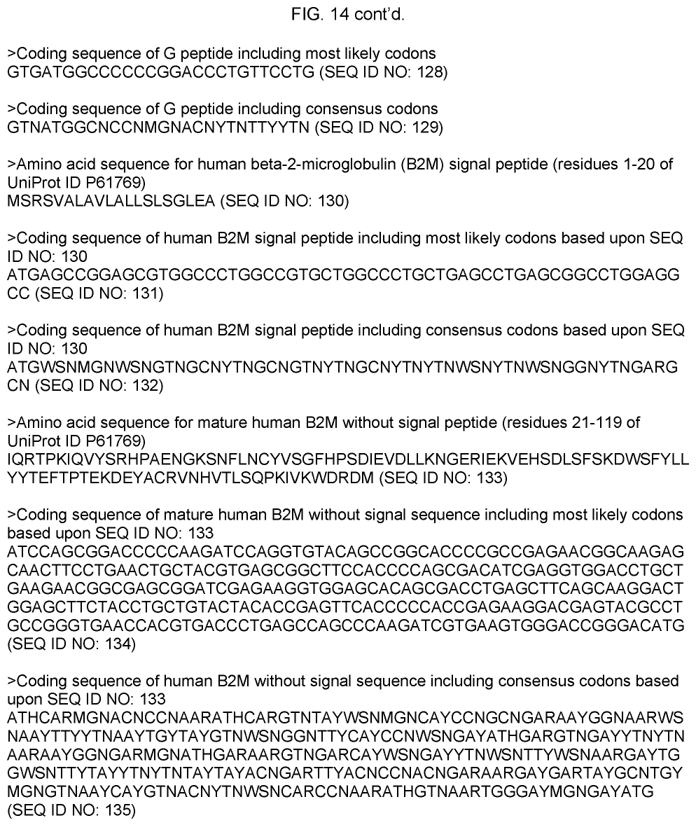

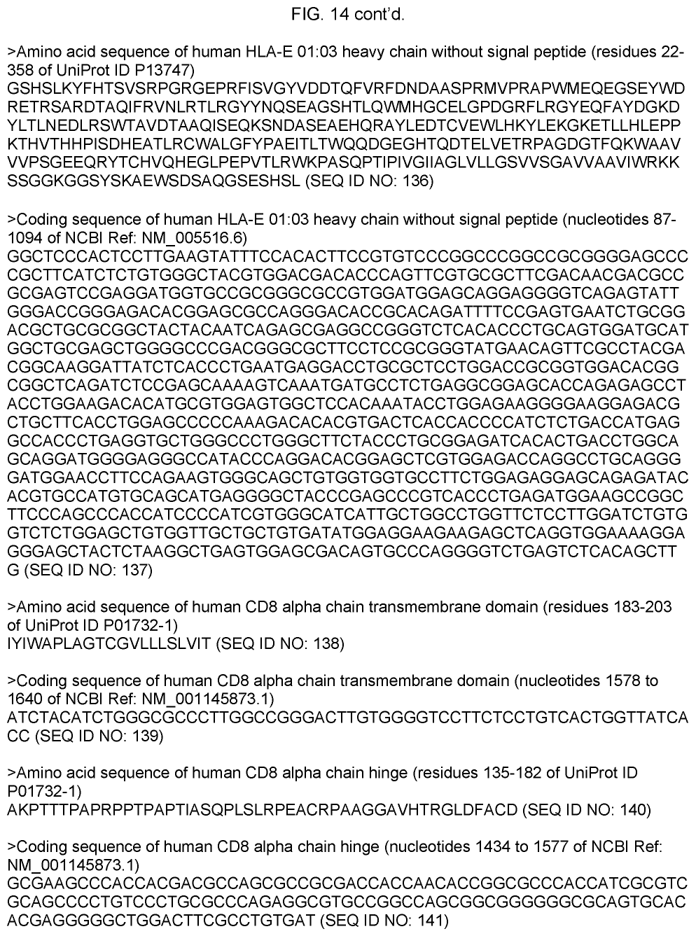

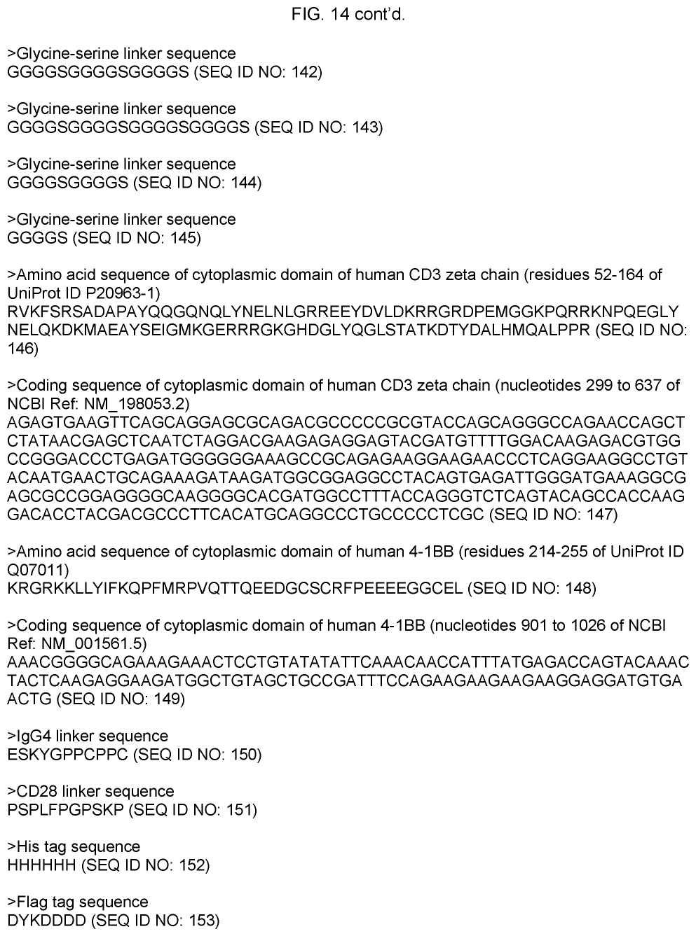

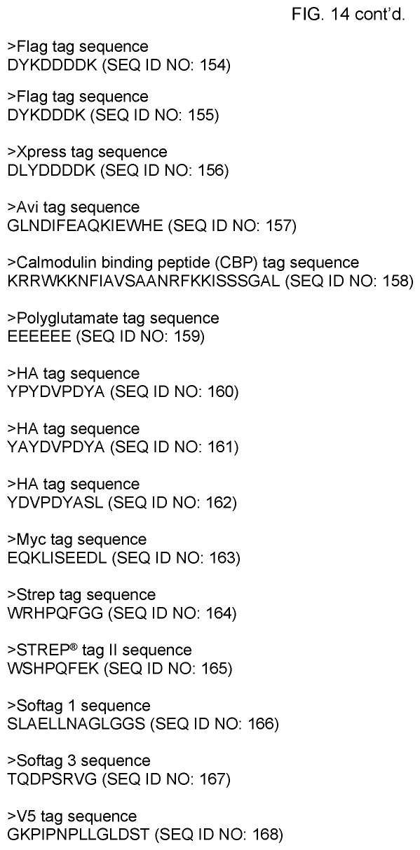

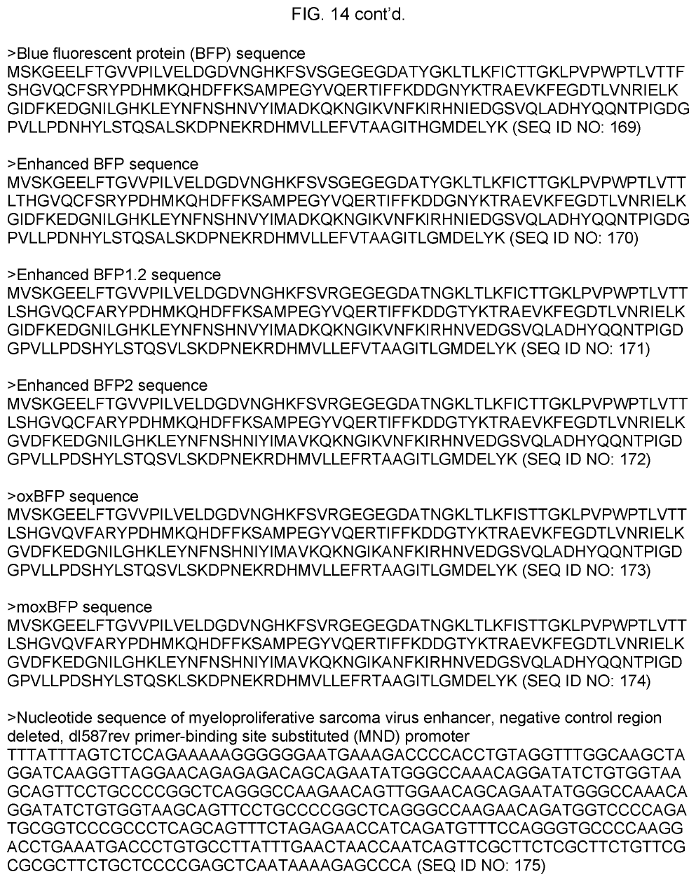

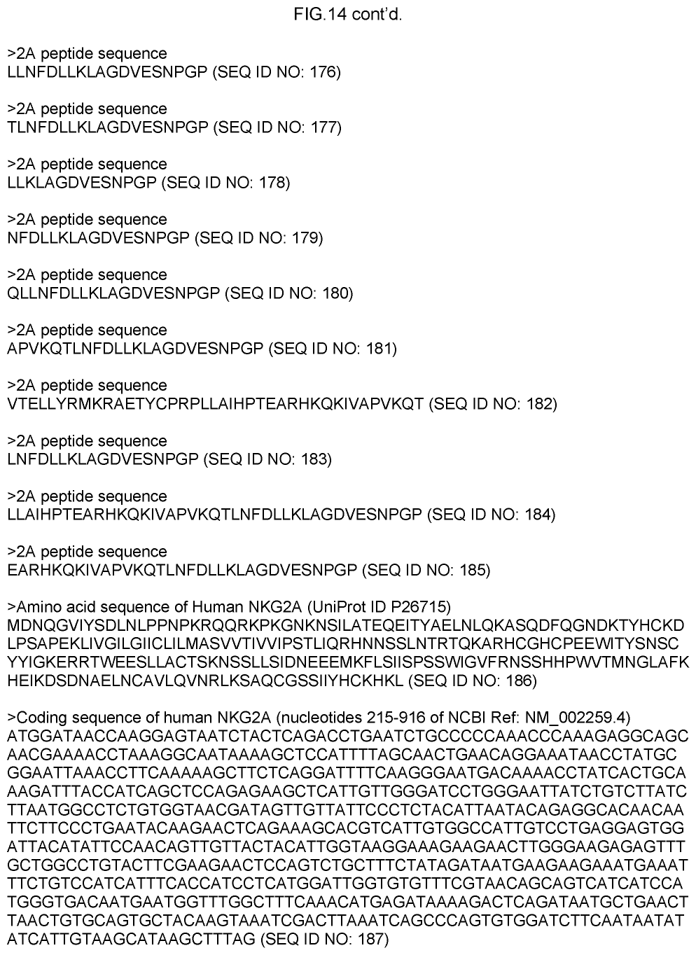

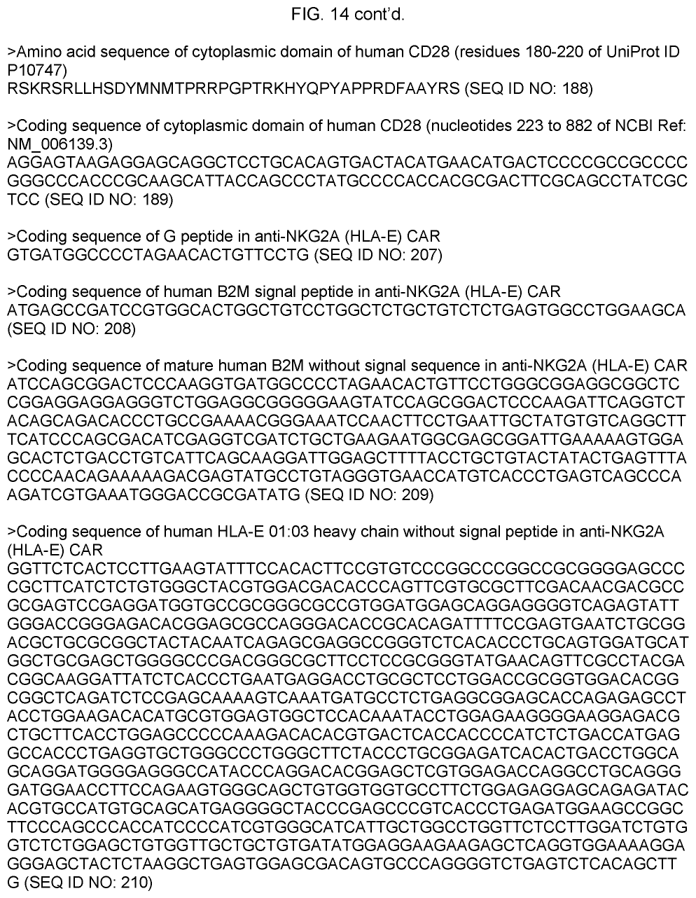

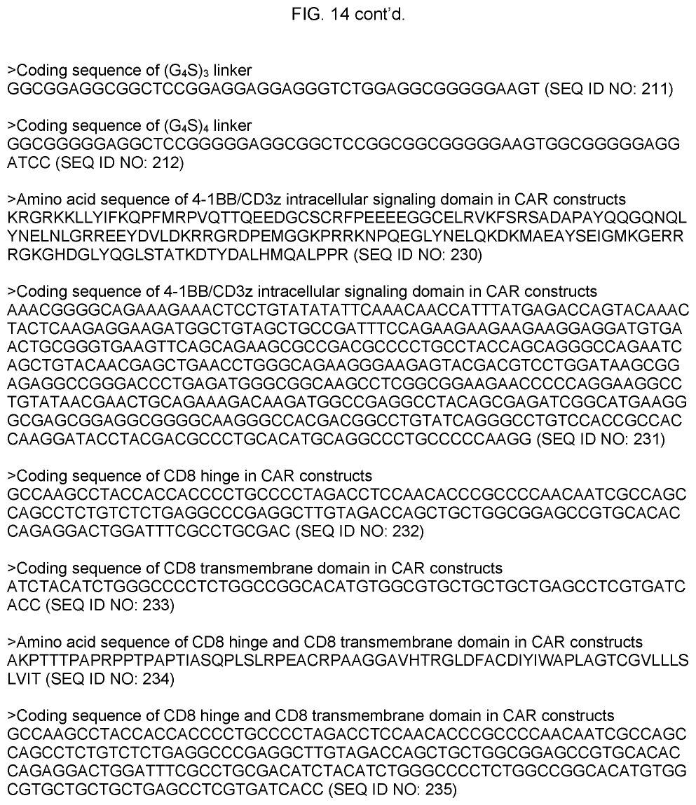

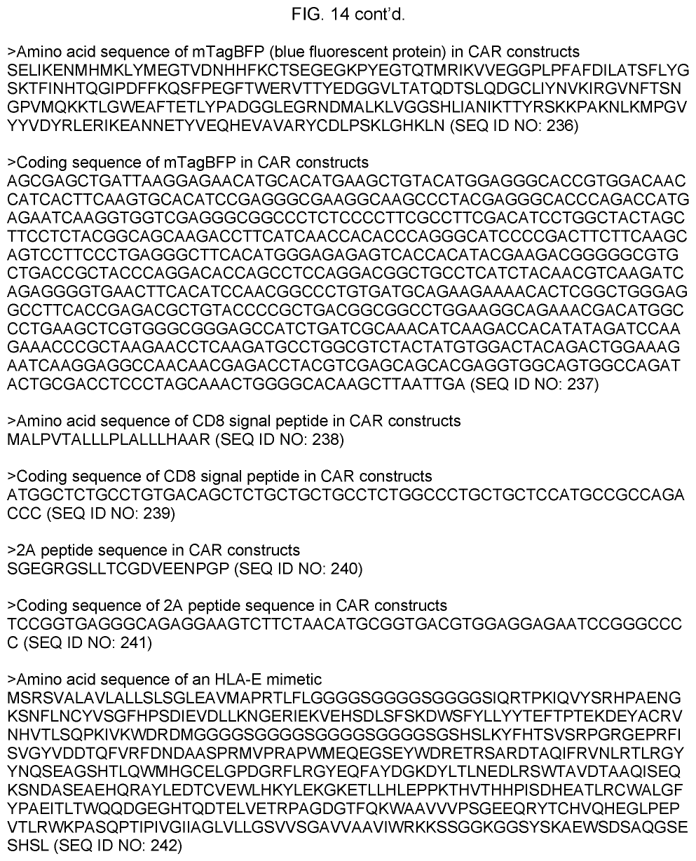

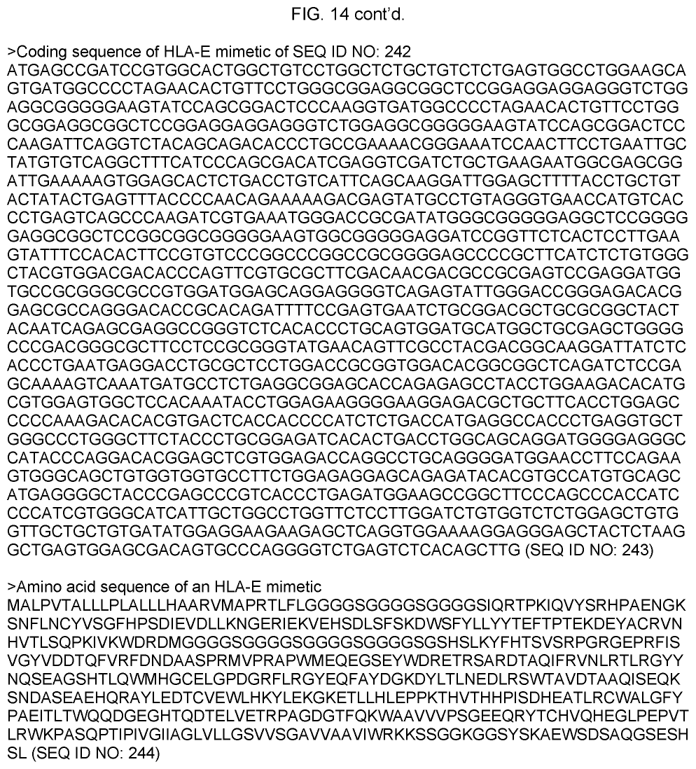

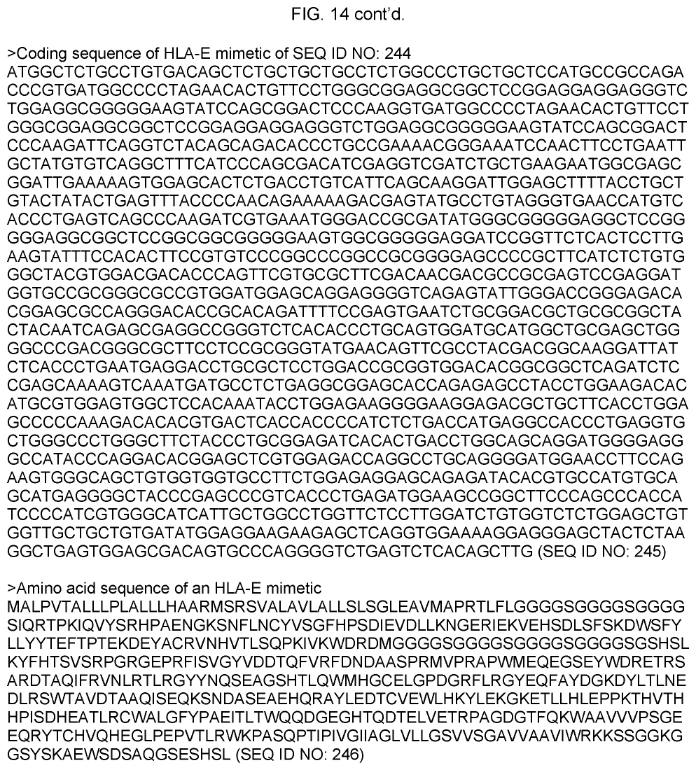

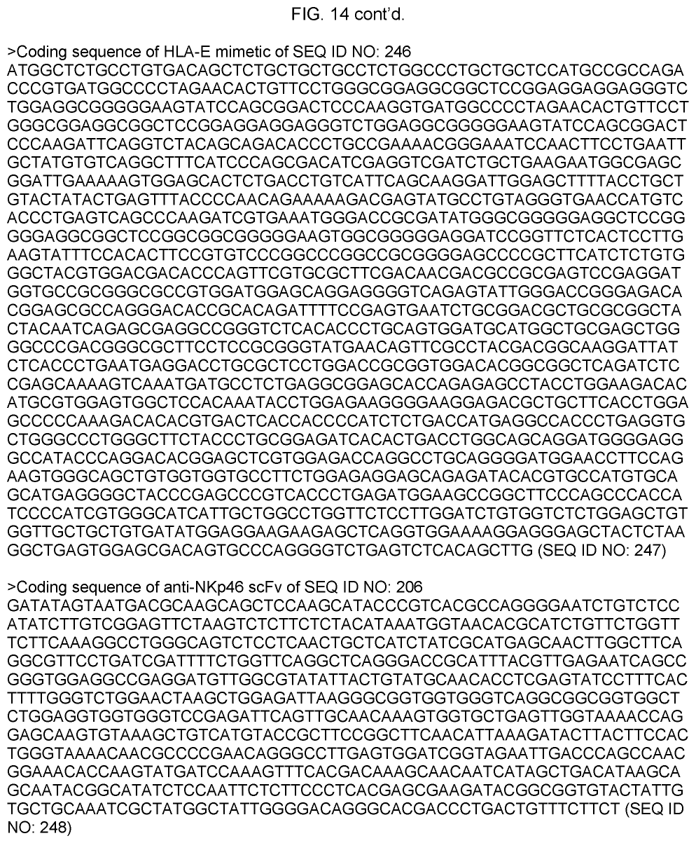

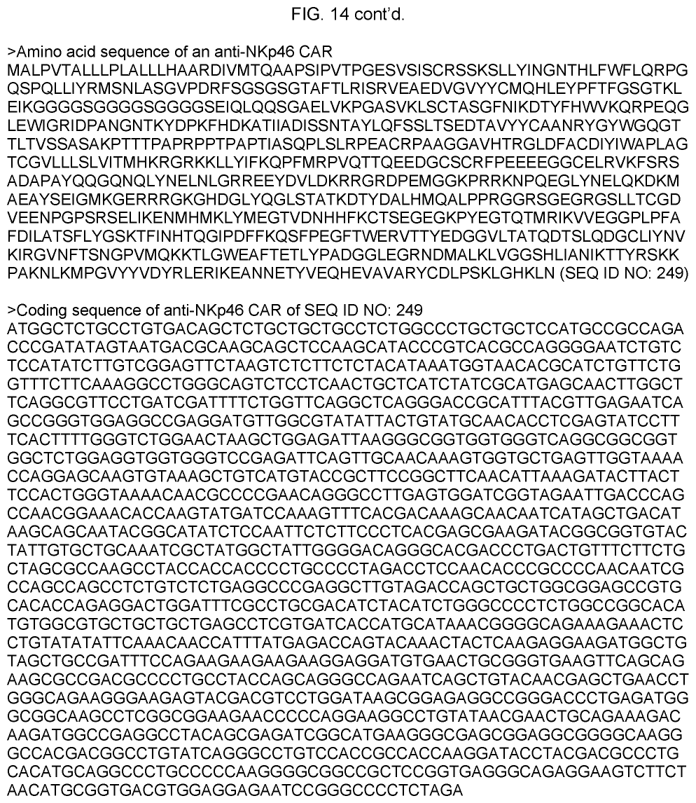

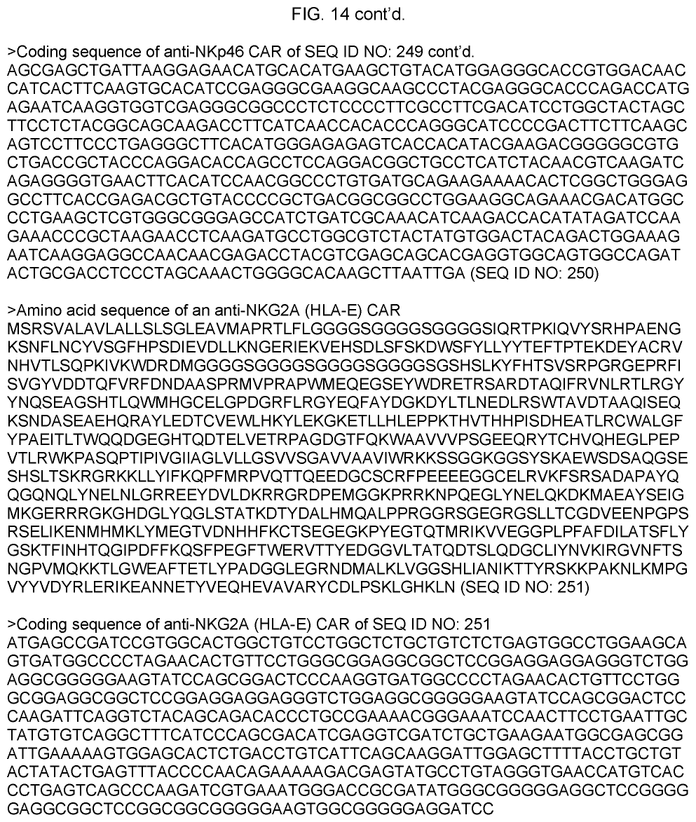

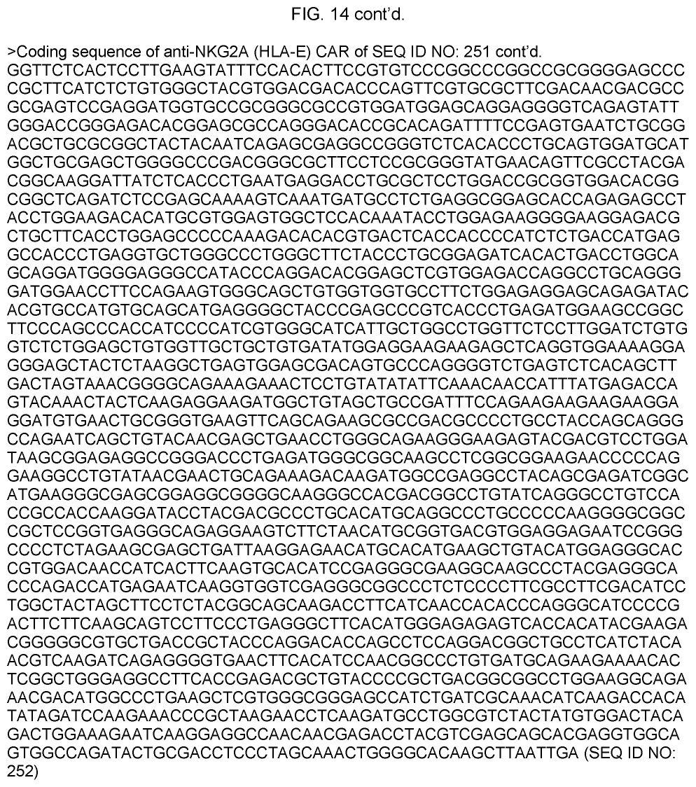

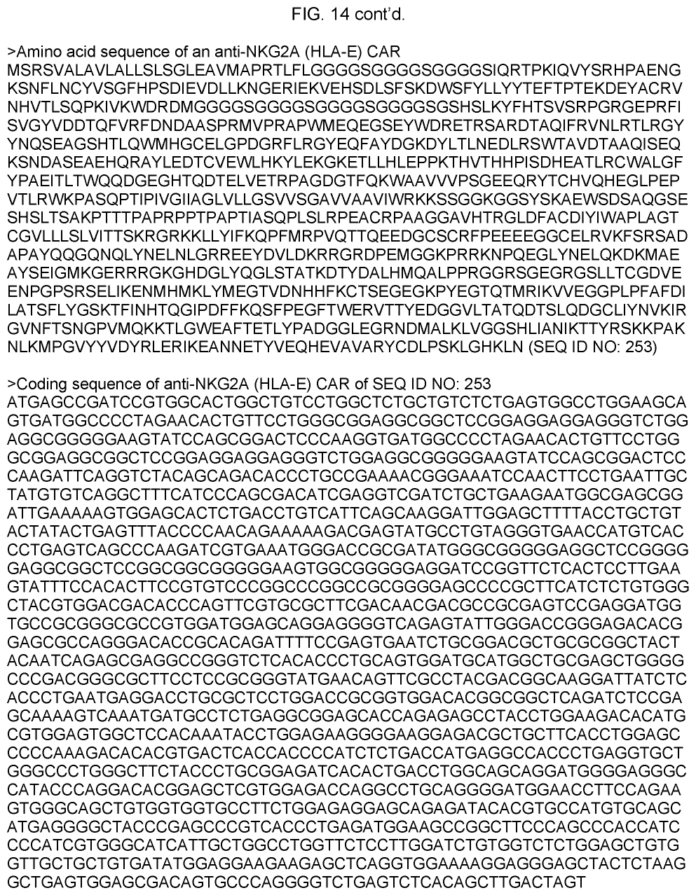

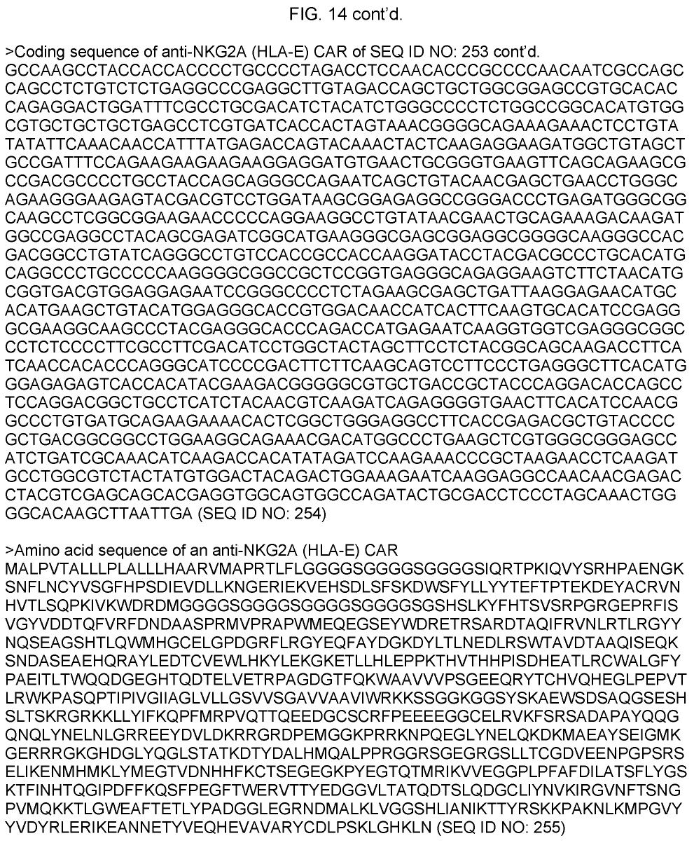

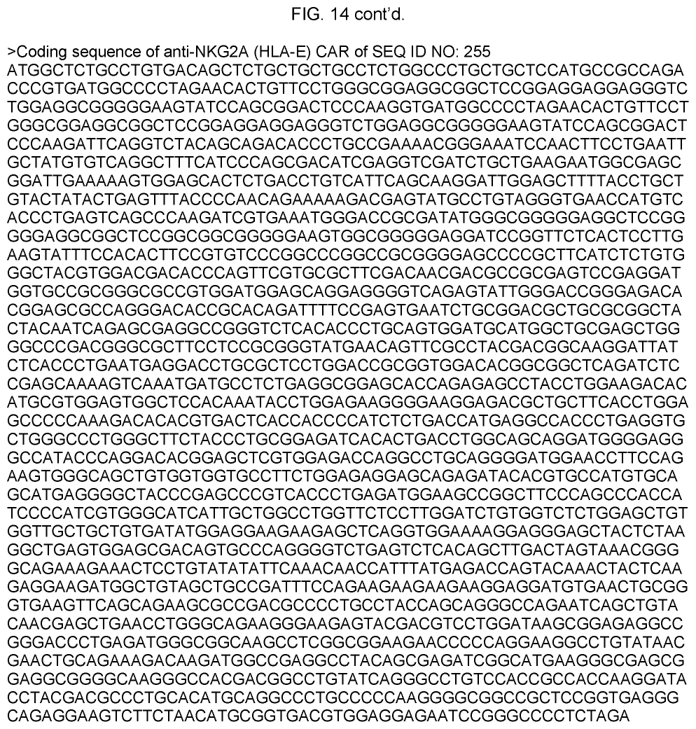

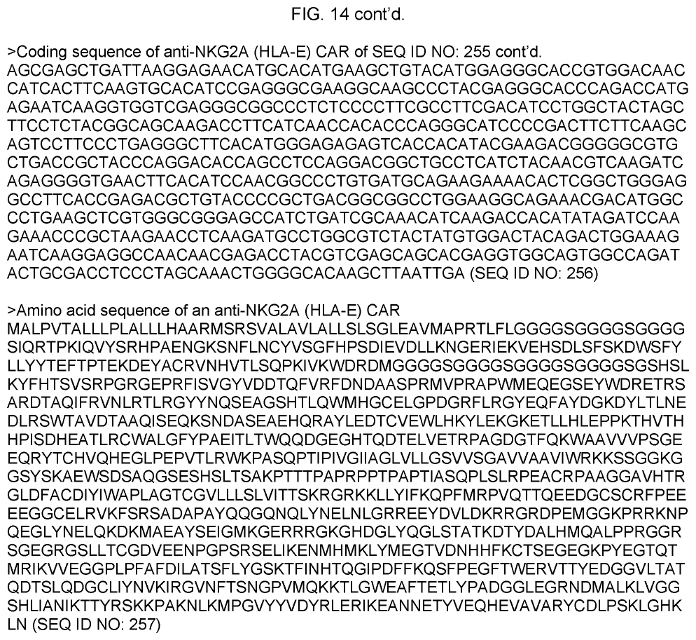

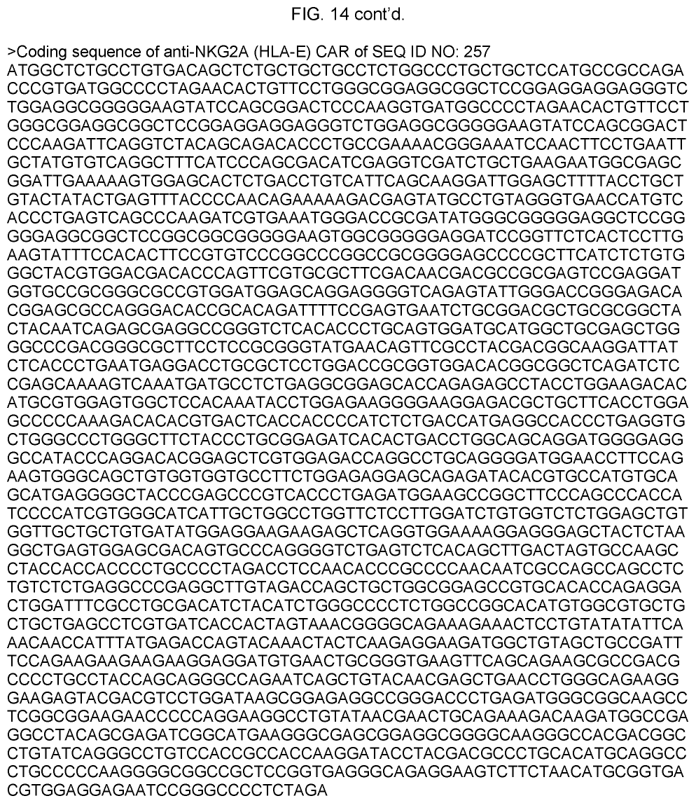

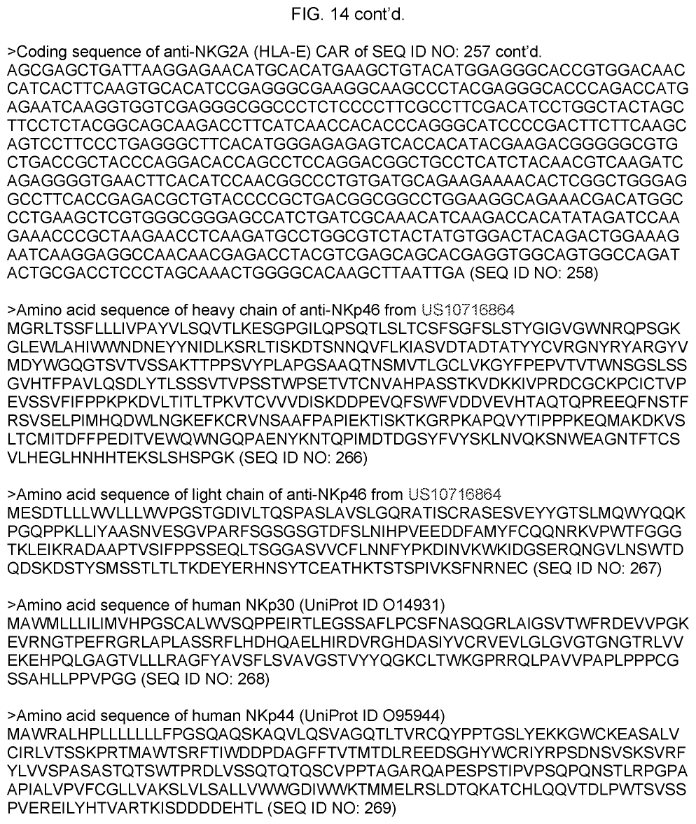

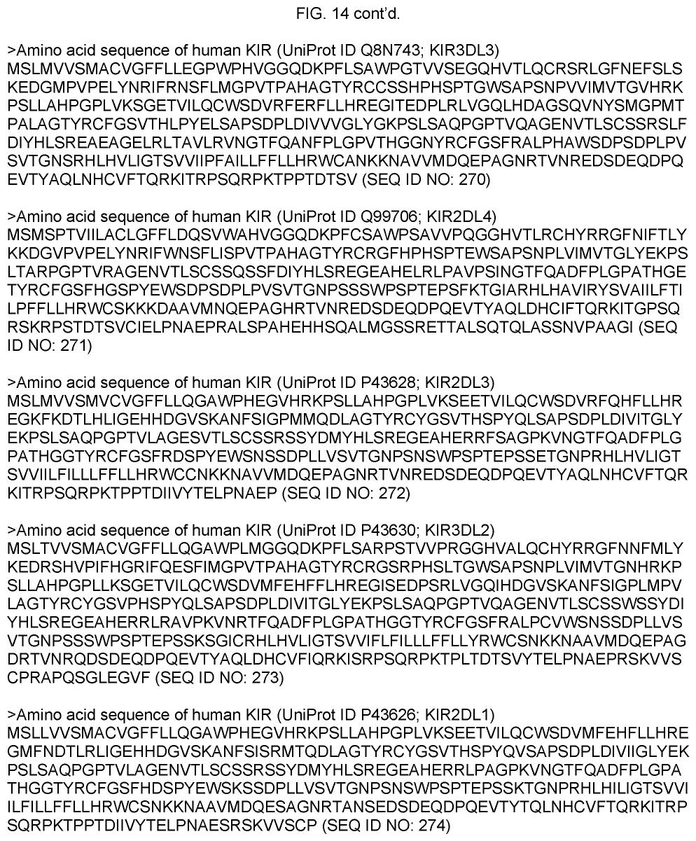

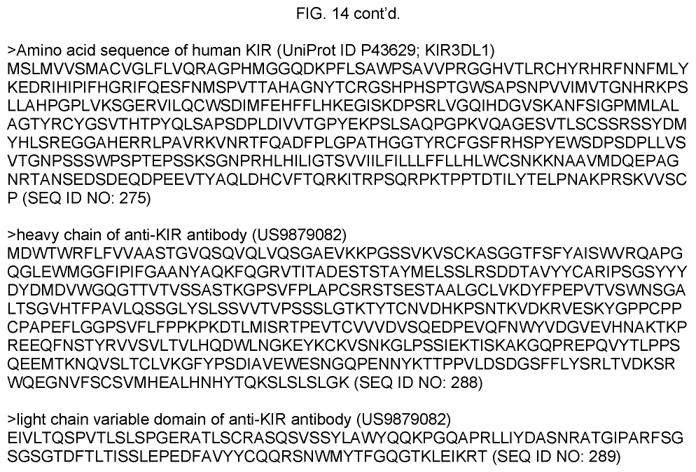

NK cells activate and lyse αNKp46 CAR T cells. Theoretically, engaging the activating receptor NKp46 could elicit a cytotoxic response in NK cells sufficient to kill αNKp46 CAR T cells. Moreover, HLA-E, the binding domain for the αNKG2A CAR, is known to predominantly bind NKG2A, but has also been identified as a ligand for NKG2C, another NK cell activating receptor (Wada et al. Eur. J. Immunol. 34, 81-90 (2004)). Therefore, experiments were conducted to assess whether the targeted NK cells could elicit NKp46- and/or NKG2C-driven killing of either CAR product. Pre-labeled αNKp46 or αNKG2A CAR T cells were mixed with autologous NK cells at a 2:1 NK-to-CAR ratio for 4 hours (FIG. 13A). Because BFP was used as a reporter of CAR expression, the percentage of BFP-expressing cells and the median BFP mean fluorescence intensity (MFI) before and after adding NK cells were used to assess NK-mediated CAR T cell death.

A 12% reduction in BFP expression and a 15% decrease in median BFP MFI was observed in αNKp46 CAR T cells cultured with autologous NK cells (FIGS. 13B-13D). αNKG2A and αCD19 CAR T cells did not show significant changes in either BFP percent expression or median MFI. Therefore, focusing on the αNKp46 CAR, experiments were conducted to confirm that CAR death was NK cell-mediated rather than a case of CAR T cell fratricide, as described in other studies (Hamieh et al. Nature 568: 112-116 (2019)). αNKp46 CAR T cells were mixed with NKp46⁺ K562 cells at a 2:1 E:T ratio for 4 hours. Despite successfully engaging the NKp46 receptor on K562 cells (FIG. 11B), αNKp46 CAR T cells showed no significant decline in either BFP percent expression or median MFI (FIG. 13C). A representative density plot analysis shows BFP expression in αNKp46 CAR T cells before and after NK cells were added (FIG. 13D).

A sub-population of BFP^{bright} cells waning by more than 50% when autologous NK cells were added was identified, perhaps suggesting a higher susceptibility of lysis for αNKp46 CAR T cells with higher surface CAR density.

Furthermore, NK cells cultured with αNKp46 CAR T cells showed a 1.8-fold increase in TNFα expression, and a 3.5-fold increase IFNγ expression, compared to NK cells cultured with αCD19 CAR T cells (FIG. 13E).

NK cell malignancies include a small but devastating subset of hematological cancers for which there are limited effective treatment options. In recent years, the potency of CAR T and CAR NK cells against T- and B-cell tumors, though this work demonstrates for the first time that T cells can be engineered to kill NK cells with comparable efficiency. Combined with the past clinical successes of CAR T cells, this work encourages their use in treating these catastrophic NK cell malignancies.

We have addressed NK cell heterogeneity and antigen specificity by devising two alternative CAR constructs targeting NK cell receptors NKp46 and NKG2A. Available data suggests that most NK malignancies express one or both of these receptors. It is of note that NKp46 is an activating receptor, while NKG2A promotes an inhibitory signal. Moreover, the αNKp46 CAR employs the more conventional scFv-based design, whereas the αNKG2A CAR makes use of a synthetic ligand, HLA-E, which has potential to be less immunogenic. Nonetheless, it should also be possible to target NKG2A with an scFv-based construct. Targeting each receptor simultaneously, either with two separate effectors, or as a single dual-CAR T cell product, has the potential advantages of mitigating antigen loss and tumor escape (Majzner & Mackall. Cancer Discov. 8, 1219-1226 (2018)).

An unforeseen result of targeting the activating NK receptor NKp46 was NK cell activation and modest (12-15%) cytotoxicity against αNKp46 CAR T cells. Simultaneously targeting the inhibitory NKG2A receptor with the αNKG2A CAR may mitigate this effect, while also granting the aforementioned advantages of dual specificity. On another front, the HLA-E ligand alone may confer benefits in the development of off-the-shelf therapies, as allogeneic CAR T cell products depleted of HLA-A and HLA-B but overexpressing HLA-E can avoid both host T and NK cell-mediated rejection (Gornalusse et al. Nat. Biotechnol. 35, 765-772 (2017); Torikai et al. Blood 122, 1341-1349 (2013)). In summary, a dual αNKG2A/αNKp46 CAR offers the benefit of 1) broadening the target landscape, 2) minimizing chances of tumor escape, and 3) supporting the development of an off-the-shelf anti-NK CAR T cell therapy that is accessible and less costly.

In addition to the inflammatory complications seen with CAR T cell therapy (Neelapu et al. Nat. Rev. Clin. Oncol. 15, 47-62 (2018)), important on-target/off-tumor toxicities are to be expected with anti-NK cell CAR T cells. Approximately 5-15% of PBMCs are NK cells. These would be semi-permanently depleted by effective anti-NK cell CAR T cell therapy. This is analogous to αCD19 CAR T cell therapy, which results in B-cell aplasia. However, hypogammaglobulinemia can be managed with immunoglobulin replacement therapy. Individuals with congenital NK cell deficiency or dysfunction are at high risk of herpesviruses and human papillomavirus infections (Mace & Orange. Immunol. Rev. 287, 202-225 (2019)), and mitigation strategies include prophylactic antiviral and/or immunostimulatory therapies (Orange, J. S. J. Allergy Clin. Immunol. 132, 515-525 (2013)). The target NKG2A is also found on a small subset of CD8+ T cells (Braud et al. Trends Immunol. 24, 162-164 (2003)), although the clinical significance of this is less clear.

Anti-NK CAR T cells also have potential for research and therapeutic applications other than NK malignancies. HLA-E is significantly overexpressed on a variety of tumors, and expression of its cognate inhibitory receptor, NKG2A, on both tumor-infiltrating NK and CD8+ T cells has been linked with poor prognoses (Kamiya et al. J. Clin. Invest. 129, 2094-2106 (2019)). Therefore, recognition checkpoint inhibitors have been developed targeting this NKG2A-HLA-E axis (Kamiya et al. J. Clin. Invest. 129, 2094-2106 (2019); van Hall et al. J. Immunother. Cancer 7, 263 (2019)). Compared to analogous monoclonal antibodies, αNKG2A CAR T cells would directly deplete suppressed cells may more effectively deplete inhibitory NK and T cells, thus revamping the tumor microenvironment, while benefiting from a longer half-life and superior tumor infiltration. Like non-cell-based approaches, this strategy would have widespread clinical applicability.

In summary, this study demonstrated that CAR T cells can be engineered to kill NK cells. Targeting two different NK receptors was effective and has practical advantages when used simultaneously. In a clinical setting, on-target toxicities would be expected and treatable, though ultimately this risk is balanced by the dire need for new treatment options for advanced-stage NK cell malignancies. Thus, the present study provides effective compositions and methods for anti-NK CAR T cell therapy.

(xvii) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST. 25 (1998), Appendix 2, Tables 1 and 3. Only one strand of each nucleic acid sequence is shown, but the complementary strand is understood as included in embodiments where it would be appropriate.

Nucleotide sequences shown in SEQ ID NOs: 128, 129, 131, 132, 134, and 135 have been reverse translated using the Sequence Manipulation Suite: Reverse Translate available from the World Wide Web at bioinformatics.org/sms2/rev_trans.html and a human codon usage table obtained from the World Wide Web at genscript.com/tools/codon-frequency-table. Reverse Translate accepts an amino acid sequence as input and generates a nucleotide sequence representing the most likely non-degenerate coding sequence given the provided codon usage table (‘most likely codons’). A consensus sequence derived from all the possible codons for each amino acid is also returned (‘consensus codons’). The terms “specific binding affinity” or “specifically binds” or “specific binding” or “specifically targets” as used herein, describe binding of one molecule to another at greater binding affinity than background binding. A binding domain (e.g., of a CAR including a binding domain) “specifically binds” to a target molecule if it binds to or associates with a target molecule with an affinity or Ka (i.e. an equilibrium association constant of a particular binding interaction with units of 1/M) of, for example, greater than or equal to 10⁵ M⁻¹. In particular embodiments, a binding domain (or CAR) binds to a target with a Ka greater than or equal to 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (Kd) of a particular binding interaction with units of M (e.g., 10⁻⁵ M to 10⁻¹³ M, or less). Affinities of binding domains and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, N.J., or optical biosensor technology such as the EPIC system or EnSpire that are available from Corning and Perkin Elmer respectively (see also, e.g., Scatchard et al. (1949) Ann. N.Y. Acad. Sci. 51:660; U.S. Pat. Nos. 5,283,173; 5,468,614).

In particular embodiments, the affinity of specific binding is 2 times greater than background binding, 5 times greater than background binding, 10 times greater than background binding, 20 times greater than background binding, 50 times greater than background binding, 100 times greater than background binding, or 1000 times greater than background binding or more.

“Derived from” as used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3ζ molecule, the intracellular signaling domain retains sufficient CD3ζ structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3ζ sequence and delete unwanted sequence, or impose mutations, to arrive at the intracellular signaling domain.

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability of a cell expressing a CAR to bind to an NK cell surface marker and target the cell expressing the NK cell surface marker.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A cell genetically modified to express a chimeric antigen receptor (CAR), wherein when expressed the CAR comprises:
an extracellular component;
an intracellular component; and
a transmembrane domain linking the extracellular component to the intracellular component,
wherein the extracellular component comprises
(A) an NKp46 binding domain comprising:
a heavy chain variable (VH) domain comprising: a CDRH1 having the sequence as set forth in SEQ ID NO: 190, a CDRH2 having the sequence as set forth in SEQ ID NO: 193, and a CDRH3 having the sequence as set forth in SEQ ID NO: 196; and a light chain variable (VL) domain comprising: a CDRL1 having the sequence as set forth in SEQ ID NO: 198, a CDRL2 having the sequence as set forth in SEQ ID NO: 201, and a CDRL3 having the sequence as set forth in SEQ ID NO: 202, according to Kabat numbering; and
(B) an NKG2A binding domain comprising an artificial human leukocyte antigen (HLA)-E mimetic comprising a heterotrimer of: i) a signal peptide of HLA-G; ii) mature beta-2 microglobulin (B2M); and iii) an HLA-E 01:03 heavy chain.

2. The cell of claim 1, wherein
the NKp46 binding domain comprises the VH domain having the sequence as set forth in SEQ ID NO: 204 and the VL domain having the sequence as set forth in SEQ ID NO: 205; and/or
the artificial HLA-E mimetic has the sequence as set forth in SEQ ID NOs: 242, 244, or 246.

3. The cell of claim 1, wherein the anti-NKp46 binding domain is a single chain variable fragment (scFv) having the sequence as set forth in SEQ ID NO: 206.

4. The cell of claim 1, wherein the intracellular component comprises an intracellular signaling domain.

5. The cell of claim 1, wherein the intracellular signaling domain comprises CD3ζ, common FcRγ, Fc γ RIIa, FcR β, CD3 γ, CD3 δ, CD3 ε, CD79a, CD79b, DAP10, and/or DAP12.

6. The cell of claim 1, wherein the cell is a T cell, an NK cell, a macrophage, a hematopoietic stem cell (HSC), or a hematopoietic progenitor cell (HPC).

7. A chimeric antigen receptor (CAR) that when expressed by a cell comprises:
an extracellular component; and
an intracellular component,
wherein the extracellular component comprises an activating NK receptor binding domain and/or an inhibitory NK receptor binding domain.

8. The CAR of claim 7, further comprising a transmembrane domain linking the extracellular component to the intracellular component.

9. The CAR of claim 7, wherein the activating NK receptor binding domain comprises an NKp30 binding domain, an NKp44 binding domain, and/or an NKp46 binding domain.

10. The CAR of claim 9, wherein the NKp30 binding domain comprises an antigen binding fragment of antibody AZ20, antibody A76, antibody Z25, antibody 15E1, antibody 9G1, antibody 15H6, antibody 9D9, antibody 3A12, antibody 12D10, antibody clone #210845, antibody clone p30-15, or antibody clone AF29-4D12.

11. The CAR of claim 9, wherein the NKp44 binding domain comprises an antigen binding fragment of antibody Z231, antibody clone #253415, antibody clone 44.189, antibody clone 1G6, or antibody clone P44-8.

12. The CAR of claim 9, wherein the NKp46 binding domain comprises:
(A) a heavy chain variable (VH) domain comprising: a CDRH1 having the sequence as set forth in SEQ ID NO: 190, a CDRH2 having the sequence as set forth in SEQ ID NO: 193, and a CDRH3 having the sequence as set forth in SEQ ID NO: 196; and a light chain variable (VL) domain comprising: a CDRL1 having the sequence as set forth in SEQ ID NO: 198, a CDRL2 having the sequence as set forth in SEQ ID NO: 201, and a CDRL3 having the sequence as set forth in SEQ ID NO: 202;
(B) a VH domain comprising: a CDRH1 having the sequence as set forth in SEQ ID NO: 3, a CDRH2 having the sequence as set forth in SEQ ID NO: 6, and a CDRH3 having the sequence as set forth in SEQ ID NO: 9; and a VL domain comprising: a CDRL1 having the sequence as set forth in SEQ ID NO: 12, a CDRL2 having the sequence as set forth in SEQ ID NO: 15, and a CDRL3 having the sequence as set forth in SEQ ID NO: 16;
(C) a VH domain comprising: a CDRH1 having the sequence as set forth in SEQ ID NO: 19, a CDRH2 having the sequence as set forth in SEQ ID NO: 22, and a CDRH3 having the sequence as set forth in SEQ ID NO: 24; and a VL domain comprising: a CDRL1 having the sequence as set forth in SEQ ID NO: 27, a CDRL2 having the sequence as set forth in SEQ ID NO: 30, and a CDRL3 having the sequence as set forth in SEQ ID NO: 31;
(D) a VH domain comprising: a CDRH1 having the sequence as set forth in SEQ ID NO: 33, a CDRH2 having the sequence as set forth in SEQ ID NO: 36, and a CDRH3 having the sequence as set forth in SEQ ID NO: 39; and a VL domain comprising: a CDRL1 having the sequence as set forth in SEQ ID NO: 42, a CDRL2 having the sequence as set forth in SEQ ID NO: 45, and a CDRL3 having the sequence as set forth in SEQ ID NO: 46;
(E) a VH domain comprising: a CDRH1 having the sequence as set forth in SEQ ID NO: 48, a CDRH2 having the sequence as set forth in SEQ ID NO: 51, and a CDRH3 having the sequence as set forth in SEQ ID NO: 54; and a VL domain comprising: a CDRL1 having the sequence as set forth in SEQ ID NO: 57, a CDRL2 having the sequence as set forth in SEQ ID NO: 60, and a CDRL3 having the sequence as set forth in SEQ ID NO: 61;
(F) a VH domain comprising: a CDRH1 having the sequence as set forth in SEQ ID NO: 63, a CDRH2 having the sequence as set forth in SEQ ID NO: 66, and a CDRH3 having the sequence as set forth in SEQ ID NO: 69; and a VL domain comprising: a CDRL1 having the sequence as set forth in SEQ ID NO: 42, a CDRL2 having the sequence as set forth in SEQ ID NO: 45, and a CDRL3 having the sequence as set forth in SEQ ID NO: 72;
(G) a VH domain comprising: a CDRH1 having the sequence as set forth in SEQ ID NO: 74, a CDRH2 having the sequence as set forth in SEQ ID NO: 77, and a CDRH3 having the sequence as set forth in SEQ ID NO: 78; and a VL domain comprising: a CDRL1 having the sequence as set forth in SEQ ID NO: 81, a CDRL2 having the sequence as set forth in SEQ ID NO: 30, and a CDRL3 having the sequence as set forth in SEQ ID NO: 82;
(H) a VH domain comprising: a CDRH1 having the sequence as set forth in SEQ ID NO: 84, a CDRH2 having the sequence as set forth in SEQ ID NO: 87, and a CDRH3 having the sequence as set forth in SEQ ID NO: 90; and a VL domain comprising: a CDRL1 having the sequence as set forth in SEQ ID NO: 93, a CDRL2 having the sequence as set forth in SEQ ID NO: 96, and a CDRL3 having the sequence as set forth in SEQ ID NO: 97; or
(I) a VH domain comprising: a CDRH1 having the sequence as set forth in SEQ ID NO: 260, a CDRH2 having the sequence as set forth in SEQ ID NO: 261, and a CDRH3 having the sequence as set forth in SEQ ID NO: 262; and a VL domain comprising: a CDRL1 having the sequence as set forth in SEQ ID NO: 263, a CDRL2 having the sequence as set forth in SEQ ID NO: 264, and a CDRL3 having the sequence as set forth in SEQ ID NO: 265,
each according to Kabat numbering.

13. The CAR of claim 9, wherein the NKp46 binding domain comprises
(A) a VH domain having at least 98% sequence identity to the sequence as set forth in SEQ ID NO: 204 and a VL domain having at least 98% sequence identity to the sequence as set forth in SEQ ID NO: 205;
(B) a VH domain having at least 98% sequence identity to the sequence as set forth in SEQ ID NO: 99 and a VL domain having at least 98% sequence identity to the sequence as set forth in SEQ ID NO: 100;
(C) a VH domain having at least 98% sequence identity to the sequence as set forth in SEQ ID NO: 101 and a VL domain having at least 98% sequence identity to the sequence as set forth in SEQ ID NO: 102;
(D) a VH domain having at least 98% sequence identity to the sequence as set forth in SEQ ID NO: 103 and a VL domain having at least 98% sequence identity to the sequence as set forth in SEQ ID NO: 104;
(E) a VH domain having at least 98% sequence identity to the sequence as set forth in SEQ ID NO: 105 and a VL domain having at least 98% sequence identity to the sequence as set forth in SEQ ID NO: 106;
(F) a VH domain having at least 98% sequence identity to the sequence as set forth in SEQ ID NO: 107 and a VL domain having at least 98% sequence identity to the sequence as set forth in SEQ ID NO: 108;
(G) a VH domain having at least 98% sequence identity to the sequence as set forth in SEQ ID NO: 109 and a VL domain having at least 98% sequence identity to the sequence as set forth in SEQ ID NO: 110;
(H) a VH domain having at least 98% sequence identity to the sequence as set forth in SEQ ID NO: 111 and a VL domain having at least 98% sequence identity to the sequence as set forth in SEQ ID NO: 112; or
(I) an antigen binding fragment having at least 98% sequence identity to the sequence as set forth in SEQ ID NO: 266 and an antigen binding fragment having at least 98% sequence identity to the sequence as set forth in SEQ ID NO: 267.

14. The CAR of claim 9, wherein the NKp46 binding domain comprises
(A) a VH domain having the sequence as set forth in SEQ ID NO: 204 and a VL domain having the sequence as set forth in SEQ ID NO: 205;
(B) a VH domain having the sequence as set forth in SEQ ID NO: 99 and a VL domain having the sequence as set forth in SEQ ID NO: 100;
(C) a VH domain having the sequence as set forth in SEQ ID NO: 101 and a VL domain having the sequence as set forth in SEQ ID NO: 102;
(D) a VH domain having the sequence as set forth in SEQ ID NO: 103 and a VL domain having the sequence as set forth in SEQ ID NO: 104;
(E) a VH domain having the sequence as set forth in SEQ ID NO: 105 and a VL domain having the sequence as set forth in SEQ ID NO: 106;
(F) a VH domain having the sequence as set forth in SEQ ID NO: 107 and a VL domain having the sequence as set forth in SEQ ID NO: 108;
(G) a VH domain having the sequence as set forth in SEQ ID NO: 109 and a VL domain having the sequence as set forth in SEQ ID NO: 110;
(H) a VH domain having the sequence as set forth in SEQ ID NO: 111 and a VL domain having the sequence as set forth in SEQ ID NO: 112; or
(I) an antigen binding fragment of a heavy chain having the sequence as set forth in SEQ ID NO: 266 and an antigen binding fragment of a light chain having the sequence as set forth in SEQ ID NO: 267.

15. The CAR of claim 9, wherein the NKp46 binding domain comprises a single chain variable fragment (scFv) having at least 98% sequence identity to the sequence as set forth in SEQ ID NO: 206, SEQ ID NO: 113, SEQ ID NO: 114, SEQ ID NO: 115, SEQ ID NO: 116, SEQ ID NO: 117, SEQ ID NO: 118, or SEQ ID NO: 119.

16. The CAR of claim 9, wherein the NKp46 binding domain comprises a single chain variable fragment (scFv) having the sequence as set forth in SEQ ID NO: 206, SEQ ID NO: 113, SEQ ID NO: 114, SEQ ID NO: 115, SEQ ID NO: 116, SEQ ID NO: 117, SEQ ID NO: 118, or SEQ ID NO: 119.

17. The CAR of claim 7, wherein the inhibitory NK receptor binding domain comprises a killer immunoglobulin receptor (KIR) binding domain and/or an NKG2A binding domain.

18. The CAR of claim 17, wherein the KIR binding domain comprises an antigen binding fragment of a heavy chain as set forth in SEQ ID NO: 288 and an antigen binding fragment of light chain as set forth in SEQ ID NO: 289.

19. The CAR of claim 17, wherein the KIR binding domain comprises an antigen binding fragment of antibody A210, antibody A803g, antibody clone #180704, and antibody clone NKVFS1.

20. The CAR of claim 17, wherein the NKG2A binding domain comprises an artificial human leukocyte antigen (HLA)-E mimetic.

21. The CAR of claim 14, wherein the HLA-E mimetic comprises a heterotrimer of: i) an HLA-E binding signal peptide; ii) mature beta-2 microglobulin (B2M); and iii) an HLA-E 01:03 heavy chain.

22. The CAR of claim 21, wherein the HLA-E binding signal peptide comprises: a signal peptide of HLA-A, HLA-B, HLA-C, or HLA-G; a peptide from a human immunodeficiency virus (HIV), a cytomegalovirus (CMV) or Epstein-Barr virus (EBV); a peptide of multidrug resistance-associated protein 7; or a leader peptide of HSP60.

23. The CAR of claim 21, wherein the HLA-E binding signal peptide comprises a sequence as set forth in SEQ ID NOs: 127, and 276-287.

24. The CAR of claim 20, wherein the HLA-E mimetic includes a sequence having at least 98% sequence identity to the sequence as set forth in SEQ ID NOs: 242, 244, or 246.

25. The CAR of claim 20, wherein the HLA-E mimetic has the sequence as set forth in SEQ ID NOs: 242, 244, or 246.

26. The CAR of claim 17, wherein the NKG2A binding domain is an scFV derived from monalizumab, antibody Z270, antibody Z199, antibody 20D5, or antibody 3S9.

27. The CAR of claim 17, wherein the NKG2A binding domain comprises:
a VH domain comprising: a CDRH1 having the sequence as set forth in SEQ ID NO: 213, a CDRH2 having the sequence as set forth in SEQ ID NO: 214, and a CDRH3 having the sequence as set forth in SEQ ID NO: 215; and a VL domain comprising: a CDRL1 having the sequence as set forth in SEQ ID NO: 216, a CDRL2 having the sequence as set forth in SEQ ID NO: 217, and a CDRL3 having the sequence as set forth in SEQ ID NO: 218, or
a VH domain comprising a CDRH1 having the sequence as set forth in SEQ ID NO: 290, a CDRH2 having the sequence as set forth in SEQ ID NO: 291, and a CDRH3 having the sequence as set forth in SEQ ID NO: 292; and a VL domain including: a CDRL1 having the sequence as set forth in SEQ ID NO: 293, a CDRL2 having the sequence as set forth in SEQ ID NO: 294, and a CDRL3 having the sequence as set forth in SEQ ID NO: 295,
each according to Kabat numbering.

28. The CAR of claim 17, wherein the NKG2A binding domain comprises a VH domain having at least 98% sequence identity to the sequence as set forth in SEQ ID NO: 219, 220, 221, 222, or 223.

29. The CAR of claim 17, wherein the NKG2A binding domain comprises a VH domain having the sequence as set forth in SEQ ID NO: 219, 220, 221, 222, or 223.

30. The CAR of claim 17, wherein the NKG2A binding domain comprises an antigen binding fragment of a heavy chain having at least 98% sequence identity to the sequence as set forth in SEQ ID NO: 224, 225, 226, 227, or 228, and/or an antigen binding fragment of a light chain having at least 98% sequence identity to the sequence as set forth in SEQ ID NO: 229.

31. The CAR of claim 17, wherein the NKG2A binding domain comprises an antigen binding fragment of a heavy chain having a sequence as set forth in SEQ ID NO: 224, 225, 226, 227, or 228, and/or an antigen binding fragment of a light chain having a sequence as set forth in SEQ ID NO: 229.

32. The CAR of claim 7, wherein the extracellular component further comprises a tag.

33. The CAR of claim 32, wherein the tag is selected from His tag, Flag tag, Xpress tag, Avi tag, Calmodulin binding peptide (CBP) tag, Polyglutamate tag, HA tag, Myc tag, Strep tag, Softag 1, Softag 3, and/or V5 tag.

34. The CAR of claim 32, wherein the tag has a sequence as set forth in SEQ ID NO: 152, 153, 154, 155, 156, 157, 158, 159, 160, 161, 162, 163, 164, 165, 166, 167, or 168.

35. The CAR of claim 7, wherein the extracellular component further comprises a hinge.

36. The CAR of claim 35, wherein the hinge comprises a human Ig hinge, a KIR2DS2 hinge, or a CD8α hinge.

37. The CAR of claim 35, wherein the hinge is a CD8α hinge.

38. The CAR of claim 35, wherein the hinge has the sequence as set forth in SEQ ID NO: 140.

39. The CAR of claim 7, wherein the extracellular component further comprises a linker.

40. The CAR of claim 39, wherein the linker is a glycine-serine linker, an IgG4 linker, or a CD28 linker.

41. The CAR of claim 39, wherein the linker has the sequence as set forth in SEQ ID NO: 142, 143, 144, 145, 150, or 151.

42. The CAR of claim 8, wherein the transmembrane domain comprises a transmembrane domain of: the α, β, or ζ chain of the T-cell receptor, CD28, CD27, CD3ε, CD45, CD4, CD5, CD8, CD9, CD16, CD22, CD33, CD37, CD64, CD80, CD86, CD134, CD137, CD154, KIRDS2, OX40, CD2, LFA-1, ICOS, 4-1BB, GITR, CD40, BAFFR, HVEM, SLAMF7, NKp80, NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1, SLAMF4, CD84, CD96, CEACAM1, CRT AM, Ly9, CD160, PSGL1, CD100, SLAMF6, SLAM, BLAME, SELPLG, LTBR, PAG/Cbp, NKG2D, NKG2C, or a combination thereof.

43. The CAR of claim 8, wherein the transmembrane domain comprises a transmembrane domain of CD8α chain.

44. The CAR of claim 8, wherein the transmembrane domain has the sequence as set forth in SEQ ID NO: 138.

45. The CAR of claim 7, wherein the intracellular component comprises an intracellular signaling domain.

46. The CAR of claim 45, wherein the intracellular signaling domain comprises CD3ζ, common FcR γ, Fc γ RIIa, FcR β, CD3 γ, CD3 δ, CD3 ε, CD79a, CD79b, DAP10, DAP12, or a combination thereof.

47. The CAR of claim 45, wherein the intracellular signaling domain comprises CD3ζ.

48. The CAR of claim 45, wherein the intracellular signaling domain has the sequence as set forth in SEQ ID NO: 146.

49. The CAR of claim 45, wherein the intracellular signaling domain comprises a costimulatory signaling domain.

50. The CAR of claim 49, wherein the costimulatory signaling domain comprises an MHC class I molecule, B and T cell lymphocyte attenuator (BTLA), a Toll ligand receptor, OX40, CD27, CD28, CDS, ICAM-1, LFA-1, ICOS, 4-1BB, GITR, BAFFR, HVEM, SLAMF7, NKp80, NKp44, NKp30, NKp46, CD160, CD19, CD4, CD8α, CD8β, IL2Rβ, IL2Rγ, IL7Rα, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, NKG2D, NKG2C, TNFR2, TRANCE/RANKL, DNAM1, SLAMF4, CD84, CD96, CEACAM1, CRTAM, Ly9, CD160, PSGL1, CD100, CD69, SLAMF6, SLAM, BLAME, SELPLG, LTBR, LAT, GADS, SLP-76, PAG/Cbp, CD19a, or a combination thereof.

51. The CAR of claim 49, wherein the costimulatory signaling domain comprises 4-1BB.

52. The CAR of claim 49, wherein the costimulatory signaling domain has the sequence as set forth in SEQ ID NO: 148.

53. The CAR of claim 45, wherein the intracellular signaling domain comprises a 4-1BB costimulatory domain and a CD3ζ stimulatory domain having an amino acid sequence as set forth in SEQ ID NO: 230.

54. The CAR of claim 7, wherein the CAR has an amino acid sequence having at least 98% sequence identity with the sequence as set forth in SEQ ID NO: 249, 251, 253, 255, or 257.

55. The CAR of claim 7, wherein the CAR has an amino acid sequence as set forth in SEQ ID NO: 249, 251, 253, 255, or 257.

56. The CAR of claim 7, wherein the CAR is encoded by a nucleotide sequence having at least 98% sequence identity with the sequence as set forth in SEQ ID NO: 250, 252, 254, 256, or 258.

57. The CAR of claim 7, wherein the CAR is encoded by a nucleotide sequence as set forth in SEQ ID NO: 250, 252, 254, 256, or 258.

58. A vector comprising a nucleotide sequence encoding the CAR of claim 7.

59. The vector of claim 58, wherein the vector further comprises a promoter operably linked to the nucleotide sequence encoding the CAR of claim 7.

60. The vector of claim 59, wherein the promoter is the MND promoter.

61. The vector of claim 60, wherein the MND promoter has the sequence as set forth in SEQ ID NO: 175.

62. The vector of claim 58, wherein the vector further comprises a nucleotide sequence encoding a transduction marker.

63. The vector of claim 62, wherein the transduction marker is co-expressed with the CAR.

64. The vector of claim 62, wherein the transduction marker is blue fluorescent protein (BFP).

65. The vector of claim 64, wherein the BFP has the sequence as set forth in SEQ ID NO: 169, 170, 171, 172, 173, 174, or 236.

66. The vector of claim 62, wherein the nucleotide sequence encoding the transduction marker is linked to the nucleotide sequence encoding the CAR by a nucleotide sequence encoding a cleavable peptide.

67. The vector of claim 66, wherein the cleavable peptide comprises porcine teschovirus-1 (P2A), Thosea asigna virus (T2A), equine rhinitis A virus (E2A), foot-and-mouth disease virus (F2A), potyvirus 2A, or cardiovirus 2A.

68. The vector of claim 66, wherein the cleavable peptide has the sequence as set forth in SEQ ID NO: 176, 177, 178, 179, 180, 181, 182, 183, 184, 185, or 240.

69. The vector of claim 58, wherein the vector further comprises a molecular safety switch.

70. The vector of claim 69, wherein the molecular safety switch comprises a suicide gene.

71. The vector of claim 70, wherein the suicide gene comprises inducible-caspase-9 (iCASP9), herpes simplex virus thymidine kinase (HSV-TK), or truncated epidermal growth factor receptor (tEGFR).

72. A cell genetically modified to express a CAR of claim 7.

73. The cell of claim 72, wherein the cell co-expresses:
(a) a CAR wherein the extracellular component comprises an NKp46 binding domain; and (b) a CAR wherein the extracellular component comprises an NKG2A binding domain, or
(c) a CAR wherein the extracellular component comprises an NKp46 binding domain; and (d) a CAR wherein the extracellular component comprises an NKG2A binding domain and the intracellular component lacks an intracellular signaling domain.

74. The cell of claim 72, wherein the cell is a T cell, an NK cell, a macrophage, a hematopoietic stem cell (HSC), or a hematopoietic progenitor cell (HPC).

75. A formulation comprising the cell of claim 72 and a pharmaceutically acceptable carrier.

76. A method of depleting natural killer (NK) cells expressing an activating NK receptor and/or an inhibitory NK receptor in a first population of NK cells, comprising:
exposing the first population of cells to a second population of cells genetically modified to express a CAR of claim 7,
wherein the exposing results in depletion of NK cells in the first population.

77. The method of claim 76, wherein the exposing comprises administering the second population of CAR expressing cells to a subject having the first population of cells.

78. The method of claim 76, wherein cells of the second population co-express:
(a) a CAR wherein the extracellular component comprises an NKp46 binding domain; and (b) a CAR wherein the extracellular component comprises an NKG2A binding domain, or
(c) a CAR wherein the extracellular component comprises an NKp46 binding domain; and (d) a CAR wherein the extracellular component comprises an NKG2A binding domain and the intracellular component lacks an intracellular signaling domain.

79. The method of claim 78, wherein expression of the CAR comprising the NKG2A binding domain in CAR expressing cells confer resistance on the CAR expressing cells to killing by the NKp46 expressing NK cells.

80. A method of treating a natural killer (NK) cell associated disease or a disease associated with cells that express NK receptors in a subject in need thereof, comprising:
administering to the subject a therapeutically effective amount of a formulation comprising cells genetically modified to express a CAR of claim 7
thereby treating the NK cell associated disease or a disease associated with cells that express NK receptors in the subject.

81. The method of claim 80, further comprising administering to the subject an anti-viral treatment or prophylaxis.

82. The method of claim 81, wherein the anti-viral treatment treats Epstein-Barr Virus (EBV) infection.

83. The method of claim 81, wherein the anti-viral treatment comprises a nucleoside analog selected from acyclovir, valacyclovir, famciclovir, ganciclovir, and/or valganciclovir.

84. The method of claim 80, wherein the NK cell associated disease is an NK cell malignancy.

85. The method of claim 84, wherein the NK cell malignancy comprises immature NK cell neoplasms, agranular CD4+/CD56+ hematodermic neoplasms, CD94 1A+/TCR− lymphoblastic lymphoma/leukemia (LBL), myeloid/NK cell acute leukemia, mature NK cell neoplasms, extranodal NK cell lymphoma, nasal type, aggressive NK cell leukemia, and chronic NK cell lymphocytosis.

86. The method of claim 80, wherein the disease associated with cells that express NK receptors comprise non-NK cell malignancies with aberrant NK receptor expression.

87. The method of claim 86, wherein the disease comprises Sezary syndrome, mature T cell neoplasms, T cell large granular lymphocytic leukemia, mycosis fungoides, and ALK+ anaplastic large cell lymphoma.

88. The method of claim 80, wherein the disease associated with cells that express NK receptors comprise infections.

89. The method of claim 88, wherein the infections are chronic.

90. The method of claim 88, wherein the infections are bacterial, viral, fungal, parasitic, and/or arthropod.

91. The method of claim 88, wherein the infections are caused by or comprise Staphylococcus spp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, Treponema pallidum, rhinovirus, influenza virus, respiratory syncytial virus (RSV), coronavirus, herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), Merkel cell polyomavirus (MCV), HHV8 (Kaposi's sarcoma), Trychophyton spp., Candida spp., Giardia, toxoplasmosis, E. vermicularis, Trypanosoma cruzi, Echinococcosis, Cysticercosis, Toxocariasis, Trichomoniasis, Amebiasis, California encephalitis, Chikungunya, dengue, Eastern equine encephalitis, Powassan, St. Louis encephalitis, West Nile, Yellow Fever, Zika, Lyme disease, and/or babesiosis.

92. The method of claim 80, wherein the formulation comprises cells expressing a CAR comprising an NKG2A binding domain.

93. The method of claim 92, wherein the NK cell associated disease is cancer.

94. The method of claim 93, wherein the cancer comprises: carcinoma of the bladder, head and neck, breast, colon, kidney, liver, lung, ovary, prostate, pancreas, stomach, cervix, thyroid or skin; squamous cell carcinoma; leukemia; acute lymphocytic leukemia; acute lymphoblastic leukemia; B cell lymphoma; T cell lymphoma; Hodgkin's lymphoma; non-Hodgkin's lymphoma; hairy cell lymphoma; Burkett's lymphoma; acute myelogenous leukemia; chronic myelogenous leukemia; promyelocytic leukemia; fibrosarcoma; rhabdomyosarcoma; osteosarcoma; neuroblastoma; glioma; astrocytoma; schwannomas; melanoma; xeroderma pigmentosum; keratoacanthoma; seminoma; thyroid follicular cancer; teratocarcinoma; T-prolymphocytic leukemia (T-PLL); large granular lymphocyte leukemia (LGL) of the T cell type; Sezary syndrome (SS); adult T cell leukemia lymphoma (ATLL); hepatosplenic T cell lymphoma; peripheral/post-thymic T cell lymphoma; angioimmunoblastic T cell lymphoma; angiocentric (nasal) T cell lymphoma; anaplastic (Ki 1+) large cell lymphoma; intestinal T cell lymphoma; and/or T-lymphoblastic lymphoma/leukemia (T-Lbly/T-ALL).

95. The method of claim 92, wherein the amount of NK cells expressing NKG2A inhibitory receptor in the subject is decreased, as compared to an amount of NK cells expressing NKG2A inhibitory receptor in the subject prior to the administering or in a reference population in need thereof not administered the formulation.

96. The method of claim 80, wherein the formulation comprises cells expressing a CAR comprising an NKp46 binding domain.

97. The method of claim 96, wherein the formulation comprises cells of
(a) a first population expressing the CAR comprising the NKp46 binding domain; and (b) a second population expressing a CAR comprising an NKG2A binding domain, or
(c) a first population expressing the CAR comprising the NKp46 binding domain; and (d) a second population expressing a CAR comprising an NKG2A binding domain and the intracellular component lacks an intracellular signaling domain or
wherein cells of the formulation co-express:
(e) the CAR comprising the NKp46 binding domain; and (f) a CAR comprising an NKG2A binding domain, or
(g) the CAR comprising the NKp46 binding domain; and (h) a CAR comprising an NKG2A binding domain and the intracellular component lacks an intracellular signaling domain.

98. The method of claim 97, wherein the NKG2A binding domain comprises human leukocyte antigen (HLA)-E or an artificial HLA-E mimetic.

99. The method of claim 97, wherein the NKG2A binding domain is an scFV derived from monalizumab, antibody Z270, antibody Z199, antibody 20D5, or antibody 3S9.

100. The method of claim 97, wherein expression of the CAR comprising the NKG2A binding domain in CAR expressing cells confer resistance on the CAR expressing cells to killing by NK cells in the NK cell associated disease.

101. The method of claim 97, wherein expression of the CAR comprising the NKG2A binding domain in CAR expressing cells reduces rejection of the CAR expressing cells by the subject.

102. The method of claim 96, wherein the NK cell associated disease is an autoimmune disease or an alloimmune disease.

103. The method of claim 102, wherein the autoimmune disease comprises Sjogren's disease; antiphospholipid syndrome; Pemphigus vulgaris; spondylarthropathies; skin diseases including psoriasis; multiple sclerosis; systemic sclerosis; Type I diabetes; juvenile idiopathic arthritis; rheumatoid arthritis; inflammatory bowel disease; autoimmune liver diseases; and/or systemic lupus erythematosus (SLE).

104. The method of claim 102, wherein the alloimmune disease comprises fetal/neonatal alloimmune thrombocytopenia (FNAIT); hematopoietic stem cell rejection; solid tissue transplant rejection; and/or chronic allograft injury after kidney transplantation.

105. The method of claim 96, wherein the amount of NK cells expressing NKp46 activating receptor in the subject is decreased, as compared to an amount of NK cells expressing NKp46 activating receptor in the subject prior to the administering or in a reference population in need thereof not administered the formulation.

106. A kit comprising a nucleotide sequence encoding a CAR of claim 7.

107. The kit of claim 106, wherein the nucleotide sequence encoding the CAR of claim 7 is within a vector.

108. The kit of claim 107, wherein the vector is a viral vector.

109. A kit of claim 106, further comprising a cell genetically modified to express the CAR of claim 7.

110. The kit of claim 106, further comprising media, artificial presenting cells, growth factors, and/or antibodies.

111. The kit of claim 106, further comprising 293 cells, 293T cells, and/or A549 cells.

112. The kit of claim 106, further comprising fibronectin-coated plates, hexadimethrine bromide (Polybrene), and/or lipofectamine.


SEQUENCE LISTING (289 sequences)

SEQ ID NO: 1 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 2 - DNA, 915 bp, Homo sapiens
atgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtccaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttga

SEQ ID NO: 3 - protein, 5 aa, Artificial Sequence
  CDRH1 of anti-NKp46 antibody NKp46-1, Kabat numbering
DYVIN

SEQ ID NO: 4 - protein, 7 aa, Artificial Sequence
  CDRH2 of anti-NKp46 antibody NKp46-1, Chothia numbering
GYTFTDY

SEQ ID NO: 5 - protein, 8 aa, Artificial Sequence
  CDRH1 of anti-NKp46 antibody NKp46-1, IMGT numbering
GYTFTDYV

SEQ ID NO: 6 - protein, 17 aa, Artificial Sequence
  CDRH2 of anti-NKp46 antibody NKp46-1, Kabat numbering
EIYPGSGTNYYNEKFKA

SEQ ID NO: 7 - protein, 4 aa, Artificial Sequence
  CDRH2 of anti-NKp46 antibody NKp46-1, Chothia numbering
PGSG

SEQ ID NO: 8 - protein, 8 aa, Artificial Sequence
  CDRH2 of anti-NKp46 antibody NKp46-1, IMGT numbering
IYPGSGTN

SEQ ID NO: 9 - protein, 11 aa, Artificial Sequence
  CDRH3 of anti-NKp46 antibody NKp46-1, Kabat numbering
RGRYGLYAMDY

SEQ ID NO: 10 - protein, 9 aa, Artificial Sequence
  CDRH3 of anti-NKp46 antibody NKp46-1, Chothia numbering
GRYGLYAMD

SEQ ID NO: 11 - protein, 13 aa, Artificial Sequence
  CDRH3 of anti-NKp46 antibody NKp46-1, IMGT numbering
ARRGRYGLYAMDY

SEQ ID NO: 12 - protein, 11 aa, Artificial Sequence
  CDRL1 of anti-NKp46 antibody NKp46-1, Kabat numbering
RASQDISNYLN

SEQ ID NO: 13 - protein, 7 aa, Artificial Sequence
  CDRL1 of anti-NKp46 antibody NKp46-1, Chothia numbering
SQDISNY

SEQ ID NO: 14 - protein, 6 aa, Artificial Sequence
  CDRL1 of anti-NKp46 antibody NKp46-1, IMGT numbering
QDISNY

SEQ ID NO: 15 - protein, 7 aa, Artificial Sequence
  CDRL2 of anti-NKp46 antibody NKp46-1, Kabat numbering
YTSRLHS

SEQ ID NO: 16 - protein, 9 aa, Artificial Sequence
  CDRL3 of anti-NKp46 antibody NKp46-1, Kabat numbering
QQGNTRPWT

SEQ ID NO: 17 - protein, 9 aa, Artificial Sequence
  CDRL3 of anti-NKp46 antibody NKp46-1, Chothia numbering
YTSGNTRPW

SEQ ID NO: 18 - protein, 12 aa, Artificial Sequence
  CDRL3 of anti-NKp46 antibody NKp46-1, IMGT numbering
YTSQQGNTRPWT

SEQ ID NO: 19 - protein, 6 aa, Artificial Sequence
  CDRH1 of anti-NKp46 antibody NKp46-2, Kabat numbering
SDYAWN

SEQ ID NO: 20 - protein, 8 aa, Artificial Sequence
  CDRH1 of anti-NKp46 antibody NKp46-2, Chothia numbering
GYSITSDY

SEQ ID NO: 21 - protein, 9 aa, Artificial Sequence
  CDRH1 of anti-NKp46 antibody NKp46-2, IMGT numbering
GYSITSDYA

SEQ ID NO: 22 - protein, 16 aa, Artificial Sequence
  CDRH2 of anti-NKp46 antibody NKp46-2, Kabat numbering
YITYSGSTSYNPSLES

SEQ ID NO: 23 - protein, 7 aa, Artificial Sequence
  CDRH2 of anti-NKp46 antibody NKp46-2, IMGT numbering
ITYSGST

SEQ ID NO: 24 - protein, 13 aa, Artificial Sequence
  CDRH3 of anti-NKp46 antibody NKp46-2, Kabat numbering
GGYYGSSWGVFAY

SEQ ID NO: 25 - protein, 11 aa, Artificial Sequence
  CDRH3 of anti-NKp46 antibody NKp46-2, Chothia numbering
GYYGSSWGVFA

SEQ ID NO: 26 - protein, 15 aa, Artificial Sequence
  CDRH3 of anti-NKp46 antibody NKp46-2, IMGT numbering
ARGGYYGSSWGVFAY

SEQ ID NO: 27 - protein, 11 aa, Artificial Sequence
  CDRL1 of anti-NKp46 antibody NKp46-2, Kabat numbering
RVSENIYSYLA

SEQ ID NO: 28 - protein, 7 aa, Artificial Sequence
  CDRL1 of anti-NKp46 antibody NKp46-2, Chothia numbering
SENIYSY

SEQ ID NO: 29 - protein, 6 aa, Artificial Sequence
  CDRL1 of anti-NKp46 antibody NKp46-2, IMGT numbering
ENIYSY

SEQ ID NO: 30 - protein, 7 aa, Artificial Sequence
  CDRL2 of anti-NKp46 antibody NKp46-2, Kabat numbering
NAKTLAE

SEQ ID NO: 31 - protein, 9 aa, Artificial Sequence
  CDRL3 of anti-NKp46 antibody NKp46-2, Kabat numbering
QHHYGTPWT

SEQ ID NO: 32 - protein, 6 aa, Artificial Sequence
  CDRL3 of anti-NKp46 antibody NKp46-2, Chothia numbering
HYGTPW

SEQ ID NO: 33 - protein, 5 aa, Artificial Sequence
  CDRH1 of anti-NKp46 antibody NKp46-3, Kabat numbering
EYTMH

SEQ ID NO: 34 - protein, 7 aa, Artificial Sequence
  CDRH1 of anti-NKp46 antibody NKp46-3, Chothia numbering
GYTFTEY

SEQ ID NO: 35 - protein, 8 aa, Artificial Sequence
  CDRH1 of anti-NKp46 antibody NKp46-3, IMGT numbering
GYTFTEYT

SEQ ID NO: 36 - protein, 17 aa, Artificial Sequence
  CDRH2 of anti-NKp46 antibody NKp46-3, Kabat numbering
GISPNIGGTSYNQKFKG

SEQ ID NO: 37 - protein, 4 aa, Artificial Sequence
  CDRH2 of anti-NKp46 antibody NKp46-3, Chothia numbering
PNIG

SEQ ID NO: 38 - protein, 8 aa, Artificial Sequence
  CDRH2 of anti-NKp46 antibody NKp46-3, IMGT numbering
ISPNIGGT

SEQ ID NO: 39 - protein, 7 aa, Artificial Sequence
  CDRH3 of anti-NKp46 antibody NKp46-3, Kabat numbering
RGGSFDY

SEQ ID NO: 40 - protein, 5 aa, Artificial Sequence
  CDRH3 of anti-NKp46 antibody NKp46-3, Chothia numbering
GGSFD

SEQ ID NO: 41 - protein, 9 aa, Artificial Sequence
  CDRH3 of anti-NKp46 antibody NKp46-3, IMGT numbering
ARRGGSFDY

SEQ ID NO: 42 - protein, 11 aa, Artificial Sequence
  CDRL1 of anti-NKp46 antibody NKp46-3, Kabat numbering
RASQSISDYLH

SEQ ID NO: 43 - protein, 7 aa, Artificial Sequence
  CDRL1 of anti-NKp46 antibody NKp46-3, Chothia numbering
SQSISDY

SEQ ID NO: 44 - protein, 6 aa, Artificial Sequence
  CDRL1 of anti-NKp46 antibody NKp46-3, IMGT numbering
QSISDY

SEQ ID NO: 45 - protein, 7 aa, Artificial Sequence
  CDRL2 of anti-NKp46 antibody NKp46-3, Kabat numbering
YASQSIS

SEQ ID NO: 46 - protein, 9 aa, Artificial Sequence
  CDRL3 of anti-NKp46 antibody NKp46-3, Kabat numbering
QNGHSFPLT

SEQ ID NO: 47 - protein, 6 aa, Artificial Sequence
  CDRL3 of anti-NKp46 antibody NKp46-3, Chothia numbering
GHSFPL

SEQ ID NO: 48 - protein, 5 aa, Artificial Sequence
  CDRH1 of anti-NKp46 antibody NKp46-4, Kabat numbering
SFTMH

SEQ ID NO: 49 - protein, 7 aa, Artificial Sequence
  CDRH1 of anti-NKp46 antibody NKp46-4, Chothia numbering
GYTFTSF

SEQ ID NO: 50 - protein, 8 aa, Artificial Sequence
  CDRH1 of anti-NKp46 antibody NKp46-4, IMGT numbering
GYTFTSFT

SEQ ID NO: 51 - protein, 17 aa, Artificial Sequence
  CDRH2 of anti-NKp46 antibody NKp46-4, Kabat numbering
YINPSSGYTEYNQKFKD

SEQ ID NO: 52 - protein, 4 aa, Artificial Sequence
  CDRH2 of anti-NKp46 antibody NKp46-4, Chothia numbering
PSSG

SEQ ID NO: 53 - protein, 8 aa, Artificial Sequence
  CDRH2 of anti-NKp46 antibody NKp46-4, IMGT numbering
INPSSGYT

SEQ ID NO: 54 - protein, 8 aa, Artificial Sequence
  CDRH3 of anti-NKp46 antibody NKp46-4, Kabat numbering
GSSRGFDY

SEQ ID NO: 55 - protein, 6 aa, Artificial Sequence
  CDRH3 of anti-NKp46 antibody NKp46-4, Chothia numbering
SSRGFD

SEQ ID NO: 56 - protein, 10 aa, Artificial Sequence
  CDRH3 of anti-NKp46 antibody NKp46-4, IMGT numbering
VRGSSRGFDY

SEQ ID NO: 57 - protein, 11 aa, Artificial Sequence
  CDRL1 of anti-NKp46 antibody NKp46-4, Kabat numbering
RASENIYSNLA

SEQ ID NO: 58 - protein, 7 aa, Artificial Sequence
  CDRL1 of anti-NKp46 antibody NKp46-4, Chothia numbering
SENIYSN

SEQ ID NO: 59 - protein, 6 aa, Artificial Sequence
  CDRL1 of anti-NKp46 antibody NKp46-4, IMGT numbering
ENIYSN

SEQ ID NO: 60 - protein, 7 aa, Artificial Sequence
  CDRL2 of anti-NKp46 antibody NKp46-4, Kabat numbering
AATNLAD

SEQ ID NO: 61 - protein, 9 aa, Artificial Sequence
  CDRL3 of anti-NKp46 antibody NKp46-4, Kabat numbering
QHFWGTPRT

SEQ ID NO: 62 - protein, 6 aa, Artificial Sequence
  CDRL3 of anti-NKp46 antibody NKp46-4, Chothia numbering
FWGTPR

SEQ ID NO: 63 - protein, 5 aa, Artificial Sequence
  CDRH1 of anti-NKp46 antibody NKp46-6, Kabat numbering
SSWMH

SEQ ID NO: 64 - protein, 7 aa, Artificial Sequence
  CDRH1 of anti-NKp46 antibody NKp46-6, Chothia numbering
GYTFTSS

SEQ ID NO: 65 - protein, 8 aa, Artificial Sequence
  CDRH1 of anti-NKp46 antibody NKp46-6, IMGT numbering
GYTFTSSW

SEQ ID NO: 66 - protein, 17 aa, Artificial Sequence
  CDRH2 of anti-NKp46 antibody NKp46-6, Kabat numbering
HIHPNSGISNYNEKFKG

SEQ ID NO: 67 - protein, 4 aa, Artificial Sequence
  CDRH2 of anti-NKp46 antibody NKp46-6, Chothia numbering
PNSG

SEQ ID NO: 68 - protein, 8 aa, Artificial Sequence
  CDRH2 of anti-NKp46 antibody NKp46-6, IMGT numbering
IHPNSGIS

SEQ ID NO: 69 - protein, 6 aa, Artificial Sequence
  CDRH3 of anti-NKp46 antibody NKp46-6, Kabat numbering
GGRFDD

SEQ ID NO: 70 - protein, 4 aa, Artificial Sequence
  CDRH3 of anti-NKp46 antibody NKp46-6, Chothia numbering
GRFD

SEQ ID NO: 71 - protein, 8 aa, Artificial Sequence
  CDRH3 of anti-NKp46 antibody NKp46-6, IMGT numbering
ARGGRFDD

SEQ ID NO: 72 - protein, 10 aa, Artificial Sequence
  CDRL3 of anti-NKp46 antibody NKp46-6, Kabat numbering
QNGHSFLMYT

SEQ ID NO: 73 - protein, 7 aa, Artificial Sequence
  CDRL3 of anti-NKp46 antibody NKp46-6, Chothia numbering
GHSFLMY

SEQ ID NO: 74 - protein, 6 aa, Artificial Sequence
  CDRH1 of anti-NKp46 antibody NKp46-9, Kabat numbering
SDYAWN

SEQ ID NO: 75 - protein, 8 aa, Artificial Sequence
  CDRH1 of anti-NKp46 antibody NKp46-9, Chothia numbering
GYSITSDY

SEQ ID NO: 76 - protein, 9 aa, Artificial Sequence
  CDRH1 of anti-NKp46 antibody NKp46-9, IMGT numbering
GYSITSDYA

SEQ ID NO: 77 - protein, 16 aa, Artificial Sequence
  CDRH2 of anti-NKp46 antibody NKp46-9, Kabat numbering
YITYSGSTNYNPSLKS

SEQ ID NO: 78 - protein, 11 aa, Artificial Sequence
  CDRH3 of anti-NKp46 antibody NKp46-9, Kabat numbering
CWDYALYAMDC

SEQ ID NO: 79 - protein, 9 aa, Artificial Sequence
  CDRH3 of anti-NKp46 antibody NKp46-9, Chothia numbering
WDYALYAMD

SEQ ID NO: 80 - protein, 13 aa, Artificial Sequence
  CDRH3 of anti-NKp46 antibody NKp46-9, IMGT numbering
ARCWDYALYAMDC

SEQ ID NO: 81 - protein, 11 aa, Artificial Sequence
  CDRL1 of anti-NKp46 antibody NKp46-9, Kabat numbering
RTSENIYSYLA

SEQ ID NO: 82 - protein, 9 aa, Artificial Sequence
  CDRL3 of anti-NKp46 antibody NKp46-9, Kabat numbering
QHHYDTPLT

SEQ ID NO: 83 - protein, 6 aa, Artificial Sequence
  CDRL3 of anti-NKp46 antibody NKp46-9, Chothia numbering
HYDTPL

SEQ ID NO: 84 - protein, 5 aa, Artificial Sequence
  CDRH1 of anti-NKp46 antibody BAB281, Kabat numbering
NYGMN

SEQ ID NO: 85 - protein, 7 aa, Artificial Sequence
  CDRH1 of anti-NKp46 antibody BAB281, Chothia numbering
GYTFTNY

SEQ ID NO: 86 - protein, 8 aa, Artificial Sequence
  CDRH1 of anti-NKp46 antibody BAB281, IMGT numbering
GYTFTNYG

SEQ ID NO: 87 - protein, 17 aa, Artificial Sequence
  CDRH2 of anti-NKp46 antibody BAB281, Kabat numbering
WINTNTGEPTYAEEFKG

SEQ ID NO: 88 - protein, 4 aa, Artificial Sequence
  CDRH2 of anti-NKp46 antibody BAB281, Chothia numbering
TNTG

SEQ ID NO: 89 - protein, 8 aa, Artificial Sequence
  CDRH2 of anti-NKp46 antibody BAB281, IMGT numbering
INTNTGEP

SEQ ID NO: 90 - protein, 8 aa, Artificial Sequence
  CDRH3 of anti-NKp46 antibody BAB281, Kabat numbering
DYLYYFDY

SEQ ID NO: 91 - protein, 6 aa, Artificial Sequence
  CDRH3 of anti-NKp46 antibody BAB281, Chothia numbering
YLYYFD

SEQ ID NO: 92 - protein, 10 aa, Artificial Sequence
  CDRH3 of anti-NKp46 antibody BAB281, IMGT numbering
ARDYLYYFDY

SEQ ID NO: 93 - protein, 11 aa, Artificial Sequence
  CDRL1 of anti-NKp46 antibody BAB281, Kabat numbering
KASENVVTYVS

SEQ ID NO: 94 - protein, 7 aa, Artificial Sequence
  CDRL1 of anti-NKp46 antibody BAB281, Chothia numbering
SENVVTY

SEQ ID NO: 95 - protein, 6 aa, Artificial Sequence
  CDRL1 of anti-NKp46 antibody BAB281, IMGT numbering
ENVVTY

SEQ ID NO: 96 - protein, 7 aa, Artificial Sequence
  CDRL2 of anti-NKp46 antibody BAB281, Kabat numbering
GASNRYT

SEQ ID NO: 97 - protein, 9 aa, Artificial Sequence
  CDRL3 of anti-NKp46 antibody BAB281, Kabat numbering
GQGYSYPYT

SEQ ID NO: 98 - protein, 6 aa, Artificial Sequence
  CDRL3 of anti-NKp46 antibody BAB281, Chothia numbering
GYSYPY

SEQ ID NO: 99 - protein, 120 aa, Artificial Sequence
  Amino acid sequence of heavy chain variable (VH) domain of anti-NKp46-1
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 100 - protein, 107 aa, Artificial Sequence
  Amino acid sequence of light chain variable (VL) domain of anti-NKp46-1 antibody
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 101 - protein, 122 aa, Artificial Sequence
  Amino acid sequence of VH domain of anti-NKp46- 2 antibody
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 102 - protein, 107 aa, Artificial Sequence
  Amino acid sequence of VL domain of anti-NKp46- 2 antibody
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 103 - protein, 116 aa, Artificial Sequence
  Amino acid sequence of VH domain of anti-NKp46- 3 antibody
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 104 - protein, 107 aa, Artificial Sequence
  Amino acid sequence of VL domain of anti-NKp46- 3 antibody
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 105 - protein, 117 aa, Artificial Sequence
  Amino acid sequence of VH domain of anti-NKp46- 4 antibody
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 106 - protein, 107 aa, Artificial Sequence
  Amino acid sequence of VL domain of anti-NKp46- 4 antibody
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 107 - protein, 105 aa, Artificial Sequence
  Amino acid sequence of VH domain of anti-NKp46- 6 antibody
LVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSS

SEQ ID NO: 108 - protein, 108 aa, Artificial Sequence
  Amino acid sequence of VL domain of anti-NKp46- 6 antibody
DIVMTQSPATLSVTPGDRVSLSCRASQSISDYLHWYQQKSHESPRLLIKYASQSISGIPSRFSGSGSGSDFTLSINSVEPEDVGVYYCQNGHSFLMYTFGGGTKLEIK

SEQ ID NO: 109 - protein, 120 aa, Artificial Sequence
  Amino acid sequence of VH domain of anti-NKp46- 9 antibody
DVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSS

SEQ ID NO: 110 - protein, 107 aa, Artificial Sequence
  Amino acid sequence of VL domain of anti-NKp46- 9 antibody
DIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 111 - protein, 117 aa, Artificial Sequence
  Amino acid sequence of VH domain of BAB281 antibody
QIQLVQSGPELQKPGETVKISCKASGYTFTNYGMNWVKQAPGKGLKWMGWINTNTGEPTYAEEFKGRFAFSLETSASTAYLQINNLKNEDTATYFCARDYLYYFDYWGQGTTLTVSS

SEQ ID NO: 112 - protein, 107 aa, Artificial Sequence
  Amino acid sequence of VL domain of BAB281 antibody
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 113 - protein, 249 aa, Artificial Sequence
  Amino acid sequence for NKp46-1 single chain variable fragment (scFv)
STGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSVEGGSGGSGGSGGSGGVDDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 114 - protein, 251 aa, Artificial Sequence
  Amino acid sequence for NKp46-2 scFv
STGSEVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSAVEGGSGGSGGSGGSGGVDDIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 115 - protein, 245 aa, Artificial Sequence
  Amino acid sequence for NKp46-3 scFv
STGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 116 - protein, 246 aa, Artificial Sequence
  Amino acid sequence for NKp46-4 scFv
STGSQVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSAVEGGSGGSGGSGGSGGVDDIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 117 - protein, 245 aa, Artificial Sequence
  Amino acid sequence for NKp46-6 scFv
STGSQVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCARGGRFDDWGAGTTVTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFLMYTFGGGTKLEIK

SEQ ID NO: 118 - protein, 249 aa, Artificial Sequence
  Amino acid sequence for NKp46-9 scFv
STGSDVQLQESGPGLVKPSQSLSLTCTVTGYSITSDYAWNWIRQFPGNKLEWMGYITYSGSTNYNPSLKSRISITRDTSKNQFFLQLNSVTTEDTATYYCARCWDYALYAMDCWGQGTSVTVSSVEGGSGGSGGSGGSGGVDDIQMTQSPASLSASVGETVTITCRTSENIYSYLAWCQQKQGKSPQLLVYNAKTLAEGVPSRFSGSGSGTHFSLKINSLQPEDFGIYYCQHHYDTPLTFGAGTKLELK

SEQ ID NO: 119 - protein, 246 aa, Artificial Sequence
  Amino acid sequence for BAB281 scFv
STGSQIQLVQSGPELQKPGETVKISCKASGYTFTNYGMNWVKQAPGKGLKWMGWINTNTGEPTYAEEFKGRFAFSLETSASTAYLQINNLKNEDTATYFCARDYLYYFDYWGQGTTLTVSSVEGGSGGSGGSGGSGGVDNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 120 - DNA, 750 bp, Artificial Sequence
  Coding sequence for scFv of antibody NKp46-1
tcgactggaagccaggtacagctgcagcagtctggccctgaactcgtcaaaccaggagcttccgtgaagatgtcctgcaaggcttcagggtacacgtttaccgactatgtgatcaattggggtaagcagcgctctgggcaaggcttggagtggattggcgagatctatcctgggagtgggaccaactattacaacgagaagttcaaggccaaagccactttgactgcagacaagagctcaaacattgcctacatgcaactgagctccctgacatcagaggattctgctgtgtacttctgtgcacgtagaggtcggtacggtctgtatgccatggactattggggccaaggcacttccgtgacagtcagctctgtggaaggaggaagtggcggttcaggaggtagcggagggtccggaggagtggatgacattcagatgacacagaccacttctagcctctccgcatcccttggggatagggtcaccatcagttgtagggctagccaggacatttccaattacctgaactggtatcagcagaaacccgatggcacagttaagcttctgatctactacacaagcagactgcactcaggggttccatctcggtttagtggaagtggctctggtaccgactattccctgaccatcaacaatctggaacaggaagatatcgccacctacttctgccaacagggcaatactcgaccctggacatttggtggcggcacgaaactcgagataaaataa

SEQ ID NO: 121 - DNA, 756 bp, Artificial Sequence
  Coding sequence for scFv of antibody NKp46-2
tcgactggatccgaggtacagttgcaggagagtgggcctggactggtcaaaccctcccaatctctgagcttgacatgcacagtcacaggctacagcatcacctccgactacgcttggaattggattcgacagtttcccggcaacaagctggaatggatgggctacatcacctatagtggtagcacttcctataatccctcacttgagagccggatttccatcactagggatacgagcaccaaccagttcttcctgcagctgaatagcgtcaccaccgaagatactgccacctattactgcgcaagaggcggttactatggcagttcatggggtgtattcgcctattggggacaggggacacttgtgacagtgtctgctgttgaaggtggatccggcggatcaggagggagtggtggcagtggaggtgttgacgacattcagatgacccaatcccctgcttctctctcagcctctgtgggagagactgtgaccataacctgtcgtgttagcgagaacatctactcctatctcgcctggtatcagcagaaacaggggaaatccccacaactgctcgtgtacaatgccaagactctggcagaaggagtgccaagccgcttttccgggtctgggtctgggacacagttctcactgaagatcaactctttgcaacctgaggattttggctcttactactgtcagcatcactatggcacaccatggacgtttggtggcggcactaagctggagattaagtaa

SEQ ID NO: 122 - DNA, 738 bp, Artificial Sequence
  Coding sequence for scFv of antibody NKp46-3
tcgactgggtccgaagtgcaactgcaacagtctggccctgagctggtcaaacccggtgcttcagtgaagatctcctgcaagacatccggctacaccttcactgagtacaccatgcactgggtcaaacagtctcacggtaagagcctggagtggataggcggaatttcacccaacattggagggacctcctataaccagaagttcaagggcaaagccacccttacagttgacaagagcagttcaactgcctacatggaactgcgctcattgacctccgaggattcagccgtgtattactgcgctagaaggggaggatccttcgattattggggccaaggcactacgcttaccgtgagcagcgttgaaggtggttctggcggctctggtggaagtggagggagtggcggggtagacgacatcgtgatgactcagagtccagcaactctgtccgttacacctggagatcgagtgtctctgagttgtcgtgcaagccagtctatctctgactatctgcactggtatcagcagaagagccatgagtcacctaggctgttgatcaagtacgcctctcagtccattagcgggattccatcccggtttagtggctctggctccggtagtgacttcacactcagcatcaatagcgtcgaaccagaggatgtaggggtgtactactgtcagaatgggcattcctttcccctcacatttggagctggtaccaaactcgagctgaaataa

SEQ ID NO: 123 - DNA, 741 bp, Artificial Sequence
  Coding sequence for scFv of antibody NKp46-4
tcgactggctcccaagtacagcttcagcagtctgccgtcgaacttgctcgaccaggagcttcagtgaagatgagctgcaaagcctctggttacaccttcacgtcctttaccatgcattgggtgaagcagcgtcctggccaaggcctggagtggattggctacatcaatccctccagcgggtataccgagtacaaccagaagttcaaggacaaaacaaccctgactgccgataagtcaagtagcacagcctatatgcagctggattccctgacatcagacgatagcgctgtgtattactgcgttaggggctctagcagagggttcgactattggggtcaaggcacactggtcacggttagtgccgttgaaggaggctctggaggcagtggaggttctggagggtcaggcggtgtggatgacattcagatgattcagagtcccgctagcttgagtgtaagcgtcggtgagacagtgaccatcacttgtcgcgcatccgaaaacatctactccaatctcgcatggttccagcagaaacagggcaaatcaccccaattgctcgtgtatgccgcaactaatctggctgatggtgtgccttccaggtttagcgggtctggatctgggactcagtactccctgaagatcaactccctccagtctgaggacttcgggatctattactgtcagcacttttggggaactccacggacctttggaggcgggaccaaactggagataaagtaa

SEQ ID NO: 124 - DNA, 696 bp, Artificial Sequence
  Coding sequence for scFv of antibody NKp46-6
ctggtgaggccaggtgcatctgtgaagctgtcatgcaaagcatccgggtacacgttcacctcttcatggatgcattgggccaaacagcgtccaggccagggccttgagtggattggacacattcaccccaatagcggcatatccaactacaacgagaagttcaagggcaaagccacactgacagtggatacttccagctctacagcctatgtggaccttagtagcttgaccagtgaggattctgccgtatactactgtgctagaggtgggcggtttgacgattggggtgctgggaccacagtcaccgtgagcagtgtcgaaggtggatcaggtggatctggaggctcaggcggttctggcggtgttgacgacatcgtgatgactcaaagccctgctactctctctgtcacacccggagatagggtaagcctcagttgtcgagcaagccagtcaatcagcgactatctgcactggtatcagcagaagtcccatgaatccccacgcttgctcatcaagtacgccagtcagtccatcagtggcattccttcccggttttctgggtctggatccgggtcagacttcactctgagcattaactccgtcgaacccgaggatgttggcgtgtattattgccagaatggacattccttcctgatgtacacctttggcggagggaccaaactggagatcaagtaa

SEQ ID NO: 125 - DNA, 750 bp, Artificial Sequence
  Coding sequence for scFv of antibody NKp46-9
tcgactgggtctgatgtgcagttgcaggagtcaggacctgggcttgtcaagccaagccagagcctcagtctcacttgcactgtcacaggctatagcatcacatccgactatgcttggaattggattaggcagtttcctggcaataagctggaatggatggggtacatcacctattccggcagtaccaactacaatcccagcttgaaatctcggatttccataacacgcgatactagcaagaaccagttcttccttcagctgaactctgtgacaacagaggataccgctacgtactattgcgccagatgctgggattacgccctgtatgccatggactgttggggtcaaggtaccagcgttactgtgtctagcgtcgaaggcggaagtggcggctcaggagggtcaggaggctcaggcggagtggatgacattcagatgacccaatctcccgcatccctgtccgcatcagtaggggagacagtgaccattacctgtcgtacttccgagaacatctactcctatcttgcctggtgtcaacagaaacaggggaaaagtccacagctgctggtgtataacgccaagaccttggcagaaggtgttcccagtcgattctctggttccggatccggtacacacttcagcctgaagatcaattctctgcaaccagaggactttggaatctactactgccagcatcactacgacactcctctgacgtttggcgctggtaccaagctcgaactgaaataa

SEQ ID NO: 126 - DNA, 741 bp, Artificial Sequence
  Coding sequence for scFv of antibody BAB281
tcgactggtagccagatacagctggtacagtcaggaccagagctgcagaaacctggagagacagtgaagatcagctgcaaggctagcgggtacaccttcacgaattacgggatgaactgggtcaagcaggctccaggcaaagggctgaagtggatgggctggattaacaccaatactggggaaccaacctatgccgaggaattcaaggggagatttgccttttccctcgaaaccagcgcctcaaccgcctatctccagatcaacaacctgaagaatgaggataccgctacctacttctgtgcaagggactacctctactacttcgactattggggccaaggtacgactcttacagtctctagtgttgagggagggagtggaggttctggaggctctggtggctctggaggcgttgacaacatcgtgatgactcagtctcccaaaagcatgagtatgagtgtgggtgaacgagttaccttgacatgcaaagcctccgagaatgtcgtgacatacgtgtcctggtatcagcagaaacccgagcaatcccctaagctgctgatctatggcgctagcaatcgctatactggcgtacctgatcggttcacaggatcaggctcagccactgactttactcttaccatttcctccgtgcaggcagaagatttggcagattaccactgtgggcaaggttactcttatccctatacatttggaggcggcacaaagctggagattaagtaa

SEQ ID NO: 127 - protein, 9 aa, Artificial Sequence
  Amino acid sequence for synthetic peptide derived from the signal sequence of HLA-G (G peptide)
VMAPRTLFL

SEQ ID NO: 128 - DNA, 27 bp, Artificial Sequence
  Coding sequence of G peptide including most likely codons
gtgatggccccccggaccctgttcctg

SEQ ID NO: 129 - DNA, 27 bp, Artificial Sequence
  Coding sequence of G peptide including consensus codons
  misc_feature (3)..(3): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (18)..(18): n is a, c, g, or t
  misc_feature (21)..(21): n is a, c, g, or t
  misc_feature (27)..(27): n is a, c, g, or t
gtnatggcnccnmgnacnytnttyytn

SEQ ID NO: 130 - protein, 20 aa, Artificial Sequence
  Amino acid sequence for human beta-2- microglobulin (B2M) signal peptide (residues 1-20 of UniProt ID P61769)
MSRSVALAVLALLSLSGLEA

SEQ ID NO: 131 - DNA, 60 bp, Artificial Sequence
  Coding sequence of human B2M signal peptide including most likely codons based upon SEQ ID NO 130
atgagccggagcgtggccctggccgtgctggccctgctgagcctgagcggcctggaggcc

SEQ ID NO: 132 - DNA, 60 bp, Artificial Sequence
  Coding sequence of human B2M signal peptide including consensus codons based upon SEQ ID NO 130
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (18)..(18): n is a, c, g, or t
  misc_feature (21)..(21): n is a, c, g, or t
  misc_feature (24)..(24): n is a, c, g, or t
  misc_feature (27)..(27): n is a, c, g, or t
  misc_feature (30)..(30): n is a, c, g, or t
  misc_feature (33)..(33): n is a, c, g, or t
  misc_feature (36)..(36): n is a, c, g, or t
  misc_feature (39)..(39): n is a, c, g, or t
  misc_feature (42)..(42): n is a, c, g, or t
  misc_feature (45)..(45): n is a, c, g, or t
  misc_feature (48)..(48): n is a, c, g, or t
  misc_feature (51)..(51): n is a, c, g, or t
  misc_feature (54)..(54): n is a, c, g, or t
  misc_feature (60)..(60): n is a, c, g, or t
atgwsnmgnwsngtngcnytngcngtnytngcnytnytnwsnytnwsnggnytngargcn

SEQ ID NO: 133 - protein, 99 aa, Artificial Sequence [sequence omitted]

SEQ ID NO: 134 - DNA, 297 bp, Artificial Sequence
  Coding sequence of mature human B2M without signal sequence including most likely codons based upon SEQ ID NO 133
atccagcggacccccaagatccaggtgtacagccggcaccccgccgagaacggcaagagcaacttcctgaactgctacgtgagcggcttccaccccagcgacatcgaggtggacctgctgaagaacggcgagcggatcgagaaggtggagcacagcgacctgagcttcagcaaggactggagcttctacctgctgtactacaccgagttcacccccaccgagaaggacgagtacgcctgccgggtgaaccacgtgaccctgagccagcccaagatcgtgaagtgggaccgggacatg

SEQ ID NO: 135 - DNA, 297 bp, Artificial Sequence
  Coding sequence of human B2M without signal sequence including consensus codons based upon SEQ ID NO 133
  misc_feature (9)..(9): n is a, c, g, or t
  misc_feature (12)..(12): n is a, c, g, or t
  misc_feature (15)..(15): n is a, c, g, or t
  misc_feature (27)..(27): n is a, c, g, or t
  misc_feature (33)..(33): n is a, c, g, or t
  misc_feature (36)..(36): n is a, c, g, or t
  misc_feature (42)..(42): n is a, c, g, or t
  misc_feature (45)..(45): n is a, c, g, or t
  misc_feature (54)..(54): n is a, c, g, or t
  misc_feature (60)..(60): n is a, c, g, or t
  misc_feature (69)..(69): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (96)..(96): n is a, c, g, or t
  misc_feature (99)..(99): n is a, c, g, or t
  misc_feature (111)..(111): n is a, c, g, or t
  misc_feature (117)..(117): n is a, c, g, or t
  misc_feature (120)..(120): n is a, c, g, or t
  misc_feature (129)..(129): n is a, c, g, or t
  misc_feature (135)..(135): n is a, c, g, or t
  misc_feature (147)..(147): n is a, c, g, or t
  misc_feature (156)..(156): n is a, c, g, or t
  misc_feature (162)..(162): n is a, c, g, or t
  misc_feature (165)..(165): n is a, c, g, or t
  misc_feature (171)..(171): n is a, c, g, or t
  misc_feature (183)..(183): n is a, c, g, or t
  misc_feature (192)..(192): n is a, c, g, or t
  misc_feature (195)..(195): n is a, c, g, or t
  misc_feature (204)..(204): n is a, c, g, or t
  misc_feature (213)..(213): n is a, c, g, or t
  misc_feature (216)..(216): n is a, c, g, or t
  misc_feature (219)..(219): n is a, c, g, or t
  misc_feature (237)..(237): n is a, c, g, or t
  misc_feature (243)..(243): n is a, c, g, or t
  misc_feature (246)..(246): n is a, c, g, or t
  misc_feature (255)..(255): n is a, c, g, or t
  misc_feature (258)..(258): n is a, c, g, or t
  misc_feature (261)..(261): n is a, c, g, or t
  misc_feature (264)..(264): n is a, c, g, or t
  misc_feature (270)..(270): n is a, c, g, or t
  misc_feature (279)..(279): n is a, c, g, or t
  misc_feature (291)..(291): n is a, c, g, or t
athcarmgnacnccnaarathcargtntaywsnmgncayccngcngaraayggnaarwsnaayttyytnaaytgytaygtnwsnggnttycayccnwsngayathgargtngayytnytnaaraayggngarmgnathgaraargtngarcaywsngayytnwsnttywsnaargaytggwsnttytayytnytntaytayacngarttyacnccnacngaraargaygartaygcntgymgngtnaaycaygtnacnytnwsncarccnaarathgtnaartgggaymgngayatg

SEQ ID NO: 136 - protein, 337 aa, Artificial Sequence
  Amino acid sequence of human HLA-E 0103 heavy chain without signal peptide (residues 22-358 of UniProt ID P13747)
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 137 - DNA, 1011 bp, Artificial Sequence
  Coding sequence of human HLA-E 0103 heavy chain without signal peptide (nucleotides 87-1094 of NCBI Ref NM_005516.6)
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 138 - protein, 21 aa, Artificial Sequence
  Amino acid sequence of human CD8 alpha chain transmembrane domain (residues 183-203 of UniProt ID P01732-1)
IYIWAPLAGTCGVLLLSLVIT

SEQ ID NO: 139 - DNA, 63 bp, Artificial Sequence
  Coding sequence of human CD8 alpha chain transmembrane domain (nucleotides 1578 to 1640 of NCBI Ref NM_001145873.1)
atctacatctgggcgcccttggccgggacttgtggggtccttctcctgtcactggttatcacc

SEQ ID NO: 140 - protein, 48 aa, Artificial Sequence
  Amino acid sequence of human CD8 alpha chain hinge (residues 135-182 of UniProt ID P01732-1)
AKPTTTPAPRPPTPAPTIASQPLSLRPEACRPAAGGAVHTRGLDFACD

SEQ ID NO: 141 - DNA, 144 bp, Artificial Sequence
  Coding sequence of human CD8 alpha chain hinge (nucleotides 1434 to 1577 of NCBI Ref NM_001145873.1)
gcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 142 - protein, 15 aa, Artificial Sequence
  glycine-serine linker sequence
GGGGSGGGGSGGGGS

SEQ ID NO: 143 - protein, 20 aa, Artificial Sequence
  Glycine-serine linker sequence
GGGGSGGGGSGGGGSGGGGS

SEQ ID NO: 144 - protein, 10 aa, Artificial Sequence
  Glycine-serine linker sequence
GGGGSGGGGS

SEQ ID NO: 145 - protein, 5 aa, Artificial Sequence
  Glycine-serine linker sequence
GGGGS

SEQ ID NO: 146 - protein, 113 aa, Artificial Sequence
  Amino acid sequence of synthesized cytoplasmic domain of human CD3 zeta chain (residues 52-164 of UniProt ID P20963-1)
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 147 - DNA, 339 bp, Artificial Sequence
  Coding sequence of synthesized cytoplasmic domain of human CD3 zeta chain (nucleotides 299 to 637 of NCBI Ref NM_198053.2)
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 148 - protein, 42 aa, Artificial Sequence
  Amino acid sequence of synthesized cytoplasmic domain of human 4-1BB (residues 214-255 of UniProt ID Q07011)
KRGRKKLLYIFKQPFMRPVQTTQEEDGCSCRFPEEEEGGCEL

SEQ ID NO: 149 - DNA, 126 bp, Artificial Sequence
  Coding sequence of synthesized cytoplasmic domain of human 4-1BB (nucleotides 901 to 1026 of NCBI Ref NM_001561.5)
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 150 - protein, 11 aa, Artificial Sequence
  IgG4 linker sequence
ESKYGPPCPPC

SEQ ID NO: 151 - protein, 11 aa, Artificial Sequence
  CD28 linker sequence
PSPLFPGPSKP

SEQ ID NO: 152 - protein, 6 aa, Artificial Sequence
  His tag sequence
HHHHHH

SEQ ID NO: 153 - protein, 7 aa, Artificial Sequence
  Flag tag sequence
DYKDDDD

SEQ ID NO: 154 - protein, 8 aa, Artificial Sequence
  Flag tag sequence
DYKDDDDK

SEQ ID NO: 155 - protein, 7 aa, Artificial Sequence
  Flag tag sequence
DYKDDDK

SEQ ID NO: 156 - protein, 8 aa, Artificial Sequence
  Xpress tag sequence
DLYDDDDK

SEQ ID NO: 157 - protein, 15 aa, Artificial Sequence
  Avi tag sequence
GLNDIFEAQKIEWHE

SEQ ID NO: 158 - protein, 26 aa, Artificial Sequence
  Calmodulin binding peptide (CBP) tag sequence
KRRWKKNFIAVSAANRFKKISSSGAL

SEQ ID NO: 159 - protein, 6 aa, Artificial Sequence
  Polyglutamate tag sequence
EEEEEE

SEQ ID NO: 160 - protein, 9 aa, Artificial Sequence
  HA tag sequence
YPYDVPDYA

SEQ ID NO: 161 - protein, 9 aa, Artificial Sequence
  HA tag sequence
YAYDVPDYA

SEQ ID NO: 162 - protein, 9 aa, Artificial Sequence
  HA tag sequence
YDVPDYASL

SEQ ID NO: 163 - protein, 10 aa, Artificial Sequence
  Myc tag sequence
EQKLISEEDL

SEQ ID NO: 164 - protein, 8 aa, Artificial Sequence
  Strep tag sequence
WRHPQFGG

SEQ ID NO: 165 - protein, 8 aa, Artificial Sequence
  Strep tag II sequence
WSHPQFEK

SEQ ID NO: 166 - protein, 13 aa, Artificial Sequence
  Softag 1 sequence
SLAELLNAGLGGS

SEQ ID NO: 167 - protein, 8 aa, Artificial Sequence
  Softag 3 sequence
TQDPSRVG

SEQ ID NO: 168 - protein, 14 aa, Artificial Sequence
  V5 tag sequence
GKPIPNPLLGLDST

SEQ ID NO: 169 - protein, 238 aa, Artificial Sequence
  synthesized Blue fluorescent protein (BFP) sequence
MSKGEELFTGVVPILVELDGDVNGHKFSVSGEGEGDATYGKLTLKFICTTGKLPVPWPTLVTTFSHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 170 - protein, 239 aa, Artificial Sequence
  synthesized Enhanced BFP sequence
MVSKGEELFTGVVPILVELDGDVNGHKFSVSGEGEGDATYGKLTLKFICTTGKLPVPWPTLVTTLTHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 171 - protein, 239 aa, Artificial Sequence
  synthesized Enhanced BFP1.2 sequence
MVSKGEELFTGVVPILVELDGDVNGHKFSVRGEGEGDATNGKLTLKFICTTGKLPVPWPTLVTTLSHGVQCFARYPDHMKQHDFFKSAMPEGYVQERTIFFKDDGTYKTRAEVKFEGDTLVNRIELKGIDFKEDGNILGHKLEYNFNSHNVYIMADKQKNGIKVNFKIRHNVEDGSVQLADHYQQNTPIGDGPVLLPDSHYLSTQSVLSKDPNEKRDHMVLLEFVTAAGITLGMDELYK

SEQ ID NO: 172 - protein, 239 aa, Artificial Sequence
  synthesized Enhanced BFP2 sequence
MVSKGEELFTGVVPILVELDGDVNGHKFSVRGEGEGDATNGKLTLKFICTTGKLPVPWPTLVTTLSHGVQCFARYPDHMKQHDFFKSAMPEGYVQERTIFFKDDGTYKTRAEVKFEGDTLVNRIELKGVDFKEDGNILGHKLEYNFNSHNIYIMAVKQKNGIKVNFKIRHNVEDGSVQLADHYQQNTPIGDGPVLLPDSHYLSTQSVLSKDPNEKRDHMVLLEFRTAAGITLGMDELYK

SEQ ID NO: 173 - protein, 239 aa, Artificial Sequence
  synthesized oxBFP sequence
MVSKGEELFTGVVPILVELDGDVNGHKFSVRGEGEGDATNGKLTLKFISTTGKLPVPWPTLVTTLSHGVQVFARYPDHMKQHDFFKSAMPEGYVQERTIFFKDDGTYKTRAEVKFEGDTLVNRIELKGVDFKEDGNILGHKLEYNFNSHNIYIMAVKQKNGIKANFKIRHNVEDGSVQLADHYQQNTPIGDGPVLLPDSHYLSTQSVLSKDPNEKRDHMVLLEFRTAAGITLGMDELYK

SEQ ID NO: 174 - protein, 239 aa, Artificial Sequence
  synthesized moxBFP sequence
MVSKGEELFTGVVPILVELDGDVNGHKFSVRGEGEGDATNGKLTLKFISTTGKLPVPWPTLVTTLSHGVQVFARYPDHMKQHDFFKSAMPEGYVQERTIFFKDDGTYKTRAEVKFEGDTLVNRIELKGVDFKEDGNILGHKLEYNFNSHNIYIMAVKQKNGIKANFKIRHNVEDGSVQLADHYQQNTPIGDGPVLLPDSHYLSTQSKLSKDPNEKRDHMVLLEFRTAAGITLGMDELYK

SEQ ID NO: 175 - DNA, 399 bp, Artificial Sequence
  Nucleotide sequence of myeloproliferative sarcoma virus enhancer, negative control region deleted, dl587rev primer-binding site substituted (MND) promoter
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 176 - protein, 19 aa, Artificial Sequence
  synthesized 2A peptide sequence
LLNFDLLKLAGDVESNPGP

SEQ ID NO: 177 - protein, 19 aa, Artificial Sequence
  synthesized 2A peptide sequence
TLNFDLLKLAGDVESNPGP

SEQ ID NO: 178 - protein, 14 aa, Artificial Sequence
  synthesized 2A peptide sequence
LLKLAGDVESNPGP

SEQ ID NO: 179 - protein, 17 aa, Artificial Sequence
  synthesized 2A peptide sequence
NFDLLKLAGDVESNPGP

SEQ ID NO: 180 - protein, 20 aa, Artificial Sequence
  synthesized 2A peptide sequence
QLLNFDLLKLAGDVESNPGP

SEQ ID NO: 181 - protein, 24 aa, Artificial Sequence
  synthesized 2A peptide sequence
APVKQTLNFDLLKLAGDVESNPGP

SEQ ID NO: 182 - protein, 40 aa, Artificial Sequence
  synthesized 2A peptide sequence
VTELLYRMKRAETYCPRPLLAIHPTEARHKQKIVAPVKQT

SEQ ID NO: 183 - protein, 18 aa, Artificial Sequence
  synthesized 2A peptide sequence
LNFDLLKLAGDVESNPGP

SEQ ID NO: 184 - protein, 40 aa, Artificial Sequence
  synthesized 2A peptide sequence
LLAIHPTEARHKQKIVAPVKQTLNFDLLKLAGDVESNPGP

SEQ ID NO: 185 - protein, 33 aa, Artificial Sequence
  synthesized 2A peptide sequence
EARHKQKIVAPVKQTLNFDLLKLAGDVESNPGP

SEQ ID NO: 186 - protein, 233 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 187 - DNA, 702 bp, Homo sapiens
atggataaccaaggagtaatctactcagacctgaatctgcccccaaacccaaagaggcagcaacgaaaacctaaaggcaataaaagctccattttagcaactgaacaggaaataacctatgcggaattaaaccttcaaaaagcttctcaggattttcaagggaatgacaaaacctatcactgcaaagatttaccatcagctccagagaagctcattgttgggatcctgggaattatctgtcttatcttaatggcctctgtggtaacgatagttgttattccctctacattaatacagaggcacaacaattcttccctgaatacaagaactcagaaagcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctttag

SEQ ID NO: 188 - protein, 41 aa, Artificial Sequence
  Amino acid sequence of synthesized cytoplasmic domain of human CD28 (residues 180-220 of UniProt ID P10747)
RSKRSRLLHSDYMNMTPRRPGPTRKHYQPYAPPRDFAAYRS

SEQ ID NO: 189 - DNA, 123 bp, Artificial Sequence
  Coding sequence of synthesized cytoplasmic domain of human CD28 (nucleotides 223 to 882 of NCBI Ref NM_006139.3)
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 190 - protein, 5 aa, Artificial Sequence
  CDRH1 of anti-NKp46 antibody 8E5B, Kabat numbering
DTYFH

SEQ ID NO: 191 - protein, 7 aa, Artificial Sequence
  CDRH1 of anti-NKp46 antibody 8E5B, Chothia numbering
GFNIKDT

SEQ ID NO: 192 - protein, 8 aa, Artificial Sequence
  CDRH1 of anti-NKp46 antibody 8E5B, IMGT numbering
GFNIKDTY

SEQ ID NO: 193 - protein, 17 aa, Artificial Sequence
  CDRH2 of anti-NKp46 antibody 8E5B, Kabat numbering
RIDPANGNTKYDPKFHD

SEQ ID NO: 194 - protein, 4 aa, Artificial Sequence
  CDRH2 of anti-NKp46 antibody 8E5B, Chothia numbering
PANG

SEQ ID NO: 195 - protein, 8 aa, Artificial Sequence
  CDRH2 of anti-NKp46 antibody 8E5B, IMGT numbering
IDPANGNT

SEQ ID NO: 196 - protein, 5 aa, Artificial Sequence
  CDRH3 of anti-NKp46 antibody 8E5B, Kabat numbering
NRYGY

SEQ ID NO: 197 - protein, 7 aa, Artificial Sequence
  CDRH3 of anti-NKp46 antibody 8E5B, IMGT numbering
AANRYGY

SEQ ID NO: 198 - protein, 16 aa, Artificial Sequence
  CDRL1 of anti-NKp46 antibody 8E5B, Kabat numbering
RSSKSLLYINGNTHLF

SEQ ID NO: 199 - protein, 12 aa, Artificial Sequence
  CDRL1 of anti-NKp46 antibody 8E5B, Chothia numbering
SKSLLYINGNTH

SEQ ID NO: 200 - protein, 11 aa, Artificial Sequence
  CDRL1 of anti-NKp46 antibody 8E5B, IMGT numbering
KSLLYINGNTH

SEQ ID NO: 201 - protein, 7 aa, Artificial Sequence
  CDRL2 of anti-NKp46 antibody 8E5B, Kabat numbering
RMSNLAS

SEQ ID NO: 202 - protein, 9 aa, Artificial Sequence
  CDRL3 of anti-NKp46 antibody 8E5B, Kabat numbering
MQHLEYPFT

SEQ ID NO: 203 - protein, 6 aa, Artificial Sequence
  CDRL3 of anti-NKp46 antibody 8E5B, Chothia numbering
HLEYPF

SEQ ID NO: 204 - protein, 114 aa, Artificial Sequence
  Amino acid sequence of VH domain of 8E5B antibody
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 205 - protein, 112 aa, Artificial Sequence
  Amino acid sequence of VL domain of 8E5B antibody
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 206 - protein, 241 aa, Artificial Sequence
  Amino acid sequence for 8E5B scFv
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIKGGGGSGGGGSGGGGSEIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 207 - DNA, 27 bp, Artificial Sequence
  Coding sequence of G peptide in anti-NKG2A (HLA-E) CAR
gtgatggcccctagaacactgttcctg

SEQ ID NO: 208 - DNA, 60 bp, Artificial Sequence
  Coding sequence of human B2M signal peptide in anti-NKG2A (HLA-E) CAR
atgagccgatccgtggcactggctgtcctggctctgctgtctctgagtggcctggaagca

SEQ ID NO: 209 - DNA, 387 bp, Artificial Sequence
  Coding sequence of mature human B2M without signal sequence in anti-NKG2A (HLA-E) CAR
atccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatg

SEQ ID NO: 210 - DNA, 1011 bp, Artificial Sequence
  Coding sequence of human HLA-E 0103 heavy chain without signal peptide in anti-NKG2A (HLA-E) CAR
ggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 211 - DNA, 45 bp, Artificial Sequence
  Coding sequence of GlySer linker
ggcggaggcggctccggaggaggagggtctggaggcgggggaagt

SEQ ID NO: 212 - DNA, 60 bp, Artificial Sequence
  Coding sequence of GlySer linker
ggcgggggaggctccgggggaggcggctccggcggcgggggaagtggcgggggaggatcc

SEQ ID NO: 213 - protein, 5 aa, Artificial Sequence
  CDRH1 of monalizumab
SYWMN

SEQ ID NO: 214 - protein, 11 aa, Artificial Sequence
  CDRH2 of monalizumab
RIDPYDSETHY

SEQ ID NO: 215 - protein, 16 aa, Artificial Sequence
  CDRH3 of monalizumab
GGYDFDVGTLYWFFDV

SEQ ID NO: 216 - protein, 11 aa, Artificial Sequence
  CDRL1 of monalizumab
RASENIYSYLA

SEQ ID NO: 217 - protein, 7 aa, Artificial Sequence
  CDRL2 of monalizumab
NAKTLAE

SEQ ID NO: 218 - protein, 9 aa, Artificial Sequence
  CDRL3 of monalizumab
QHHYGTPRT

SEQ ID NO: 219 - protein, 124 aa, Artificial Sequence
  Amino acid sequence of VH domain of monalizumab (VH6)
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVS

SEQ ID NO: 220 - protein, 124 aa, Artificial Sequence
  Amino acid sequence of VH domain of monalizumab (VH1)
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVS

SEQ ID NO: 221 - protein, 124 aa, Artificial Sequence
  Amino acid sequence of VH domain of monalizumab (VH5)
EVQLVQSGAEVKKPGESLRISCKGSGYSFTSYWMNWVRQMPGKGLEWMGRIDPYDSETHYSPSFQGHVTISADKSISTAYLQWSSLKASDTAMYYCARGGYDFDVGTLYWFFDVWGQGTTVTVS

SEQ ID NO: 222 - protein, 124 aa, Artificial Sequence
  Amino acid sequence of VH domain of monalizumab (VH7)
EVQLVQSGAEVKKPGATVKISCKVSGYTFTSYWMNWVQQAPGKGLEWMGRIDPYDSETHYAEKFQGRVTITADTSTDTAYMELSSLRSEDTAVYYCATGGYDFDVGTLYWFFDVWGQGTTVTVS

SEQ ID NO: 223 - protein, 124 aa, Artificial Sequence
  Amino acid sequence of VH domain of monalizumab (VH8)
QVQLVQSGAEVKKPGASVKVSCKASGYTFTSYWMNWVRQAPGQGLEWMGRIDPYDSETHYAQKFQGRVTMTRDTSTSTVYMELSSLRSEDTAVYYCARGGYDFDVGTLYWFFDVWGQGTTVTVS

SEQ ID NO: 224 - protein, 452 aa, Artificial Sequence
  Amino acid sequence of VH6 heavy chain of monalizumab
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 225 - protein, 452 aa, Artificial Sequence
  Amino acid sequence of VH1 heavy chain of monalizumab
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 226 - protein, 452 aa, Artificial Sequence
  Amino acid sequence of VH5 heavy chain of monalizumab
EVQLVQSGAEVKKPGESLRISCKGSGYSFTSYWMNWVRQMPGKGLEWMGRIDPYDSETHYSPSFQGHVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 227 - protein, 452 aa, Artificial Sequence
  Amino acid sequence of VH7 heavy chain of monalizumab
EVQLVQSGAEVKKPGATVKISCKVSGYTFTSYWMNWVQQAPGKGLEWMGRIDPYDSETHYAEKFQGRVTITADTSTDTAYMELSSLRSEDTAVYYCATGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 452 aa, Artificial Sequence
  Amino acid sequence of VH8 heavy chain of monalizumab
QVQLVQSGAEVKKPGASVKVSCKASGYTFTSYWMNWVRQAPGQGLEWMGRIDPYDSETHYAQKFQGRVTMTRDTSTSTVYMELSSLRSEDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 229 - protein, 214 aa, Artificial Sequence
  Amino acid sequence of a light chain of monalizumab
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 154 aa, Artificial Sequence
  Amino acid sequence of 4-1BB/CD3z intracellular signaling domain in CAR constructs
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 231 - DNA, 462 bp, Artificial Sequence
  Coding sequence of 4-1BB/CD3z intracellular signaling domain in CAR constructs
aaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 232 - DNA, 144 bp, Artificial Sequence
  Coding sequence of CD8 hinge in CAR constructs
gccaagcctaccaccacccctgcccctagacctccaacacccgccccaacaatcgccagccagcctctgtctctgaggcccgaggcttgtagaccagctgctggcggagccgtgcacaccagaggactggatttcgcctgcgac

SEQ ID NO: 233 - DNA, 63 bp, Artificial Sequence
  Coding sequence of CD8 transmembrane domain in CAR constructs
atctacatctgggcccctctggccggcacatgtggcgtgctgctgctgagcctcgtgatcacc

SEQ ID NO: 234 - protein, 69 aa, Artificial Sequence
  Amino acid sequence of CD8 hinge and CD8 transmembrane domain in CAR constructs
AKPTTTPAPRPPTPAPTIASQPLSLRPEACRPAAGGAVHTRGLDFACDIYIWAPLAGTCGVLLLSLVIT

SEQ ID NO: 235 - DNA, 207 bp, Artificial Sequence
  Coding sequence of CD8 hinge and CD8 transmembrane domain in CAR constructs
gccaagcctaccaccacccctgcccctagacctccaacacccgccccaacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcacc

SEQ ID NO: 236 - protein, 232 aa, Artificial Sequence
  Amino acid sequence of mTagBFP (blue fluorescent protein) in CAR constructs
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 237 - DNA, 699 bp, Artificial Sequence
  Coding sequence of mTagBFP in CAR constructs
agcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattga

SEQ ID NO: 238 - protein, 20 aa, Artificial Sequence
  Amino acid sequence of CD8 signal peptide in CAR constructs
MALPVTALLLPLALLLHAAR

SEQ ID NO: 239 - DNA, 63 bp, Artificial Sequence
  Coding sequence of CD8 signal peptide in CAR constructs
atggctctgcctgtgacagctctgctgctgcctctggccctgctgctccatgccgccagaccc

SEQ ID NO: 240 - protein, 20 aa, Artificial Sequence
  2A peptide sequence in CAR constructs
SGEGRGSLLTCGDVEENPGP

SEQ ID NO: 241 - DNA, 60 bp, Artificial Sequence
  Coding sequence of 2A peptide sequence in CAR constructs
tccggtgagggcagaggaagtcttctaacatgcggtgacgtggaggagaatccgggcccc

SEQ ID NO: 242 - protein, 500 aa, Artificial Sequence
  Amino acid sequence of an HLA-E mimetic
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 243 - DNA, 1590 bp, Artificial Sequence
  Coding sequence of HLA-E mimetic of SEQ ID NO 242
atgagccgatccgtggcactggctgtcctggctctgctgtctctgagtggcctggaagcagtgatggcccctagaacactgttcctgggcggaggcggctccggaggaggagggtctggaggcgggggaagtatccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 244 - protein, 500 aa, Artificial Sequence
  Amino acid sequence of an HLA-E mimetic
MALPVTALLLPLALLLHAARVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 245 - DNA, 1593 bp, Artificial Sequence
  Coding sequence of HLA-E mimetic of SEQ ID NO 244
atggctctgcctgtgacagctctgctgctgcctctggccctgctgctccatgccgccagacccgtgatggcccctagaacactgttcctgggcggaggcggctccggaggaggagggtctggaggcgggggaagtatccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 246 - protein, 520 aa, Artificial Sequence
  Amino acid sequence of an HLA-E mimetic
MALPVTALLLPLALLLHAARMSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 247 - DNA, 1593 bp, Artificial Sequence; identical to SEQ ID NO: 245
  Coding sequence of HLA-E mimetic of SEQ ID NO 246

SEQ ID NO: 248 - DNA, 723 bp, Artificial Sequence
  Coding sequence of anti-NKp46 scFv of SEQ ID NO 206
gatatagtaatgacgcaagcagctccaagcatacccgtcacgccaggggaatctgtctccatatcttgtcggagttctaagtctcttctctacataaatggtaacacgcatctgttctggtttcttcaaaggcctgggcagtctcctcaactgctcatctatcgcatgagcaacttggcttcaggcgttcctgatcgattttctggttcaggctcagggaccgcatttacgttgagaatcagccgggtggaggccgaggatgttggcgtatattactgtatgcaacacctcgagtatcctttcacttttgggtctggaactaagctggagattaagggcggtggtgggtcaggcggcggtggctctggaggtggtgggtccgagattcagttgcaacaaagtggtgctgagttggtaaaaccaggagcaagtgtaaagctgtcatgtaccgcttccggcttcaacattaaagatacttacttccactgggtaaaacaacgccccgaacagggccttgagtggatcggtagaattgacccagccaacggaaacaccaagtatgatccaaagtttcacgacaaagcaacaatcatagctgacataagcagcaatacggcatatctccaattctcttccctcacgagcgaagatacggcggtgtactattgtgctgcaaatcgctatggctattggggacagggcacgaccctgactgtttcttct

SEQ ID NO: 249 - protein, 745 aa, Artificial Sequence
  Amino acid sequence of an anti-NKp46 CAR
MALPVTALLLPLALLLHAARDIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIKGGGGSGGGGSGGGGSEIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSSASAKPTTTPAPRPPTPAPTIASQPLSLRPEACRPAAGGAVHTRGLDFACDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGRSGEGRGSLLTCGDVEENPGPSRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 250 - DNA, 2241 bp, Artificial Sequence
  Coding sequence of anti-NKp46 CAR of SEQ ID NO 249
atggctctgcctgtgacagctctgctgctgcctctggccctgctgctccatgccgccagacccgatatagtaatgacgcaagcagctccaagcatacccgtcacgccaggggaatctgtctccatatcttgtcggagttctaagtctcttctctacataaatggtaacacgcatctgttctggtttcttcaaaggcctgggcagtctcctcaactgctcatctatcgcatgagcaacttggcttcaggcgttcctgatcgattttctggttcaggctcagggaccgcatttacgttgagaatcagccgggtggaggccgaggatgttggcgtatattactgtatgcaacacctcgagtatcctttcacttttgggtctggaactaagctggagattaagggcggtggtgggtcaggcggcggtggctctggaggtggtgggtccgagattcagttgcaacaaagtggtgctgagttggtaaaaccaggagcaagtgtaaagctgtcatgtaccgcttccggcttcaacattaaagatacttacttccactgggtaaaacaacgccccgaacagggccttgagtggatcggtagaattgacccagccaacggaaacaccaagtatgatccaaagtttcacgacaaagcaacaatcatagctgacataagcagcaatacggcatatctccaattctcttccctcacgagcgaagatacggcggtgtactattgtgctgcaaatcgctatggctattggggacagggcacgaccctgactgtttcttctgctagcgccaagcctaccaccacccctgcccctagacctccaacacccgccccaacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccatgcataaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcggccgctccggtgagggcagaggaagtcttctaacatgcggtgacgtggaggagaatccgggcccctctagaagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattga

SEQ ID NO: 251 - protein, 913 aa, Artificial Sequence
  Amino acid sequence of an anti-NKG2A (HLA-E) CAR
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLTSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGRSGEGRGSLLTCGDVEENPGPSRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 252 - DNA, 2832 bp, Artificial Sequence
  Coding sequence of anti-NKG2A (HLA-E) CAR of SEQ ID NO 251
atgagccgatccgtggcactggctgtcctggctctgctgtctctgagtggcctggaagcagtgatggcccctagaacactgttcctgggcggaggcggctccggaggaggagggtctggaggcgggggaagtatccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgactagtaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcggccgctccggtgagggcagaggaagtcttctaacatgcggtgacgtggaggagaatccgggcccctctagaagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattga

SEQ ID NO: 253 - protein, 984 aa, Artificial Sequence
  Amino acid sequence of an anti-NKG2A (HLA-E) CAR
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLTSAKPTTTPAPRPPTPAPTIASQPLSLRPEACRPAAGGAVHTRGLDFACDIYIWAPLAGTCGVLLLSLVITTSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGRSGEGRGSLLTCGDVEENPGPSRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 254 - DNA, 3045 bp, Artificial Sequence
  Coding sequence of anti-NKG2A (HLA-E) CAR of SEQ ID NO 253
atgagccgatccgtggcactggctgtcctggctctgctgtctctgagtggcctggaagcagtgatggcccctagaacactgttcctgggcggaggcggctccggaggaggagggtctggaggcgggggaagtatccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgactagtgccaagcctaccaccacccctgcccctagacctccaacacccgccccaacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccactagtaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcggccgctccggtgagggcagaggaagtcttctaacatgcggtgacgtggaggagaatccgggcccctctagaagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattga

SEQ ID NO: 255 - protein, 913 aa, Artificial Sequence
  Amino acid sequence of an anti-NKG2A (HLA-E) CAR
MALPVTALLLPLALLLHAARVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLTSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGRSGEGRGSLLTCGDVEENPGPSRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 256 - DNA, 2835 bp, Artificial Sequence
  Coding sequence of anti-NKG2A (HLA-E) CAR of SEQ ID NO 255
atggctctgcctgtgacagctctgctgctgcctctggccctgctgctccatgccgccagacccgtgatggcccctagaacactgttcctgggcggaggcggctccggaggaggagggtctggaggcgggggaagtatccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgactagtaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcggccgctccggtgagggcagaggaagtcttctaacatgcggtgacgtggaggagaatccgggcccctctagaagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattga

SEQ ID NO: 257 - protein, 1004 aa, Artificial Sequence
  Amino acid sequence of an anti-NKG2A (HLA-E) CAR
MALPVTALLLPLALLLHAARMSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSLTSAKPTTTPAPRPPTPAPTIASQPLSLRPEACRPAAGGAVHTRGLDFACDIYIWAPLAGTCGVLLLSLVITTSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGRSGEGRGSLLTCGDVEENPGPSRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 258 - DNA, 3048 bp, Artificial Sequence
  Coding sequence of anti-NKG2A (HLA-E) CAR of SEQ ID NO 257
atggctctgcctgtgacagctctgctgctgcctctggccctgctgctccatgccgccagacccgtgatggcccctagaacactgttcctgggcggaggcggctccggaggaggagggtctggaggcgggggaagtatccagcggactcccaaggtgatggcccctagaacactgttcctgggcggaggcggctccggaggaggagggtctggaggcgggggaagtatccagcggactcccaagattcaggtctacagcagacaccctgccgaaaacgggaaatccaacttcctgaattgctatgtgtcaggctttcatcccagcgacatcgaggtcgatctgctgaagaatggcgagcggattgaaaaagtggagcactctgacctgtcattcagcaaggattggagcttttacctgctgtactatactgagtttaccccaacagaaaaagacgagtatgcctgtagggtgaaccatgtcaccctgagtcagcccaagatcgtgaaatgggaccgcgatatgggcgggggaggctccgggggaggcggctccggcggcgggggaagtggcgggggaggatccggttctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgactagtgccaagcctaccaccacccctgcccctagacctccaacacccgccccaacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccactagtaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcggccgctccggtgagggcagaggaagtcttctaacatgcggtgacgtggaggagaatccgggcccctctagaagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattga

SEQ ID NO: 259 - protein, 24 aa, Artificial Sequence
  Amino acid sequence of transmembrane domain of an HLA-E alpha chain found in mature B2M set forth in SEQ ID NO 136
VGIIAGLVLLGSVVSGAVVAAVIW

SEQ ID NO: 260 - protein, 7 aa, Artificial Sequence
  CDRH1 of anti-NKp46 antibody
TYGIGVG

SEQ ID NO: 261 - protein, 16 aa, Artificial Sequence
  CDRH2 of anti-NKp46 antibody
HIWWNDNEYYNIDLKS

SEQ ID NO: 262 - protein, 13 aa, Artificial Sequence
  CDRH3 of anti-NKp46 antibody
GNYRYARGYVMDY

SEQ ID NO: 263 - protein, 15 aa, Artificial Sequence
  CDRL1 of anti-NKp46 antibody
RASESVEYYGTSLMQ

SEQ ID NO: 264 - protein, 7 aa, Artificial Sequence
  CDRL2 of anti-NKp46 antibody
AASNVES

SEQ ID NO: 265 - protein, 9 aa, Artificial Sequence
  CDRL3 of anti-NKp46 antibody
QQNRKVPWT

SEQ ID NO: 266 - protein, 466 aa, Artificial Sequence
  Amino acid sequence of heavy chain of anti- NKp46 from US10716864
MGRLTSSFLLLIVPAYVLSQVTLKESGPGILQPSQTLSLTCSFSGFSLSTYGIGVGWNRQPSGKGLEWLAHIWWNDNEYYNIDLKSRLTISKDTSNNQVFLKIASVDTADTATYYCVRGNYRYARGYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 267 - protein, 238 aa, Artificial Sequence
  Amino acid sequence of light chain of anti- NKp46 from US10716864
MESDTLLLWVLLLWVPGSTGDIVLTQSPASLAVSLGQRATISCRASESVEYYGTSLMQWYQQKPGQPPKLLIYAASNVESGVPARFSGSGSGTDFSLNIHPVEEDDFAMYFCQQNRKVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 268 - protein, 201 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 269 - protein, 276 aa, Homo sapiens
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 270 - protein, 410 aa, Homo sapiens
MSLMVVSMACVGFFLLEGPWPHVGGQDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 271 - protein, 377 aa, Homo sapiens
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHAVIRYSVAIILFTILPFFLLHRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 272 - protein, 341 aa, Homo sapiens
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

SEQ ID NO: 273 - protein, 455 aa, Homo sapiens
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 274 - protein, 348 aa, Homo sapiens [sequence omitted]

SEQ ID NO: 275 - protein, 444 aa, Homo sapiens
MSLMVVSMACVGLFLVQRAGPHMGGQDKPFLSAWPSAVVPRGGHVTLRCHYRHRFNNFMLYKEDRIHIPIFHGRIFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTHTPYQLSAPSDPLDIVVTGPYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 276 - protein, 9 aa, Artificial Sequence
  amino acid consensus sequence of a synthesized peptide from the signal sequence of a classical MHC class I molecule
VMAPRTLLL

SEQ ID NO: 277 - protein, 9 aa, Artificial Sequence
  synthesized peptide from the signal sequence of a classical MHC class I molecule
VMAPRTLVL

SEQ ID NO: 278 - protein, 9 aa, Artificial Sequence
  synthesized peptide from the signal sequence of a classical MHC class I molecule
VMAPRTVLL

SEQ ID NO: 279 - protein, 9 aa, Artificial Sequence
  synthesized peptide from the signal sequence of a classical MHC class I molecule
VMAPRTLIL

SEQ ID NO: 280 - protein, 9 aa, Artificial Sequence
  synthesized peptide from the signal sequence of a classical MHC class I molecule
VMAPRTVFL

SEQ ID NO: 281 - protein, 9 aa, Artificial Sequence
  synthesized peptide from the signal sequence of a classical MHC class I molecule
VMPPRTLLL

SEQ ID NO: 282 - protein, 9 aa, Artificial Sequence
  synthesized peptide from the signal sequence of a classical MHC class I molecule
VTAPRTVLL

SEQ ID NO: 283 - protein, 9 aa, Artificial Sequence
  synthesized peptide from the signal sequence of a classical MHC class I molecule
VTAPRTLLL

SEQ ID NO: 284 - protein, 9 aa, Artificial Sequence
  synthesized peptide from the signal sequence of a classical MHC class I molecule
VMAPRALLL

SEQ ID NO: 285 - protein, 9 aa, Artificial Sequence
  synthesized peptide from the signal sequence of a classical MHC class I molecule
VMAPQALLL

SEQ ID NO: 286 - protein, 9 aa, Artificial Sequence
  a peptide of multidrug resistance-associated protein 7
ALALVRMLI

SEQ ID NO: 287 - protein, 9 aa, Artificial Sequence
  a leader peptide of HSP60
QMRPVSRVL

SEQ ID NO: 288 - protein, 469 aa, Artificial Sequence
  heavy chain of anti-KIR antibody (US9879082)
MDWTWRFLFVVAASTGVQSQVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 289 - protein, 109 aa, Artificial Sequence
  light chain variable domain of anti-KIR antibody (US9879082)
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT